(19) United States
(12) Patent Application Publication
(10) Pub. No.: US 2026/0226170 A1
(43) Pub. Date: Aug. 6, 2026

(54) MULTISPECIFIC POLYPEPTIDES AND METHODS OF USE THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US); John LAUDENSLAGER, La Mesa, CA (US)

(21) Appl. No.: 19/652,533

(22) Filed: Apr. 20, 2026

Related U.S. Application Data
(63) Continuation of application No. PCT/US2026/014042, filed on Feb. 5, 2026.
(60) Provisional application No. 63/754,282, filed on Feb. 5, 2025.
(60) Provisional application No. 63/754,238, filed on Feb. 5, 2025.
(60) Provisional application No. 63/781,651, filed on Apr. 1, 2025.
(60) Provisional application No. 63/849,701, filed on Jul. 23, 2025.

(51) Int. Cl. C07K 16/28

(52) U.S. Cl. CPC C07K 16/2809; C07K 16/2803; C07K 2317/31; C07K 2317/622

(57) ABSTRACT

Provided herein are designs of multi-specific polypeptides for binding target cells and engaging T cells. In some embodiments, the multi-specific polypeptide comprises a first binding domain that specifically binds to a T cell receptor beta variable region (TCRβV), or a T cell receptor alpha variable region (TCRαV) expressed on the T cell, and a second binding domain that specifically binds to an antigen (e.g., CD19) on the target cell, and a Fc region. In some embodiments, both the first and the second binding domains are located on the same polypeptide chain of the Fc region (e.g., a Fc region polypeptide chain comprising a Knob mutation). These multi-specific polypeptides are not only effective in killing the target cells, but also expands early memory populations that maintains and/or replenishes effector populations over time.

### CROSS REFERENCE TO RELATED APPLICATIONS

This application is a continuation of PCT Application No. PCT/US2026/014042, filed Feb. 5, 2026, which claims the benefit of and the priority from U.S. Provisional Application No. 63/754,282, filed Feb. 5, 2025; U.S. Provisional Application No. 63/754,238, filed Feb. 5, 2025; U.S. Provisional Application No. 63/781,651, filed Apr. 1, 2025; and U.S. Provisional Application No. 63/849,701, filed Jul. 23, 2025, the disclosure of each of which is incorporated by reference in its entirety.

### FIELD

The present disclosure relates to designs of multi-specific polypeptides for binding CD19 and engaging T cells. The present disclosure also relates to methods of expanding and activating T cells and methods of treating a disease or disorder (e.g., a cancer or an autoimmune disease) using the same.

### REFERENCE TO AN ELECTRONIC SEQUENCE LISTING

The contents of the electronic sequence listing (JANX_004_05US_SeqList_ST26.xml; Size: 1,224,007 bytes; and Date of Creation: Apr. 1, 2026) are herein incorporated by reference in their entirety.

### BACKGROUND

T cell engagers are a class of immunotherapeutic molecules that redirect the patient's immune response to recognize and kill target cells (e.g., B cells expressing CD19). While T cell engagers have shown to elicit potent immune activity, these therapeutics have faced challenges due to overactivation of the immune system leading to cytokine release syndrome (CRS), on-target, healthy tissue toxicities, T cell exhaustion, as well as depletion of effector T cell populations with minimal expansion of memory T cell populations that can limit duration of immune activity. Thus, there is a need in the art to improve the design of T cell engagers to improve the duration of immune activity as well as to reduce the toxicity and side effects associated with T cell engagers.

### SUMMARY

Provided herein is a multi-specific polypeptide comprising: a first binding domain that specifically binds T cell receptor alpha variable region (TCRαV), or T cell receptor beta variable region (TCRβV); and a second binding domain that specifically binds CD19. Also provided herein is a multi-specific polypeptide comprising: a first binding domain that specifically binds CD3; and a second binding domain that specifically binds CD19.

In some embodiments, the multi-specific polypeptide further comprises a half-life extension moiety.

In some embodiments, the CD19 comprises an amino acid sequence that is at least 80%, at least 85%, at least 90%, at least 95%, at least 97%, at least 98%, at least 99%, or 100% identical to SEQ ID NO: 151.

In some embodiments, the second binding domain comprises: an immunoglobulin heavy chain variable (VH) region comprising a heavy chain CDR1 (HCDR1), a heavy chain CDR2 (HCDR2), and a heavy chain CDR3 (HCDR3), and an immunoglobulin light chain variable (VL) region comprising a light chain CDR1 (LCDR1), a light chain CDR2 (LCDR2), and a light chain CDR3 (LCDR3).

In some embodiments, the second binding domain is a single chain variable fragment (scFv), Fab, CrossFab, Fab′, or F(ab′)2.

In some embodiments, the second binding domain is a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region. In some embodiments, the second binding domain is a CrossFab comprising the VH region, the VL region, a first CH1 region, and a CL region, wherein the C-terminus of the VH region is connected to the N-terminus of the CL region, and the C-terminus of the VL region is connected to the N-terminus of the CH1 region. In some embodiments, the CH1 region and the CL region are connected by a disulfide bond. In some embodiments, the second binding domain is a charge steering Fab or charge steering CrossFab. In some embodiments, a CH1 region of the charge steering Fab or charge steering CrossFab comprises mutations K147E/K213E according to EU numbering, and a CL region of the charge steering Fab or charge steering CrossFab comprises mutations E123R/Q124K according to EU numbering.

In some embodiments, the second binding domain is an scFv comprising a VH region and a VL region. In some embodiments, the C-terminus of the VH region is linked to the N-terminus of the VL region. In some embodiments, the C-terminus of the VL region is linked to the N-terminus of the VH region. In some embodiments, the VH region and the VL region are linked by a peptide linker (PL2). In some embodiments, the peptide linker (PL2) comprises the formula of (Gly₄Ser)ₙ, wherein n=1-5. In some embodiments, the peptide linker (PL2) is (Gly₄Ser)₃. In some embodiments, the peptide linker (PL2) is (Gly₄Ser)₁.

In some embodiments, the HCDR1, the HCDR2, the HCDR3, the LCDR1, the LCDR2, and the LCDR3 of the second binding domain are according to one of the combinations of complementarity determining regions (CDRs) of Table 14 or Table 15. In some embodiments, the HCDR 1 comprises an amino acid sequence of SEQ ID NO: 303, the HCDR2 comprises an amino acid sequence of SEQ ID NO: 311, the HCDR3 comprises an amino acid sequence of SEQ ID NO: 317, the LCDR1 comprises an amino acid sequence of SEQ ID NO: 323, the LCDR2 comprises an amino acid sequence of SEQ ID NO: 329, and the LCDR3 comprises an amino acid sequence of SEQ ID NO: 334, wherein the CDRs correspond to the second binding domain and are numbered with reference to the Kabat numbering scheme. In some embodiments, the HCDR1 comprises an amino acid sequence of SEQ ID NO: 304, the HCDR2 comprises an amino acid sequence of SEQ ID NO: 312, the HCDR3 comprises an amino acid sequence of SEQ ID NO: 318, the LCDR1 comprises an amino acid sequence of SEQ ID NO: 324, the LCDR2 comprises an amino acid sequence of SEQ ID NO: 328, and the LCDR3 comprises an amino acid sequence of SEQ ID NO: 335, wherein the CDRs correspond to the second binding domain and are numbered with reference to the Kabat numbering scheme. In some embodiments, the HCDR1 comprises an amino acid sequence of SEQ ID NO: 306, the HCDR2 comprises an amino acid sequence of SEQ ID NO: 314, the HCDR3 comprises an amino acid sequence of SEQ ID NO: 318, the LCDR1 comprises an amino acid sequence of SEQ ID NO: 326, the LCDR2 comprises an amino acid sequence of SEQ ID NO: 328, and the LCDR3 comprises an amino acid sequence of SEQ ID NO: 335, wherein the CDRs correspond to the second binding domain and are numbered with reference to the Kabat numbering scheme. In some embodiments, the HCDR1 comprises an amino acid sequence of SEQ ID NO: 306, the HCDR2 comprises an amino acid sequence of SEQ ID NO: 314, the HCDR3 comprises an amino acid sequence of SEQ ID NO: 320, the LCDR1 comprises an amino acid sequence of SEQ ID NO: 326, the LCDR2 comprises an amino acid sequence of SEQ ID NO: 331, and the LCDR3 comprises an amino acid sequence of SEQ ID NO: 335, wherein the CDRs correspond to the second binding domain and are numbered with reference to the Kabat numbering scheme.

In some embodiments, the VH region of the second binding domain comprises an amino acid sequence that is at least 80%, at least 85%, at least 90%, at least 95%, at least 97%, at least 98%, at least 99%, or 100% identical to any one of SEQ ID NOs: 365, 367, 369, 371, 373, 375, 377, 379, 381, and 383, and wherein the VL region of the second binding domain comprises an amino acid sequence that is at least 80%, at least 85%, at least 90%, at least 95%, at least 97%, at least 98%, at least 99%, or 100% identical to SEQ ID NO: 366, 368, 370, 372, 374, 376, 378, 380, 382, and 384.

In some embodiments, the second binding domain comprises an scFv comprising an amino acid sequence that is at least 80%, at least 85%, at least 90%, at least 95%, at least 97%, at least 98%, at least 99%, or 100% identical to any one of SEQ ID NOs: 385-386.

In some embodiments, the second binding domain comprises a Fab or CrossFab comprising (i) a heavy chain comprising an amino acid sequence that is at least 80%, at least 85%, at least 90%, at least 95%, at least 97%, at least 98%, at least 99%, or 100% identical to any one of SEQ ID NOs: 387, 389, 391, 393, 395, 397, 399, 401, 403, and 405; and (ii) a light chain comprising an amino acid sequence that is at least 80%, at least 85%, at least 90%, at least 95%, at least 97%, at least 98%, at least 99%, or 100% identical to any one of SEQ ID NOs: 388, 390, 392, 394, 396, 398, 400, 402, 404, and 406.

In some embodiments, the first binding domain specifically binds TCRβV. In some embodiments, the TCRβV is selected from the group consisting of: TCRβV-5-5, TCRβV-5-6, TCRβV6-5, TCRβV-12-3, TCRβV-12-4, and TCRβV-20-1. In some embodiments, the TCRβV comprises an amino acid sequence that is at least 80%, at least 85%, at least 90%, at least 95%, at least 97%, at least 98%, at least 99%, or 100% identical to any one of SEQ ID NOs: 100-116. In some embodiments, the first binding domain comprises complementarity determining regions (CDRs) from a binding molecule selected from the group consisting of: IMMU 546, FIN9, AMKB1-2, ZOE, 3G5, ZIZOU4, IMMU 157, LC4, 3D11, 1C1, W112, MH3-2, 4H11, 36213, BAM13, H131, A.H-53, H132, IMMU 222, JU-74, JU74.3, OT145, BL37.2, S511, VER2.32.1, IG125, 56C5, 56C5.2, 16G8, MX-6, JR2, AF-23, AF23, AHUT7, TAMAYA1.2, BA62, BA62.6, C1, E17.5F3, E17.5F3.15.13, MPB2D5, C21, CAS1.1.3, CH92, 8F10, JOVI-3, WJF24, ELL1.4, JOVI-1, SP305, LG.3A10, 5A8A10, 5D9F1, and KFN, wherein the CDRs from a binding molecule are each determined according to the Kabat numbering scheme, the Chothia numbering scheme, or the ImMunoGeneTics Information System (IMGT).

In some embodiments, the first binding domain specifically binds TCRαV. In some embodiments, the TCRαV is TCRαV 1-2. In some embodiments, the TCRαV comprises an amino acid sequence that is at least 80%, at least 85%, at least 90%, at least 95%, at least 97%, at least 98%, at least 99%, or 100% identical to SEQ ID NO: 149. In some embodiments, the first binding domain comprises CDRs from a binding molecule selected from the group consisting of: C15, 6B11, SFB280, 3C10, F1, 6D6.6, and clone 34, wherein the CDRs are each determined according to the Kabat numbering scheme, the Chothia numbering scheme, or the ImMunoGeneTics Information System (IMGT).

In some embodiments, the first binding domain comprises: a VH region comprising an HCDR1, an HCDR2, and an HCDR3, and a VL region comprising an LCDR1, an LCDR2, and an LCDR3.

In some embodiments, the first binding domain is a single chain variable fragment (scFv), Fab, CrossFab, Fab′, or F(ab′)₂.

In some embodiments, the first binding domain is a Fab comprising the VH region, the VL region, a CH1 region, and a CL region, wherein the C-terminus of the VH region is connected to the N-terminus of the CH1 region, and the C-terminus of the VL region is connected to the N-terminus of the CL region. In some embodiments, the first binding domain is a CrossFab comprising the VH region, the VL region, a CH1 region, and a CL region, wherein the C-terminus of the VH region is connected to the N-terminus of the CL region, and the C-terminus of the VL region is connected to the N-terminus of the CH1 region. In some embodiments, the CH1 region and the CL region are connected by a disulfide bond. In some embodiments, the first binding domain is a charge steering Fab or charge steering CrossFab. In some embodiments, a CH1 region of the charge steering Fab or charge steering CrossFab comprises mutations K147E/K213E according to EU numbering, and a CL region of the charge steering Fab or charge steering CrossFab comprises mutations E123R/Q124K according to EU numbering.

In some embodiments, the first binding domain is an scFv. In some embodiments, the C-terminus of the VH region is linked to the N-terminus of the VL region. In some embodiments, the C-terminus of the VL region is linked to the N-terminus of the VH region.

In some embodiments, the VH region and the VL region is linked by one or more engineered disulfide bond(s). In some embodiments, one cysteine that forms the engineered disulfide bond is located in the VH region, and wherein the other cysteine that forms the engineered disulfide bond is located in the VL region. In some embodiments, the positions of the one or more engineered disulfide bond(s) are selected from the group consisting of VH44-VL100, VH100B-VL49, VH100-VL50, VH101-VL46, and VH105-VL43. In some embodiments, the VH region and the VL region are linked by a peptide linker (PL1). In some embodiments, the peptide linker (PL1) comprises the formula of (Gly₄Ser)ₙ, wherein n=1-5. In some embodiments, the peptide linker (PL1) is (Gly₄Ser)₃. In some embodiments, the peptide linker (PL1) is (Gly₄Ser)₁.

In some embodiments, the HCDR1, the HCDR2, the HCDR3, the LCDR1, the LCDR2, and the LCDR3 of the first binding domain are according to one of the combinations of complementarity determining regions (CDRs) of Table 1, Table 2, Table 6, or Table 7. In some embodiments, the HCDR1 comprises an amino acid sequence of SEQ ID NO: 1138, the HCDR2 comprises an amino acid sequence of SEQ ID NO: 2, the HCDR3 comprises an amino acid sequence of SEQ ID NO: 1139, the LCDR1 comprises an amino acid sequence of SEQ ID NO: 1140, the LCDR2 comprises an amino acid sequence of SEQ ID NO: 5, and the LCDR3 comprises an amino acid sequence of SEQ ID NO: 1141, wherein the CDRs correspond to the first binding domain and are numbered with reference to the Kabat numbering scheme. In some embodiments, the HCDR1 comprises an amino acid sequence of SEQ ID NO: 1, the HCDR2 comprises an amino acid sequence of SEQ ID NO: 1142, the HCDR3 comprises an amino acid sequence of SEQ ID NO: 1139, the LCDR1 comprises an amino acid sequence of SEQ ID NO: 1140, the LCDR2 comprises an amino acid sequence of SEQ ID NO: 5, and the LCDR3 comprises an amino acid sequence of SEQ ID NO: 1141, wherein the CDRs correspond to the first binding domain and are numbered with reference to the Kabat numbering scheme. In some embodiments, the HCDR1 comprises an amino acid sequence of SEQ ID NO: 1143, the HCDR2 comprises an amino acid sequence of SEQ ID NO: 18, the HCDR3 comprises an amino acid sequence of SEQ ID NO: 1144, the LCDR1 comprises an amino acid sequence of SEQ ID NO: 1145, the LCDR2 comprises an amino acid sequence of SSS, and the LCDR3 comprises an amino acid sequence of SEQ ID NO: 1146, wherein the CDRs correspond to the first binding domain and are numbered with reference to the IMGT numbering scheme. In some embodiments, the HCDR1 comprises an amino acid sequence of SEQ ID NO: 17, the HCDR2 comprises an amino acid sequence of SEQ ID NO: 1147, the HCDR3 comprises an amino acid sequence of SEQ ID NO: 1144, the LCDR1 comprises an amino acid sequence of SEQ ID NO: 1145, the LCDR2 comprises an amino acid sequence of SSS, and the LCDR3 comprises an amino acid sequence of SEQ ID NO: 1146, wherein the CDRs correspond to the first binding domain and are numbered with reference to the IMGT numbering scheme. In some embodiments, the VH region of the first binding domain comprises an amino acid sequence that is at least 80%, at least 85%, at least 90%, at least 95%, at least 97%, at least 98%, at least 99%, or 100% identical to one of SEQ ID NOs listed in the VH column of Table 3 or Table 8, and wherein the VL region of the first binding domain comprises an amino acid sequence that is at least 80%, at least 85%, at least 90%, at least 95%, at least 97%, at least 98%, at least 99%, or 100% identical to the corresponding VL SEQ ID NO listed in the same row of Table 3 or Table 8. In some embodiments, the VH region of the first binding domain comprises an amino acid sequence that is at least 80%, at least 85%, at least 90%, at least 95%, at least 97%, at least 98%, at least 99%, or 100% identical to SEQ ID NO: 1148, and wherein the VL region of the first binding domain comprises an amino acid sequence that is at least 80%, at least 85%, at least 90%, at least 95%, at least 97%, at least 98%, at least 99%, or 100% identical to SEQ ID NO: 1149. In some embodiments, the VH region of the first binding domain comprises an amino acid sequence that is at least 80%, at least 85%, at least 90%, at least 95%, at least 97%, at least 98%, at least 99%, or 100% identical to SEQ ID NO: 1150, and wherein the VL region of the first binding domain comprises an amino acid sequence that is at least 80%, at least 85%, at least 90%, at least 95%, at least 97%, at least 98%, at least 99%, or 100% identical to SEQ ID NO: 1149.

In some embodiments, the HCDR1 comprises an amino acid sequence of SEQ ID NO: 201, the HCDR2 comprises an amino acid sequence of SEQ ID NO: 202, the HCDR3 comprises an amino acid sequence of SEQ ID NO: 203, the LCDR1 comprises an amino acid sequence of SEQ ID NO: 204, the LCDR2 comprises an amino acid sequence of SEQ ID NO: 205, and the LCDR3 comprises an amino acid sequence of SEQ ID NO: 206, wherein the CDRs correspond to the first binding domain and are numbered with reference to the Kabat numbering scheme. In some embodiments, the VH region of the first binding domain comprises an amino acid sequence that is at least 80%, at least 85%, at least 90%, at least 95%, at least 97%, at least 98%, at least 99%, or 100% identical to one of SEQ ID NO: 213, and wherein the VL region of the first binding domain comprises an amino acid sequence that is at least 80%, at least 85%, at least 90%, at least 95%, at least 97%, at least 98%, at least 99%, or 100% identical to the corresponding VL SEQ ID NO: 214.

In some embodiments, the first binding domain comprises an scFv comprising an amino acid sequence that is at least 80%, at least 85%, at least 90%, at least 95%, at least 97%, at least 98%, at least 99%, or 100% identical to any one of SEQ ID NOs in Table 4 or Table 9. In some embodiments, the first binding domain comprises an scFv comprising an amino acid sequence that is at least 80%, at least 85%, at least 90%, at least 95%, at least 97%, at least 98%, at least 99%, or 100% identical to SEQ ID NO: 1151. In some embodiments, the first binding domain comprises an scFv comprising an amino acid sequence that is at least 80%, at least 85%, at least 90%, at least 95%, at least 97%, at least 98%, at least 99%, or 100% identical to SEQ ID NO: 1152. In some embodiments, the first binding domain comprises an scFv comprising an amino acid sequence that is at least 80%, at least 85%, at least 90%, at least 95%, at least 97%, at least 98%, at least 99%, or 100% identical to SEQ ID NO: 1153. In some embodiments, the first binding domain comprises an scFv comprising an amino acid sequence that is at least 80%, at least 85%, at least 90%, at least 95%, at least 97%, at least 98%, at least 99%, or 100% identical to SEQ ID NO: 1154.

In some embodiments, the first binding domain comprises a Fab or CrossFab comprising (i) a heavy chain comprising an amino acid sequence that is at least 80%, at least 85%, at least 90%, at least 95%, at least 97%, at least 98%, at least 99%, or 100% identical to any one of SEQ ID NOs: 243, 245, 1071-1086, 1112-1123, 1129, 1130, 1155, and 1157; and (ii) a light chain comprising an amino acid sequence that is at least 80%, at least 85%, at least 90%, at least 95%, at least 97%, at least 98%, at least 99%, or 100% identical to any one of SEQ ID NOs: 244, 246, 1055-1070, 1100-1111, 1127, 1128, 1156, and 1158.

In some embodiments, the half-life extension moiety is selected from the group consisting of a fragment crystallizable (Fc) region (e.g., a single chain Fc), an albumin (e.g., human serum albumin), transferrin, a binding domain that specifically binds to transferrin or albumin (e.g., an albumin binding domain (ABD)), an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising Proline, Alanine and Serine (PAS), or a derivative or functional fragment thereof.

In some embodiments, the half-life extension moiety is a fragment crystallizable (Fc) region. In some embodiments, the Fc region comprises two polypeptides. In some embodiments, the Fc region has a knobs-into-holes format, wherein the first polypeptide of the Fc region comprises a hole mutation, and wherein second polypeptide of the Fc region comprises a knob mutation. In some embodiments, the first polypeptide of the Fc region comprises mutations T366S, L368A and Y407V according to EU numbering, and wherein the second polypeptide of the Fc region comprises a mutation T366W according to EU numbering. In some embodiments, the first polypeptide of the Fc region comprising the hole mutation further comprises a mutation Y349C according to EU numbering, wherein the second polypeptide of the Fc region comprising the knob mutation further comprises a mutation S354C according to EU numbering, and wherein the Fc region comprises an engineered disulfide bond formed between these two cysteines. In some embodiments, one or both of the polypeptides of the Fc region comprise mutations L234A, L235A, and P329G according to EU numbering. In some embodiments, one or both of the polypeptides of the Fc region comprise the mutation N297G according to EU numbering. In some embodiments, one or both of the polypeptides of the Fc region comprise mutations H435R and Y436F according to EU numbering. In some embodiments, the Fc region lacks one or more effector functions and/or lacks glycosylation. In some embodiments, the effector function is selected from the group consisting of antibody-dependent cellular cytotoxicity (ADCC), antibody-dependent cellular phagocytosis (ADCP), complement-dependent cytotoxicity (CDC), binding to a Fcγ receptor (FcγR), or any combination thereof. In some embodiments, the first polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 154, and wherein the second polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 155. In some embodiments, the first polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 156, and wherein the second polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 157.

In some embodiments, the C-terminus of the VH region of the first binding domain is connected to the N-terminus of the VH region of the second binding domain. In some embodiments, the C-terminus of the VH region of the first binding domain is connected to the N-terminus of the VL region of the second binding domain. In some embodiments, the C-terminus of the VL region of the first binding domain is connected to the N-terminus of the VH region of the second binding domain. In some embodiments, the C-terminus of the VL region of the first binding domain is connected to the N-terminus of the VL region of the second binding domain. In some embodiments, the C-terminus of the CH1 region of the first binding domain is connected to the N-terminus of the VH region of the second binding domain. In some embodiments, the C-terminus of the CH1 region of the first binding domain is connected to the N-terminus of the VL region of the second binding domain. In some embodiments, the C-terminus of the CL region of the first binding domain is connected to the N-terminus of the VH region of the second binding domain. In some embodiments, the C-terminus of the CL region of the first binding domain is connected to the N-terminus of the VL region of the second binding domain. In some embodiments, the C-terminus of the VH region of the second binding domain is connected to the N-terminus of either the first polypeptide or the second polypeptide of the Fc region. In some embodiments, the C-terminus of the VL region of the second binding domain is connected to the N-terminus of either the first polypeptide or the second polypeptide of the Fc region. In some embodiments, the C-terminus of the CH1 region of the second binding domain is connected to the N-terminus of either the first polypeptide or the second polypeptide of the Fc region. In some embodiments, the C-terminus of the CL region of the second binding domain is connected to the N-terminus of either the first polypeptide or the second polypeptide of the Fc region.

In some embodiments, the C-terminus of the VH region of the second binding domain is connected to the N-terminus of the VH region of the first binding domain. In some embodiments, the C-terminus of the VH region of the second binding domain is connected to the N-terminus of the VL region of the first binding domain. In some embodiments, the C-terminus of the VL region of the second binding domain is connected to the N-terminus of the VH region of the first binding domain. In some embodiments, the C-terminus of the VL region of the second binding domain is connected to the N-terminus of the VL region of the first binding domain. In some embodiments, the C-terminus of the CH1 region of the second binding domain is connected to the N-terminus of the VH region of the first binding domain. In some embodiments, the C-terminus of the CH1 region of the second binding domain is connected to the N-terminus of the VL region of the first binding domain. In some embodiments, the C-terminus of the CL region of the second binding domain is connected to the N-terminus of the VH region of the first binding domain. In some embodiments, the C-terminus of the CL region of the second binding domain is connected to the N-terminus of the VL region of the first binding domain. In some embodiments, the C-terminus of the VH region of the first binding domain is connected to the N-terminus of either the first polypeptide or the second polypeptide of the Fc region. In some embodiments, the C-terminus of the VL region of the first binding domain is connected to the N-terminus of either the first polypeptide or the second polypeptide of the Fc region. In some embodiments, the C-terminus of the CH1 region of the first binding domain is connected to the N-terminus of either the first polypeptide or the second polypeptide of the Fc region. In some embodiments, the C-terminus of the CL region of the first binding domain is connected to the N-terminus of either the first polypeptide or the second polypeptide of the Fc region.

In some embodiments, the second binding domain is connected to the second polypeptide of the Fc region comprising the knob mutation. In some embodiments, the second binding domain is connected to the first polypeptide of the Fc region comprising the hole mutation. In some embodiments, the first binding domain is connected to the second polypeptide of the Fc region comprising the knob mutation. In some embodiments, the first binding domain is connected to the first polypeptide of the Fc region comprising the hole mutation. In some embodiments, the other polypeptide of the Fc region is not connected to any binding domain.

In some embodiments, the first binding domain is an scFv, wherein the second binding domain is a Fab, wherein the C-terminus of the first binding domain is connected to the N-terminus of the VH region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

In some embodiments, the first binding domain is an scFv, wherein the second binding domain is a Fab, wherein the C-terminus of the first binding domain is connected to the N-terminus of the VL region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

In some embodiments, the first binding domain is an scFv, wherein the second binding domain is a Fab, wherein the N-terminus of the first binding domain is connected to the C-terminus of the CH1 region of the second binding domain via a peptide linker (PLB), and wherein the C-terminus of the first binding domain is connected to the N-terminus of the second polypeptide of the Fc region comprising a knob mutation via a peptide linker (PLE).

In some embodiments, the first binding domain is a Fab, wherein the second binding domain is an scFv, wherein the C-terminus of the CH1 region of the first binding domain is connected to the N-terminus of the second binding domain via a peptide linker (PLB), and wherein the C-terminus of the second binding domain is connected to the N-terminus of the second polypeptide of the Fc region comprising a knob mutation via a peptide linker (PLE).

In some embodiments, the first binding domain is a Fab, wherein the second binding domain is an scFv, wherein the N-terminus of the VH region of the first binding domain is connected to the C-terminus of the second binding domain via a peptide linker (PLB), and wherein the C-terminus of the CH1 region of the first binding domain is connected to the N-terminus of the second polypeptide of the Fc region comprising a knob mutation via a peptide linker (PLE).

In some embodiments, the first binding domain is a Fab, wherein the second binding domain is an scFv, wherein the N-terminus of the VL region of the first binding domain is connected to the C-terminus of the second binding domain via a peptide linker (PLB), and wherein the C-terminus of the CH1 region of the first binding domain is connected to the N-terminus of the second polypeptide of the Fc region comprising a knob mutation via a peptide linker (PLE).

In some embodiments, the first binding domain is a CrossFab, wherein the second binding domain is a charge steering Fab, wherein the N-terminus of the VL region of the first binding domain is connected to the C-terminus of the CH1 region of the second binding domain via a peptide linker (PLB), and wherein the C-terminus of the CH1 region of the first binding domain is connected to the N-terminus of the second polypeptide of the Fc region comprising a knob mutation via a peptide linker (PLE).

In some embodiments, the first binding domain is a CrossFab, wherein the second binding domain is a charge steering Fab, wherein the C-terminus of the CH1 region of the first binding domain is connected to the N-terminus of the VL region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

In some embodiments, the first binding domain and the second binding domain are connected via a peptide linker (PLB). In some embodiments, the peptide linker (PLB) is at least 5 amino acids in length. In some embodiments, the peptide linker (PLB) comprises the formula of (Gly₄Ser)ₙ, wherein n=1-5. In some embodiments, the peptide linker (PLB) is (Gly₄Ser)₃. In some embodiments, the peptide linker (PLB) is (Gly₄Ser)₂. In some embodiments, the peptide linker (PLB) is (Gly₄Ser)₁.

In some embodiments, the first binding domain is linked to either the first polypeptide or the second polypeptide of the Fc region, and wherein the second binding domain is linked to the other polypeptide of the Fc region. In some embodiments, the first binding domain is linked to the second polypeptide of the Fc region comprising a knob mutation, and wherein the second binding domain is linked to the first polypeptide of the Fc region comprising a hole mutation. In some embodiments, the first binding domain is a Fab or CrossFab, and wherein the second binding domain is an scFv. In some embodiments, the first binding domain is a CrossFab connecting to the Fc region via the C-terminus of its CH1 region, and wherein the second binding domain is a Fab connecting to the Fc region via the C-terminus of its CH1 region. In some embodiments, the first binding domain is a CrossFab connecting to the Fc region via the C-terminus of its CH1 region, and wherein the second binding domain is a charge steering Fab connecting to the Fc region via the C-terminus of its CH1 region. In some embodiments, the first binding domain is a CrossFab connecting to the Fc region via the C-terminus of its CL region, and wherein the second binding domain is a Fab connecting to the Fc region via the C-terminus of its CH1 region. In some embodiments, the C-terminus of the scFv or the CH1 region of the binding domain is linked to the N-terminus of the first or the second polypeptide of the Fc region. In some embodiments, the CL region of the binding domain is linked to the N-terminus of the first or the second polypeptide of the Fc region.

In some embodiments, the binding domain and the half-life extension moiety are connected via a peptide linker (PLE). In some embodiments, the peptide linker (PLE) is at least 5 amino acids in length. In some embodiments, the peptide linker (PLE) is between 5-10 amino acids, between 11-15 amino acids, between 16-20 amino acids, between 21-25 amino acids, or between 26-30 amino acids, in length. In some embodiments, the peptide linker (PLE) comprises an amino acid sequence having the formula of (Gly₄Ser)ₙ, wherein n=1-5. In some embodiments, the peptide linker (PLE) comprises (Gly₄Ser)₂. In some embodiments, the peptide linker (PLE) comprises or consists of the amino acid sequence KTHTCP (SEQ ID NO: 158). In some embodiments, the peptide linker (PLE) comprises or consists of the amino acid sequence DKTHTCP (SEQ ID NO: 160). In some embodiments, the peptide linker (PLE) comprises or consists of the amino acid sequence GGGGSGGGGSKTHTCP (SEQ ID NO: 159).

In some embodiments, the multi-specific polypeptide comprises an amino acid sequence that is at least 80%, at least 85%, at least 90%, at least 95%, at least 97%, at least 98%, at least 99%, or 100% identical to any one of SEQ ID NOs: 407-991 and 1159-1179.

In some embodiments, the multi-specific polypeptide comprises three non-identical polypeptides. In some embodiments, the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab light chain; and the third polypeptide comprises: (i) an anti-CD19 Fab heavy chain; (ii) a PLE linker; and (v) an Fc heavy chain comprising a knob mutation. In some embodiments, the first polypeptide comprises: (i) a PLE linker; and (ii) an Fc heavy chain comprising a hole mutation; the second polypeptide comprises: (i) an anti-CD19 Fab light chain; and the third polypeptide comprises: (i) an anti-TCRβ scFv; (ii) a PLB linker; (iii) an anti-CD19 Fab heavy chain; (iv) a PLE linker; and (iii) an Fc heavy chain comprising a knob mutation. In some embodiments, the first polypeptide comprises: (i) a PLE linker; and (ii) an Fc heavy chain comprising a hole mutation; the second polypeptide comprises: (i) an anti-CD19 Fab heavy chain; (ii) a PLE linker; and (iii) an Fc heavy chain comprising a knob mutation; and the third polypeptide comprises: (i) an anti-TCRβ scFv; (ii) a PLB linker; (iii) an anti-CD19 Fab light chain. In some embodiments, the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab heavy chain; (iv) a PLE linker; and (v) an Fc heavy chain comprising a knob mutation; and the third polypeptide comprises an anti-CD19 Fab light chain. In some embodiments, the first polypeptide comprises an amino acid sequence that is at least 80%, at least 85%, at least 90%, at least 95%, at least 97%, at least 98%, at least 99%, or 100% identical to SEQ ID NO: 1159; the second polypeptide comprises an amino acid sequence that is at least 80%, at least 85%, at least 90%, at least 95%, at least 97%, at least 98%, at least 99%, or 100% identical to SEQ ID NO: 1166; and the third polypeptide comprises an amino acid sequence that is at least 80%, at least 85%, at least 90%, at least 95%, at least 97%, at least 98%, at least 99%, or 100% identical to SEQ ID NO: 1173. In some embodiments, the first polypeptide comprises an amino acid sequence that is at least 80%, at least 85%, at least 90%, at least 95%, at least 97%, at least 98%, at least 99%, or 100% identical to SEQ ID NO: 1160; the second polypeptide comprises an amino acid sequence that is at least 80%, at least 85%, at least 90%, at least 95%, at least 97%, at least 98%, at least 99%, or 100% identical to SEQ ID NO: 1167; and the third polypeptide comprises an amino acid sequence that is at least 80%, at least 85%, at least 90%, at least 95%, at least 97%, at least 98%, at least 99%, or 100% identical to SEQ ID NO: 1174. In some embodiments, the first polypeptide comprises an amino acid sequence that is at least 80%, at least 85%, at least 90%, at least 95%, at least 97%, at least 98%, at least 99%, or 100% identical to SEQ ID NO: 1161; the second polypeptide comprises an amino acid sequence that is at least 80%, at least 85%, at least 90%, at least 95%, at least 97%, at least 98%, at least 99%, or 100% identical to SEQ ID NO: 1168; and the third polypeptide comprises an amino acid sequence that is at least 80%, at least 85%, at least 90%, at least 95%, at least 97%, at least 98%, at least 99%, or 100% identical to SEQ ID NO: 1175. In some embodiments, the first polypeptide comprises an amino acid sequence that is at least 80%, at least 85%, at least 90%, at least 95%, at least 97%, at least 98%, at least 99%, or 100% identical to SEQ ID NO: 1162; the second polypeptide comprises an amino acid sequence that is at least 80%, at least 85%, at least 90%, at least 95%, at least 97%, at least 98%, at least 99%, or 100% identical to SEQ ID NO: 1169; and the third polypeptide comprises an amino acid sequence that is at least 80%, at least 85%, at least 90%, at least 95%, at least 97%, at least 98%, at least 99%, or 100% identical to SEQ ID NO: 1176. In some embodiments, the first polypeptide comprises an amino acid sequence that is at least 80%, at least 85%, at least 90%, at least 95%, at least 97%, at least 98%, at least 99%, or 100% identical to SEQ ID NO: 1163; the second polypeptide comprises an amino acid sequence that is at least 80%, at least 85%, at least 90%, at least 95%, at least 97%, at least 98%, at least 99%, or 100% identical to SEQ ID NO: 1170; and the third polypeptide comprises an amino acid sequence that is at least 80%, at least 85%, at least 90%, at least 95%, at least 97%, at least 98%, at least 99%, or 100% identical to SEQ ID NO: 1177. In some embodiments, the first polypeptide comprises an amino acid sequence that is at least 80%, at least 85%, at least 90%, at least 95%, at least 97%, at least 98%, at least 99%, or 100% identical to SEQ ID NO: 1164; the second polypeptide comprises an amino acid sequence that is at least 80%, at least 85%, at least 90%, at least 95%, at least 97%, at least 98%, at least 99%, or 100% identical to SEQ ID NO: 1171; and the third polypeptide comprises an amino acid sequence that is at least 80%, at least 85%, at least 90%, at least 95%, at least 97%, at least 98%, at least 99%, or 100% identical to SEQ ID NO: 1178. In some embodiments, the first polypeptide comprises an amino acid sequence that is at least 80%, at least 85%, at least 90%, at least 95%, at least 97%, at least 98%, at least 99%, or 100% identical to SEQ ID NO: 1165; the second polypeptide comprises an amino acid sequence that is at least 80%, at least 85%, at least 90%, at least 95%, at least 97%, at least 98%, at least 99%, or 100% identical to SEQ ID NO: 1172; and the third polypeptide comprises an amino acid sequence that is at least 80%, at least 85%, at least 90%, at least 95%, at least 97%, at least 98%, at least 99%, or 100% identical to SEQ ID NO: 1179.

In some embodiments, the VH and VL regions are numbered with reference to the Kabat numbering scheme.

Also provided herein is a recombinant nucleic acid molecule encoding the multi-specific polypeptide disclosed herein.

Also provided herein is a vector comprising the recombinant nucleic acid molecule disclosed herein.

Also provided herein is a pharmaceutical composition comprising (i) the multi-specific polypeptide disclosed herein, the recombinant nucleic acid molecule disclosed herein, or the vector disclosed herein, and (ii) a pharmaceutically acceptable carrier, diluent, or excipient.

Also provided herein is a method of producing a multi-specific polypeptide, comprising introducing the recombinant nucleic acid molecule disclosed herein, or the vector disclosed herein into a host cell under conditions to express the multi-specific polypeptide complex in the cell, wherein the method is ex vivo.

Also provided herein is a method of expanding a T cell population, wherein the method comprises contacting an initial T cell population with the multi-specific polypeptide disclosed herein or the pharmaceutical composition disclosed herein, thereby producing an expanded population of T cells; wherein the initial T cell population expresses TCRβV or TCRαV on the cell surface, and wherein the first binding domain of the multi-specific polypeptide specifically binds the TCRβV or TCRαV. In some embodiments, the method depletes B cells.

In some embodiments, the method expands T cells in vitro or ex vivo. In some embodiments, the method expands T cells in vivo in a subject.

In some embodiments, the initial population of T cells comprises a plurality of effector T cells and/or memory T cells.

In some embodiments, the expanded population of T cells has an increased expansion of the effector T cells and/or memory T cells compared to that of a control T cell population contacted with a control multi-specific polypeptide comprising a first binding domain that specifically binds to CD3 instead of TCRβV or TCRαV. In some embodiments, the effector T cells are selected from the group consisting of effector memory T cells (TEM), T effector cells (TEFF), T effector cells re-expressing CD45RA (TEMRA), and a combination thereof. In some embodiments, the memory T cells are selected from the group consisting of central memory T cells (Tcm), T memory stem cells (Tscm), and a combination thereof.

In some embodiments, the expanded population of T cells has an increased percentage of effector T cells and/or memory T cells compared to that of a control T cell population contacted with a control multi-specific polypeptide comprising a first binding domain that specifically binds to CD3 instead of TCRβV or TCRαV. In some embodiments, the effector T cells are effector memory T cells (TEM). In some embodiments, the memory T cells are central memory T cells (Tcm).

In some embodiments, the expanded population of T cells has an increased and/or prolonged cytotoxicity compared to that of a control T cell population contacted with a control multi-specific polypeptide comprising a first binding domain that specifically binds to CD3 instead of TCRβV or TCRαV.

In some embodiments, the comparison between the expanded population of T cells expanded by the multi-specific polypeptide and control T cell population contacted with the control multi-specific polypeptide is performed 1, 2, 3, 4, 5, 6, 7, 8, 9, 10, or 11 days after an initial contact of the T cells.

Also provided herein is a method of depleting B cells in a subject having a disease or disorder related to the B cells, comprising administering to the subject an effective amount of the multi-specific polypeptide disclosed herein or the pharmaceutical composition disclosed herein. In some embodiments, the method decreases the number of B cells by at least 50%, at least 55%, at least 60%, at least 65%, at least 70%, at least 75%, at least 80%, at least 85%, at least 90%, at least 95%, at least 98%, at least 99%, at least 99.5%, at least 99.8%, at least 99.9%, or 100%, in the subject. In some embodiments, the B cells are autoreactive B cells. In some embodiments, the B cells are autoreactive memory B cells. In some embodiments, the method reduces autoantibodies.

Also provided herein is a method of repopulating naive B cells in a subject, comprising administering to the subject an effective amount of the multi-specific polypeptide disclosed herein or the pharmaceutical composition disclosed herein. In some embodiments, the subject has a disease or disorder related to B cells. In some embodiments, the method increases the number of naive B cells by at least 50%, at least 55%, at least 60%, at least 65%, at least 70%, at least 75%, at least 80%, at least 85%, at least 90%, at least 95%, at least 98%, at least 99%, or 100%, in the subject.

Also provided herein is a method of treating a disease or disorder in a subject in need thereof, comprising administering to the subject an effective amount of the multi-specific polypeptide disclosed herein or the pharmaceutical composition disclosed herein.

In some embodiments, the disease or disorder is a cancer. In some embodiments, the cancer is a blood cancer. In some embodiments, the blood cancer is chronic lymphocytic leukemia or non-Hodgkin lymphoma. In some embodiments, the disease or disorder is an autoimmune disease. In some embodiments, the autoimmune disease is rheumatoid arthritis or Systemic lupus erythematosus. In some embodiments, the subject has previously been treated with a multi-specific polypeptide comprising a first binding domain specifically binds to CD3 instead of TCRβV or TCRαV. In some embodiments, the subject is relapsing or refractory to a treatment comprising a multi-specific polypeptide comprising a first binding domain specifically binds to CD3 instead of TCRβV or TCRαV.

### BRIEF DESCRIPTION OF THE FIGURES

FIG. 1A illustrates non-limiting examples of T-cell engager molecules comprising (i) an anti-CD19 Fab and (ii) an anti-TCR (e.g., TRBV or TRAV) or anti-CD3 scFv. The scFv may have the format of N′-VH-VL-C′, or N′-VL-VH-C′, and optionally contain an engineered disulfide bond. FIG. 1B illustrates the binding domain format of Blincyto/Blinatumomab, as a non-limiting example of T-cell engager molecules comprising (i) an anti-CD19 scFv and (ii) an anti-CD3 scFv.

FIG. 2A illustrates non-limiting examples of T-cell engager molecules comprising (i) an anti-TCR (e.g., TRBV or TRAV) or anti-CD3 scFv; (ii) an anti-CD19 Fab; and (iii) an Fc region of knob-into-hole format. The scFv may have the format of N′-VH-VL-C′, or N′-VL-VH-C′, and optionally contain an engineered disulfide bond. The C-terminus of anti-CD19 Fab C H1 region is connected to the Fc chain comprising the knob mutation. (Molecule on the Left) the C-terminus of anti-TCR scFv is connected to the VH region of the anti-CD19 Fab; (Molecule on the Right) the C-terminus of anti-TCR scFv is connected to the VL region of the anti-CD19 Fab. FIG. 2B illustrate a non-limiting example of T-cell engager molecules comprising (i) an anti-CD19 Fab; (ii) an anti-TCR (e.g., TRBV or TRAV) or anti-CD3 scFv; and (iii) an Fc region of knob-into-hole format. The scFv may have the format of N′-VH-VL-C′, or N′-VL-VH-C′, and optionally contain an engineered disulfide bond. The C-terminus of anti-CD19 Fab C H1 region is connected to the N-terminus of scFv. FIG. 2C illustrate a non-limiting example of T-cell engager molecules comprising (i) a T cell binding Fab (e.g., with specific binding to CD3, TRBV or TRAV); (ii) an anti-CD19 scFv; and (iii) an Fc region of knob-into-hole format. The C-terminus of T cell binding Fab C H1 region may be connected to the N-terminus of the anti-CD19 scFv via a peptide linker. FIG. 2D illustrates non-limiting examples of T-cell engager molecules comprising (i) an anti-CD19 scFv; (ii) a T cell binding Fab (e.g., with specific binding to CD3, TRBV or TRAV); and (iii) an Fc region of knob-into-hole format. The C-terminus of T cell binding Fab C H1 region is connected to the Fc chain comprising the knob mutation. (Molecule on the Left) The C-terminus of the anti-CD19 scFv is connected to the VH region of the T cell binding Fab. (Molecule on the Right) The C-terminus of the anti-CD19 scFv is connected to the VL region of the T cell binding Fab. FIG. 2E illustrates non-limiting examples of T-cell engager molecules comprising (i) an anti-CD19 Fab with charge steering to enrich desired light chain pairing; (ii) a T cell binding CrossFab (e.g., with specific binding to CD3, TRBV or TRAV) to enrich desired light chain pairing; and (iii) an Fc region of knob-into-hole format. The Fab is connected to the Fc chain comprising the knob mutation via the C-terminus of the CH1 region. (Molecule on the Left) The C-terminus of the CH1 region of the anti-CD19 Fab with charge steering is connected to the N-terminus of the VL of the T cell binding CrossFab. (Molecule on the Right) The C-terminus of the CH1 region of the T cell binding CrossFab is connected to the N-terminus of the VH of the anti-CD19 Fab with charge steering.

FIG. 3A illustrates a non-limiting example of T-cell engager molecules comprising (i) an anti-CD19 scFv connected to the Fc chain comprising the hole mutation and (ii) a T cell binding Fab or CrossFab (e.g., with specific binding to CD3, TRBV or TRAV) connected to the Fc chain comprising the knob mutation. FIG. 3B illustrates non-limiting examples of T-cell engager molecules comprising (i) an anti-CD19 Fab connected to the Fc chain comprising the hole mutation via its CH1 region and (ii) a T cell binding CrossFab (e.g., with specific binding to CD3, TRBV or TRAV) connected to the Fc chain comprising the knob mutation. The anti-CD19 Fab may be a regular Fab or a charge steering Fab, and the T cell binding CrossFab may be connected to Fc chain via its CH1 region or its CL region.

FIG. 4A contains charts showing the results of B cell depletion and T cell expansion using TCE-94 or TCE-52, after 7-day culture in healthy donor PBMCs. FIG. 4B contains charts showing the results of B cell depletion and T cell expansion using TCE-9, TCE-26, or TCE-11, after 7-day culture in healthy donor PBMCs

FIG. 5A-5B contain charts showing the results of B cell depletion and T cell expansion using TCE-9, TCE-10, TCE-11, or TCE-12, after 7-day culture in healthy donor PBMCs. FIG. 5C-5D contain charts showing the results of B cell depletion and T cell expansion using TCE-17, TCE-18, TCE-19, or TCE-20, after 7-day culture in healthy donor PBMCs.

FIG. 6 contains charts showing the results of B cell depletion and T cell expansion using TCE-52, after 7-day culture in healthy donor PBMCs.

FIG. 7A-7B contain charts showing the results of B cell depletion and T cell expansion using TCE-13 or TCE-14, after 7-day culture in healthy donor PBMCs. FIG. 7C-7D contain charts showing the results of B cell depletion and T cell expansion using TCE-21 or TCE-22, after 7-day culture in healthy donor PBMCs.

FIG. 8A-8B contain charts showing the results of B cell depletion and T cell expansion using TCE-26 or TCE-27, after 7-day culture in healthy donor PBMCs.

FIG. 9 is a chart summarizing the results of B cell depletion and T cell expansion studies using the indicated T cell engagers.

FIG. 10A-10E contain charts showing the results of B cell depletion and T cell expansion using the indicated T cell engagers, after 7-day culture in healthy donor PBMCs.

FIG. 11 is a chart summarizing the results of B cell depletion and T cell expansion studies using the indicated T cell engagers.

FIG. 12A-12B contain charts showing the results of B cell depletion and T cell expansion using TCE-98 or TCE-99, after 7-day culture in healthy donor PBMCs.

FIG. 13A-13C contain charts showing the results of B cell depletion and T cell expansion using TCE-100 or TCE-101, after 7-day culture in healthy donor PBMCs.

FIG. 14A-14F contain charts showing the results of B cell depletion and T cell expansion using TCE-92, TCE-93, TCE-94, or TCE-95, after 7-day culture in healthy donor PBMCs.

FIG. 15 contains charts showing the results of B cell depletion and T cell expansion using TCE-167 or TCE-168, after 7-day culture in healthy donor PBMCs.

FIG. 16 contains charts showing the results of B cell depletion and T cell expansion using TCE-116 or TCE-118, after 7-day culture in healthy donor PBMCs.

FIG. 17A-17B contain charts showing the results of B cell depletion and T cell expansion using TCE-93, TCE-95, TCE-117, or TCE-119, after 7-day culture in healthy donor PBMCs.

FIG. 18A-18B contain charts showing the results of B cell depletion and T cell expansion using TCE-102 or TCE-103, after 7-day culture in healthy donor PBMCs.

FIG. 19A-19D contain charts showing the results of B cell depletion and T cell expansion using TCE-80, TCE-81, TCE-84, TCE-85, TCE-88, or TCE-89, after 7-day culture in healthy donor PBMCs.

FIG. 20A-20D contain charts showing the results of B cell depletion and T cell expansion using TCE-96, TCE-97, TCE-82, TCE-86, or TCE-90, after 7-day culture in healthy donor PBMCs.

FIG. 21A-21B contain charts showing the results of B cell depletion and T cell expansion using TCE-83, TCE-87, or TCE-91, after 7-day culture in healthy donor PBMCs.

FIG. 22A-22C are charts summarizing the results of B cell depletion and T cell expansion studies using the indicated T cell engagers.

FIG. 23A-23B contain charts showing the changes of B cells, CD8+ T cells, CD4+ T cells, and Treg cells in SLE PBMCs over time in the presence of TCE-9 or TCE-11. FIG. 23C-23H contain charts showing the change of TRBV− or TRBV+ cells in different T cell populations over time in the presence of different concentrations of TCE-9 or TCE-11.

FIG. 24A-24F contain plots showing the changes over time of different naïve, memory, and effector T cell populations from PBMCs of SLE patients in the presence of different concentrations of TCE-9 or TCE-11.

FIG. 25A are charts summarizing B-cell depletion in SLE patient PBMCs using TCE-9 or TCE-11. FIG. 25B is a chart summarizing the maximum fold change expansion of T cell subsets in SLE donor PBMCs using TCE-9 or TCE-11.

FIG. 26 contains charts showing B cell depletion in CLL patient PBMCs in the presence of different concentrations of TCE-9 or TCE-11.

FIG. 27 contains charts showing the changes of CD8+ and CD4+ T cell populations in CLL patient PBMCs over time in the presence of different concentrations of TCE-9 or TCE-11.

FIGS. 28A-28H show ELISA binding results for exemplary anti-CD19 and anti-TRBV bispecific T cell engagers CLY-018 to CLY-024. FIG. 28A shows a summary of binding results for CLY-018 to CLY-024 against human CD19 (5 μg/mL) at concentrations from 0-5 μM. FIG. 28B summarizes the results of FIG. 28A. FIG. 28C shows a summary of binding results for CLY-018 to CLY-024 against cyno CD19 (5 μg/mL) at concentrations from 0-5 μM. FIG. 28D summarizes the results of FIG. 28C. FIG. 28E shows a summary of binding results for CLY-018 to CLY-024 against human TRBV6-5 at 5 μg/mL. FIG. 28F summarizes the results of FIG. 28E.

FIG. 28G shows a summary of binding results for CLY-018 to CLY-024 against cyno TRBV6-2 at 5 μg/mL. FIG. 28H summarizes the results of FIG. 28G.

FIGS. 29A-29D show results of human cell binding of exemplary anti-CD19 and anti-TRBV bispecific T cell engagers CLY-018 to CLY-024 by flow cytometry. FIG. 29A shows the human B cell binding results. FIG. 29B summarizes the results of FIG. 29A. FIG. 29C shows the human T cell binding results. FIG. 29D summarizes the results of FIG. 29C.

FIGS. 30A-30D show results of cyno cell binding of exemplary anti-CD19 and anti-TRBV bispecific T cell engagers CLY-018 to CLY-024 by flow cytometry. FIG. 30A shows the cyno B cell binding results. FIG. 30B summarizes the results of FIG. 30A. FIG. 30C shows the cyno T cell binding results. FIG. 30D summarizes the results of FIG. 30C.

FIGS. 31A-FIG. 31U show results of functionality assays for exemplary anti-CD19 and anti-TRBV bispecific T cell engagers CLY-018 to CLY-024. FIG. 31A shows normalized B cell count for human PBMC sample and the exemplary T cell engagers. FIG. 31B summarizes the EC50 values for human B cell killing shown in FIG. 31A. FIG. 31C shows results for CD8+TRBV+ for human PBMC sample and the exemplary T cell engagers. FIG. 31D shows results for non-Treg CD4+TRBV+ for human PBMC sample and the exemplary T cell engagers.

FIG. 31E shows results for T regs for human PBMC sample and the exemplary T cell engagers.

FIG. 31F shows expansion of CD8+TRBV+ for the CLY-023 T cell engager from human PBMCs.

FIG. 31G shows expansion of CD8+TRBV+ for the CLY-018 T cell engager from human PBMCs.

FIG. 31H shows expansion of CD8+TRBV+ for the CLY-019 T cell engager from human PBMCs.

FIG. 31I shows expansion of CD8+TRBV+ for the CLY-020 T cell engager from human PBMCs.

FIG. 31J shows expansion of CD8+TRBV+ for the CLY-021 T cell engager from human PBMCs.

FIG. 31K shows expansion of CD8+TRBV+ for the CLY-022 T cell engager from human PBMCs.

FIG. 31L shows B cell count after treatment of cyno PBMCs (donor “326”) with CLY-020, CLY-021, or CLY-024. FIG. 31M summarizes the results of FIG. 31L. FIG. 31N shows B cell count after treatment of cyno PBMCs (donor “1611”) with CLY-020, CLY-021, or CLY-024. FIG. 31O summarizes the results of FIG. 31N. FIG. 31P shows CD8+TRBV+ T cell fold change for cyno PBMCs (donor “326”) treated with CLY-020, CLY-021, or CLY-024. FIG. 31Q shows CD8+TRBV+ T cell fold change for cyno PBMCs (donor “1611”) treated with CLY-020, CLY-021, or CLY-024. FIG. 31R shows CD4+TRBV+ T cell fold change for cyno PBMCs (donor “326”) treated with CLY-020, CLY-021, or CLY-024. FIG. 31S shows CD4+TRBV+ T cell fold change for cyno PBMCs (donor “1611”) treated with CLY-020, CLY-021, or CLY-024. FIG. 31T shows CD8+TRBV+ T cell fold change for cyno PBMCs (donor “326”) treated with CLY-020. FIG. 31U shows CD8+TRBV+ T cell fold change for cyno PBMC (donor “1611”) treated with CLY-020. FIG. 31V shows B cell fold change at Days 1~4 and 7 following treatment of healthy PBMCs (donor “1”) with various doses of CLY-018 (left panel) or CLY-019 (right panel). FIG. 31W shows B cell fold change at Days 1~4 and 7 following treatment of healthy PBMCs (donor “2”) with various doses of CLY-018 (left panel) or CLY-019 (right panel). FIG. 31X shows CD8+TRBV+ T cell count at Day 4 (left panel) and Day 7 (right panel) following treatment of healthy PBMCs (donor “1”) with various doses of CLY-018 or CLY-019. FIG. 31Y shows CD8+TRBV+ T cell count at Day 4 (left panel) and Day 7 (right panel) following treatment of healthy PBMCs (donor “2”) with various doses of CLY-018 or CLY-019. FIG. 31Z shows B cell fold change at Days 1-3 and 7 following treatment of healthy bone marrow aspirates from donor “1” (left panel) and donor “2” (right panel) with various doses of CLY-018. FIG. 31AA shows B cell fold change at Days 1, 3, 4 and 7 following treatment of healthy splenocytes from donor “1” (left panel), donor “2” (middle panel), or donor “3” (right panel) with various doses of CLY-018.

FIGS. 32A-32F show functionality results for assays utilizing exemplary anti-CD19 and anti-TRBV bispecific T cell engagers CLY-023, CLY-018, CLY-019, and CLY-022. FIG. 32A shows average TRBV+CD8+ T cell count for 3 donors treated with CLY-023, CLY-018, CLY-019, or CLY-022 in a 7-day culture. FIG. 32B shows average TRBV+CD4+ T cell count for 3 donors treated with CLY-023, CLY-018, CLY-019, or CLY-022 in a 7-day culture. FIG. 32C shows expansion of TRBV+CD8+ T cells after a 7-day culture with CLY-022, CLY-019, CLY-018, or CLY-023 enriched for TRBV6-1, 6-2, 6-5, and 6-9 (n=3 donor CD8+ T cells). Note that CLY-022 also expanded TRBV10-3 T cells. FIG. 32D shows expansion of TRBV+CD4+ T cells after a 7-day culture with CLY-022, CLY-019, CLY-018, or CLY-023 enriched for TRBV6-1, 6-2, 6-5, and 6-9 (n=2-3 donor CD4+ T cells). Note that CLY-022 also expanded TRBV10-3 T cells.

FIG. 32E shows expansion of TRAV+CD8+ T cells after a 7-day culture with CLY-022, CLY-019, CLY-018, or CLY-023 indicating no change in relative abundance of TRAVs with TRBV stimulation (n=2-3 donor CD4+ T cells). FIG. 32F shows expansion of TRAV+CD4+ T cells after a 7-day culture with CLY-022, CLY-019, CLY-018, or CLY-023 indicating no change in relative abundance of TRAVs with TRBV stimulation (n=2-3 donor CD4+ T cells).

FIGS. 33A-33R show results of in vivo activity of exemplary anti-CD19 and anti-TRBV bispecific T cell engagers in a cynomolgus monkey model. FIG. 33A shows the plasma concentration of CLY-023 for monkeys treated with CLY-023. FIG. 33B shows CD20+ B cell count in the blood of monkeys treated with CLY-023. Note that minimal B cell depletion was observed in monkeys dosed up to 1 mg/kg IV. FIG. 33C shows cytokine induction results for monkeys treated with CLY-023 at 0.1 mg/kg IV. FIG. 33D shows cytokine induction results for monkeys treated with CLY-023 at 1 mg/kg IV. Of note is that no in life observations or signs of cytokine release syndrome in monkeys dosed up to 1 mg/kg IV were detected. FIG. 33E shows the plasma concentration of CLY-024 for monkeys treated with CLY-024 at various concentrations. FIG. 33F shows CD20+ B cell count in the blood of monkeys treated with CLY-024. Of note is that moderate B cell depletion observed in monkey dosed at 1 mg/kg IV. Minimal B cell depletion observed at lower doses (<1 mg/kg IV). FIG. 33G shows cytokine induction results for monkeys treated with CLY-024 at 0.01 mg/kg, iv. FIG. 33H shows cytokine induction results for monkeys treated with CLY-024 at 0.1 mg/kg, iv. FIG. 33I shows cytokine induction results for monkeys treated with CLY-024 at 1 mg/kg, iv. No in life observations or signs of cytokine release syndrome in cynomolgus monkeys dosed up to 1 mg/kg IV. FIG. 33J shows plasma concentration of CLY-020 in monkeys treated with CLY-020. FIG. 33K shows CD20+ B cell count in the blood of monkeys treated with CLY-020 over 28 days. FIG. 33L shows cytokine induction in CLY-020-treated monkeys for IL-2, IFN gamma, IL-6 and TNF. FIG. 33M shows plasma concentration of CLY-018 in monkeys treated with CLY-018. FIG. 33N shows CD20+ B cell count in monkeys treated with CLY-018. FIG. 33O shows cytokine induction for monkeys treated with CLY-018 at a concentration of 0.1 mg/kg IV. FIG. 33P shows cytokine induction for monkeys treated with CLY-018 at 1 mg/kg IV. FIG. 33Q shows cytokine induction for monkeys treated with CLY-018 at 2.5 mg/kg IV. FIG. 33R shows cytokine induction for monkeys treated with CLY-018 at 9 mg/kg. No in life observations or signs of cytokine release syndrome in cynomolgus monkeys dosed up to 9 mg/kg IV was observed. FIG. 33S shows CD20+ B cell count in the blood of monkeys over time with various dosing regimens of CLY-018. FIG. 33T shows plasma concentration of CLY-018 in monkeys over time following treatment with various dosing regimens of CLY-018. FIG. 33U shows peak IL-6 cytokine induction in monkeys following treatment with various dosing regimens of CLY-018. FIG. 33V shows percent change in B cells in the blood of monkeys with various dosing regimens of CLY-018. Percent change was calculated by determining B cell count before treatment compared to Day 7 after treatment with CLY-018. FIG. 33W shows peak IL-6 cytokine induction in monkeys following treatment with various dosing regimens of CLY-018. FIG. 33X shows percentage of CD20+ B cells in the blood, bone marrow, spleen, and lymph node before (Day 0) and after (Day 17) treatment with various dosing regimens of CLY-018. The data shown are for animals that maintained PK exposure through Day 17 with no impact from potential anti-drug antibodies. FIG. 33Y shows the number of switched memory, unswitched memory, naïve, and immature/transitional/double negative B cells in the blood before and after treatment with various dosing regimens of CLY-018. Data is consistent with an immune reset. FIG. 33Z shows a schematic of a B cell depletion phase and a B cell recovery phase following administration of a CD19xTRBV T cell engager (TCE). Treatment with the TCE results in rapid and complete B cell depletion within several days (B cell depletion phase) followed by an immune reset characterized by B cell repopulation of predominantly naïve B cells, sustained depletion of autoreactive memory cells, and reduction of autoantibodies (B cell recovery phase). IV, intravenous; SC, subcutaneous; QW, once weekly.

FIGS. 34A-34T show activity of the CLY-018 T cell engager in healthy human PBMCs and PBMCs of autoimmune subjects (e.g., RA, SLE, and MG). FIG. 34A shows average B-cell fold change/killing of healthy human PBMC treated with CLY-018 over the course of 7 days. FIG. 34B shows average non-Treg CD4+ T cell fold change of healthy human PBMCs treated with CLY-018 over the course of 7 days. FIG. 34C shows average CD8+ T cell fold change of healthy human PBMC treated with CLY-018 over the course of 7 days. FIG. 34D shows average T reg fold change of healthy human PBMC treated with CLY-018 over the course of 7 days. FIG. 34E shows results of CD8+ T cell expansion for the Tn, Tscm, Ttm, Tcm, Tem, Teff, and Temra subsets of healthy human PBMCs treated with CLY-018. FIG. 34F shows average B-cell fold change/killing of PBMCs from subjects with RA treated with CLY-018 over 7 days. FIG. 34G shows average non-Treg CD4+ T cell fold change of PBMCs from subjects with RA treated with CLY-018 over 7 days. FIG. 34H shows average CD8+ T cell fold change of PBMCs from subjects with RA treated with CLY-018. FIG. 34I shows average T reg fold change of PBMCs from subjects with RA treated with CLY-018. FIG. 34J shows results of CD8+ T cell expansion for the Tn, Tscm, Ttm, Tcm, Tem, Teff, and Temra subsets from PBMCs of RA subjects incubated with CLY-018. FIG. 34K shows average B-cell fold change/killing of PBMCs from 3 SLE subjects treated with CLY-018 over 7 days. FIG. 34L shows average non-Treg CD4+ T cell fold change of PBMCs from 3 SLE subjects treated with CLY-018 over 7 days. FIG. 34M shows average CD8+ T cell fold change of PBMCs from 3 SLE subjects treated with CLY-018. FIG. 34N shows average T reg fold change of PBMCs from 3 SLE subjects treated with CLY-018. FIG. 34O shows results of CD8+ T cell expansion for the Tn, Tscm, Ttm, Tom, Tem, Teff, and Temra subsets from PBMCs of SLE subjects incubated with CLY-018. FIG. 34P shows average B-cell fold change of PBMCs from 3 MG subjects treated with CLY-018 over 7 days. FIG. 34Q shows average non-Treg CD4+ T cell fold change of PBMCs from 3 MG subjects treated with CLY-018 over 7 days. FIG. 34R shows average CD8+ T cell fold change of PBMCs from 3 MG subjects treated with CLY-018. FIG. 34S shows average T reg fold change of PBMCs from 3 MG subjects treated with CLY-018. FIG. 34T shows results of CD8+ T cell expansion for the Tn, Tscm, Ttm, Tcm, Tem, Teff, and Temra subsets of the PBMCs from MG subjects incubated with CLY-018. FIG. 34U shows relative abundance of TRBV+ T cell subsets (Tn, Tscm, Ttm, Tcm, Tem, Teff, and Temra) in human PBMCs from healthy donors, SLE donors, RA donors, and MG donors before (Day 0) and after (Day 7) treatment with 1 nM of CLY-018. FIG. 34V shows B cell counts (left y-axis) and CD8+TRBV+ T cell fold change (right y-axis) of human PBMCs from healthy donors, RA donors, SLE donors, and MG donors treated with various doses of CLY-018 or TCE-52. RA, rheumatoid arthritis; SLE, systemic lupus erythematosus; MG, myasthenia gravis.

FIG. 35 shows percent lysis of Raji B cells over time in T cell co-culture upon treatment with CLY-018 or TCE-160.

FIGS. 36A-36B depict relative B cell depletion and IL-6 cytokine release of the CD19-TCRβV bispecifics of the present disclosure (Tx 1) compared to other B cell therapeutics (CLN-978 (Tx 2), Blincyto (Tx 3), Epcoritamab (Tx 4), Glofitamab (Tx 5), and Mosunetuzumab (Tx 6)). those of other B-cell therapeutics by Cullinan, Amgen, Genmab, Roche and Genentech. FIG. 36A depicts B cell depletion as percent change of measured B cell population 1 week prior to dose with either CD19-TCRβV bispecifics of the present disclosure or other competing therapeutics. FIG. 36B depicts B cell depletion as percent change of measured B cell population 1 week prior to dose with either CD19-TCRβV bispecifics of the present disclosure (Tx 1) or other B cell therapeutics (Tx 2 to Tx 6).

FIGS. 37A-37E depict potency of B cell depletion by CD19-TCRβV bispecifics of the present disclosure. FIG. 37A shows EC50 of B cell depletion of different cell populations from disease patients. FIG. 37B shows percentage of B cell killing within cell populations from disease patients. FIGS. 37C, 37D, and 37E depict B cell depletion and T cell expansion after treatment with CD19-TCRβV bispecifics of the present disclosure. SLE-systemic lupus; RA-rheumatoid arthritis; MG-myasthenia gravis (MG); IIM-idiopathic inflammatory myopathy.

FIG. 38 shows the design of a clinical trial designed to further validate human treatments using CD19-TCRβV bispecifics of the present disclosure.

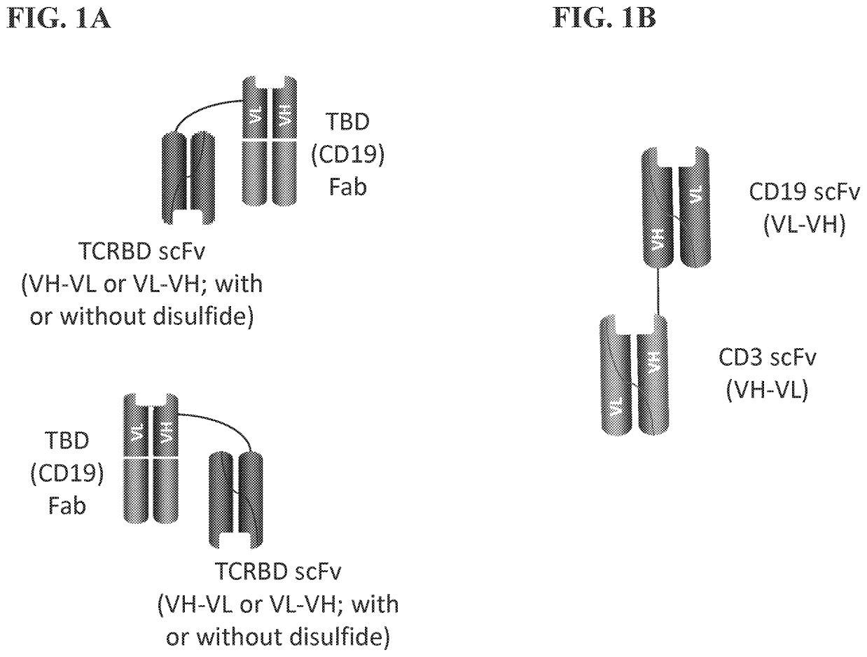

### DETAILED DESCRIPTION

In some embodiments, the present disclosure provides designs of multi-specific polypeptides for binding target cells and engaging T cells with improved efficacy. In some embodiments, such multi-specific polypeptides not only are effective in killing the target cells, but also induce larger increases in early memory populations that maintains and/or replenishes effector populations over time. In some embodiments, the multi-specific polypeptide comprises a first binding domain that specifically binds to a T cell receptor beta variable region (TCRβV) or a T cell receptor alpha variable region (TCRαV) expressed on the T cell. In some embodiments, the multi-specific polypeptide comprises a first binding domain that specifically binds to CD3. In some embodiments, the multi-specific polypeptide comprises a second binding domain that specifically binds to an antigen (e.g., CD19) on the target cell. In some embodiments, the multi-specific polypeptide comprises a Fc region, and both the first and the second binding domains are located on the same polypeptide chain of the Fc region (e.g., a Fc region polypeptide chain comprising a Knob mutation). In some embodiments, the multi-specific polypeptide comprises the first binding domain (e.g., an scFv) linked to the second binding domain (e.g., a Fab), and the second binding domain in turn linked to a polypeptide chain of the Fc region. In some embodiments, the multi-specific polypeptide does not comprise a cytokine.

In some embodiments, the present disclosure provides methods of expanding T cells, comprising contacting a T cell with a multi-specific polypeptide, thereby inducing a production of a population of T cells comprising a plurality of memory T cells and effector T cells. In some embodiments, the methods disclosed herein are advantageous because the resulting population of T cells has an increased durability than that of a CD3 binder-expanded population of T cells. Specifically, the methods effectively expands both effector T cells (such as effector memory T cells (TEM), T effector cells (TEFF), and/or T effector cells re-expressing CD45RA (TEMRA)) and memory T cells (such as central memory T cells (TCM) and/or T memory stem cells (TSCM)). It is noteworthy that the methods bias the expanded T cell composition towards both towards TCM and TEM. In the meantime, the methods bias the expanded T cell composition towards CD8+ and CD4+ (non-Treg) T cells and away from regulatory T cells. The resulting population of T cells thus has an increased and/or prolonged cytotoxicity than that of a CD3 binder-expanded population of T cells.

In some embodiments, the methods disclosed herein can reduce the toxicity and side effects associated with cytokine release syndrome (CRS) because the resulting population of T cells has reduced cytokine release than that of a CD3 binder-expanded population of T cells.

Given the advantageous features of the methods of expanding T cells disclosed herein, the present disclosure also provided methods of treating a disease or disorder in a subject in need thereof, comprising administering a therapeutically effective amount of a pharmaceutical composition comprising a multi-specific polypeptide to the subject, wherein the multi-specific polypeptide contacts a T cell of the subject, thereby inducing a production of a population of T cells, wherein the multi-specific polypeptide comprises a first binding domain that specifically binds to a T cell receptor beta variable region (TCRβV) or a T cell receptor alpha variable region (TCRαV) expressed on the T cell, and wherein the population of T cells comprises a plurality of memory T cells and effector T cells. The disease or disorder can be a cancer (e.g., chronic lymphocytic leukemia) or an autoimmune disease (e.g., rheumatoid arthritis, and Systemic lupus erythematosus).

### 1.1. Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa unless the content clearly dictates otherwise. In the event that any description of a term set forth conflicts with any document incorporated herein by reference, the description of the term set forth below shall control.

The terms “a”, “an”, and “the”, as used herein, include plural references unless the context clearly dictates otherwise.

The term “about” or “approximately”, as used herein, in reference to a number or range of numbers, is understood to mean the stated number and numbers +/−10% thereof, or 10% below the lower listed limit and 10% above the higher listed limit for the values listed for a range (except where such number would exceed 100% of a possible value or go below 0%). It is used to indicate that a value includes the inherent variation of error for the device or the method being employed to determine the value, or the variation that exists among the samples being measured.

The term “between”, as used in a phrase as such “between A and B” or “between A-B” refers to a range including both A and B.

The terms “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The terms “including”, “includes”, “included”, and other forms, as used herein, are not limiting.

The terms “or” and “and/or”, as used herein, include any, and all, combinations of one or more of the associated listed items.

In the present description, any concentration range, percentage range, ratio range, or integer range is to be understood to include the value of any integer within the recited range and, when appropriate, fractions thereof (such as one tenth and one hundredth of an integer), unless otherwise indicated.

The phrase “any range or subrange therebetween” or equivalents, are used herein to denote the intention that disclosure of any range or series of possible values, inherently also discloses all ranges and subranges encompassed by the highest and lowest values disclosed. This term includes the entire range from highest to lowest disclosed values, as well as subranges from any two or more disclosed points. This term is also intended to disclose any subranges encompassed anywhere within the highest and lowest disclosed values, including between two points that are explicitly recited in the document, up to one decimal point. Thus, disclosure of values 0, 5, 10, 15, 20, including all ranges and subranges therebetween, should be interpreted as also encompassing a range from 0-20, a range from 0-5 or 5-15, as well as a range from 2-16, or 3.1 to 19.8, etc. Unless otherwise indicated, it is to be understood that all numbers expressing quantities, ratios, and numerical properties of ingredients, reaction conditions, and so forth, used in the specification are contemplated to be able to be modified in all instances by the term “including all ranges and subranges therebetween”.

The term “administer”, “administration”, or “administering”, as used herein refers to the act of injecting or otherwise physically delivering a substance (e.g., a pharmaceutical composition provided herein) to a subject (e.g., human), such as by oral, mucosal, topical, intradermal, parenteral, intravenous, intravitreal, intraarticular, subretinal, intramuscular, intrathecal delivery and/or any other method of physical delivery described herein or known in the art. The delivery can be systemic or to a specific tissue.

The terms “amino-terminal” and “carboxyl-terminal” are used herein to denote positions within polypeptides. Where the context allows, these terms are used with reference to a particular sequence or portion of a polypeptide to denote proximity or relative position.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multi-specific antibodies (e.g., bispecific antibodies so long as they exhibit the desired biological activity). A conventional antibody is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). An antibody can be human, humanized, chimeric and/or affinity matured, as well as an antibody from other species, for example, mouse and rabbit, etc. Antibodies also include, but are not limited to, synthetic antibodies, recombinantly produced antibodies, camelized antibodies or their humanized variants, and intrabodies. An antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). An antibody can comprise kappa or lambda light chain constant sequences.

The term “antigen”, as used herein, refers to a structure to which an antibody or an antibody fragment can specifically bind. An antigen may be a polypeptide, carbohydrate, nucleic acid, lipid, hapten, or other naturally occurring or synthetic compound.

The term “antigen-binding domain”, (e.g., a CD3 or TCRβV-binding domain) as used herein, refers to a portion of a binding molecule that specifically binds a target antigen or target epitope. Antigen-binding domains may comprise antibodies or antigen-binding fragments thereof. The term “antigen-binding fragment”, as used herein, refers to a portion of an antibody heavy and/or light chain polypeptide that retains some or all of the binding activity of the antibody from which the fragment was derived. An antigen-binding fragment may comprise 1, 2, 3, 4, 5, or all 6 CDRs of a variable heavy chain (VH) and/or variable light chain (VL) sequence. Non-limiting examples of functional fragments include single-chain Fvs (scFv), Fab fragments, CrossFab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. Such functional antigen-binding fragment can be found in, for example, Harlow and Lane, Antibodies: A Laboratory Manual (1989); Mol. Biology and Biotechnology: A Comprehensive Desk Reference (Myers, ed., 1995); Huston, et al, 1993, Cell Biophysics 22:189-224; Plückthun and Skerra, 1989, Meth. Enzymol. 178:497-515; and Day, Advanced Immunochemistry (2d ed. 1990).

The term “binds” or “binding”, as used herein, refers to a covalent or non-covalent interaction between molecules (e.g., forming a complex by interactions). Exemplary non-covalent interactions include hydrogen bonds, ionic bonds, hydrophobic interactions, and/or van der Waals interactions. Terms such as “specific binding,” “specifically binds to,” or “is specific for” means that a binding domain or a binding molecule comprising a binding domain binds the target with an affinity or Kₐ (i.e., an equilibrium association constant of a particular binding interaction with units of 1/M) equal to or greater than 105 M-1, while not significantly binding other components present in a test sample.

The term “binding affinity”, as used herein, refers to the strength of the sum total of noncovalent interactions between a binding domain X and its binding partner Y. Unless indicated otherwise, as used herein, “binding affinity” refers to intrinsic binding affinity which reflects a 1:1 interaction between members of a binding pair (e.g., antibody and antigen). Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹ “Low affinity” binding domains refer to those binding domains with a Kₐ of up to 10⁷ M⁻¹, up to 10⁶ M⁻¹, up to 10⁵ M⁻¹. High-affinity binding domains generally bind the binding partners faster and tend to remain bound longer, whereas low-affinity binding domains generally bind the binding partners slowly and tend to dissociate readily. Affinity can also be defined as an equilibrium dissociation constant (K_{d}) of a particular binding interaction with units of M (e.g., 10⁻⁵ M to 10⁻¹³, or about 1000 nM, about 900 nM, about 800 nM, about 700 nM, about 600 nM, about 500 nM, about 300 nM, about 250 nM, about 200 nM, about 150 nM, about 100 nM, about 50 nM, about 25 nM, about 10 nM, or about 5 nM, including any ranges or subranges therebetween). Affinities of binding domains according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

The term “binding molecule”, as used herein, refers to a polypeptide or complex of polypeptides comprising at least one binding domain that specifically binds to a target. In some embodiments, the binding molecule is a multi-specific polypeptide. As used herein, a “multi-specific polypeptide” refers to a polypeptide comprising two or more binding domains each capable of specifically binding to a target. For example, the polypeptides described herein may comprise 2, 3, 4, or more binding domains and may be able to bind 2, 3, 4, or more targets. In some embodiments, the multi-specific polypeptide comprises a first binding domain that specifically binds to a T cell receptor beta variable region (TCRβV) or a T cell receptor alpha variable region (TCRαV) expressed on the T cell. In some embodiments, the multi-specific polypeptide comprises a first binding domain that specifically binds to CD3. In some embodiments, the multi-specific polypeptide further comprises a second binding domain specifically binding to a protein expressed on a B cell (e.g., CD19).

The term “CD3 binder-expanded population of T cells”, as used herein, refers to a population of T cells expanded from a T cell after a polypeptide specifically binding to CD3 expressed on the T cell.

The term “coding sequence” or a polynucleotide which “encodes” a polypeptide, as used herein, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The term “constant region” or “constant domain”, as used herein, refers to a carboxy terminal portion of the light and heavy chain which is not directly involved in binding of the antibody to antigen but exhibits various effector function, such as interaction with the Fc receptor. This portion has a more conserved amino acid sequence relative to the variable region. The constant region may contain the CH1, CH2, and CH3 regions of the heavy chain and the CL region of the light chain.

The term “contact”, as used in the context of contacting a target cell with a compound (e.g., a multi-specific polypeptide), is intended to include incubating the target cell and the compound together.

The term “cytokine”, as used herein, refers to small proteins, a functional fragment therefore, a functional variant thereof, and a combination thereof that act as signaling molecules to regulate immunity, inflammation, and hematopoiesis. Cytokines include chemokines, interferons, interleukins, lymphokines, and tumor necrosis factors. Specific examples of cytokines include interleukin-2 (IL-2), interleukin-7 (IL-7), interleukin-10 (IL-10), interleukin-12 (IL-12), interleukin-15 (IL-15), interleukin-18 (IL-18), interleukin-21 (IL-21), interferon gamma, a functional fragment therefore, a functional variant thereof, and a combination thereof.

The term “cytotoxicity”, as used herein, refers to an ability of an effector cell to kill a target cell. In some embodiments, the effector cell is a TCRβV+ T cell. In some embodiments, the effector cell is a TCRαV+ T cell. In some embodiments, the target cell is a B cell, such as a CD19-positive B cell.

The term “disulfide bond”, as used herein, refers to the covalent linkage between the thiol groups of two cysteine residues. The specific position/location of the disulfide bond can be defined using the location of the two cysteine residues within a protein or polypeptide. For instance, a VH44-VL100 disulfide bond of the TCRβV-binding domain refers to a disulfide bond formed between the cysteine residue at amino acid position 44 of the VH region of the TCRβV-binding domain and the cysteine residue at amino acid position 100 of the VL region of the TCRβV-binding domain. Similarly, a VH100B-VL49 disulfide bond of the TCRβV-binding domain refers to a disulfide bond formed between the cysteine residue at amino acid position 100B of the VH region of the TCRβV-binding domain and the cysteine residue at amino acid position 49 of the VL region of the TCRβV-binding domain. It is well known to a skilled artisan that antibodies have different HCDR3 lengths and additional residues may occur between positions VH100 and VH101 that can be numbered with a letter from A to K according to the Kabat numbering scheme (e.g., VH100A, VH100B, VH100C . . . H100K).

The term “durability”, as used herein, refers to the ability of a population of T cells to maintain their effector function (i.e., ability to kill a target cell) and the capacity to expand and differentiate.

The term “effective amount”, as used herein, refers to an amount of a therapeutic (e.g., a pharmaceutical composition provided herein) which is sufficient to prevent, diagnose, treat, delay the onset of, reduce and/or ameliorate the severity, duration, advancement, and/or recurrence of a given condition, disorder or disease and/or a symptom related thereto. The term also encompasses an amount necessary to improve or enhance the prophylactic or therapeutic effect(s) of another therapy or to serve as a bridge to another therapy.

The term “effector T cell”, as used herein, refers to active form of T cells that have relatively short lifespans and carry out the functions of an immune response. Effector T cells can be cytotoxic (CD8+), helper (CD4+, non-reg), and regulatory T cells. Specific types of effector T cells include effector memory T cells (TEM), T effector cells (TEFF), and T effector cells re-expressing CD45RA (TEMRA). In some embodiments, the effector T cell is TCRβV+. In some embodiments, the effector T cell is TCRαV+.

The term “Fab” or “Fab region”, as used herein, refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders.

The term “heavy chain”, when used in reference to an antibody, refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha, delta, epsilon, gamma, and mu, based on the amino acid sequence of the heavy chain constant region. The distinct heavy chains differ in size: alpha, delta, and gamma contain approximately 450 amino acids, while epsilon and mu contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain”, when used in reference to an antibody, refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa or lambda based on the amino acid sequence of the constant domains.

The term “peptide linker”, as used herein, generally refers to a short polypeptide sequence connecting two sub-domains of a polypeptide. Non-limiting examples of peptide linkers include flexible linkers comprising glycine-serine repeats, and peptide linkers derived from (a) an interdomain region of a transmembrane protein (e.g., a type I transmembrane protein); (b) a stalk region of a type II C-lectin; or (c) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In some embodiments, the linker connects two or more binding domains (e.g., the TCRβV-binding domain and the CD19-binding domain) and is referred to herein as a peptide linker “PLB” (a peptide linker for binding domains). In some embodiments, the linker connects a binding domain with a half-life extension moiety and is referred to herein as a peptide linker “PLE” (a peptide linker to an extension moiety). In some embodiments, the linker connects a VH region of an scFv and a VL region of an scFv and is referred to herein as a “scFv linker”.

The term “memory T cell”, as used herein, refers to long-lived T cells that remain in the body after a pathogen has been cleared. Memory T cells are highly capable of responding to antigens upon reintroduction. Specific types of effector T cells include central memory T cells (TCM), T memory stem cells (TSCM), and transitional memory T cells (TTM).

The term “operatively linked” and similar phrases (e.g., operably linked, genetically fused), as used herein, refer to the operational linkage of nucleic acid sequences or amino acid sequences placed in functional relationships with each other. For example, a promoter operatively linked to a polynucleotide encoding a polypeptide result in the transcription of the polynucleotide and ultimately the expression of the polypeptide. As another example, an operatively linked peptide is one in which the functional domains are placed with appropriate distance from each other to impart the intended function of each domain.

The term “pharmaceutically acceptable excipient, carrier or diluent”, as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy, 23rd edition, A. Adejare, ed., Academic Press, 2020).

The term “pharmaceutical composition” or “therapeutic composition”, as used herein, refers to a composition capable of being administered to a subject for the treatment of a particular disease or disorder.

The term “polynucleotide” or “nucleic acid”, as used herein, refers to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides can comprise modified nucleotides or bases, and/or their analogs, or any substrate that can be incorporated into a polymer by DNA or RNA polymerase or by a synthetic reaction. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction.

The terms “polypeptide” and “peptide” and “protein”, as used herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art.

The term “population” of cells, as used herein, refers to any number of cells greater than 1, for example, at least about 1×10³ cells, at least about 1×10⁴ cells, at least about 1×10⁵ cells, at least about 1×10⁶ cells, at least about 1×10⁷ cells, at least about 1×10⁸ cells, at least about 1×10⁹ cells, at least about 1×10¹⁰ cells, at least about 1×10¹¹ or more cells. A population of cells may refer to an in vitro population (e.g., a population of cells in culture) or an in vivo population (e.g., a population of cells residing in a particular tissue).

The term “prevent”, as used herein, refers to a pharmaceutical or other intervention regimen for reducing the likelihood of the onset (or recurrence) of a disease, disorder, condition, or associated symptom(s).

The term “single-chain variable fragment (scFv)”, as used herein, refers to a fusion protein of the variable heavy chain (VH) and the variable light chain (VL) of an antibody, connected with a short scFv linker peptide. In some embodiments, the scFv linker is about 5 amino acids to about 30 amino acids in length. In some embodiments, the scFv linker is rich in glycine for flexibility, as well as serine or threonine for solubility, and can either connect the N-terminus of the VH region with the C-terminus of the VL region, or vice versa. scFvs retain the specificity of the original antibody, despite removal of the constant regions and the introduction of the scFv linker. scFvs are, described in e.g., Houston, J. S., Methods in Enzymol. 203 (1991) 46-96).

The term “sequence identity”, as used herein, refers to the percentage of bases or amino acids between two polynucleotide or polypeptide sequences that are the same, when compared and aligned for maximum correspondence over a comparison window or designated region. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. BLAST nucleotide searches can be performed with the NBLAST nucleotide program parameters set, e.g., for score 100, word length-2 to obtain nucleotide sequences homologous to a nucleic acid molecule described herein. BLAST protein searches can be performed with the XBLAST program parameters set, e.g., to score 50, word length-3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Unless noted otherwise, the term “sequence identity” refers to sequence identity as calculated by Clustal Omega® using default parameters.

The term “subject”, as used herein, refers to an “animal” and in particular a “mammal” such as a non-primate (e.g., mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks)) or a primate (e.g., monkeys, baboons, chimpanzees, and human). The term may be used interchangeably with the term “patient” or “individual”. In some embodiments, the subject is a mammal, e.g., a human, diagnosed with a disease or disorder provided herein. In some embodiments, the subject is a mammal, e.g., a human, at risk of developing a disease or disorder provided herein. In some embodiments, the subject is human. The term does not denote a particular age or sex. Thus, individuals of all ages, from newborn to adult, whether male or female, are intended to be covered.

The term “substantially similar,” as used herein, denotes at least about 40% similar, at least about 50% similar, at least about 60% similar, at least about 70% similar, at least about 80% similar, at least about 90% similar, at least about 95% similar, at least about 96% similar, at least about 97% similar, at least about 98% similar or at least about 99% similar.

The term “variable region”, “variable domain”, “V region”, or “V domain”, as used herein, refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or “complementarity determining regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al, Sequences of Proteins of Immunological Interest (5th ed. 1991)).

The complementarity determining regions (CDRs) have been defined by well-known numbering systems. For example, the Kabat Complementarity Determining Regions (CDRs) are based on sequence variability and are the most commonly used (see, e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Rontermann and Diibel, eds., 2d ed. 2010)). The “contact” CDRs are based on an analysis of the available complex crystal structures. Another universal numbering system that has been developed and widely adopted is ImMunoGeneTics (IMGT) Information System® (Lafranc, et al, Dev. Comp. Immunol., 2003, 27 (1): 55-77). IMGT is an integrated information system specializing in immunoglobulins (IG), T-cell receptors (TCR), and major histocompatibility complex (MHC) of human and other vertebrates. An additional numbering system (AHon) has been developed by Honegger and Pluckthun, J. Mol. Biol., 2001, 309:657-70. Correspondence between the numbering system, including, for example, the Kabat numbering and the IMGT unique numbering system, is well known to one skilled in the art (see, e.g., Kabat, supra, Chothia and Lesk, supra; Martin, supra, Lefranc, et al., supra).

The boundaries of a given CDR may vary depending on the scheme used for identification. Thus, unless otherwise specified, the CDRs of a given antibody or region thereof, such as a variable region, should be understood to encompass the complementary determining region as defined by any of the known schemes described herein. In some instances, the scheme for identification of a particular CDR or CDRs is specified, such as the CDR as defined by the Kabat, Chothia, or Contact method. In other cases, the particular amino acid sequence of a CDR is given. In some embodiments, a combination of CDR numbering systems may be used. In such embodiments, CDR sequences of a given binding molecule as determined by multiple numbering systems (e.g., a CDR1 sequence as determined by the Kabat, IMGT, and Chothia numbering systems) are compiled into a single sequence that encompasses the entirety of each of the CDR amino acid ranges in the variable region.

As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody.

The term “variant”, when used in relation to polypeptide, refers to a polypeptide comprising one or more (such as, for example, about 1 to about 25, about 1 to about 20, about 1 to about 15, about 1 to about 10, or about 1 to about 5, including any ranges or subranges therebetween) amino acid sequence substitutions, deletions, and/or additions as compared to a native or unmodified sequence. Variants may be naturally occurring, such as allelic or splice variants, or may be artificially constructed. Polypeptide variants may be prepared from the corresponding nucleic acid molecules encoding the variants.

The term “vector”, as used herein, refers to a substance that is used to carry or introduce a nucleic acid sequence (e.g., a nucleic acid sequence encoding an antibody as described herein) into a host cell. Vectors applicable for use include, for example, plasmids, phage vectors, viral vectors, episomes, and artificial chromosomes. A vector may include sequences that direct autonomous replication in a cell, or may include sequences sufficient to allow integration into host cell DNA. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### 1.2. Multi-Specific Polypeptides

Provided herein are multi-specific polypeptides. In some embodiments, such multi-specific polypeptides are suitable for binding target cells and engaging T cells. In some embodiments, the multi-specific polypeptide comprises a first binding domain and a second binding domain. In some embodiments, the first binding domain specifically binds T cell receptor alpha variable region (TCRαV). In some embodiments, the first binding domain specifically binds T cell receptor beta variable region (TCRβV). In some embodiments, the first binding domain specifically binds CD3. In some embodiments, the second binding domain specifically binds CD19.

In some embodiments, the multi-specific polypeptide further comprises a half-life extension moiety. In some embodiment, the half-life extension moiety comprises a fragment crystallizable (Fc) region.

In some embodiments, the multi-specific polypeptides of the disclosure do not comprise a cytokine. In some embodiments, the cytokine is selected from the group consisting of interleukin-2 (IL-2), interleukin-7 (IL-7), interleukin-10 (IL-10), interleukin-12 (IL-12), interleukin-15 (IL-15), interleukin-18 (IL-18), interleukin-21 (IL-21), interferon gamma, a functional fragment therefore, a functional variant thereof, and a combination thereof.

### 1.2.1. First Binding Domain

In some embodiments, the first binding domain specifically binds a T cell receptor. The T cell receptor (TCR) is expressed on the surface of T cells and is responsible for recognizing antigens bound to major histocompatibility complex (MHC) molecules. When the TCR engages with antigenic peptide and MHC (peptide/MHC), the T cell is activated through a series of biochemical events mediated by associated enzymes, co-receptors, specialized adaptor molecules, and activated or released transcription factors. The TCR is a disulfide-linked membrane-anchored heterodimer normally consisting of the highly variable alpha (α) and beta (β) chains expressed as part of a complex with the invariant CD3 chain molecules. T cells expressing this receptor are referred to as α:β (or αβ) T cells (~95% total T-cells). Each a and B chain is composed of two extracellular domains: a variable region and a constant region. The constant region is proximal to the cell membrane, followed by a transmembrane region and a short cytoplasmic tail, while the variable region binds to the peptide/MHC complex.

In some embodiments, the multi-specific polypeptides used in the methods of expanding T cells and the methods of treatment comprises a first binding domain that specifically binds to a T cell receptor beta variable region (TCRβV) (i.e., a TCRβV-binding domain). In some embodiments, the multi-specific polypeptides used in the methods of expanding T cells and the methods of treatment comprises a first binding domain that specifically binds to a T cell receptor alpha variable region (TCRαV) (i.e., a TCRαV-binding domain).

In some embodiments, the TCRβV-binding domain comprises an immunoglobulin molecule. In some embodiments, the TCRβV-binding domain is an antibody (e.g., a polyclonal or monoclonal antibody). In some embodiments, the TCRβV-binding domain is a chimeric antibody. In some embodiments, the TCRβV-binding domain is a humanized antibody. In some embodiments, the TCRβV-binding domain is a human antibody. In some embodiments, the TCRαV-binding domain comprises an immunoglobulin molecule. In some embodiments, the TCRαV-binding domain is an antibody (e.g., a polyclonal or monoclonal antibody). In some embodiments, the TCRαV-binding domain is a chimeric antibody. In some embodiments, the TCRαV-binding domain is a humanized antibody. In some embodiments, the TCRαV-binding domain is a human antibody.

In some embodiments, the TCRβV-binding domain is an antigen-binding fragment thereof. In some embodiments, the TCRαV-binding domain is an antigen-binding fragment thereof. Non-limiting examples of functional fragments include single-chain Fvs (scFv), Fab fragments, CrossFab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In some embodiments, the TCRβV-binding domain is an scFv. In some embodiments, the TCRαV-binding domain is an scFv. In some embodiments, the TCRβV-binding domain is a Fab. In some embodiments, the TCRαV-binding domain is a Fab. In some embodiments, the TCRβV-binding domain is a CrossFab. In some embodiments, the TCRαV-binding domain is a CrossFab.

In some embodiments, the VH region and the VL region are numbered with reference to the Kabat numbering scheme. In some embodiments, the VH region and the VL region are numbered with reference to the IMGT numbering scheme. In some embodiments, the VH region and the VL region are numbered with reference to the Chothia numbering scheme. In some embodiments, the VH region and the VL region are numbered with reference to the Martin (Enhanced Chothia or AbM) numbering scheme. In some embodiments, the VH region and the VL region are numbered with reference to the Honneger's (AHo) numbering scheme.

In some embodiments, the TCRβV-binding domain comprises an immunoglobulin heavy chain variable (VH) region and an immunoglobulin light chain variable (VL) region. In some embodiments, the VH region comprises a heavy chain CDR1 (HCDR1), a heavy chain CDR2 (HCDR2), and a heavy chain CDR3 (HCDR3). In some embodiments, the VL region comprises a light chain CDR1 (LCDR1), a light chain CDR2 (LCDR2), and a light chain CDR3 (LCDR3).

In some embodiments, the TCRβV-binding domain is a Fab comprising the VH region, the VL region, a first heavy chain constant region (CH1), and a light chain constant region (CL), wherein the C-terminus of the VH region is connected to the N-terminus of the CH1 region (e.g., a polypeptide chain comprising N′-VH-CH1-C′), and the C-terminus of the VL region is connected to the N-terminus of the CL region (e.g., a polypeptide chain comprising N′-VL-CL-C′). In some embodiments, the CH1 region and the CL region of the Fab are connected by a disulfide bond. In some embodiments, the Fab is a charge steering Fab.

In some embodiments, the TCRβV-binding domain is a CrossFab comprising the VH region, the VL region, a first heavy chain constant region (CH1), and a light chain constant region (CL), wherein the C-terminus of the VH region is connected to the N-terminus of the CL region (e.g., a polypeptide chain comprising N′-VH-CL-C′), and the C-terminus of the VL region is connected to the N-terminus of the CH1 region (e.g., a polypeptide chain comprising N′-VL-CH1-C′). In some embodiments, the CH1 region and the CL region of the CrossFab are connected by a disulfide bond. In some embodiments, the CrossFab is a charge steering CrossFab.

Exemplary anti-TCRβV CDRs are provided in Table 1 (Kabat numbering scheme) and Table 2 (IMGT numbering scheme) below.

TABLE 1
TRBV CDRs (Kabat Numberings)

| Target | | | | | | |
|---|---|---|---|---|---|---|
| Description | HCDR1 | HCDR2 | HCDR3 | LCDR1 | LCDR2 | LCDR3 |
| TRBV6-5 | TYYIH | WFFPGS | SYYSYD | KASQNV | SSSHRYS | QQFKSYP |
| | (SEQ ID | GNIKYNE | VLDY | GINVV | (SEQ ID | LT |
| | NO: 1) | KFKG | (SEQ ID | (SEQ ID | NO: 5) | (SEQ ID |
| | | (SEQ ID | NO: 3) | NO: 4) | | NO: 6) |
| | | NO: 2) | | | | |
| TRBV6-5 | LTYIH | WFFPGS | SYYSYD | KASQNV | SSSHRYS | QQFKSYP |
| | (SEQ ID | GNIKYNE | VLDY | GINVV | (SEQ ID | LT |
| | NO: 7) | KFKG | (SEQ ID | (SEQ ID | NO: 5) | (SEQ ID |
| | | (SEQ ID | NO: 3) | NO: 4) | | NO: 6) |
| | | NO: 2) | | | | |
| TRBV6-5 | LTYIH | RVSAGS | SYYSYD | KASQNV | SSSHRYK | QQFKSYP |
| | (SEQ ID | GNVKYN | VLDY | ADRVV | (SEQ ID | LT |
| | NO: 7) | EKFKG | (SEQ ID | (SEQ ID | NO: 10) | (SEQ ID |
| | | (SEQ ID | NO: 3) | NO: 9) | | NO: 6) |
| | | NO: 8) | | | | |
| TRBV6-5 | LYYIH | WFFPGS | SYYSFDV | KASQNV | SSSHRYS | QQFKSYP |
| | | GNIKYNE | LDY (SEQ | GIRVV | | IT (SEQ |
| | (SEQ ID | KFKG | ID NO: | (SEQ ID | (SEQ ID | ID NO: |
| | NO: 1138) | (SEQ ID | 1139) | NO: 1140) | NO: 5) | 1141) |
| | | NO: 2) | | | | |
| TRBV6-5 | TYYIH | WFFPRSG | SYYSFDV | KASQNV | SSSHRYS | QQFKSYP |
| | (SEQ ID | NIKYNEK | LDY (SEQ | GIRVV | (SEQ ID | IT (SEQ |
| | | FKG (SEQ | ID NO: | (SEQ ID | | ID NO: |
| | NO: 1) | ID NO: | 1139) | NO: 1140) | NO: 5) | 1141) |
| | | 1142) | | | | |
| TRBV6-5 | TYYIH | WIFPGSG | SYYSYD | KASQNV | SSSHRYS | QQFKSYP |
| | (SEQ ID | NIKYNEK | VLDY | GNNVA | (SEQ ID | LT (SEQ |
| | NO: 1) | FKG (SEQ | (SEQ ID | (SEQ ID | NO: 5) | ID NO: 6) |
| | | ID NO: | NO: 3) | NO: 1182) | | |
| | | 1180) | | | | |
| TRBV6-5 | TYYIH | WIYPGSG | SYYSYD | KASQNV | SSSHRYS | QQFKSYP |
| | (SEQ ID | NIKYNEK | VLDY | GINVV | (SEQ ID | LT (SEQ |
| | NO: 1) | FKG (SEQ | (SEQ ID | (SEQ ID | NO: 5) | ID NO: 6) |
| | | ID NO: | NO: 3) | NO: 4) | | |
| | | 1181) | | | | |
| TRBV6-5 | TYYIH | WIYPGSG | SYYSYD | KASQNV | SSSHRYS | QQFKSYP |
| | (SEQ ID | NIKYNEK | VLDY | GNNVA | (SEQ ID | LT (SEQ |
| | NO: 1) | FKG (SEQ | (SEQ ID | (SEQ ID | NO: 5) | ID NO: 6) |
| | | ID NO: | NO: 3) | NO: 1182) | | |
| | | 1181) | | | | |
| TRBV6-5 | TYYIH | WIYPGSG | SYYSYD | RASQNV | SSSHRYS | QQFKSYP |
| | (SEQ ID | NIKYNEK | VLDY | GNNVA | (SEQ ID | LT (SEQ |
| | NO: 1) | FKG (SEQ | (SEQ ID | (SEQ ID | NO: 5) | ID NO: 6) |
| | | ID NO: | NO: 3) | NO: 1183) | | |
| | | 1181) | | | | |
| TRBV12- | NFGMH | YISSGSS | RGEGAM | RASSSVN | YTSNLAP | QQFTSSP |
| 3/4 | (SEQ ID | TIYYADT | DY | YIY | (SEQ ID | FT |
| | NO: 11) | LKG | (SEQ ID | (SEQ ID | NO: 15) | (SEQ ID |
| | | (SEQ ID | NO: 13) | NO: 14) | | NO: 16) |
| | | NO: 12) | | | | |
| TRBV12- | NFGMH | YISSGSS | RGEGAM | RASQSV | YTSNLAP | QQYTSSP |
| 3/4 | (SEQ ID | TIYYADT | DY (SEQ | NYIY | (SEQ ID | FT (SEQ |
| | NO: 11) | LKG (SEQ | ID NO: | (SEQ ID | NO: 15) | ID NO: |
| | | ID NO: | 13) | NO: 223) | | 224) |
| | | 12) | | | | |
| TRBV12- | NFGMH | YISSGSS | RGEGAM | RASQSV | YTSNLAP | QQYTSSP |
| 3/4 | (SEQ ID | TIYYADT | DY (SEQ | NYLY | (SEQ ID | FT (SEQ |
| | NO: 11) | LKG (SEQ | ID NO: | (SEQ ID | NO: 15) | ID NO: |
| | | ID NO: | 13) | NO: 225) | | 224) |
| | | 12) | | | | |
| TRBV12- | NYGMH | YISSGSS | RGEGAM | RASQSV | YTSNLAP | QQYTSSP |
| 3/4 | (SEQ ID | TIYYADA | DY (SEQ | NYLY | (SEQ ID | FT (SEQ |
| | NO: 221) | LKG (SEQ | ID NO: | (SEQ ID | NO: 15) | ID NO: |
| | | ID NO: | 13) | NO: 225) | | 224) |
| | | 222) | | | | |
| TRBV20-1 | SAYMH | RIDPATG | SLNWDY | RASKSVS | AASNLES | QQSIEDP |
| | (SEQ ID | KTKYAP | GLDY | ILGTHLI | (SEQ ID | WT (SEQ |
| | NO: 228) | KFQA | (SEQ ID | H (SEQ ID | NO: 232) | ID NO: |
| | | (SEQ ID | NO: 230) | NO: 231) | | 233) |
| | | NO: 229) | | | | |

TABLE 2
TRBV CDRs (IMGT Numberings)

| Target | | | | | | |
|---|---|---|---|---|---|---|
| Description | HCDR1 | HCDR2 | HCDR3 | LCDR1 | LCDR2 | LCDR3 |
| TRBV6-5 | GYSFTTY | FFPGSGN | AGSYYS | QNVGIN | SSS | QQFKSYP |
| | Y | I | YDVLDY | (SEQ ID | | LT |
| | (SEQ ID | (SEQ ID | (SEQ ID | NO: 20) | | (SEQ ID |
| | NO: 17) | NO: 18) | NO: 19) | | | NO: 22) |
| TRBV6-5 | GYSFRLT | FFPGSGN | AGSYYS | QNVGIN | SSS | QQFKSYP |
| | Y | I | YDVLDY | (SEQ ID | | LT |
| | (SEQ ID | (SEQ ID | (SEQ ID | NO: 20) | | (SEQ ID |
| | NO: 23) | NO: 18) | NO: 19) | | | NO: 22) |
| TRBV6-5 | GHDFRL | VSAGSG | AVSYYS | QNVADR | SSS | QQFKSYP |
| | TY | NV | YDVLDY | (SEQ ID | | LT |
| | | | | NO: 27) | | |
| | (SEQ ID | (SEQ ID | (SEQ ID | | | (SEQ ID |
| | NO: 24) | NO: 25) | NO: 26) | | | NO: 22) |
| TRBV6-5 | GYSFTLY | FFPGSGN | AASYYSF | QNVGIR | SSS | QQFKSYP |
| | Y (SEQ ID | I (SEQ ID | DVLDY | (SEQ ID | | IT (SEQ |
| | NO: 1143) | NO: 18) | (SEQ ID | NO: 1145) | | ID NO: |
| | | | NO: 1144) | | | 1146) |
| TRBV6-5 | GYSFTTY | FFPRSGN | AASYYSF | QNVGIR | SSS | QQFKSYP |
| | Y (SEQ ID | I (SEQ ID | DVLDY | (SEQ ID | | IT (SEQ |
| | NO: 17) | NO: 1147) | (SEQ ID | NO: 1145) | | ID NO: |
| | | | NO: 1144) | | | 1146) |
| TRBV6-5 | GYSFTTY | IFPGSGNI | AGSYYS | QNVGNN | SSS | QQFKSYP |
| | Y (SEQ ID | (SEQ ID | YDVLDY | (SEQ ID | | LT (SEQ |
| | NO: 17) | NO: 1184) | (SEQ ID | NO: 1187) | | ID NO: |
| | | | NO: 19) | | | 22) |
| TRBV6-5 | GYSFTTY | IYPGSGN | AGSYYS | QNVGIN | SSS | QQFKSYP |
| | Y (SEQ ID | I (SEQ ID | YDVLDY | (SEQ ID | | LT (SEQ |
| | NO: 17) | NO: 1185) | (SEQ ID | NO: 20) | | ID NO: |
| | | | NO: 19) | | | 22) |
| TRBV6-5 | GYSFTTY | IYPGSGN | ARSYYS | QNVGNN | SSS | QQFKSYP |
| | Y (SEQ ID | I (SEQ ID | YDVLDY | (SEQ ID | | LT (SEQ |
| | NO: 17) | NO: 1185) | (SEQ ID | NO: 1187) | | ID NO: |
| | | | NO: 1186) | | | 22) |
| TRBV12- | GFTFSNF | ISSGSSTI | ARRGEG | SSVNY | YTS | QQFTSSP |
| 3/4 | G | (SEQ ID | AMDY | (SEQ ID | | FT |
| | (SEQ ID | NO: 29) | (SEQ ID | NO: 31) | | (SEQ ID |
| | NO: 28) | | NO: 30) | | | NO: 33) |
| TRBV12- | GFTFSNF | ISSGSSTI | ARRGEG | QSVNY | YTS | QQYTSSP |
| 3/4 | G (SEQ ID | (SEQ ID | AMDY | (SEQ ID | | FT (SEQ |
| | NO: 28) | NO: 29) | (SEQ ID | NO: 227) | | ID NO: |
| | | | NO: 30) | | | 224) |
| TRBV12- | GFTFSNY | ISSGSSTI | ARRGEG | QSVNY | YTS | QQYTSSP |
| 3/4 | G (SEQ ID | (SEQ ID | AMDY | (SEQ ID | | FT (SEQ |
| | NO: 226) | NO: 29) | (SEQ ID | NO: 227) | | ID NO: |
| | | | NO: 30) | | | 224) |
| TRBV20-1 | GFNIKSA | IDPATGK | TRSLNW | KSVSILG | AAS | QQSIEDP |
| | Y (SEQ ID | T (SEQ ID | DYGLDY | THL (SEQ | | WT (SEQ |
| | NO: 234) | NO: 235) | (SEQ ID | ID NO: | | ID NO: |
| | | | NO: 236) | 237) | | 239) |

In some embodiments, the HCDR1 comprises an amino acid sequence of TYYIH (SEQ ID NO: 1), LTYIH (SEQ ID NO: 7), LYYIH (SEQ ID NO: 1138), NFGMH (SEQ ID NO: 11), GYSFTTYY (SEQ ID NO: 17), GYSFRLTY (SEQ ID NO: 23), GHDFRLTY (SEQ ID NO: 24), GYSFTLYY (SEQ ID NO: 1143), GFTFSNFG (SEQ ID NO: 28), NYGMH (SEQ ID NO: 221), GFTFSNYG (SEQ ID NO: 226), SAYMH (SEQ ID NO: 228), or GFNIKSAY (SEQ ID NO: 234). In some embodiments, the HCDR1 comprises 1, 2, 3, or more amino acid substitutions.

In some embodiments, the HCDR2 comprises an amino acid sequence of WFFPGSGNIKYNEKFKG (SEQ ID NO: 2), WIFPGSGNIKYNEKFKG (SEQ ID NO: 1180), WIYPGSGNIKYNEKFKG (SEQ ID NO: 1181), IFPGSGNI (SEQ ID NO: 1184), IYPGSGNI (SEQ ID NO: 1185), RVSAGSGNVKYNEKFKG (SEQ ID NO: 8), WFFPRSGNIKYNEKFKG (SEQ ID NO: 1142), YISSGSSTIYYADTLKG (SEQ ID NO: 12), FFPGSGNI (SEQ ID NO: 18), VSAGSGNV (SEQ ID NO: 25), FFPRSGNI (SEQ ID NO: 1147), ISSGSSTI (SEQ ID NO: 29), YISSGSSTIYYADALKG (SEQ ID NO: 222), RIDPATGKTKYAPKFQA (SEQ ID NO: 229), or IDPATGKT (SEQ ID NO: 235). In some embodiments, the HCDR2 comprises 1, 2, 3, or more amino acid substitutions.

In some embodiments, the HCDR3 comprises an amino acid sequence of SYYSYDVLDY (SEQ ID NO: 3), SYYSFDVLDY (SEQ ID NO: 1139), RGEGAMDY (SEQ ID NO: 13), ARSYYSYDVLDY (SEQ ID NO: 1186), AGSYYSYDVLDY (SEQ ID NO: 19), AVSYYSYDVLDY (SEQ ID NO: 26), AASYYSFDVLDY (SEQ ID NO: 1144), ARRGEGAMDY (SEQ ID NO: 30), SLNWDYGLDY (SEQ ID NO: 230), or TRSLNWDYGLDY (SEQ ID NO: 236). In some embodiments, the HCDR3 comprises 1, 2, 3, or more amino acid substitutions.

In some embodiments, the LCDR1 comprises an amino acid sequence of KASQNVGINVV (SEQ ID NO: 4), KASQNVGNNVA (SEQ ID NO: 1182), RASQNVGNNVA (SEQ ID NO: 1183), QNVGNN (SEQ ID NO: 1187), KASQNVADRVV (SEQ ID NO: 9), KASQNVGIRVV (SEQ ID NO: 1140), RASSSVNYIY (SEQ ID NO: 14), QNVGIN (SEQ ID NO: 20), QNVADR (SEQ ID NO: 27), QNVGIR (SEQ ID NO: 1145), SSVNY (SEQ ID NO: 31), RASQSVNYIY (SEQ ID NO: 223), RASQSVNYLY (SEQ ID NO: 225), QSVNY (SEQ ID NO: 227), RASKSVSILGTHLIH (SEQ ID NO: 231), or KSVSILGTHL (SEQ ID NO: 237). In some embodiments, the LCDR1 comprises 1, 2, 3, or more amino acid substitutions.

In some embodiments, the LCDR2 comprises an amino acid sequence of SSSHRYS (SEQ ID NO: 5), SSSHRYK (SEQ ID NO: 10), YTSNLAP (SEQ ID NO: 15), SSS, YTS, AASNLES (SEQ ID NO: 232), or AAS. In some embodiments, the LCDR2 comprises 1, 2, 3, or more amino acid substitutions.

In some embodiments, the LCDR3 comprises an amino acid sequence of QQFKSYPLT (SEQ ID NO: 6), QQFKSYPIT (SEQ ID NO: 1141), QQFTSSPFT (SEQ ID NO: 16), QQFKSYPLT (SEQ ID NO: 22), QQFKSYPIT (SEQ ID NO: 1146), QQFTSSPFT (SEQ ID NO: 33), QQYTSSPFT (SEQ ID NO: 224), QQSIEDPWT (SEQ ID NO: 233), or QQSIEDPWT (SEQ ID NO: 239). In some embodiments, the LCDR3 comprises 1, 2, 3, or more amino acid substitutions.

In some embodiments, the HCDR1 comprises an amino acid sequence of TYYIH (SEQ ID NO: 1), the HCDR2 comprises an amino acid sequence of WFFPGSGNIKYNEKFKG (SEQ ID NO: 2), the HCDR3 comprises an amino acid sequence of SYYSYDVLDY (SEQ ID NO: 3), the LCDR1 comprises an amino acid sequence of KASQNVGINVV (SEQ ID NO: 4), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of TYYIH (SEQ ID NO: 1), the HCDR2 comprises an amino acid sequence of WIFPGSGNIKYNEKFKG (SEQ ID NO: 1180), the HCDR3 comprises an amino acid sequence of SYYSYDVLDY (SEQ ID NO: 3), the LCDR1 comprises an amino acid sequence of KASQNVGNNVA (SEQ ID NO: 1182), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of GYSFTTYY (SEQ ID NO: 17), the HCDR2 comprises an amino acid sequence of IFPGSGNI (SEQ ID NO: 1184), the HCDR3 comprises an amino acid sequence of AGSYYSYDVLDY (SEQ ID NO: 19), the LCDR1 comprises an amino acid sequence of QNVGNN (SEQ ID NO: 1187), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of TYYIH (SEQ ID NO: 1), the HCDR2 comprises an amino acid sequence of WIYPGSGNIKYNEKFKG (SEQ ID NO: 1181), the HCDR3 comprises an amino acid sequence of SYYSYDVLDY (SEQ ID NO: 3), the LCDR1 comprises an amino acid sequence of KASQNVGINVV (SEQ ID NO: 4), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of GYSFTTYY (SEQ ID NO: 17), the HCDR2 comprises an amino acid sequence of IYPGSGNI (SEQ ID NO: 1185), the HCDR3 comprises an amino acid sequence of AGSYYSYDVLDY (SEQ ID NO: 19), the LCDR1 comprises an amino acid sequence of QNVGIN (SEQ ID NO: 20), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of TYYIH (SEQ ID NO: 1), the HCDR2 comprises an amino acid sequence of WIYPGSGNIKYNEKFKG (SEQ ID NO: 1181), the HCDR3 comprises an amino acid sequence of SYYSYDVLDY (SEQ ID NO: 3), the LCDR1 comprises an amino acid sequence of KASQNVGNNVA (SEQ ID NO: 1182), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of TYYIH (SEQ ID NO: 1), the HCDR2 comprises an amino acid sequence of WIYPGSGNIKYNEKFKG (SEQ ID NO: 1181), the HCDR3 comprises an amino acid sequence of SYYSYDVLDY (SEQ ID NO: 3), the LCDR1 comprises an amino acid sequence of RASQNVGNNVA (SEQ ID NO: 1183), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of GYSFTTYY (SEQ ID NO: 17), the HCDR2 comprises an amino acid sequence of IYPGSGNI (SEQ ID NO: 1185), the HCDR3 comprises an amino acid sequence of ARSYYSYDVLDY (SEQ ID NO: 1186), the LCDR1 comprises an amino acid sequence of QNVGNN (SEQ ID NO: 1187), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of LTYIH (SEQ ID NO: 7), the HCDR2 comprises an amino acid sequence of WFFPGSGNIKYNEKFKG (SEQ ID NO: 2), the HCDR3 comprises an amino acid sequence of SYYSYDVLDY (SEQ ID NO: 3), the LCDR1 comprises an amino acid sequence of KASQNVGINVV (SEQ ID NO: 4), the LCDR2 comprises an amino acid sequence of SSSHRYS (SEQ ID NO: 5), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of LTYIH (SEQ ID NO: 7), the HCDR2 comprises an amino acid sequence of RVSAGSGNVKYNEKFKG (SEQ ID NO: 8), the HCDR3 comprises an amino acid sequence of SYYSYDVLDY (SEQ ID NO: 3), the LCDR1 comprises an amino acid sequence of KASQNVADRVV (SEQ ID NO: 9), the LCDR2 comprises an amino acid sequence of SSSHRYK (SEQ ID NO: 10), and the LCDR3 comprises an amino acid sequence of QQFKSYPLT (SEQ ID NO: 6). In some embodiments, the CDRs comprise 1, 2, 3, or more amino acid substitutions.

In some embodiments, the HCDR1 comprises an amino acid sequence of LYYIH (SEQ ID NO: 1138), the HCDR2 comprises an amino acid sequence of WFFPGSGNIKYNEKFKG (SEQ ID NO: 2), the HCDR3 comprises an amino acid sequence of SYYSFDVLDY (SEQ ID NO: 1139), the LCDR1 comprises an amino acid sequence of KASQNVGIRVV (SEQ ID NO: 1140), the LCDR2 comprises an amino acid sequence of SSSHRYS (SEQ ID NO: 5), and the LCDR3 comprises an amino acid sequence of QQFKSYPIT (SEQ ID NO: 1141). In some embodiments, the CDRs comprise 1, 2, 3, or more amino acid substitutions.

In some embodiments, the HCDR1 comprises an amino acid sequence of TYYIH (SEQ ID NO: 1), the HCDR2 comprises an amino acid sequence of WFFPRSGNIKYNEKFKG (SEQ ID NO: 1142), the HCDR3 comprises an amino acid sequence of SYYSFDVLDY (SEQ ID NO: 1139), the LCDR1 comprises an amino acid sequence of KASQNVGIRVV (SEQ ID NO: 1140), the LCDR2 comprises an amino acid sequence of SSSHRYS (SEQ ID NO: 5), and the LCDR3 comprises an amino acid sequence of QQFKSYPIT (SEQ ID NO: 1141). In some embodiments, the CDRs comprise 1, 2, 3, or more amino acid substitutions.

In some embodiments, the HCDR1 comprises an amino acid sequence of GYSFTTYY (SEQ ID NO: 17), the HCDR2 comprises an amino acid sequence of FFPGSGNI (SEQ ID NO: 18), the HCDR3 comprises an amino acid sequence of AGSYYSYDVLDY (SEQ ID NO: 19), the LCDR1 comprises an amino acid sequence of QNVGIN (SEQ ID NO: 20), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of GYSFRLTY (SEQ ID NO: 23), the HCDR2 comprises an amino acid sequence of FFPGSGNI (SEQ ID NO: 18), the HCDR3 comprises an amino acid sequence of AGSYYSYDVLDY (SEQ ID NO: 19), the LCDR1 comprises an amino acid sequence of QNVGIN (SEQ ID NO: 20), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of GHDFRLTY (SEQ ID NO: 24), the HCDR2 comprises an amino acid sequence of VSAGSGNV (SEQ ID NO: 25), the HCDR3 comprises an amino acid sequence of AVSYYSYDVLDY (SEQ ID NO: 26), the LCDR1 comprises an amino acid sequence of QNVADR (SEQ ID NO: 27), the LCDR2 comprises an amino acid sequence of SSS, and the LCDR3 comprises an amino acid sequence of QQFKSYPLT (SEQ ID NO: 22). In some embodiments, the CDRs comprise 1, 2, 3, or more amino acid substitutions.

In some embodiments, the HCDR1 comprises an amino acid sequence of GYSFTLYY (SEQ ID NO: 1143), the HCDR2 comprises an amino acid sequence of FFPGSGNI (SEQ ID NO: 18), the HCDR3 comprises an amino acid sequence of AASYYSFDVLDY (SEQ ID NO: 1144), the LCDR1 comprises an amino acid sequence of QNVGIR (SEQ ID NO: 1145), the LCDR2 comprises an amino acid sequence of SSS, and the LCDR3 comprises an amino acid sequence of QQFKSYPIT (SEQ ID NO: 1146). In some embodiments, the CDRs comprise 1, 2, 3, or more amino acid substitutions.

In some embodiments, the HCDR1 comprises an amino acid sequence of GYSFTTYY (SEQ ID NO: 17), the HCDR2 comprises an amino acid sequence of FFPRSGNI (SEQ ID NO: 1147), the HCDR3 comprises an amino acid sequence of AASYYSFDVLDY (SEQ ID NO: 1144), the LCDR1 comprises an amino acid sequence of QNVGIR (SEQ ID NO: 1145), the LCDR2 comprises an amino acid sequence of SSS, and the LCDR3 comprises an amino acid sequence of QQFKSYPIT (SEQ ID NO: 1146). In some embodiments, the CDRs comprise 1, 2, 3, or more amino acid substitutions.

In some embodiments, the HCDR1 comprises an amino acid sequence of NFGMH (SEQ ID NO: 11), the HCDR2 comprises an amino acid sequence of YISSGSSTIYYADTLKG (SEQ ID NO: 12), the HCDR3 comprises an amino acid sequence of RGEGAMDY (SEQ ID NO: 13), the LCDR1 comprises an amino acid sequence of RASSSVNYIY (SEQ ID NO: 14), the LCDR2 comprises an amino acid sequence of YTSNLAP (SEQ ID NO: 15), and the LCDR3 comprises an amino acid sequence of QQFTSSPFT (SEQ ID NO: 16). In some embodiments, the CDRs comprise 1, 2, 3, or more amino acid substitutions.

In some embodiments, the HCDR 1 comprises an amino acid sequence of NFGMH (SEQ ID NO: 11), the HCDR2 comprises an amino acid sequence of YISSGSSTIYYADTLKG (SEQ ID NO: 12), the HCDR3 comprises an amino acid sequence of RGEGAMDY (SEQ ID NO: 13), the LCDR1 comprises an amino acid sequence of RASQSVNYIY (SEQ ID NO: 223), the LCDR2 comprises an amino acid sequence of YTSNLAP (SEQ ID NO: 15), and the LCDR3 comprises an amino acid sequence of QQYTSSPFT (SEQ ID NO: 224). In some embodiments, the CDRs comprise 1, 2, 3, or more amino acid substitutions.

In some embodiments, the HCDR 1 comprises an amino acid sequence of NFGMH (SEQ ID NO: 11), the HCDR2 comprises an amino acid sequence of YISSGSSTIYYADTLKG (SEQ ID NO: 12), the HCDR3 comprises an amino acid sequence of RGEGAMDY (SEQ ID NO: 13), the LCDR1 comprises an amino acid sequence of RASQSVNYLY (SEQ ID NO: 225), the LCDR2 comprises an amino acid sequence of YTSNLAP (SEQ ID NO: 15), and the LCDR3 comprises an amino acid sequence of QQYTSSPFT (SEQ ID NO: 224). In some embodiments, the CDRs comprise 1, 2, 3, or more amino acid substitutions.

In some embodiments, the HCDR1 comprises an amino acid sequence of NYGMH (SEQ ID NO: 221), the HCDR2 comprises an amino acid sequence of YISSGSSTIYYADALKG (SEQ ID NO: 222), the HCDR3 comprises an amino acid sequence of RGEGAMDY (SEQ ID NO: 13), the LCDR1 comprises an amino acid sequence of RASQSVNYLY (SEQ ID NO: 225), the LCDR2 comprises an amino acid sequence of YTSNLAP (SEQ ID NO: 15), and the LCDR3 comprises an amino acid sequence of QQYTSSPFT (SEQ ID NO: 224). In some embodiments, the CDRs comprise 1, 2, 3, or more amino acid substitutions.

In some embodiments, the HCDR1 comprises an amino acid sequence of SAYMH (SEQ ID NO: 228), the HCDR2 comprises an amino acid sequence of RIDPATGKTKYAPKFQA (SEQ ID NO: 229), the HCDR3 comprises an amino acid sequence of SLNWDYGLDY (SEQ ID NO: 230), the LCDR1 comprises an amino acid sequence of RASKSVSILGTHLIH (SEQ ID NO: 231), the LCDR2 comprises an amino acid sequence of AASNLES (SEQ ID NO: 232), and the LCDR3 comprises an amino acid sequence of QQSIEDPWT (SEQ ID NO: 233). In some embodiments, the CDRs comprise 1, 2, 3, or more amino acid substitutions.

In some embodiments, the HCDR1 comprises an amino acid sequence of GFTFSNFG (SEQ ID NO: 28), the HCDR2 comprises an amino acid sequence of ISSGSSTI (SEQ ID NO: 29), the HCDR3 comprises an amino acid sequence of ARRGEGAMDY (SEQ ID NO: 30), the LCDR1 comprises an amino acid sequence of SSVNY (SEQ ID NO: 31), the LCDR2 comprises an amino acid sequence of YTS, and the LCDR3 comprises an amino acid sequence of QQFTSSPFT (SEQ ID NO: 33). In some embodiments, the CDRs comprise 1, 2, 3, or more amino acid substitutions.

In some embodiments, the HCDR1 comprises an amino acid sequence of GFTFSNFG (SEQ ID NO: 28), the HCDR2 comprises an amino acid sequence of ISSGSSTI (SEQ ID NO: 29), the HCDR3 comprises an amino acid sequence of ARRGEGAMDY (SEQ ID NO: 30), the LCDR1 comprises an amino acid sequence of QSVNY (SEQ ID NO: 227), the LCDR2 comprises an amino acid sequence of YTS, and the LCDR3 comprises an amino acid sequence of QQYTSSPFT (SEQ ID NO: 224). In some embodiments, the CDRs comprise 1, 2, 3, or more amino acid substitutions.

In some embodiments, the HCDR1 comprises an amino acid sequence of GFTFSNYG (SEQ ID NO: 226), the HCDR2 comprises an amino acid sequence of ISSGSSTI (SEQ ID NO: 29), the HCDR3 comprises an amino acid sequence of ARRGEGAMDY (SEQ ID NO: 30), the LCDR1 comprises an amino acid sequence of QSVNY (SEQ ID NO: 227), the LCDR2 comprises an amino acid sequence of YTS, and the LCDR3 comprises an amino acid sequence of QQYTSSPFT (SEQ ID NO: 224). In some embodiments, the CDRs comprise 1, 2, 3, or more amino acid substitutions.

In some embodiments, the HCDR1 comprises an amino acid sequence of GFNIKSAY (SEQ ID NO: 234), the HCDR2 comprises an amino acid sequence of IDPATGKT (SEQ ID NO: 235), the HCDR3 comprises an amino acid sequence of TRSLNWDYGLDY (SEQ ID NO: 236), the LCDR1 comprises an amino acid sequence of KSVSILGTHL (SEQ ID NO: 237), the LCDR2 comprises an amino acid sequence of AAS, and the LCDR3 comprises an amino acid sequence of QQSIEDPWT (SEQ ID NO: 239). In some embodiments, the CDRs comprise 1, 2, 3, or more amino acid substitutions.

In some embodiments, the VH region of the TCRβV-binding domain is the VH region of an IgA, IgD, IgE, IgG, or IgM. In some embodiments, the VH region of the TCRβV-binding domain is the VH region of a subclass of an IgA, IgD, IgE, IgG, or IgM, such as, e.g., IgG1, IgG2, IgG3, IgG4, IgA1, and IgA2. In some embodiments, the VL region of the TCRβV-binding domain is the VL region of a kappa light chain. In some embodiments, the VL region of the TCRβV-binding domain is the VL region of a lambda light chain.

Exemplary anti-TCRβV VHs and VLs are provided in Table 3. TRBV VH and VL

TABLE 3
TRBV VH and VL

| Target | VH | VL |
|---|---|---|
| Description | | |
| TRBV6-5 | QVQLVQSGAEVKKPGSSVKVSCK | DIQMTQSPSFLSASVGDRVTITCK |
| | ASGYSFTTYYIHWVRQAPGQGLE | ASQNVGINVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPL |
| | VYYCAGSYYSYDVLDYWGQGTT | TFGQGTKLEIK (SEQ ID NO: 35) |
| | VTVSS (SEQ ID NO: 34) | |
| TRBV6-5 | QVQLVQSGAEVKKPGASVKVSC | DIQMTQSPSFLSASVGDRVTITCK |
| | KASGYSFTTYYIHWVRQAPGQGL | ASQNVGINVVWHQQKPGKAPKA |
| | EWMGWFFPGSGNIKYNEKFKGR | LIYSSSHRYSGVPSRFSGSGSGTEF |
| | VTITADTSTSTAYMELSSLRSEDT | TLTISSLQPEDFATYFCQQFKSYPL |
| | AVYYCAGSYYSYDVLDYWGQGT | TFGQGTKLEIK (SEQ ID NO: 35) |
| | LVTVSS (SEQ ID NO: 36) | |
| TRBV6-5 | QVQLVQSGAEVKKPGSSVKVSCK | DIQMTQSPSFLSASVGDRVTITCK |
| | ASGYSFRLTYIHWVRQAPGQGLE | ASQNVGINVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPL |
| | VYYCAGSYYSYDVLDYWGQGTT | TFGQGTKLEIK (SEQ ID NO: 35) |
| | VTVSS (SEQ ID NO: 37) | |
| TRBV6-5 | QVQLVQSGAEVKKPGSSVKVSCK | DIQMTQSPSFLSASVGDRVTITCK |
| | ASGHDFRLTYIHWVRQAPGQGLE | ASQNVADRVVWHQQKPGKAPKA |
| | WMGRVSAGSGNVKYNEKFKGRV | LIYSSSHRYKGVPSRFSGSGSGTEF |
| | TITADTSTSTAYMELSSLRSEDTA | TLTISSLQPEDFATYFCQQFKSYPL |
| | VYYCAVSYYSYDVLDYWGQGTT | TFGQGTKLEIK (SEQ ID NO: 39) |
| | VTVSS (SEQ ID NO: 38) | |
| TRBV6-5 | QVQLVQSGAEVKKPGSSVKVSCK | DIQMTQSPSFLSASVGDRVTITCK |
| | ASGYSFTTYYIHWVRQAPGQGLE | ASQNVGINVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPL |
| | VYYCAGSYYSYDVLDYWGQGTT | TFGQGTKLEIK (SEQ ID NO: 1001) |
| | VTVSS (SEQ ID NO: 1009) | |
| TRBV6-5 | QVQLVQSGAEVKKPGASVKVSC | DIQMTQSPSFLSASVGDRVTITCK |
| | KASGYSFTTYYIHWVRQAPGQGL | ASQNVGINVVWHQQKPGKAPKA |
| | EWMGWFFPGSGNIKYNEKFKGR | LIYSSSHRYSGVPSRFSGSGSGTEF |
| | VTITADTSTSTAYMELSSLRSEDT | TLTISSLQPEDFATYFCQQFKSYPL |
| | AVYYCAGSYYSYDVLDYWGQGT | TFGQGTKLEIK (SEQ ID NO: 1002) |
| | LVTVSS (SEQ ID NO: 1010) | |
| TRBV6-5 | QVQLVQSGAEVKKPGASVKVSC | DIQMTQSPSFLSASVGDRVTITCK |
| | KASGYSFTTYYIHWVRQAPGQGL | ASQNVGNNVAWYQQKPGKAPKL |
| | EWMGWIFPGSGNIKYNEKFK GRV | LIYSSSHRYSGVPSRFSGSGSGTEF |
| | TITADTSTSTAYMELSSLRSEDTA | TLTISSLQPEDFATYFCQQFKSYPL |
| | VYYCAGSYYSYDVLDYWGQGTL | TFGQGTKLEIK (SEQ ID NO: 1003) |
| | VTVSS (SEQ ID NO: 1011) | |
| TRBV6-5 | QVQLVQSGAEVKKPGASVKVSC | DIQMTQSPSSLSASVGDRVTITCK |
| | KASGYSFTTYYIHWVRQAPGQGL | ASQNVGINVVWYQQKPGKAPKL |
| | EWMGWIYPGSGNIKYNEKFK GR | LIYSSSHRYSGVPSRFSGSGSGTEF |
| | VTITADTSTSTAYMELSSLRSEDT | TLTISSLQPEDFATYYCQQFKSYP |
| | AVYYCAGSYYSYDVLDYWGQGT | LTFGQGTKLEIK (SEQ ID NO: |
| | LVTVSS (SEQ ID NO: 1012) | 1004) |
| TRBV6-5 | QVQLVQSGAEVKKPGASVKVSC | DIQMTQSPSSLSASVGDRVTITCK |
| | KASGYSFTTYYIHWVRQAPGQGL | ASQNVGNNVAWYQQKPGKAPKL |
| | EWMGWIYPGSGNIKYNEKFK GR | LIYSSSHRYSGVPSRFSGSGSGTEF |
| | VTITADTSTSTAYMELSSLRSEDT | TLTISSLQPEDFATYYCQQFKSYP |
| | AVYYCARSYYSYDVLDYWGQGT | LTFGQGTKLEIK (SEQ ID NO: |
| | LVTVSS (SEQ ID NO: 1013) | 1005) |
| TRBV6-5 | QVQLVQSGAEVKKPGASVKVSC | DIQMTQSPSSLSASVGDRVTITCR |
| | KASGYSFTTYYIHWVRQAPGQGL | ASQNVGNNVAWYQQKPGKAPKL |
| | EWMGWIYPGSGNIKYNEKFK GR | LIYSSSHRYSGVPSRFSGSGSGTEF |
| | VTITADTSTSTAYMELSSLRSEDT | TLTISSLQPEDFATYYCQQFKSYP |
| | AVYYCARSYYSYDVLDYWGQGT | LTFGQGTKLEIK (SEQ ID NO: |
| | LVTVSS (SEQ ID NO: 1014) | 1006) |
| TRBV6-5 | QVQLVQSGAEVKKPGSSVKVSCK | DIQMTQSPSFLSASVGDRVTITCK |
| | ASGYSFRLTYIHWVRQAPGQGLE | ASQNVGINVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPL |
| | VYYCAGSYYSYDVLDYWGQGTT | TFGQGTKLEIK (SEQ ID NO: 1007) |
| | VTVSS (SEQ ID NO: 1015) | |
| TRBV6-5 | QVQLVQSGAEVKKPGSSVKVSCK | DIQMTQSPSFLSASVGDRVTITCK |
| | ASGHDFRLTYIHWVRQAPGQGLE | ASQNVADRVVWHQQKPGKAPKA |
| | WMGRVSAGSGNVKYNEKFKGRV | LIYSSSHRYKGVPSRFSGSGSGTEF |
| | TITADTSTSTAYMELSSLRSEDTA | TLTISSLQPEDFATYFCQQFKSYPL |
| | VYYCAVSYYSYDVLDYWGQGTT | TFGQGTKLEIK (SEQ ID NO: 1008) |
| | VTVSS (SEQ ID NO: 1016) | |
| TRBV6-5 | QVQLVQSGAEVKKPGSSVKVSCK | DIQMTQSPSFLSASVGDRVTITCK |
| | ASGYSFTLYYIHWVRQAPGQGLE | ASQNVGIRVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPI |
| | VYYCAASYYSFDVLDYWGQGTT | TFGQGTKLEIK (SEQ ID NO: 1149) |
| | VTVSS (SEQ ID NO: 1148) | |
| TRBV6-5 | QVQLVQSGAEVKKPGSSVKVSCK | DIQMTQSPSFLSASVGDRVTITCK |
| | ASGYSFTTYYIHWVRQAPGQGLE | ASQNVGIRVVWHQQKPGKAPKA |
| | WMGWFFPRSGNIKYNEKFKGRV | LIYSSSHRYSGVPSRFSGSGSGTEF |
| | TITADTSTSTAYMELSSLRSEDTA | TLTISSLQPEDFATYFCQQFKSYPI |
| | VYYCAASYYSFDVLDYWGQGTT | TFGQGTKLEIK (SEQ ID NO: 1149) |
| | VTVSS (SEQ ID NO: 1150) | |
| TRBV6-5 | QVQLVQSGAEVKKPGSSVKVSCK | DIQMTQSPSFLSASVGDRVTITCK |
| | ASGYSFTLYYIHWVRQAPGQCLE | ASQNVGIRVVWHQQKPGKAPKA |
| | WMGWFFPGSGNIKYNEKFKGRV | LIYSSSHRYSGVPSRFSGSGSGTEF |
| | TITADTSTSTAYMELSSLRSEDTA | TLTISSLQPEDFATYFCQQFKSYPI |
| | VYYCAASYYSFDVLDYWGQGTT | TFGCGTKLEIK (SEQ ID NO: 1192) |
| | VTVSS (SEQ ID NO: 1190) | |
| TRBV6-5 | QVQLVQSGAEVKKPGSSVKVSCK | DIQMTQSPSFLSASVGDRVTITCK |
| | ASGYSFTTYYIHWVRQAPGQCLE | ASQNVGIRVVWHQQKPGKAPKA |
| | WMGWFFPRSGNIKYNEKFKGRV | LIYSSSHRYSGVPSRFSGSGSGTEF |
| | TITADTSTSTAYMELSSLRSEDTA | TLTISSLQPEDFATYFCQQFKSYPI |
| | VYYCAASYYSFDVLDYWGQGTT | TFGCGTKLEIK (SEQ ID NO: 1192) |
| | VTVSS (SEQ ID NO: 1191) | |
| TRBV12-3/4 | EVOLVESGGGLVQPGGSLRLSCA | DNQLTQSPSFLSASVGDRVTITCR |
| | ASGFTFSNFGMHWVRQAPGKGL | ASSSVNYIYWYQQKPGKAPKLLI |
| | EWVSYISSGSSTIYYADTLKGRFTI | YYTSNLAPGVPSRFSGSGSGNEYT |
| | SRDNSKNTLYLQMNSLRAEDTAV | LTISSLQPEDFATYYCQQFTSSPFT |
| | YYCARRGEGAMDYWGQGTTVT | FGQGTKLEIK (SEQ ID NO: 41) |
| | VSS (SEQ ID NO: 40) | |
| TRBV12-3/4 | EVOLVESGGGLVQPGGSLRLSCA | DNQLTQSPSFLSASVGDRVTITCR |
| | ASGFTFSNFGMHWVRQAPGKGL | ASSSVNYIYWYQQKPGKAPKLLI |
| | EWVSYISSGSSTIYYADTLKGRFTI | YYTSNLAPGVPSRFSGSGSGNEYT |
| | SRDNSKNTLYLQMNSLRAEDTAV | LTISSLQPEDFATYYCQQFTSSPFT |
| | YYCARRGEGAMDYWGQGTLVT | FGQGTKLEIK (SEQ ID NO: 41) |
| | VSS (SEQ ID NO: 42) | |
| TRBV12-3/4 | EVOLVESGGGLVQPGGSLRLSCA | DNQLTQSPSFLSASVGDRVTITCR |
| | ASGFTFSNFGMHWVRQAPGKGL | ASSSVNYIYWYQQKPGKAPKLLI |
| | EWVSYISSGSSTIYYADTLKGRFTI | YYTSNLAPGVPSRFSGSGSGNEYT |
| | SRDNSKNTLYLQMNSLRAEDTAV | LTISSLQPEDFATYYCQQFTSSPFT |
| | YYCARRGEGAMDYWGQGTTVT | FGQGTKLEIK (SEQ ID NO: 1087) |
| | VSS (SEQ ID NO: 1093) | |
| TRBV12-3/4 | EVOLVESGGGLVQPGGSLRLSCA | DNQLTQSPSFLSASVGDRVTITCR |
| | ASGFTFSNFGMHWVRQAPGKGL | ASSSVNYIYWYQQKPGKAPKLLI |
| | EWVSYISSGSSTIYYADTLKGRFTI | YYTSNLAPGVPSRFSGSGSGNEYT |
| | SRDNSKNTLYLQMNSLRAEDTAV | LTISSLQPEDFATYYCQQFTSSPFT |
| | YYCARRGEGAMDYWGQGTLVT | FGQGTKLEIK (SEQ ID NO: 1088) |
| | VSS (SEQ ID NO: 1094) | |
| TRBV12-3/4 | EVOLVESGGGLVQPGGSLRLSCA | DIQLTQSPSFLSASVGDRVTITCRA |
| | ASGFTFSNFGMHWVRQAPGKGL | SSSVNYIYWYQQKPGKAPKLLIY |
| | EWVSYISSGSSTIYYADTLKGRFTI | YTSNLAPGVPSRFSGSGSGTEFTL |
| | SRDNSKNTLYLQMNSLRAEDTAV | TISSLQPEDFATYYCQQYTSSPFTF |
| | YYCARRGEGAMDYWGQGTLVT | GQGTKLEIK (SEQ ID NO: 1089) |
| | VSS (SEQ ID NO: 1095) | |
| TRBV12-3/4 | EVOLVESGGGLVQPGGSLRLSCA | DIQLTQSPSFLSASVGDRVTITCRA |
| | ASGFTFSNFGMHWVRQAPGKGL | SQSVNYIYWYQQKPGKAPKLLIY |
| | EWVSYISSGSSTIYYADTLKGRFTI | YTSNLAPGVPSRFSGSGSGTEFTL |
| | SRDNSKNTLYLQMNSLRAEDTAV | |
| | YYCARRGEGAMDYWGQGTLVT | TISSLQPEDFATYYCQQYTSSPFTF |
| | VSS (SEQ ID NO: 1096) | GQGTKLEIK (SEQ ID NO: 1090) |
| TRBV12-3/4 | EVOLVESGGGLVQPGGSLRLSCA | DIQLTQSPSFLSASVGDRVTITCRA |
| | ASGFTFSNFGMHWVRQAPGKGL | SQSVNYLYWYQQKPGKAPKLLIY |
| | EWVSYISSGSSTIYYADTLKGRFTI | YTSNLAPGVPSRFSGSGSGTEFTL |
| | SRDNSKNTLYLQMNSLRAEDTAV | TISSLQPEDFATYYCQQYTSSPFTF |
| | YYCARRGEGAMDYWGQGTLVT | GQGTKLEIK (SEQ ID NO: 1091) |
| | VSS (SEQ ID NO: 1097) | |
| TRBV12-3/4 | EVOLVESGGGLVQPGGSLRLSCA | DIQLTQSPSFLSASVGDRVTITCRA |
| | ASGFTFSNYGMHWVRQAPGKGL | SQSVNYLYWYQQKPGKAPKLLIY |
| | EWVSYISSGSSTIYYADALKGRFT | YTSNLAPGVPSRFSGSGSGTEFTL |
| | ISRDNSKNTLYLQMNSLRAEDTA | TISSLQPEDFATYYCQQYTSSPFTF |
| | VYYCARRGEGAMDYWGQGTLV | GQGTKLEIK (SEQ ID NO: 1092) |
| | TVSS (SEQ ID NO: 1098) | |
| TRBV20-1 | EVQLQQSVADLVRPGASLKLSCT | DIVLTQSPASLAVSLGQRATISCR |
| | ASGFNIKSA YMHWVIQRPDQGPE | ASKSVSILGTHLIHWYQQKPGQPP |
| | CLGRIDPATGKTKYAPKFQAKATI | KLLIYAASNLESGVPARFSGSGSE |
| | TADTSSNTAYLQLSSLTSEDTAIY | TVFTLNIHPVEEEDAATYFCQQSI |
| | YCTRSLNWDYGLDYWGQGTSVT | EDPWTFGGGTKLGIK |
| | VSS (SEQ ID NO: 240) | (SEQ ID NO: 241) |

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, 1150, 1190, and 1191. In some embodiments, the VH region comprises or consists of an amino acid sequence of any one of SEQ ID NOs: 34, 36, 37, 38, 40, 42, 240, 1009-1016, 1093-1098, 1148, 1150, 1190, and 1191.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, and 1150 and comprises a cysteine residue at amino acid position 44. In some embodiments, the VH region comprises or consists of an amino acid sequence of any one of SEQ ID NOs: 34, 36, 37, 38, 40, 42, 240, 1009-1016, 1093-1098, 1148, and 1150 and comprises a cysteine substitution at amino acid position 44.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, and 1150 and comprises a cysteine residue at amino acid position 100. In some embodiments, the VH region comprises or consists of an amino acid sequence of any one of SEQ ID NOs: 34, 36, 37, 38, 40, 42, 240, 1009-1016, 1093-1098, 1148, and 1150 and comprises a cysteine substitution at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, and 1150 and comprises a cysteine residue at amino acid position 100B. In some embodiments, the VH region comprises or consists of an amino acid sequence of any one of SEQ ID NOs: 34, 36, 37, 38, 40, 42, 240, 1009-1016, 1093-1098, 1148, and 1150 and comprises a cysteine substitution at amino acid position 100B.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, and 1150 and comprises a cysteine residue at amino acid position 101. In some embodiments, the VH region comprises or consists of an amino acid sequence of any one of SEQ ID NOs: 34, 36, 37, 38, 40, 42, 240, 1009-1016, 1093-1098, 1148, and 1150 and comprises a cysteine substitution at amino acid position 101.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 34, 36, 37, 38, 40, 42, 240, 1009-1016, 1093-1098, 1148, and 1150 and comprises a cysteine residue at amino acid position 105. In some embodiments, the VH region comprises or consists of an amino acid sequence of any one of SEQ ID NOs: 34, 36, 37, 38, 40, 42, 240, 1009-1016, 1093-1098, 1148, and 1150 and comprises a cysteine substitution at amino acid position 105.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, 1087-1092, 1149, and 1192. In some embodiments, the VL region comprises or consists of an amino acid sequence of any one of SEQ ID NOs: 35, 39, 41, 241, 1001-1008, 1087-1092, 1149, and 1192.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, 1087-1092, and 1149 and comprises a cysteine residue at amino acid position 100. In some embodiments, the VL region comprises or consists of an amino acid sequence of any one of SEQ ID NOs: 35, 39, 41, 241, 1001-1008, 1087-1092, and 1149 and comprises a cysteine substitution at amino acid position 100.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, 1087-1092, and 1149 and comprises a cysteine residue at amino acid position 49. In some embodiments, the VL region comprises or consists of an amino acid sequence of any one of SEQ ID NOs: 35, 39, 41, 241, 1001-1008, 1087-1092, and 1149 and comprises a cysteine substitution at amino acid position 49.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, and 1087-1092, and 1149 and comprises a cysteine residue at amino acid position 50. In some embodiments, the VL region comprises or consists of an amino acid sequence of any one of SEQ ID NOs: 35, 39, 41, 241, 1001-1008, 1087-1092, and 1149 and comprises a cysteine substitution at amino acid position 50.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, and 1087-1092, and 1149 and comprises a cysteine residue at amino acid position 46. In some embodiments, the VL region comprises or consists of an amino acid sequence of any one of SEQ ID NOs: 35, 39, 41, 241, 1001-1008, 1087-1092, and 1149 and comprises a cysteine substitution at amino acid position 46.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 35, 39, 41, 241, 1001-1008, 1087-1092, and 1149 and comprises a cysteine residue at amino acid position 43. In some embodiments, the VL region comprises or consists of an amino acid sequence of any one of SEQ ID NOs: 35, 39, 41, 241, 1001-1008, 1087-1092, and 1149 and comprises a cysteine substitution at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 34 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 36 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 37 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 35 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 38 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 39 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1148 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1150 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1149 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1190, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1192.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1191, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1192.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 40 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 42 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 41 and comprises a cysteine residue at amino acid position 43.

In some embodiments, the VH region comprises or consists of an amino acid sequence of SEQ ID NO: 34, and the VL region comprises or consists of an amino acid sequence of SEQ ID NO: 35.

In some embodiments, the VH region comprises an amino acid sequence of SEQ ID NO: 34 with a cysteine substitution at amino acid position 44, and the VL region comprises an amino acid sequence of SEQ ID NO: 35 with a cysteine substitution at amino acid position 100.

In some embodiments, the VH region comprises an amino acid sequence of SEQ ID NO: 34 with a cysteine substitution at amino acid position 100B, and the VL region comprises an amino acid sequence of SEQ ID NO: 35 with a cysteine substitution at amino acid position 49.

In some embodiments, the VH region comprises an amino acid sequence of SEQ ID NO: 34 with a cysteine substitution at amino acid position 100, and the VL region comprises an amino acid sequence of SEQ ID NO: 35 with a cysteine substitution at amino acid position 50.

In some embodiments, the VH region comprises an amino acid sequence of SEQ ID NO: 34 with a cysteine substitution at amino acid position 101, and the VL region comprises an amino acid sequence of SEQ ID NO: 35 with a cysteine substitution at amino acid position 46.

In some embodiments, the VH region comprises an amino acid sequence of SEQ ID NO: 34 with a cysteine substitution at amino acid position 105, and the VL region comprises an amino acid sequence of SEQ ID NO: 35 with a cysteine substitution at amino acid position 43.

In some embodiments, the VH region comprises or consists of an amino acid sequence of SEQ ID NO: 36, and the VL region comprises or consists of an amino acid sequence of SEQ ID NO: 35.

In some embodiments, the VH region comprises an amino acid sequence of SEQ ID NO: 36 with a cysteine substitution at amino acid position 44, and the VL region comprises an amino acid sequence of SEQ ID NO: 35 with a cysteine substitution at amino acid position 100.

In some embodiments, the VH region comprises an amino acid sequence of SEQ ID NO: 36 with a cysteine substitution at amino acid position 100B, and the VL region comprises an amino acid sequence of SEQ ID NO: 35 with a cysteine substitution at amino acid position 49.

In some embodiments, the VH region comprises an amino acid sequence of SEQ ID NO: 36 with a cysteine substitution at amino acid position 100, and the VL region comprises an amino acid sequence of SEQ ID NO: 35 with a cysteine substitution at amino acid position 50.

In some embodiments, the VH region comprises an amino acid sequence of SEQ ID NO: 36 with a cysteine substitution at amino acid position 101, and the VL region comprises an amino acid sequence of SEQ ID NO: 35 with a cysteine substitution at amino acid position 46.

In some embodiments, the VH region comprises an amino acid sequence of SEQ ID NO: 36 with a cysteine substitution at amino acid position 105, and the VL region comprises an amino acid sequence of SEQ ID NO: 35 with a cysteine substitution at amino acid position 43.

In some embodiments, the VH region comprises or consists of an amino acid sequence of SEQ ID NO: 37, and the VL region comprises or consists of an amino acid sequence of SEQ ID NO: 35.

In some embodiments, the VH region comprises an amino acid sequence of SEQ ID NO: 37 with a cysteine substitution at amino acid position 44, and the VL region comprises an amino acid sequence of SEQ ID NO: 35 with a cysteine substitution at amino acid position 100.

In some embodiments, the VH region comprises an amino acid sequence of SEQ ID NO: 37 with a cysteine substitution at amino acid position 100B, and the VL region comprises an amino acid sequence of SEQ ID NO: 35 with a cysteine substitution at amino acid position 49.

In some embodiments, the VH region comprises an amino acid sequence of SEQ ID NO: 37 with a cysteine substitution at amino acid position 100, and the VL region comprises an amino acid sequence of SEQ ID NO: 35 with a cysteine substitution at amino acid position 50.

In some embodiments, the VH region comprises an amino acid sequence of SEQ ID NO: 37 with a cysteine substitution at amino acid position 101, and the VL region comprises an amino acid sequence of SEQ ID NO: 35 with a cysteine substitution at amino acid position 46.

In some embodiments, the VH region comprises an amino acid sequence of SEQ ID NO: 37 with a cysteine substitution at amino acid position 105, and the VL region comprises an amino acid sequence of SEQ ID NO: 35 with a cysteine substitution at amino acid position 43.

In some embodiments, the VH region comprises or consists of an amino acid sequence of SEQ ID NO: 38, and the VL region comprises or consists of an amino acid sequence of SEQ ID NO: 39.

In some embodiments, the VH region comprises an amino acid sequence of SEQ ID NO: 38 with a cysteine substitution at amino acid position 44, and the VL region comprises an amino acid sequence of SEQ ID NO: 39 with a cysteine substitution at amino acid position 100.

In some embodiments, the VH region comprises an amino acid sequence of SEQ ID NO: 38 with a cysteine substitution at amino acid position 100B, and the VL region comprises an amino acid sequence of SEQ ID NO: 39 with a cysteine substitution at amino acid position 49.

In some embodiments, the VH region comprises an amino acid sequence of SEQ ID NO: 38 with a cysteine substitution at amino acid position 100, and the VL region comprises an amino acid sequence of SEQ ID NO: 39 with a cysteine substitution at amino acid position 50.

In some embodiments, the VH region comprises an amino acid sequence of SEQ ID NO: 38 with a cysteine substitution at amino acid position 101, and the VL region comprises an amino acid sequence of SEQ ID NO: 39 with a cysteine substitution at amino acid position 46.

In some embodiments, the VH region comprises an amino acid sequence of SEQ ID NO: 38 with a cysteine substitution at amino acid position 105, and the VL region comprises an amino acid sequence of SEQ ID NO: 39 with a cysteine substitution at amino acid position 43.

In some embodiments, the VH region comprises or consists of an amino acid sequence of SEQ ID NO: 1148, and the VL region comprises or consists of an amino acid sequence of SEQ ID NO: 1149.

In some embodiments, the VH region comprises an amino acid sequence of SEQ ID NO: 1148 with a cysteine substitution at amino acid position 44, and the VL region comprises an amino acid sequence of SEQ ID NO: 1149 with a cysteine substitution at amino acid position 100.

In some embodiments, the VH region comprises an amino acid sequence of SEQ ID NO: 1148 with a cysteine substitution at amino acid position 100B, and the VL region comprises an amino acid sequence of SEQ ID NO: 1149 with a cysteine substitution at amino acid position 49.

In some embodiments, the VH region comprises an amino acid sequence of SEQ ID NO: 1148 with a cysteine substitution at amino acid position 100, and the VL region comprises an amino acid sequence of SEQ ID NO: 1149 with a cysteine substitution at amino acid position 50.

In some embodiments, the VH region comprises an amino acid sequence of SEQ ID NO: 1148 with a cysteine substitution at amino acid position 101, and the VL region comprises an amino acid sequence of SEQ ID NO: 1149 with a cysteine substitution at amino acid position 46.

In some embodiments, the VH region comprises an amino acid sequence of SEQ ID NO: 1148 with a cysteine substitution at amino acid position 105, and the VL region comprises an amino acid sequence of SEQ ID NO: 1149 with a cysteine substitution at amino acid position 43.

In some embodiments, the VH region comprises or consists of an amino acid sequence of SEQ ID NO: 1150, and the VL region comprises or consists of an amino acid sequence of SEQ ID NO: 1149.

In some embodiments, the VH region comprises an amino acid sequence of SEQ ID NO: 1150 with a cysteine substitution at amino acid position 44, and the VL region comprises an amino acid sequence of SEQ ID NO: 1149 with a cysteine substitution at amino acid position 100.

In some embodiments, the VH region comprises an amino acid sequence of SEQ ID NO: 1150 with a cysteine substitution at amino acid position 100B, and the VL region comprises an amino acid sequence of SEQ ID NO: 1149 with a cysteine substitution at amino acid position 49.

In some embodiments, the VH region comprises an amino acid sequence of SEQ ID NO: 1150 with a cysteine substitution at amino acid position 100, and the VL region comprises an amino acid sequence of SEQ ID NO: 1149 with a cysteine substitution at amino acid position 50.

In some embodiments, the VH region comprises an amino acid sequence of SEQ ID NO: 1150 with a cysteine substitution at amino acid position 101, and the VL region comprises an amino acid sequence of SEQ ID NO: 1149 with a cysteine substitution at amino acid position 46.

In some embodiments, the VH region comprises an amino acid sequence of SEQ ID NO: 1150 with a cysteine substitution at amino acid position 105, and the VL region comprises an amino acid sequence of SEQ ID NO: 1149 with a cysteine substitution at amino acid position 43.

In some embodiments, the VH region comprises or consists of an amino acid sequence of SEQ ID NO: 40, and the VL region comprises or consists of an amino acid sequence of SEQ ID NO: 41.

In some embodiments, the VH region comprises an amino acid sequence of SEQ ID NO: 40 with a cysteine substitution at amino acid position 44, and the VL region comprises an amino acid sequence of SEQ ID NO: 41 with a cysteine substitution at amino acid position 100.

In some embodiments, the VH region comprises an amino acid sequence of SEQ ID NO: 40 with a cysteine substitution at amino acid position 100B, and the VL region comprises an amino acid sequence of SEQ ID NO: 41 with a cysteine substitution at amino acid position 49.

In some embodiments, the VH region comprises an amino acid sequence of SEQ ID NO: 40 with a cysteine substitution at amino acid position 100, and the VL region comprises an amino acid sequence of SEQ ID NO: 41 with a cysteine substitution at amino acid position 50.

In some embodiments, the VH region comprises an amino acid sequence of SEQ ID NO: 40 with a cysteine substitution at amino acid position 101, and the VL region comprises an amino acid sequence of SEQ ID NO: 41 with a cysteine substitution at amino acid position 46.

In some embodiments, the VH region comprises an amino acid sequence of SEQ ID NO: 40 with a cysteine substitution at amino acid position 105, and the VL region comprises an amino acid sequence of SEQ ID NO: 41 with a cysteine substitution at amino acid position 43.

In some embodiments, the VH region comprises or consists of an amino acid sequence of SEQ ID NO: 42, and the VL region comprises or consists of an amino acid sequence of SEQ ID NO: 41.

In some embodiments, the VH region comprises an amino acid sequence of SEQ ID NO: 42 with a cysteine substitution at amino acid position 44, and the VL region comprises an amino acid sequence of SEQ ID NO: 41 with a cysteine substitution at amino acid position 100.

In some embodiments, the VH region comprises an amino acid sequence of SEQ ID NO: 42 with a cysteine substitution at amino acid position 100B, and the VL region comprises an amino acid sequence of SEQ ID NO: 41 with a cysteine substitution at amino acid position 49.

In some embodiments, the VH region comprises an amino acid sequence of SEQ ID NO: 42 with a cysteine substitution at amino acid position 100, and the VL region comprises an amino acid sequence of SEQ ID NO: 41 with a cysteine substitution at amino acid position 50.

In some embodiments, the VH region comprises an amino acid sequence of SEQ ID NO: 42 with a cysteine substitution at amino acid position 101, and the VL region comprises an amino acid sequence of SEQ ID NO: 41 with a cysteine substitution at amino acid position 46.

In some embodiments, the VH region comprises an amino acid sequence of SEQ ID NO: 42 with a cysteine substitution at amino acid position 105, and the VL region comprises an amino acid sequence of SEQ ID NO: 41 with a cysteine substitution at amino acid position 43.

In some embodiments, one engineered disulfide bond connects the VH region and the VL region of the TCRβV-binding domain. In some embodiments, two engineered disulfide bonds connect the VH region and the VL region of the TCRβV-binding domain. In some embodiments, three engineered disulfide bonds connect the VH region and the VL region of the TCRβV-binding domain. In some embodiments, four engineered disulfide bonds connect the VH region and the VL region of the TCRβV-binding domain. In some embodiments, five engineered disulfide bonds connect the VH region and the VL region of the TCRβV-binding domain.

In some embodiments, the TCRβV-binding domain comprises a disulfide bond at the position of VH44-VL100. In some embodiments, the TCRβV-binding domain comprises a disulfide bond at the position of VH100B-VL49. In some embodiments, the TCRβV-binding domain comprises a disulfide bond at the position of VH100-VL50. In some embodiments, the TCRβV-binding domain comprises a disulfide bond at the position of VH101-VL46. In some embodiments, the TCRβV-binding domain comprises a disulfide bond at the position of VH105-VL43.

In some embodiments, the TCRβV-binding domain is an scFv. Exemplary scFvs are provided in Table 4 below.

TABLE 4
TRBV scFvs

| | | | SEQ ID |
|---|---|---|---|
| ID | Description | Amino Acid Sequence | NO |
| TCRBD-1 | TRBV6-5 | QVQLVQSGAEVKKPGSSVKVSCKASGYSFTTYYI | 43 |
| | scFv | HWVRQAPGQGLEWMGWFFPGSGNIKYNEKFKG | |
| | (VH-VL) | RVTITADTSTSTAYMELSSLRSEDTAVYYCAGSY | |
| | | YSYDVLDYWGQGTTVTVSSGGGGSGGGGSGGG | |
| | | GSDIQMTQSPSFLSASVGDRVTITCKASQNVGIN | |
| | | VVWHQQKPGKAPKALIYSSSHRYSGVPSRFSGSG | |
| | | SGTEFTLTISSLQPEDFATYFCQQFKSYPLTFGQG | |
| | | TKLEIK | |
| TCRBD-2 | TRBV6-5 | DIQMTQSPSFLSASVGDRVTITCKASQNVGINVV | 44 |
| | scFv | WHQQKPGKAPKALIYSSSHRYSGVPSRFSGSGSG | |
| | (VL-VH) | TEFTLTISSLQPEDFATYFCQQFKSYPLTFGQGTK | |
| | | LEIKGGGGSGGGGSGGGGSQVQLVQSGAEVKKP | |
| | | GSSVKVSCKASGYSFTTYYIHWVRQAPGQGLEW | |
| | | MGWFFPGSGNIKYNEKFKGRVTITADTSTSTAY | |
| | | MELSSLRSEDTAVYYCAGSYYSYDVLDYWGQG | |
| | | TTVTVSS | |
| TCRBD-3 | TRBV6-5 | QVQLVQSGAEVKKPGSSVKVSCKASGYSFTTYYI | 45 |
| | scFv | HWVRQAPGQCLEWMGWFFPGSGNIKYNEKFKG | |
| | (VH-VL; | RVTITADTSTSTAYMELSSLRSEDTAVYYCAGSY | |
| | C44:C100) | YSYDVLDYWGQGTTVTVSSGGGGSGGGGSGGG | |
| | | GSDIQMTQSPSFLSASVGDRVTITCKASQNVGIN | |
| | | VVWHQQKPGKAPKALIYSSSHRYSGVPSRFSGSG | |
| | | SGTEFTLTISSLQPEDFATYFCQQFKSYPLTFGCG | |
| | | TKLEIK | |
| TCRBD-4 | TRBV6-5 | DIQMTQSPSFLSASVGDRVTITCKASQNVGINVV | 46 |
| | scFv | WHQQKPGKAPKALIYSSSHRYSGVPSRFSGSGSG | |
| | (VL-VH; | TEFTLTISSLQPEDFATYFCQQFKSYPLTFGCGTK | |
| | C44:C100) | LEIKGGGGSGGGGSGGGGSQVQLVQSGAEVKKP | |
| | | GSSVKVSCKASGYSFTTYYIHWVRQAPGQCLEW | |
| | | MGWFFPGSGNIKYNEKFKGRVTITADTSTSTAY | |
| | | MELSSLRSEDTAVYYCAGSYYSYDVLDYWGQG | |
| | | TTVTVSS | |
| TCRBD-5 | TRBV6-5 | QVQLVQSGAEVKKPGSSVKVSCKASGYSFTTYYI | 47 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C100B:C49) | YSYDVCDYWGQGTTVTVSSGGGGSGGGGSGGG | |
| | | GSDIQMTQSPSFLSASVGDRVTITCKASQNVGIN | |
| | | VVWHQQKPGKAPKALICSSSHRYSGVPSRFSGSG | |
| | | SGTEFTLTISSLQPEDFATYFCQQFKSYPLTFGQG | |
| | | TKLEIK | |
| TCRBD-6 | TRBV6-5 | DIQMTQSPSFLSASVGDRVTITCKASQNVGINVV | 48 |
| | scFv | WHQQKPGKAPKALICSSSHRYSGVPSRFSGSGSG | |
| | (VL-VH; | TEFTLTISSLQPEDFATYFCQQFKSYPLTFGQGTK | |
| | C100B:C49) | LEIKGGGGSGGGGSGGGGSQVQLVQSGAEVKKP | |
| | | GSSVKVSCKASGYSFTTYYIHWVRQAPGQGLEW | |
| | | MGWFFPGSGNIKYNEKFKGRVTITADTSTSTAY | |
| | | MELSSLRSEDTAVYYCAGSYYSYDVCDYWGQG | |
| | | TTVTVSS | |
| TCRBD-7 | TRBV6-5 | QVQLVQSGAEVKKPGSSVKVSCKASGYSFTTYYI | 49 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C100:C50) | YSYCVLDYWGQGTTVTVSSGGGGSGGGGSGGG | |
| | | GSDIQMTQSPSFLSASVGDRVTITCKASQNVGIN | |
| | | VVWHQQKPGKAPKALIYCSSHRYSGVPSRFSGS | |
| | | GSGTEFTLTISSLQPEDFATYFCQQFKSYPLTFGQ | |
| | | GTKLEIK | |
| TCRBD-8 | TRBV6-5 | DIQMTQSPSFLSASVGDRVTITCKASQNVGINVV | 50 |
| | scFv | WHQQKPGKAPKALIYCSSHRYSGVPSRFSGSGSG | |
| | (VL-VH; | TEFTLTISSLQPEDFATYFCQQFKSYPLTFGQGTK | |
| | C100:C50) | LEIKGGGGSGGGGSGGGGSQVQLVQSGAEVKKP | |
| | | GSSVKVSCKASGYSFTTYYIHWVRQAPGQGLEW | |
| | | MGWFFPGSGNIKYNEKFKGRVTITADTSTSTAY | |
| | | MELSSLRSEDTAVYYCAGSYYSYCVLDYWGQG | |
| | | TTVTVSS | |
| TCRBD-9 | TRBV6-5 | QVQLVQSGAEVKKPGSSVKVSCKASGYSFTTYYI | 51 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C105:C43) | YSYDVLDYWGCGTTVTVSSGGGGSGGGGSGGG | |
| | | GSDIQMTQSPSFLSASVGDRVTITCKASQNVGIN | |
| | | VVWHQQKPGKCPKALIYSSSHRYSGVPSRFSGSG | |
| | | SGTEFTLTISSLQPEDFATYFCQQFKSYPLTFGQG | |
| | | TKLEIK | |
| TCRBD-10 | TRBV6-5 | DIQMTQSPSFLSASVGDRVTITCKASQNVGINVV | 52 |
| | scFv | WHQQKPGKCPKALIYSSSHRYSGVPSRFSGSGSG | |
| | (VL-VH; | TEFTLTISSLQPEDFATYFCQQFKSYPLTFGQGTK | |
| | C105:C43) | LEIKGGGGSGGGGSGGGGSQVQLVQSGAEVKKP | |
| | | GSSVKVSCKASGYSFTTYYIHWVRQAPGQGLEW | |
| | | MGWFFPGSGNIKYNEKFKGRVTITADTSTSTAY | |
| | | MELSSLRSEDTAVYYCAGSYYSYDVLDYWGCG | |
| | | TTVTVSS | |
| TCRBD-11 | TRBV6-5 | QVQLVQSGAEVKKPGSSVKVSCKASGYSFTTYYI | 53 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C101:C46) | YSYDVLCYWGQGTTVTVSSGGGGSGGGGSGGG | |
| | | GSDIQMTQSPSFLSASVGDRVTITCKASQNVGIN | |
| | | VVWHQQKPGKAPKCLIYSSSHRYSGVPSRFSGSG | |
| | | SGTEFTLTISSLQPEDFATYFCQQFKSYPLTFGQG | |
| | | TKLEIK | |
| TCRBD-12 | TRBV6-5 | DIQMTQSPSFLSASVGDRVTITCKASQNVGINVV | 54 |
| | scFv | WHQQKPGKAPKCLIYSSSHRYSGVPSRFSGSGSG | |
| | (VL-VH; | TEFTLTISSLQPEDFATYFCQQFKSYPLTFGQGTK | |
| | C101:C46) | LEIKGGGGSGGGGSGGGGSQVQLVQSGAEVKKP | |
| | | GSSVKVSCKASGYSFTTYYIHWVRQAPGQGLEW | |
| | | MGWFFPGSGNIKYNEKFKGRVTITADTSTSTAY | |
| | | MELSSLRSEDTAVYYCAGSYYSYDVLCYWGQG | |
| | | TTVTVSS | |
| TCRBD-13 | TRBV6-5 | QVQLVQSGAEVKKPGASVKVSCKASGYSFTTYY | 55 |
| | scFv | IHWVRQAPGQGLEWMGWFFPGSGNIKYNEKFK | |
| | (VH-VL) | GRVTITADTSTSTAYMELSSLRSEDTAVYYCAGS | |
| | | YYSYDVLDYWGQGTLVTVSSGGGGSGGGGSGG | |
| | | GGSDIQMTQSPSFLSASVGDRVTITCKASQNVGI | |
| | | NVVWHQQKPGKAPKALIYSSSHRYSGVPSRFSGS | |
| | | GSGTEFTLTISSLQPEDFATYFCQQFKSYPLTFGQ | |
| | | GTKLEIK | |
| TCRBD-14 | TRBV6-5 | QVQLVQSGAEVKKPGASVKVSCKASGYSFTTYY | 56 |
| | scFv | IHWVRQAPGQCLEWMGWFFPGSGNIKYNEKFK | |
| | (VH-VL; | GRVTITADTSTSTAYMELSSLRSEDTAVYYCAGS | |
| | C44:C100) | YYSYDVLDYWGQGTLVTVSSGGGGSGGGGSGG | |
| | | GGSDIQMTQSPSFLSASVGDRVTITCKASQNVGI | |
| | | NVVWHQQKPGKAPKALIYSSSHRYSGVPSRFSGS | |
| | | GSGTEFTLTISSLQPEDFATYFCQQFKSYPLTFGC | |
| | | GTKLEIK | |
| TCRBD-15 | TRBV6-5 | QVQLVQSGAEVKKPGSSVKVSCKASGYSFRLTYI | 57 |
| | scFv | HWVRQAPGQGLEWMGWFFPGSGNIKYNEKFKG | |
| | (VH-VL) | RVTITADTSTSTAYMELSSLRSEDTAVYYCAGSY | |
| | | YSYDVLDYWGQGTTVTVSSGGGGSGGGGSGGG | |
| | | GSDIQMTQSPSFLSASVGDRVTITCKASQNVGIN | |
| | | VVWHQQKPGKAPKALIYSSSHRYSGVPSRFSGSG | |
| | | SGTEFTLTISSLQPEDFATYFCQQFKSYPLTFGQG | |
| | | TKLEIK | |
| TCRBD-16 | TRBV6-5 | DIQMTQSPSFLSASVGDRVTITCKASQNVGINVV | 58 |
| | scFv | WHQQKPGKAPKALIYSSSHRYSGVPSRFSGSGSG | |
| | (VL-VH) | TEFTLTISSLQPEDFATYFCQQFKSYPLTFGQGTK | |
| | | LEIKGGGGSGGGGSGGGGSQVQLVQSGAEVKKP | |
| | | GSSVKVSCKASGYSFRLTYIHWVRQAPGQGLEW | |
| | | MGWFFPGSGNIKYNEKFKGRVTITADTSTSTAY | |
| | | MELSSLRSEDTAVYYCAGSYYSYDVLDYWGQG | |
| | | TTVTVSS | |
| TCRBD-17 | TRBV6-5 | QVQLVQSGAEVKKPGSSVKVSCKASGYSFRLTYI | 59 |
| | scFv | HWVRQAPGQCLEWMGWFFPGSGNIKYNEKFKG | |
| | (VH-VL; | RVTITADTSTSTAYMELSSLRSEDTAVYYCAGSY | |
| | C44:C100) | YSYDVLDYWGQGTTVTVSSGGGGSGGGGSGGG | |
| | | GSDIQMTQSPSFLSASVGDRVTITCKASQNVGIN | |
| | | VVWHQQKPGKAPKALIYSSSHRYSGVPSRFSGSG | |
| | | SGTEFTLTISSLQPEDFATYFCQQFKSYPLTFGCG | |
| | | TKLEIK | |
| TCRBD-18 | TRBV6-5 | DIQMTQSPSFLSASVGDRVTITCKASQNVGINVV | 60 |
| | scFv | WHQQKPGKAPKALIYSSSHRYSGVPSRFSGSGSG | |
| | (VL-VH; | TEFTLTISSLQPEDFATYFCQQFKSYPLTFGCGTK | |
| | C44:C100) | LEIKGGGGSGGGGSGGGGSQVQLVQSGAEVKKP | |
| | | GSSVKVSCKASGYSFRLTYIHWVRQAPGQCLEW | |
| | | MGWFFPGSGNIKYNEKFKGRVTITADTSTSTAY | |
| | | MELSSLRSEDTAVYYCAGSYYSYDVLDYWGQG | |
| | | TTVTVSS | |
| TCRBD-19 | TRBV6-5 | QVQLVQSGAEVKKPGSSVKVSCKASGYSFRLTYI | 61 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C100B:C49) | YSYDVCDYWGQGTTVTVSSGGGGSGGGGSGGG | |
| | | GSDIQMTQSPSFLSASVGDRVTITCKASQNVGIN | |
| | | VVWHQQKPGKAPKALICSSSHRYSGVPSRFSGSG | |
| | | SGTEFTLTISSLQPEDFATYFCQQFKSYPLTFGQG | |
| | | TKLEIK | |
| TCRBD-20 | TRBV6-5 | DIQMTQSPSFLSASVGDRVTITCKASQNVGINVV | 62 |
| | scFv | WHQQKPGKAPKALICSSSHRYSGVPSRFSGSGSG | |
| | (VL-VH; | TEFTLTISSLQPEDFATYFCQQFKSYPLTFGQGTK | |
| | C100B:C49) | LEIKGGGGSGGGGSGGGGSQVQLVQSGAEVKKP | |
| | | GSSVKVSCKASGYSFRLTYIHWVRQAPGQGLEW | |
| | | MGWFFPGSGNIKYNEKFKGRVTITADTSTSTAY | |
| | | MELSSLRSEDTAVYYCAGSYYSYDVCDYWGQG | |
| | | TTVTVSS | |
| TCRBD-21 | TRBV6-5 | QVQLVQSGAEVKKPGSSVKVSCKASGYSFRLTYI | 63 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C100:C50) | YSYCVLDYWGQGTTVTVSSGGGGSGGGGSGGG | |
| | | GSDIQMTQSPSFLSASVGDRVTITCKASQNVGIN | |
| | | VVWHQQKPGKAPKALIYCSSHRYSGVPSRFSGS | |
| | | GSGTEFTLTISSLQPEDFATYFCQQFKSYPLTFGQ | |
| | | GTKLEIK | |
| TCRBD-22 | TRBV6-5 | DIQMTQSPSFLSASVGDRVTITCKASQNVGINVV | 64 |
| | scFv | WHQQKPGKAPKALIYCSSHRYSGVPSRFSGSGSG | |
| | (VL-VH; | TEFTLTISSLQPEDFATYFCQQFKSYPLTFGQGTK | |
| | C100:C50) | LEIKGGGGSGGGGSGGGGSQVQLVQSGAEVKKP | |
| | | GSSVKVSCKASGYSFRLTYIHWVRQAPGQGLEW | |
| | | MGWFFPGSGNIKYNEKFKGRVTITADTSTSTAY | |
| | | MELSSLRSEDTAVYYCAGSYYSYCVLDYWGQG | |
| | | TTVTVSS | |
| TCRBD-23 | TRBV6-5 | QVQLVQSGAEVKKPGSSVKVSCKASGYSFRLTYI | 65 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C105:C43) | YSYDVLDYWGCGTTVTVSSGGGGSGGGGSGGG | |
| | | GSDIQMTQSPSFLSASVGDRVTITCKASQNVGIN | |
| | | VVWHQQKPGKCPKALIYSSSHRYSGVPSRFSGSG | |
| | | SGTEFTLTISSLQPEDFATYFCQQFKSYPLTFGQG | |
| | | TKLEIK | |
| TCRBD-24 | TRBV6-5 | DIQMTQSPSFLSASVGDRVTITCKASQNVGINVV | 66 |
| | scFv | WHQQKPGKCPKALIYSSSHRYSGVPSRFSGSGSG | |
| | (VL-VH; | TEFTLTISSLQPEDFATYFCQQFKSYPLTFGQGTK | |
| | C105:C43) | LEIKGGGGSGGGGSGGGGSQVQLVQSGAEVKKP | |
| | | GSSVKVSCKASGYSFRLTYIHWVRQAPGQGLEW | |
| | | MGWFFPGSGNIKYNEKFKGRVTITADTSTSTAY | |
| | | MELSSLRSEDTAVYYCAGSYYSYDVLDYWGCG | |
| | | TTVTVSS | |
| TCRBD-25 | TRBV6-5 | QVQLVQSGAEVKKPGSSVKVSCKASGYSFRLTYI | 67 |
| | scFv | HWVRQAPGQGLEWMGWFFPGSGNIKYNEKFKG | |
| | (VH-VL; | RVTITADTSTSTAYMELSSLRSEDTAVYYCAGSY | |
| | C101:C46) | YSYDVLCYWGQGTTVTVSSGGGGGGGGSGGG | |
| | | GSDIQMTQSPSFLSASVGDRVTITCKASQNVGIN | |
| | | VVWHQQKPGKAPKCLIYSSSHRYSGVPSRFSGSG | |
| | | SGTEFTLTISSLQPEDFATYFCQQFKSYPLTFGQG | |
| | | TKLEIK | |
| TCRBD-26 | TRBV6-5 | DIQMTQSPSFLSASVGDRVTITCKASQNVGINVV | 68 |
| | scFv | WHQQKPGKAPKCLIYSSSHRYSGVPSRFSGSGSG | |
| | (VL-VH; | TEFTLTISSLQPEDFATYFCQQFKSYPLTFGQGTK | |
| | C101:C46) | LEIKGGGGSGGGGSGGGGSQVQLVQSGAEVKKP | |
| | | GSSVKVSCKASGYSFRLTYIHWVRQAPGQGLEW | |
| | | MGWFFPGSGNIKYNEKFKGRVTITADTSTSTAY | |
| | | MELSSLRSEDTAVYYCAGSYYSYDVLCYWGQG | |
| | | TTVTVSS | |
| TCRBD-27 | TRBV6-5 | QVQLVQSGAEVKKPGSSVKVSCKASGHDFRLTY | 69 |
| | scFv | IHWVRQAPGQGLEWMGRVSAGSGNVKYNEKFK | |
| | (VH-VL) | GRVTITADTSTSTAYMELSSLRSEDTAVYYCAVS | |
| | | YYSYDVLDYWGQGTTVTVSSGGGGSGGGGSGG | |
| | | GGSDIQMTQSPSFLSASVGDRVTITCKASQNVGI | |
| | | NVVWHQQKPGKAPKALIYSSSHRYSGVPSRFSGS | |
| | | GSGTEFTLTISSLQPEDFATYFCQQFKSYPLTFGQ | |
| | | GTKLEIK | |
| TCRBD-28 | TRBV6-5 | DIQMTQSPSFLSASVGDRVTITCKASQNVGINVV | 70 |
| | scFv | WHQQKPGKAPKALIYSSSHRYSGVPSRFSGSGSG | |
| | (VL-VH) | TEFTLTISSLQPEDFATYFCQQFKSYPLTFGQGTK | |
| | | LEIKGGGGSGGGGSGGGGSQVQLVQSGAEVKKP | |
| | | GSSVKVSCKASGHDFRLTYIHWVRQAPGQGLEW | |
| | | MGRVSAGSGNVKYNEKFKGRVTITADTSTSTAY | |
| | | MELSSLRSEDTAVYYCAVSYYSYDVLDYWGQG | |
| | | TTVTVSS | |
| TCRBD-29 | TRBV6-5 | QVQLVQSGAEVKKPGSSVKVSCKASGHDFRLTY | 71 |
| | scFv | IHWVRQAPGQCLEWMGRVSAGSGNVKYNEKFK | |
| | (VH-VL; | GRVTITADTSTSTAYMELSSLRSEDTAVYYCAVS | |
| | C44:C100) | YYSYDVLDYWGQGTTVTVSSGGGGSGGGGSGG | |
| | | GGSDIQMTQSPSFLSASVGDRVTITCKASQNVGI | |
| | | NVVWHQQKPGKAPKALIYSSSHRYSGVPSRFSGS | |
| | | GSGTEFTLTISSLQPEDFATYFCQQFKSYPLTFGC | |
| | | GTKLEIK | |
| TCRBD-30 | TRBV6-5 | DIQMTQSPSFLSASVGDRVTITCKASQNVGINVV | 72 |
| | scFv | WHQQKPGKAPKALIYSSSHRYSGVPSRFSGSGSG | |
| | (VL-VH; | TEFTLTISSLQPEDFATYFCQQFKSYPLTFGCGTK | |
| | C44:C100) | LEIKGGGGSGGGGSGGGGSQVQLVQSGAEVKKP | |
| | | GSSVKVSCKASGHDFRLTYIHWVRQAPGQCLEW | |
| | | MGRVSAGSGNVKYNEKFKGRVTITADTSTSTAY | |
| | | MELSSLRSEDTAVYYCAVSYYSYDVLDYWGQG | |
| | | TTVTVSS | |
| TCRBD-31 | TRBV6-5 | QVQLVQSGAEVKKPGSSVKVSCKASGHDFRLTY | 73 |
| | scFv | IHWVRQAPGQGLEWMGRVSAGSGNVKYNEKFK | |
| | (VH-VL; | GRVTITADTSTSTAYMELSSLRSEDTAVYYCAVS | |
| | C100B:C49) | YYSYDVCDYWGQGTTVTVSSGGGGSGGGGSGG | |
| | | GGSDIQMTQSPSFLSASVGDRVTITCKASQNVGI | |
| | | NVVWHQQKPGKAPKALICSSSHRYSGVPSRFSGS | |
| | | GSGTEFTLTISSLQPEDFATYFCQQFKSYPLTFGQ | |
| | | GTKLEIK | |
| TCRBD-32 | TRBV6-5 | DIQMTQSPSFLSASVGDRVTITCKASQNVGINVV | 74 |
| | scFv | WHQQKPGKAPKALICSSSHRYSGVPSRFSGSGSG | |
| | (VL-VH; | TEFTLTISSLQPEDFATYFCQQFKSYPLTFGQGTK | |
| | C100B:C49) | LEIKGGGGSGGGGSGGGGSQVQLVQSGAEVKKP | |
| | | GSSVKVSCKASGHDFRLTYIHWVRQAPGQGLEW | |
| | | MGRVSAGSGNVKYNEKFKGRVTITADTSTSTAY | |
| | | MELSSLRSEDTAVYYCAVSYYSYDVCDYWGQG | |
| | | TTVTVSS | |
| TCRBD-33 | TRBV6-5 | QVQLVQSGAEVKKPGSSVKVSCKASGHDFRLTY | 75 |
| | scFv | IHWVRQAPGQGLEWMGRVSAGSGNVKYNEKFK | |
| | (VH-VL; | GRVTITADTSTSTAYMELSSLRSEDTAVYYCAVS | |
| | C100:C50) | YYSYCVLDYWGQGTTVTVSSGGGGSGGGGSGG | |
| | | GGSDIQMTQSPSFLSASVGDRVTITCKASQNVGI | |
| | | NVVWHQQKPGKAPKALIYCSSHRYSGVPSRFSG | |
| | | SGSGTEFTLTISSLQPEDFATYFCQQFKSYPLTFG | |
| | | QGTKLEIK | |
| TCRBD-34 | TRBV6-5 | DIQMTQSPSFLSASVGDRVTITCKASQNVGINVV | 76 |
| | scFv | WHQQKPGKAPKALIYCSSHRYSGVPSRFSGSGSG | |
| | (VL-VH; | TEFTLTISSLQPEDFATYFCQQFKSYPLTFGQGTK | |
| | C100:C50) | LEIKGGGGSGGGGSGGGGSQVQLVQSGAEVKKP | |
| | | GSSVKVSCKASGHDFRLTYIHWVRQAPGQGLEW | |
| | | MGRVSAGSGNVKYNEKFKGRVTITADTSTSTAY | |
| | | MELSSLRSEDTAVYYCAVSYYSYCVLDYWGQG | |
| | | TTVTVSS | |
| TCRBD-35 | TRBV6-5 | QVQLVQSGAEVKKPGSSVKVSCKASGHDFRLTY | 77 |
| | scFv | IHWVRQAPGQGLEWMGRVSAGSGNVKYNEKFK | |
| | (VH-VL; | GRVTITADTSTSTAYMELSSLRSEDTAVYYCAVS | |
| | C105:C43) | YYSYDVLDYWGCGTTVTVSSGGGGSGGGGSGG | |
| | | GGSDIQMTQSPSFLSASVGDRVTITCKASQNVGI | |
| | | NVVWHQQKPGKCPKALIYSSSHRYSGVPSRFSGS | |
| | | GSGTEFTLTISSLQPEDFATYFCQQFKSYPLTFGQ | |
| | | GTKLEIK | |
| TCRBD-36 | TRBV6-5 | DIQMTQSPSFLSASVGDRVTITCKASQNVGINVV | 78 |
| | scFv | WHQQKPGKCPKALIYSSSHRYSGVPSRFSGSGSG | |
| | (VL-VH; | TEFTLTISSLQPEDFATYFCQQFKSYPLTFGQGTK | |
| | C105:C43) | LEIKGGGGSGGGGSGGGGSQVQLVQSGAEVKKP | |
| | | GSSVKVSCKASGHDFRLTYIHWVRQAPGQGLEW | |
| | | MGRVSAGSGNVKYNEKFKGRVTITADTSTSTAY | |
| | | MELSSLRSEDTAVYYCAVSYYSYDVLDYWGCG | |
| | | TTVTVSS | |
| TCRBD-37 | TRBV6-5 | QVQLVQSGAEVKKPGSSVKVSCKASGHDFRLTY | 79 |
| | scFv | IHWVRQAPGQGLEWMGRVSAGSGNVKYNEKFK | |
| | (VH-VL; | GRVTITADTSTSTAYMELSSLRSEDTAVYYCAVS | |
| | C101:C46) | YYSYDVLCYWGQGTTVTVSSGGGGSGGGGSGG | |
| | | GGSDIQMTQSPSFLSASVGDRVTITCKASQNVGI | |
| | | NVVWHQQKPGKAPKCLIYSSSHRYSGVPSRFSGS | |
| | | GSGTEFTLTISSLQPEDFATYFCQQFKSYPLTFGQ | |
| | | GTKLEIK | |
| TCRBD-38 | TRBV6-5 | DIQMTQSPSFLSASVGDRVTITCKASQNVGINVV | 80 |
| | scFv | WHQQKPGKAPKCLIYSSSHRYSGVPSRFSGSGSG | |
| | (VL-VH; | TEFTLTISSLQPEDFATYFCQQFKSYPLTFGQGTK | |
| | C101:C46) | LEIKGGGGSGGGGSGGGGSQVQLVQSGAEVKKP | |
| | | GSSVKVSCKASGHDFRLTYIHWVRQAPGQGLEW | |
| | | MGRVSAGSGNVKYNEKFKGRVTITADTSTSTAY | |
| | | MELSSLRSEDTAVYYCAVSYYSYDVLCYWGQG | |
| | | TTVTVSS | |
| CLY-172 | TRBV6-5 | QVQLVQSGAEVKKPGSSVKVSCKASGYSFTLYYI | 1151 |
| | scFv (VH- | HWVRQAPGQGLEWMGWFFPGSGNIKYNEKFKG | |
| | VL) | RVTITADTSTSTAYMELSSLRSEDTAVYYCAASY | |
| | | YSFDVLDYWGQGTTVTVSSGGGGSGGGGSGGG | |
| | | GSDIQMTQSPSFLSASVGDRVTITCKASQNVGIRV | |
| | | VWHQQKPGKAPKALIYSSSHRYSGVPSRFSGSGS | |
| | | GTEFTLTISSLQPEDFATYFCQQFKSYPITFGQGT | |
| | | KLEIK | |
| CLY-190 | TRBV6-5 | QVQLVQSGAEVKKPGSSVKVSCKASGYSFTLYYI | 1152 |
| | scFv (VH- | HWVRQAPGQCLEWMGWFFPGSGNIKYNEKFKG | |
| | VL; | RVTITADTSTSTAYMELSSLRSEDTAVYYCAASY | |
| | C44:C100) | YSFDVLDYWGQGTTVTVSSGGGGSGGGGSGGG | |
| | | GSDIQMTQSPSFLSASVGDRVTITCKASQNVGIRV | |
| | | VWHQQKPGKAPKALIYSSSHRYSGVPSRFSGSGS | |
| | | GTEFTLTISSLQPEDFATYFCQQFKSYPITFGCGTK | |
| | | LEIK | |
| CLY-175 | TRBV6-5 | QVQLVQSGAEVKKPGSSVKVSCKASGYSFTTYYI | 1153 |
| | scFv (VH- | HWVRQAPGQGLEWMGWFFPRSGNIKYNEKFKG | |
| | VL) | RVTITADTSTSTAYMELSSLRSEDTAVYYCAASY | |
| | | YSFDVLDYWGQGTTVTVSSGGGGSGGGGSGGG | |
| | | GSDIQMTQSPSFLSASVGDRVTITCKASQNVGIRV | |
| | | VWHQQKPGKAPKALIYSSSHRYSGVPSRFSGSGS | |
| | | GTEFTLTISSLQPEDFATYFCQQFKSYPITFGQGT | |
| | | KLEIK | |
| CLY-193 | TRBV6-5 | QVQLVQSGAEVKKPGSSVKVSCKASGYSFTTYYI | 1154 |
| | scFv (VH- | HWVRQAPGQCLEWMGWFFPRSGNIKYNEKFKG | |
| | VL; | RVTITADTSTSTAYMELSSLRSEDTAVYYCAASY | |
| | C44:C100) | YSFDVLDYWGQGTTVTVSSGGGGSGGGGSGGG | |
| | | GSDIQMTQSPSFLSASVGDRVTITCKASQNVGIRV | |
| | | VWHQQKPGKAPKALIYSSSHRYSGVPSRFSGSGS | |
| | | GTEFTLTISSLQPEDFATYFCQQFKSYPITFGCGTK | |
| | | LEIK | |
| TCRBD-39 | TRBV12-3/4 | EVQLVESGGGLVQPGGSLRLSCAASGFTFSNFGM | 81 |
| | scFv | HWVRQAPGKGLEWVSYISSGSSTIYYADTLKGR | |
| | (VH-VL) | FTISRDNSKNTLYLQMNSLRAEDTAVYYCARRG | |
| | | EGAMDYWGQGTTVTVSSGGGGSGGGGSGGGGS | |
| | | DNQLTQSPSFLSASVGDRVTITCRASSSVNYIYW | |
| | | YQQKPGKAPKLLIYYTSNLAPGVPSRFSGSGSGN | |
| | | EYTLTISSLQPEDFATYYCQQFTSSPFTFGQGTKL | |
| | | EIK | |
| TCRBD-40 | TRBV12-3/4 | DNQLTQSPSFLSASVGDRVTITCRASSSVNYIYW | 82 |
| | scFv | YQQKPGKAPKLLIYYTSNLAPGVPSRFSGSGSGN | |
| | (VL-VH) | EYTLTISSLQPEDFATYYCQQFTSSPFTFGQGTKL | |
| | | EIKGGGGSGGGGSGGGGSEVQLVESGGGLVQPG | |
| | | GSLRLSCAASGFTFSNFGMHWVRQAPGKGLEW | |
| | | VSYISSGSSTIYYADTLKGRFTISRDNSKNTLYLQ | |
| | | MNSLRAEDTAVYYCARRGEGAMDYWGQGTTV | |
| | | TVSS | |
| TCRBD-41 | TRBV12-3/4 | EVQLVESGGGLVQPGGSLRLSCAASGFTFSNFGM | 83 |
| | scFv | HWVRQAPGKCLEWVSYISSGSSTIYYADTLKGRF | |
| | (VH-VL; | TISRDNSKNTLYLQMNSLRAEDTAVYYCARRGE | |
| | C44:C100) | GAMDYWGQGTTVTVSSGGGGSGGGGSGGGGSD | |
| | | NQLTQSPSFLSASVGDRVTITCRASSSVNYIYWY | |
| | | QQKPGKAPKLLIYYTSNLAPGVPSRFSGSGSGNE | |
| | | YTLTISSLQPEDFATYYCQQFTSSPFTFGCGTKLEI | |
| | | K | |
| TCRBD-42 | TRBV12-3/4 | DNQLTQSPSFLSASVGDRVTITCRASSSVNYIYW | 84 |
| | scFv | YQQKPGKAPKLLIYYTSNLAPGVPSRFSGSGSGN | |
| | (VL-VH; | EYTLTISSLQPEDFATYYCQQFTSSPFTFGCGTKL | |
| | C44:C100) | EIKGGGGSGGGGSGGGGSEVQLVESGGGLVQPG | |
| | | GSLRLSCAASGFTFSNFGMHWVRQAPGKCLEWV | |
| | | SYISSGSSTIYYADTLKGRFTISRDNSKNTLYLQM | |
| | | NSLRAEDTAVYYCARRGEGAMDYWGQGTTVTV | |
| | | SS | |
| TCRBD-43 | TRBV12-3/4 | EVQLVESGGGLVQPGGSLRLSCAASGFTFSNFGM | 85 |
| | scFv | HWVRQAPGKGLEWVSYISSGSSTIYYADTLKGR | |
| | (VH-VL; | FTISRDNSKNTLYLQMNSLRAEDTAVYYCARRG | |
| | C100:C50) | EGACDYWGQGTTVTVSSGGGGSGGGGSGGGGS | |
| | | DNQLTQSPSFLSASVGDRVTITCRASSSVNYIYW | |
| | | YQQKPGKAPKLLIYCTSNLAPGVPSRFSGSGSGN | |
| | | EYTLTISSLQPEDFATYYCQQFTSSPFTFGQGTKL | |
| | | EIK | |
| TCRBD-44 | TRBV12-3/4 | DNQLTQSPSFLSASVGDRVTITCRASSSVNYIYW | 86 |
| | scFv | YQQKPGKAPKLLIYCTSNLAPGVPSRFSGSGSGN | |
| | (VL-VH; | EYTLTISSLQPEDFATYYCQQFTSSPFTFGQGTKL | |
| | C100:C50) | EIKGGGGSGGGGSGGGGSEVQLVESGGGLVQPG | |
| | | GSLRLSCAASGFTFSNFGMHWVRQAPGKGLEW | |
| | | VSYISSGSSTIYYADTLKGRFTISRDNSKNTLYLQ | |
| | | MNSLRAEDTAVYYCARRGEGACDYWGQGTTVT | |
| | | VSS | |
| TCRBD-45 | TRBV12-3/4 | EVQLVESGGGLVQPGGSLRLSCAASGFTFSNFGM | 87 |
| | scFv | HWVRQAPGKGLEWVSYISSGSSTIYYADTLKGR | |
| | (VH-VL; | FTISRDNSKNTLYLQMNSLRAEDTAVYYCARRG | |
| | C105:C43) | EGAMDYWGCGTTVTVSSGGGGSGGGGSGGGGS | |
| | | DNQLTQSPSFLSASVGDRVTITCRASSSVNYIYW | |
| | | YQQKPGKCPKLLIYYTSNLAPGVPSRFSGSGSGN | |
| | | EYTLTISSLQPEDFATYYCQQFTSSPFTFGQGTKL | |
| | | EIK | |
| TCRBD-46 | TRBV12-3/4 | DNQLTQSPSFLSASVGDRVTITCRASSSVNYIYW | 88 |
| | scFv | YQQKPGKCPKLLIYYTSNLAPGVPSRFSGSGSGN | |
| | (VL-VH; | EYTLTISSLQPEDFATYYCQQFTSSPFTFGQGTKL | |
| | C105:C43) | EIKGGGGSGGGGSGGGGSEVQLVESGGGLVQPG | |
| | | GSLRLSCAASGFTFSNFGMHWVRQAPGKGLEW | |
| | | VSYISSGSSTIYYADTLKGRFTISRDNSKNTLYLQ | |
| | | MNSLRAEDTAVYYCARRGEGAMDYWGCGTTV | |
| | | TVSS | |
| TCRBD-47 | TRBV12-3/4 | EVQLVESGGGLVQPGGSLRLSCAASGFTFSNFGM | 89 |
| | scFv | HWVRQAPGKGLEWVSYISSGSSTIYYADTLKGR | |
| | (VH-VL; | FTISRDNSKNTLYLQMNSLRAEDTAVYYCARRG | |
| | C101:C46) | EGAMCYWGQGTTVTVSSGGGGSGGGGSGGGGS | |
| | | DNQLTQSPSFLSASVGDRVTITCRASSSVNYIYW | |
| | | YQQKPGKAPKCLIYYTSNLAPGVPSRFSGSGSGN | |
| | | EYTLTISSLQPEDFATYYCQQFTSSPFTFGQGTKL | |
| | | EIK | |
| TCRBD-48 | TRBV12-3/4 | DNQLTQSPSFLSASVGDRVTITCRASSSVNYIYW | 90 |
| | scFv | YQQKPGKAPKCLIYYTSNLAPGVPSRFSGSGSGN | |
| | (VL-VH; | EYTLTISSLQPEDFATYYCQQFTSSPFTFGQGTKL | |
| | C101:C46) | EIKGGGGSGGGGSGGGGSEVQLVESGGGLVQPG | |
| | | GSLRLSCAASGFTFSNFGMHWVRQAPGKGLEW | |
| | | VSYISSGSSTIYYADTLKGRFTISRDNSKNTLYLQ | |
| | | MNSLRAEDTAVYYCARRGEGAMCYWGQGTTV | |
| | | TVSS | |
| TCRBD-49 | TRBV12-3/4 | EVQLVESGGGLVQPGGSLRLSCAASGFTFSNFGM | 91 |
| | scFv (VH- | HWVRQAPGKGLEWVSYISSGSSTIYYADTLKGR | |
| | VL) | FTISRDNSKNTLYLQMNSLRAEDTAVYYCARRG | |
| | | EGAMDYWGQGTLVTVSSGGGGSGGGGSGGGGS | |
| | | DNQLTQSPSFLSASVGDRVTITCRASSSVNYIYW | |
| | | YQQKPGKAPKLLIYYTSNLAPGVPSRFSGSGSGN | |
| | | EYTLTISSLQPEDFATYYCQQFTSSPFTFGQGTKL | |
| | | EIK | |
| TCRBD-50 | TRBV12-3/4 | EVQLVESGGGLVQPGGSLRLSCAASGFTFSNFGM | 92 |
| | scFv (VH- | HWVRQAPGKCLEWVSYISSGSSTIYYADTLKGRF | |
| | VL; | TISRDNSKNTLYLQMNSLRAEDTAVYYCARRGE | |
| | C44:C100) | GAMDYWGQGTLVTVSSGGGGSGGGGSGGGGSD | |
| | | NQLTQSPSFLSASVGDRVTITCRASSSVNYIYWY | |
| | | QQKPGKAPKLLIYYTSNLAPGVPSRFSGSGSGNE | |
| | | YTLTISSLQPEDFATYYCQQFTSSPFTFGCGTKLEI | |
| | | K | |
| TCRBD-55 | TRBV12-3/4 | EVQLVESGGGLVQPGGSLRLSCAASGFTFSNFGM | 1099 |
| | scFv (VH- | HWVRQAPGKGLEWVSYISSGSSTIYYADTLKGR | |
| | VL) | FTISRDNSKNTLYLQMNSLRAEDTAVYYCARRG | |
| | | EGAMDYWGQGTTVTVSSGGGGSGGGGSGGGGS | |
| | | DNQLTQSPSFLSASVGDRVTITCRASSSVNYIYW | |
| | | YQQKPGKAPKLLIYYTSNLAPGVPSRFSGSGSGN | |
| | | EYTLTISSLQPEDFATYYCQQFTSSPFTFGQGTKL | |
| | | EIK | |
| TCRBD-68 | TRBV20-1 | EVQLQQSVADLVRPGASLKLSCTASGFNIKSAY | 242 |
| | scFv (VH- | MHWVIQRPDQGPECLGRIDPATGKTKYAPKFQA | |
| | VL) | KATITADTSSNTAYLQLSSLTSEDTAIYYCTRSLN | |
| | | WDYGLDYWGQGTSVTVSSGGGGSGGGGSGGG | |
| | | GSDIVLTQSPASLAVSLGQRATISCRASKSVSILG | |
| | | THLIHWYQQKPGQPPKLLIYAASNLESGVPARFS | |
| | | GSGSETVFTLNIHPVEEEDAATYFCQQSIEDPWTF | |
| | | GGGTKLGIK | |

In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 45. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 46. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 47. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 48. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 49. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 50. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 51. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 52. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 53. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 54. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 56. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 59. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 60. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 61. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 62. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 63. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 64. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 65. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 66. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 67. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 68. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 71. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 72. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 73. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 74. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 75. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 76. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 77. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 78. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 79. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 80. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1151. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1152. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1153. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1154. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 83. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 84. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 85. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 86. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 87. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 88. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 89. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 90. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 92. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 242. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1017. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1018. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1019. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1020. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1021. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1022. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1023. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1024. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1025. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1026. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1027. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1028. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1029. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1030. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1031. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1032. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1033. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1034. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1035. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1036. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1037. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1038. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1039. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1040. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1041. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1042. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1043. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1044. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1045. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1046. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1047. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1048. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1049. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1050. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1051. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1052. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1053. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1054. In some embodiments, the TCRβV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1099.

In some embodiments, the TCRβV-binding domain comprises or consists of an amino acid sequence of SEQ ID NO: 45. In some embodiments, the TCRβV-binding domain comprises or consists of an amino acid sequence of SEQ ID NO: 46. In some embodiments, the TCRβV-binding domain comprises or consists of an amino acid sequence of SEQ ID NO: 47. In some embodiments, the TCRβV-binding domain comprises or consists of an amino acid sequence of SEQ ID NO: 48. In some embodiments, the TCRβV-binding domain comprises or consists of an amino acid sequence of SEQ ID NO: 49. In some embodiments, the TCRβV-binding domain comprises or consists of an amino acid sequence of SEQ ID NO: 50. In some embodiments, the TCRβV-binding domain comprises or consists of an amino acid sequence of SEQ ID NO: 51. In some embodiments, the TCRβV-binding domain comprises or consists of an amino acid sequence of SEQ ID NO: 52. In some embodiments, the TCRβV-binding domain comprises or consists of an amino acid sequence of SEQ ID NO: 53. In some embodiments, the TCRβV-binding domain comprises or consists of an amino acid sequence of SEQ ID NO: 54. In some embodiments, the TCRβV-binding domain comprises or consists of an amino acid sequence of SEQ ID NO: 56. In some embodiments, the TCRβV-binding domain comprises or consists of an amino acid sequence of SEQ ID NO: 59. In some embodiments, the TCRβV-binding domain comprises or consists of an amino acid sequence of SEQ ID NO: 60. In some embodiments, the TCRβV-binding domain comprises or consists of an amino acid sequence of SEQ ID NO: 61. In some embodiments, the TCRβV-binding domain comprises or consists of an amino acid sequence of SEQ ID NO: 62. In some embodiments, the TCRβV-binding domain comprises or consists of an amino acid sequence of SEQ ID NO: 63. In some embodiments, the TCRβV-binding domain comprises or consists of an amino acid sequence of SEQ ID NO: 64. In some embodiments, the TCRβV-binding domain comprises or consists of an amino acid sequence of SEQ ID NO: 65. In some embodiments, the TCRβV-binding domain comprises or consists of an amino acid sequence of SEQ ID NO: 66. In some embodiments, the TCRβV-binding domain comprises or consists of an amino acid sequence of SEQ ID NO: 67. In some embodiments, the TCRβV-binding domain comprises or consists of an amino acid sequence of SEQ ID NO: 68. In some embodiments, the TCRβV-binding domain comprises or consists of an amino acid sequence of SEQ ID NO: 71. In some embodiments, the TCRβV-binding domain comprises or consists of an amino acid sequence of SEQ ID NO: 72. In some embodiments, the TCRβV-binding domain comprises or consists of an amino acid sequence of SEQ ID NO: 73. In some embodiments, the TCRβV-binding domain comprises or consists of an amino acid sequence of SEQ ID NO: 74. In some embodiments, the TCRβV-binding domain comprises or consists of an amino acid sequence of SEQ ID NO: 75. In some embodiments, the TCRβV-binding domain comprises or consists of an amino acid sequence of SEQ ID NO: 76. In some embodiments, the TCRβV-binding domain comprises or consists of an amino acid sequence of SEQ ID NO: 77. In some embodiments, the TCRβV-binding domain comprises or consists of an amino acid sequence of SEQ ID NO: 78. In some embodiments, the TCRβV-binding domain comprises or consists of an amino acid sequence of SEQ ID NO: 79. In some embodiments, the TCRβV-binding domain comprises or consists of an amino acid sequence of SEQ ID NO: 80. In some embodiments, the TCRβV-binding domain comprises or consists of an amino acid sequence of SEQ ID NO: 1151. In some embodiments, the TCRβV-binding domain comprises or consists of an amino acid sequence of SEQ ID NO: 1152. In some embodiments, the TCRβV-binding domain comprises or consists of an amino acid sequence of SEQ ID NO: 1153. In some embodiments, the TCRβV-binding domain comprises or consists of an amino acid sequence of SEQ ID NO: 1154. In some embodiments, the TCRβV-binding domain comprises or consists of an amino acid sequence of SEQ ID NO: 83. In some embodiments, the TCRβV-binding domain comprises or consists of an amino acid sequence of SEQ ID NO: 84. In some embodiments, the TCRβV-binding domain comprises or consists of an amino acid sequence of SEQ ID NO: 85. In some embodiments, the TCRβV-binding domain comprises or consists of an amino acid sequence of SEQ ID NO: 86. In some embodiments, the TCRβV-binding domain comprises or consists of an amino acid sequence of SEQ ID NO: 87. In some embodiments, the TCRβV-binding domain comprises or consists of an amino acid sequence of SEQ ID NO: 88. In some embodiments, the TCRβV-binding domain comprises or consists of an amino acid sequence of SEQ ID NO: 89. In some embodiments, the TCRβV-binding domain comprises or consists of an amino acid sequence of SEQ ID NO: 90. In some embodiments, the TCRβV-binding domain comprises or consists of an amino acid sequence of SEQ ID NO: 92. In some embodiments, the TCRβV-binding domain comprises or consists of an amino acid sequence of SEQ ID NO: 242. In some embodiments, the TCRβV-binding domain comprises or consists of an amino acid sequence of SEQ ID NO: 1017. In some embodiments, the TCRβV-binding domain comprises or consists of an amino acid sequence of SEQ ID NO: 1018. In some embodiments, the TCRβV-binding domain comprises or consists of an amino acid sequence of SEQ ID NO: 1019. In some embodiments, the TCRβV-binding domain comprises or consists of an amino acid sequence of SEQ ID NO: 1020. In some embodiments, the TCRβV-binding domain comprises or consists of an amino acid sequence of SEQ ID NO: 1021. In some embodiments, the TCRβV-binding domain comprises or consists of an amino acid sequence of SEQ ID NO: 1022. In some embodiments, the TCRβV-binding domain comprises or consists of an amino acid sequence of SEQ ID NO: 1023. In some embodiments, the TCRβV-binding domain comprises or consists of an amino acid sequence of SEQ ID NO: 1024. In some embodiments, the TCRβV-binding domain comprises or consists of an amino acid sequence of SEQ ID NO: 1025. In some embodiments, the TCRβV-binding domain comprises or consists of an amino acid sequence of SEQ ID NO: 1026. In some embodiments, the TCRβV-binding domain comprises or consists of an amino acid sequence of SEQ ID NO: 1027. In some embodiments, the TCRβV-binding domain comprises or consists of an amino acid sequence of SEQ ID NO: 1028. In some embodiments, the TCRβV-binding domain comprises or consists of an amino acid sequence of SEQ ID NO: 1029. In some embodiments, the TCRβV-binding domain comprises or consists of an amino acid sequence of SEQ ID NO: 1030. In some embodiments, the TCRβV-binding domain comprises or consists of an amino acid sequence of SEQ ID NO: 1031. In some embodiments, the TCRβV-binding domain comprises or consists of an amino acid sequence of SEQ ID NO: 1032. In some embodiments, the TCRβV-binding domain comprises or consists of an amino acid sequence of SEQ ID NO: 1033. In some embodiments, the TCRβV-binding domain comprises or consists of an amino acid sequence of SEQ ID NO: 1034. In some embodiments, the TCRβV-binding domain comprises or consists of an amino acid sequence of SEQ ID NO: 1035. In some embodiments, the TCRβV-binding domain comprises or consists of an amino acid sequence of SEQ ID NO: 1036. In some embodiments, the TCRβV-binding domain comprises or consists of an amino acid sequence of SEQ ID NO: 1037. In some embodiments, the TCRβV-binding domain comprises or consists of an amino acid sequence of SEQ ID NO: 1038. In some embodiments, the TCRβV-binding domain comprises or consists of an amino acid sequence of SEQ ID NO: 1039. In some embodiments, the TCRβV-binding domain comprises or consists of an amino acid sequence of SEQ ID NO: 1040. In some embodiments, the TCRβV-binding domain comprises or consists of an amino acid sequence of SEQ ID NO: 1041. In some embodiments, the TCRβV-binding domain comprises or consists of an amino acid sequence of SEQ ID NO: 1042. In some embodiments, the TCRβV-binding domain comprises or consists of an amino acid sequence of SEQ ID NO: 1043. In some embodiments, the TCRβV-binding domain comprises or consists of an amino acid sequence of SEQ ID NO: 1044. In some embodiments, the TCRβV-binding domain comprises or consists of an amino acid sequence of SEQ ID NO: 1045. In some embodiments, the TCRβV-binding domain comprises or consists of an amino acid sequence of SEQ ID NO: 1046. In some embodiments, the TCRβV-binding domain comprises or consists of an amino acid sequence of SEQ ID NO: 1047. In some embodiments, the TCRβV-binding domain comprises or consists of an amino acid sequence of SEQ ID NO: 1048. In some embodiments, the TCRβV-binding domain comprises or consists of an amino acid sequence of SEQ ID NO: 1049. In some embodiments, the TCRβV-binding domain comprises or consists of an amino acid sequence of SEQ ID NO: 1050. In some embodiments, the TCRβV-binding domain comprises or consists of an amino acid sequence of SEQ ID NO: 1051. In some embodiments, the TCRβV-binding domain comprises or consists of an amino acid sequence of SEQ ID NO: 1052. In some embodiments, the TCRβV-binding domain comprises or consists of an amino acid sequence of SEQ ID NO: 1053. In some embodiments, the TCRβV-binding domain comprises or consists of an amino acid sequence of SEQ ID NO: 1054. In some embodiments, the TCRβV-binding domain comprises or consists of an amino acid sequence of SEQ ID NO: 1099.

In some embodiments, the TCRβV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs listed in the heavy chain column of Table 5.

In some embodiments, the TCRβV-binding domain comprises a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs listed in the light chain column of Table 5.

In some embodiments, the TCRβV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to one of SEQ ID NOs listed in the heavy chain column of Table 5, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the corresponding light chain SEQ ID NO listed in the same row of Table 5.

In some embodiments, the TCRβV-binding domain comprises a heavy chain comprising one of SEQ ID NOs listed in the heavy chain column of Table 5, and a light chain comprising the corresponding light chain SEQ ID NO listed in the same row of Table 5. In some embodiments, the TCRβV-binding domain comprises a heavy chain comprising one of SEQ ID NOs listed in the heavy chain column of Table 5 with 1, 2, 3, 4, 5, 6, 7, 8, 9, 10, or more mutations therein. In some embodiments, the TCRβV-binding domain comprises a light chain comprising the corresponding light chain SEQ ID NO listed in the same row of Table 5 with 1, 2, 3, 4, 5, 6, 7, 8, 9, 10, or more mutations therein. In some embodiments, the TCRβV-binding domain is a Fab or a Cross-Fab.

In some embodiments, the TCRβV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1155, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1156.

In some embodiments, the TCRβV-binding domain comprises a heavy chain comprising SEQ ID NO: 1155, and a light chain comprising SEQ ID NO: 1156. In some embodiments, the TCRβV-binding domain comprises a heavy chain comprising SEQ ID NO: 1155 with 1, 2, 3, 4, 5, 6, 7, 8, 9, 10, or more mutations therein. In some embodiments, the TCRβV-binding domain comprises a light chain comprising SEQ ID NO: 1156 with 1, 2, 3, 4, 5, 6, 7, 8, 9, 10, or more mutations therein. In some embodiments, the TCRβV-binding domain is a Fab or a Cross-Fab.

In some embodiments, the TCRβV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1157, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1158.

In some embodiments, the TCRβV-binding domain comprises a heavy chain comprising SEQ ID NO: 1157, and a light chain comprising SEQ ID NO: 1158. In some embodiments, the TCRβV-binding domain comprises a heavy chain comprising SEQ ID NO: 1157 with 1, 2, 3, 4, 5, 6, 7, 8, 9, 10, or more mutations therein. In some embodiments, the TCRβV-binding domain comprises a light chain comprising SEQ ID NO: 1158 with 1, 2, 3, 4, 5, 6, 7, 8, 9, 10, or more mutations therein. In some embodiments, the TCRβV-binding domain is a Fab or a Cross-Fab.

TABLE 5
TRBV Fab or CrossFab

| | | SEQ ID | SEQ ID NO |
|---|---|---|---|
| | | NO for | for Light |
| | | Heavy Chain | Chain |
| | | Amino | Amino Acid |
| ID | Description | Acid Sequence | Sequence |
| TCRBD-39 | TRBV6-5 Fab 1 | 1071 | 1055 |
| TCRBD-40 | TRBV6-5 Fab2 | 1072 | 1056 |
| TCRBD-41 | TRBV6-5 Fab3 | 1073 | 1057 |
| TCRBD-42 | TRBV6-5 Fab4 | 1074 | 1058 |
| TCRBD-43 | TRBV6-5 Fab5 | 1075 | 1059 |
| TCRBD-44 | TRBV6-5 Fab6 | 1076 | 1060 |
| TCRBD-45 | TRBV6-5 Fab7 | 1077 | 1061 |
| TCRBD-46 | TRBV6-5 Fab8 | 1078 | 1062 |
| TCRBD-47 | TRBV6-5 CrossFab1 | 1079 | 1063 |
| TCRBD-48 | TRBV6-5 CrossFab2 | 1080 | 1064 |
| TCRBD-49 | TRBV6-5 CrossFab3 | 1081 | 1065 |
| TCRBD-50 | TRBV6-5 CrossFab4 | 1082 | 1066 |
| TCRBD-51 | TRBV6-5 CrossFab5 | 1083 | 1067 |
| TCRBD-52 | TRBV6-5 CrossFab6 | 1084 | 1068 |
| TCRBD-53 | TRBV6-5 CrossFab7 | 1085 | 1069 |
| TCRBD-54 | TRBV6-5 CrossFab8 | 1086 | 1070 |
| TCRBD-56 | TRBV12-3 Fab1 | 1112 | 1100 |
| TCRBD-57 | TRBV12-3 Fab2 | 1113 | 1101 |
| TCRBD-58 | TRBV12-3 Fab3 | 1114 | 1102 |
| TCRBD-59 | TRBV12-3 Fab4 | 1115 | 1103 |
| TCRBD-60 | TRBV12-3 Fab5 | 1116 | 1104 |
| TCRBD-61 | TRBV12-3 Fab6 | 1117 | 1105 |
| TCRBD-62 | TRBV12-3 CrossFab1 | 1118 | 1106 |
| TCRBD-63 | TRBV12-3 CrossFab2 | 1119 | 1107 |
| TCRBD-64 | TRBV12-3 CrossFab3 | 1120 | 1108 |
| TCRBD-65 | TRBV12-3 CrossFab4 | 1121 | 1109 |
| TCRBD-66 | TRBV12-3 CrossFab5 | 1122 | 1110 |
| TCRBD-67 | TRBV12-3 CrossFab6 | 1123 | 1111 |
| TCRBD-69 | TRBV20-1 Fab1 | 243 | 244 |
| TCRBD-70 | TRBV20-1 CrossFab1 | 245 | 246 |
| CLY-010 | TRBV6-5 | 1155 | 1156 |
| CLY-013 | TRBV6-5 | 1157 | 1158 |

Additional exemplary CDRs, VL, VH, scFvs, Fabs, CrossFab, and/or other binding fragments that can be comprised within the first binding domain can be found, for example, in US 2023/0227552 and US 2021/0277119, the content of each of which is hereby incorporated by reference in its entirety. In some embodiments, the first binding domain comprises CDRs, a VL, a VH, an scFv, a Fab, a CrossFab, and/or other binding fragments from one of the TCR-binding molecules selected from IMMU 546, FIN9, AMKB1-2, ZOE, 3G5, ZIZOU4, IMMU 157, LC4, 3D11, 1C1, W112, MH3-2, 4H11, 36213, BAM13, H131, H132, IMMU 222, JU-74, JU74.3, OT145, BL37.2, S511, VER2.32.1, IG125, 56C5, 56C5.2, 16G8, MX-6, JR2, AF-23, AF23, AHUT7, TAMAYA1.2, BA62, BA62.6, C1, E17.5F3, E17.5F3.15.13, MPB2D5, C21, CAS1.1.3, CH92, 8F10, JOVI-3, WJF24, ELL1.4, JOVI-1, SP305, LG.3A10, 5A8A10, 5D9F1, KFN, A-H.1, A-H.2, A-H.3, A-H.4, A-H.5, A-H.6, A-H.7, A-H.8, A-H.9, A-H.10, A-H. 11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.41, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, A-H.85, B-H. 1A, B-H. 1B, B-H.1C, B-H.1D, B-H. 1E, B-H.1F, B-H.1G, B-H.1H, B-H.1, B-H.1, B-H.2, B-H.3, B-H.4, B-H.5, B-H.6, C—H. 1, C—H.2, C—H.3, C—H.4, C—H.5, C—H.6, C—H.7, C—H.8, C—H.9, C—H. 10, C—H.11, C—H.12, C—H. 13, C—H. 14, C—H.15, C—H.16, C—H.17, C—H.18, C—H.19, C—H.20, C—H.21, C—H.22, C—H.23, C—H.24, C—H.25, C—H.26, C-H.27, C—H.28, C—H.29, C—H.30, C—H.31, C—H.32, C—H.33, C—H.34, C—H.35, C—H.36, C—H.37, C-H.38, C—H.39, C—H.40, C—H.41, C—H.42, C—H.43, C—H.44, C—H.45, C—H.46, C—H.47, C—H.48, C-H.49, C—H.50, D-H.1, D-H.2, D-H.3, D-H.4, D-H.5, D-H.6, D-H.7, D-H.8, D-H.9, D-H.10, D-H.11, D-H.12, D-H.13, D-H.14, D-H.15, D-H.16, D-H.17, D-H.18, D-H. 19, D-H.20, D-H.21, D-H.22, D-H.23, D-H.24, D-H.25, D-H.26, D-H.27, D-H.28, D-H.29, D-H.30, D-H.31, D-H.32, D-H.33, D-H.34, D-H.35, D-H.36, D-H.37, D-H.38, D-H.39, D-H.40, D-H.41, D-H.42, D-H.43, D-H.44, D-H.45, D-H.46, D-H.47, D-H.48, D-H.49, D-H.50, E-H. 1, E-H.2, E-H.3, E-H.4, E-H.5, E-H.6, E-H.7, E-H.8, E-H.9, E-H. 10, E-H.11, E-H. 12, E-H.13, E-H.14, E-H. 15, E-H.16, E-H.17, E-H.18, E-H.19, E-H.20, E-H.21, E-H.22, E-H.23, E-H.24, E-H.25, E-H.26, E-H.27, E-H.28, E-H.29, E-H.30, E-H.31, E-H.32, E-H.33, E-H.34, E-H.35, E-H.36, E-H.37, E-H.38, E-H.39, E-H.40, E-H.41, E-H.42, E-H.43, E-H.44, E-H.45, E-H.46, E-H.47, E-H.48, E-H.49, and E-H.50.

In some embodiments, the TCRαV-binding domain comprises an immunoglobulin heavy chain variable (VH) region and an immunoglobulin light chain variable (VL) region. In some embodiments, the VH region comprises a heavy chain CDR1 (HCDR1), a heavy chain CDR2 (HCDR2), and a heavy chain CDR3 (HCDR3). In some embodiments, the VL region comprises a light chain CDR1 (LCDR1), a light chain CDR2 (LCDR2), and a light chain CDR3 (LCDR3).

In some embodiments, the TCRαV-binding domain is a Fab comprising the VH region, the VL region, a first heavy chain constant region (CH1), and a light chain constant region (CL), wherein the C-terminus of the VH region is connected to the N-terminus of the CH1 region (e.g., a polypeptide chain comprising N′-VH-CH1-C′), and the C-terminus of the VL region is connected to the N-terminus of the CL region (e.g., a polypeptide chain comprising N′-VL-CL-C′). In some embodiments, the CH1 region and the CL region of the Fab are connected by a disulfide bond. In some embodiments, the Fab is a charge steering Fab.

In some embodiments, the TCRαV-binding domain is a CrossFab comprising the VH region, the VL region, a first heavy chain constant region (CH1), and a light chain constant region (CL), wherein the C-terminus of the VH region is connected to the N-terminus of the CL region (e.g., a polypeptide chain comprising N′-VH-CL-C′), and the C-terminus of the VL region is connected to the N-terminus of the CH1 region (e.g., a polypeptide chain comprising N′-VL-CH1-C′). In some embodiments, the CH1 region and the CL region of the CrossFab are connected by a disulfide bond. In some embodiments, the CrossFab is a charge steering CrossFab.

Exemplary anti-TCRαV CDRs are provided in Table 6 (Kabat numbering scheme) and Table 7 (IMGT numbering scheme) below.

TABLE 6
TRAV CDRs (Kabat Numberings)

| Target | | | | | | |
|---|---|---|---|---|---|---|
| Description | HCDR1 | HCDR2 | HCDR3 | LCDR1 | LCDR2 | LCDR3 |
| TRAV1-2 | DTHMH | RTDPASG | YYRDDV | KARQNV | SSSFRYS | QQYNTY |
| | (SEQ ID | DTKYDP | NYAMDY | GSNVA | (SEQ ID | PYT |
| | NO: 134) | KFQG | (SEQ ID | (SEQ ID | NO: 138) | (SEQ |
| | | (SEQ ID | NO: 136) | NO: 137) | | ID NO: |
| | | NO: 135) | | | | 139) |

TABLE 7
TRAV CDRs (IMGT Numberings)

| Target | | | | | | |
|---|---|---|---|---|---|---|
| Description | HCDR1 | HCDR2 | HCDR3 | LCDR1 | LCDR2 | LCDR3 |
| TRAV1-2 | GFNIKDT | TDPASG | AHYYRD | QNVGSN | SSS | QQYNTY |
| | H (SEQ | DT (SEQ | DVNYAM | (SEQ ID | (SEQ | PYT |
| | ID NO: | ID NO: | DY (SEQ | NO: 143) | ID NO: | (SEQ ID |
| | 140) | 141) | ID NO: | | 144) | NO: 145) |
| | | | 142) | | | |

In some embodiments, the HCDR1 comprises an amino acid sequence of DTHMH (SEQ ID NO: 134) or GFNIKDTH (SEQ ID NO: 140).

In some embodiments, the HCDR2 comprises an amino acid sequence of RTDPASGDTKYDPKFQG (SEQ ID NO: 135) or TDPASGDT (SEQ ID NO: 141).

In some embodiments, the HCDR3 comprises an amino acid sequence of YYRDDVNYAMDY (SEQ ID NO: 136) or AHYYRDDVNYAMDY (SEQ ID NO: 142).

In some embodiments, the LCDR1 comprises an amino acid sequence of KARQNVGSNVA (SEQ ID NO: 137) or QNVGSN (SEQ ID NO: 143).

In some embodiments, the LCDR2 comprises an amino acid sequence of SSSFRYS (SEQ ID NO: 138) or SSS (SEQ ID NO: 144).

In some embodiments, the LCDR3 comprises an amino acid sequence of QQYNTYPYT (SEQ ID NO: 139) or QQYNTYPYT (SEQ ID NO: 145).

In some embodiments, the HCDR1 comprises an amino acid sequence of DTHMH (SEQ ID NO: 134), the HCDR2 comprises an amino acid sequence of RTDPASGDTKYDPKFQG (SEQ ID NO: 135), the HCDR3 comprises an amino acid sequence of YYRDDVNYAMDY (SEQ ID NO: 136), the LCDR1 comprises an amino acid sequence of KARQNVGSNVA (SEQ ID NO: 137), the LCDR2 comprises an amino acid sequence of SSSFRYS (SEQ ID NO: 138), the LCDR3 comprises an amino acid sequence of QQYNTYPYT (SEQ ID NO: 139). In some embodiments, the HCDR1 comprises an amino acid sequence of GFNIKDTH (SEQ ID NO: 140), the HCDR2 comprises an amino acid sequence of TDPASGDT (SEQ ID NO: 141), the HCDR3 comprises an amino acid sequence of AHYYRDDVNYAMDY (SEQ ID NO: 142), the LCDR1 comprises an amino acid sequence of QNVGSN (SEQ ID NO: 143), the LCDR2 comprises an amino acid sequence of SSS (SEQ ID NO: 144), the LCDR3 comprises an amino acid sequence of QQYNTYPYT (SEQ ID NO: 145). In some embodiments, the CDRs comprise 1, 2, 3, or more amino acid substitutions.

In some embodiments, the VH region of the TCRαV-binding domain is the VH region of an IgA, IgD, IgE, IgG, or IgM. In some embodiments, the VH region of the TCRαV-binding domain is the VH region of a subclass of an IgA, IgD, IgE, IgG, or IgM, such as, e.g., IgG₁, IgG₂, IgG₃, IgG₄, IgA₁, and IgA₂. In some embodiments, the VL region of the TCRαV-binding domain is the VL region of a kappa light chain. In some embodiments, the VL region of the TCRαV-binding domain is the VL region of a lambda light chain.

Exemplary anti-TCRαV VH and VL are provided in Table 8 below.

TABLE 8
TRAV VH and VL

| Target | | |
|---|---|---|
| Description | VH | VL |
| TRAV1-2 | EVQLQQSGAELVKPGASVKLSCT | DIVMTQSQKFLSTSVGDRVSVTC |
| | ASGFNIKDTHMHWVKQRPEQGLE | KARQNVGSNVAWYQQKPGQSPKS |
| | WIGRTDPASGDTKYDPKFQGKAT | LIYSSSFRYSGVPDRFTGSGSGT |
| | ITADTSSNTAYLHLSSLTSEDTA | DFTLTISNVQSEDLAEYFCQQYN |
| | VYYCAHYYRDDVNYAMDYWGQGT | TYPYTFGGGTELEIK (SEQ ID |
| | TVTVSS (SEQ ID NO: 146) | NO: 147) |

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146. In some embodiments, the VH region comprises or consists of an amino acid sequence of SEQ ID NO: 146.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 44. In some embodiments, the VH region comprises or consists of an amino acid sequence of SEQ ID NO: 146 and comprises a cysteine substitution at amino acid position 44.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 100. In some embodiments, the VH region comprises or consists of an amino acid sequence of SEQ ID NO: 146 and comprises a cysteine substitution at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 100B. In some embodiments, the VH region comprises or consists of an amino acid sequence of SEQ ID NO: 146 and comprises a cysteine substitution at amino acid position 100B.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 101. In some embodiments, the VH region comprises or consists of an amino acid sequence of SEQ ID NO: 146 and comprises a cysteine substitution at amino acid position 101.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 105. In some embodiments, the VH region comprises or consists of an amino acid sequence of SEQ ID NO: 146 and comprises a cysteine substitution at amino acid position 105.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147. In some embodiments, the VL region comprises or consists of an amino acid sequence of SEQ ID NO: 147.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 100. In some embodiments, the VL region comprises or consists of an amino acid sequence of SEQ ID NO: 147 and comprises a cysteine substitution at amino acid position 100.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 49. In some embodiments, the VL region comprises or consists of an amino acid sequence of SEQ ID NO: 147 and comprises a cysteine substitution at amino acid position 49.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 50. In some embodiments, the VL region comprises or consists of an amino acid sequence of SEQ ID NO: 147 and comprises a cysteine substitution at amino acid position 50.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 46. In some embodiments, the VL region comprises or consists of an amino acid sequence of SEQ ID NO: 147 and comprises a cysteine substitution at amino acid position 46.

In some embodiments, the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 43. In some embodiments, the VL region comprises or consists of an amino acid sequence of SEQ ID NO: 147 and comprises a cysteine substitution at amino acid position 43.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 44, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 100.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 100B, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 49.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 100, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 50.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 101, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 46.

In some embodiments, the VH region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 146 and comprises a cysteine residue at amino acid position 105, and the VL region comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 147 and comprises a cysteine residue at amino acid position 43.

In some embodiments, the VH region comprises or consists of an amino acid sequence of SEQ ID NO: 146, and the VL region comprises or consists of an amino acid sequence of SEQ ID NO: 147.

In some embodiments, the VH region comprises an amino acid sequence of SEQ ID NO: 146 with a cysteine substitution at amino acid position 44, and the VL region comprises an amino acid sequence of SEQ ID NO: 147 with a cysteine substitution at amino acid position 100.

In some embodiments, the VH region comprises an amino acid sequence of SEQ ID NO: 146 with a cysteine substitution at amino acid position 100B, and the VL region comprises an amino acid sequence of SEQ ID NO: 147 with a cysteine substitution at amino acid position 49.

In some embodiments, the VH region comprises an amino acid sequence of SEQ ID NO: 146 with a cysteine substitution at amino acid position 100, and the VL region comprises an amino acid sequence of SEQ ID NO: 147 with a cysteine substitution at amino acid position 50.

In some embodiments, the VH region comprises an amino acid sequence of SEQ ID NO: 146 with a cysteine substitution at amino acid position 101, and the VL region comprises an amino acid sequence of SEQ ID NO: 147 with a cysteine substitution at amino acid position 46.

In some embodiments, the VH region comprises an amino acid sequence of SEQ ID NO: 146 with a cysteine substitution at amino acid position 105, and the VL region comprises an amino acid sequence of SEQ ID NO: 147 with a cysteine substitution at amino acid position 43.

In some embodiments, one engineered disulfide bond connects the VH region and the VL region of the TCRαV-binding domain. In some embodiments, two engineered disulfide bonds connect the VH region and the VL region of the TCRαV-binding domain. In some embodiments, three engineered disulfide bonds connect the VH region and the VL region of the TCRαV-binding domain. In some embodiments, four engineered disulfide bonds connect the VH region and the VL region of the TCRαV-binding domain. In some embodiments, five engineered disulfide bonds connect the VH region and the VL region of the TCRαV-binding domain.

In some embodiments, the TCRαV-binding domain comprises a disulfide bond at the position of VH44-VL100. In some embodiments, the TCRαV-binding domain comprises a disulfide bond at the position of VH100B-VL49. In some embodiments, the TCRαV-binding domain comprises a disulfide bond at the position of VH100-VL50. In some embodiments, the TCRαV-binding domain comprises a disulfide bond at the position of VH101-VL46. In some embodiments, the TCRαV-binding domain comprises a disulfide bond at the position of VH105-VL43.

In some embodiments, the TCRαV-binding domain is an scFv. An exemplary scFv is provided in Table 9 below.

TABLE 9
TRAV scFv

| | | | SEQ |
|---|---|---|---|
| ID | Description | Amino Acid Sequence | ID NO |
| TCRBD- | TRAV1-2 | EVQLQQSGAELVKPGASVKLSCTASGFNIKDTHM | 148 |
| 71 | scFv | HWVKQRPEQGLEWIGRTDPASGDTKYDPKFQGKA | |
| | (VH-VL) | TITADTSSNTAYLHLSSLTSEDTAVYYCAHYYRD | |
| | | DVNYAMDYWGQGTTVTVSSGGGGSGGGGSGGGGS | |
| | | DIVMTQSQKFLSTSVGDRVSVTCKARQNVGSNVA | |
| | | WYQQKPGQSPKSLIYSSSFRYSGVPDRFTGSGSG | |
| | | TDFTLTISNVQSEDLAEYFCQQYNTYPYTFGGGT | |
| | | ELEIK | |

In some embodiments, the TCRαV-binding domain comprises an amino acid sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 148. In some embodiments, the TCRαV-binding domain comprises or consists of an amino acid sequence of SEQ ID NO: 148.

In some embodiments, the TCRαV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 1129 and 1130.

In some embodiments, the TCRαV-binding domain comprises a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 1127 and 1128.

In some embodiments, the TCRαV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1129, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1127. In some embodiments, the TCRαV-binding domain comprises a heavy chain comprising SEQ ID NO: 1129, and a light chain comprising SEQ ID NO: 1127. In some embodiments, the TCRαV-binding domain comprises a heavy chain comprising SEQ ID NO: 1129 with 1, 2, 3, 4, 5, 6, 7, 8, 9, 10, or more mutations therein. In some embodiments, the TCRαV-binding domain comprises a light chain comprising SEQ ID NO: 1127 with 1, 2, 3, 4, 5, 6, 7, 8, 9, 10, or more mutations therein. In some embodiments, the TCRαV-binding domain is a Fab.

In some embodiments, the TCRαV-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1130, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1128. In some embodiments, the TCRαV-binding domain comprises a heavy chain comprising SEQ ID NO: 1130, and a light chain comprising SEQ ID NO: 1128. In some embodiments, the TCRαV-binding domain comprises a heavy chain comprising SEQ ID NO: 1130 with 1, 2, 3, 4, 5, 6, 7, 8, 9, 10, or more mutations therein. In some embodiments, the TCRαV-binding domain comprises a light chain comprising SEQ ID NO: 1128 with 1, 2, 3, 4, 5, 6, 7, 8, 9, 10, or more mutations therein. In some embodiments, the TCRαV-binding domain is a Cross-Fab.

In some embodiments, the TCRβV-binding domain is an scFv and the C-terminus of the VH region is linked to the N-terminus of the VL region. In some embodiments, the TCRβV-binding domain is an scFv and the C-terminus of the VL region is linked to the N-terminus of the VH region. In some embodiments, the VH region and the VL region are linked by a linker.

In some embodiments, the TCRαV-binding domain is an scFv and the C-terminus of the VH region is linked to the N-terminus of the VL region. In some embodiments, the TCRαV-binding domain is an scFv and the C-terminus of the VL region is linked to the N-terminus of the VH region. In some embodiments, the VH region and the VL region are linked by a linker. In some embodiments, the TCRβV-binding domain is an antibody selected from the group consisting of: S511, VER2.32.1, IG125, 56C5, 16G8, MX-6, JR2, AF23, AHUT7, TAMAYA1.2, BA62.6, C1, E17.5F3.15.13, IMMU546, MPB2D5, C21, CAS1.1.3, CH92, 8F10, JOVI-3, WJF24, ELL1.4, FIN9, AMKB1-2, ZOE, 3G5, ZIZOU4, IMMU157, LC4, 3D11, 1C1, W112, MH3-2, 36213, BAM13, H132, IMMU222, H131, JU-74, JU743, OT145, and BL37.2, or a fragment or variant thereof. In some embodiments, the TCRβV-binding domain comprises a VH region from an antibody selected from the group consisting of: S511, VER2.32.1, IG125, 56C5, 16G8, MX-6, JR2, AF23, AHUT7, TAMAYA1.2, BA62.6, C1, E17.5F3.15.13, IMMU546, MPB2D5, C21, CAS1.1.3, CH92, 8F10, JOVI-3, WJF24, ELL1.4, FIN9, AMKB1-2, ZOE, 3G5, ZIZOU4, IMMU157, LC4, 3D11, 1C1, W112, MH3-2, 36213, BAM13, H132, IMMU222, H131, JU-74, JU743, OT145, and BL37.2, or a fragment or variant thereof. In some embodiments, the TCRβV-binding domain comprises a VL region from an antibody selected from the group consisting of: S511, VER2.32.1, IG125, 56C5, 16G8, MX-6, JR2, AF23, AHUT7, TAMAYA1.2, BA62.6, C1, E17.5F3.15.13, IMMU546, MPB2D5, C21, CAS1.1.3, CH92, 8F10, JOVI-3, WJF24, ELL1.4, FIN9, AMKB1-2, ZOE, 3G5, ZIZOU4, IMMU157, LC4, 3D11, 1C1, W112, MH3-2, 36213, BAM13, H132, IMMU222, H131, JU-74, JU743, OT145, and BL37.2, or a fragment or variant thereof. In some embodiments, the TCRβV-binding domain comprises a VH region and a VL region from an antibody selected from the group consisting of: S511, VER2.32.1, IG125, 56C5, 16G8, MX-6, JR2, AF23, AHUT7, TAMAYA1.2, BA62.6, C1, E17.5F3.15.13, IMMU546, MPB2D5, C21, CAS1.1.3, CH92, 8F10, JOVI-3, WJF24, ELL1.4, FIN9, AMKB1-2, ZOE, 3G5, ZIZOU4, IMMU157, LC4, 3D11, 1C1, W112, MH3-2, 36213, BAM13, H132, IMMU222, H131, JU-74, JU743, OT145, and BL37.2, or a fragment or variant thereof. In some embodiments, the TCRβV-binding domain comprises CDRs from an antibody selected from the group consisting of: S511, VER2.32.1, IG125, 56C5, 16G8, MX-6, JR2, AF23, AHUT7, TAMAYA1.2, BA62.6, C1, E17.5F3.15.13, IMMU546, MPB2D5, C21, CAS1.1.3, CH92, 8F10, JOVI-3, WJF24, ELL1.4, FIN9, AMKB1-2, ZOE, 3G5, ZIZOU4, IMMU157, LC4, 3D11, 1C1, W112, MH3-2, 36213, BAM13, H132, IMMU222, H131, JU-74, JU743, OT145, and BL37.2, or a fragment or variant thereof. In some embodiments, the CDRs comprise 1, 2, 3, or more amino acid substitutions.

In some embodiments, the TCRαV-binding domain is an antibody selected from the group consisting of: C15, 6B11, SFB280, 3C10, F1, 6D6.6, clone 34, or a fragment or variant thereof. In some embodiments, the TCRαV-binding domain comprises a VH region from an antibody selected from the group consisting of: C15, 6B11, SFB280, 3C10, F1, 6D6.6, clone 34, or a fragment or variant thereof. In some embodiments, the TCRαV-binding domain comprises a VL region from an antibody selected from the group consisting of: C15, 6B11, SFB280, 3C10, F1, 6D6.6, clone 34, or a fragment or variant thereof. In some embodiments, the TCRαV-binding domain comprises a VH region and a VL region from an antibody selected from the group consisting of: C15, 6B11, SFB280, 3C10, F1, 6D6.6, clone 34, or a fragment or variant thereof. In some embodiments, the TCRαV-binding domain comprises CDRs from an antibody selected from the group consisting of: C15, 6B11, SFB280, 3C10, F1, 6D6.6, clone 34, or a fragment or variant thereof. In some embodiments, the CDRs comprise 1, 2, 3, or more amino acid substitutions.

In some embodiments, the TCRβV-binding domain specifically binds to human TCRβV (e.g., a TCRβV family, subfamily, or variant thereof). TCRβV families and subfamilies are known in the art, e.g., as described in Yassai et al., (2009) Immunogenetics 61 (7) pp: 493-502; and Wei S. and Concannon P. (1994) Human Immunology 41 (3) pp: 201-206. Different gene nomenclatures for any one of the TCRβV families and subfamilies described herein are also encompassed by the present disclosure. For example, the TCRβV nomenclature as described by Folch, G. and Lefranc, M.-P., Exp. Clin. Immunogenet., 17, 42-54 (2000); Lefranc, M.-P. and Lefranc, G., The T Cell Receptor FactsBook, Academic Press, Harcourt, 398 pages (2001); Slightom, J. L. et al., Genomics, 20, 149-168 (1994); Wei et al., Immunogenetics, 40, 27-36 (1994); Arden, B. et al., Immunogenetics, 42, 455-500 (1995); Rowen, L. et al., Science, 272, 1755-1762 (1996); and Wilson, R. K. et al., Immunological Reviews 101, 149-172 (1988) are encompassed by the present invention, each of which are hereby incorporated by reference in their entirety.

### TCR Targets

In some embodiments, the TCRβV-binding domain specifically binds to any one of: TCRβV1, TCRβV2, TCRβV3, TCRβV4, TCRβV5, TCRβV6, TCRβV7, TCRβV8, TCRβV9, TCRβV10, TCRβV11, TCRβV12, TCRβV13, TCRβV14, TCRβV15, TCRβV16, TCRβV17, TCRβV18, TCRβV19, TCRβV20, TCRβV21, TCRβV22, TCRβV23, TCRβV24, TCRβV25, TCRβV26, TCRβV27, TCRβV28, TCRβV29, TCRβV30, TCRβVA, TCRβVB, TCRβVC, or a variant thereof. See, also, Folch and Lefranc, Exp. Clin. Immunogenet, 17, 42-54 (2000).

In some embodiments, the TCRβV-binding domain specifically binds to any one of TCRβV2*01, TCRβV2*02, TCRβV2*03, TCRβV3-1*01, TCRβV3-1*02, TCRβV4-1*01, TCRβV4-1*02, TCRβV4-2*01, TCRβV4-2*02, TCRβV4-3*01, TCRβV4-3*02, TCRβV4-3*03, TCRβV4-3*04, TCRβV5-1*01, TCRβV5-1*02, TCRβV5-4*01, TCRβV5-4*02, TCRβV5-4*03, TCRβV5-4*04, TCRβV5-5*01, TCRβV5-5*02, TCRβV5-5*03, TCRβV5-6*01, TCRβV5-8*01, TCRβV5-8*02, TCRβV6-1*01, TCRβV6-2*01, TCRβV6-3*01, TCRβV6-4*01, TCRβV6-4*02, TCRβV6-5*01, TCRβV6-6*01, TCRβV6-6*02, TCRβV6-6*03, TCRβV6-6*04, TCRβV6-6*05, TCRβV6-8*01, TCRβV6-9*01, TCRβV7-2*01, TCRβV7-2*02, TCRβV7-2*03, TCRβV7-2*04, TCRβV7-3*01, TCRβV7-3*04, TCRβV7-3*05, TCRβV7-4*01, TCRβV7-6*01, TCRβV7-6*02, TCRβV7-7*01, TCRβV7-7*02, TCRβV7-8*01, TCRβV7-8*02, TCRβV7-8*03, TCRβV7-9*01, TCRβV7-9*02, TCRβV7-9*03, TCRβV7-9*04, TCRβV7-9*05, TCRβV7-9*06, TCRβV7-9*07, TCRβV9*01, TCRβV9*02, TCRβV9*03, TCRβV10-1*01, TCRβV10-1*02, TCRβV10-2*01, TCRβV10-2*02, TCRβV10-3*01, TCRβV10-3*02, TCRβV10-3*03, TCRβV10-3*04, TCRβV11-1*01, TCRβV11-2*01, TCRβV11-2*02, TCRβV11-2*03, TCRβV11-3*01, TCRβV11-3*02, TCRβV11-3*03, TCRβV11-3*04, TCRβV12-3*01, TCRβV12-4*01, TCRβV12-4*02, TCRβV12-5*01, TCRβV13*01, TCRβV13*02, TCRβV14*01, TCRβV14*02, TCRβV15*01, TCRβV15*02, TCRβV15*03, TCRβV16*01, TCRβV16*03, TCRβV18*01, TCRβV19*01, TCRβV19*02, TCRβV19*03, TCRβV20-1*01, TCRβV20-1*02, TCRβV20-1*03, TCRβV20-1*04, TCRβV20-1*05, TCRβV20-1*06, TCRβV20-1*07, TCRβV24-1*01, TCRβV25-1*01, TCRβV27*01, TCRβV28*01, TCRβV29-1*01, TCRβV29-1*02, TCRβV29-1*03, TCRβV30*01, TCRβV30*02, TCRβV30*03, TCRβV30*04, or a variant thereof.

In some embodiments, the TCRαV-binding domain specifically binds to human TCRαV (e.g., a TCRαV family, subfamily, or variant thereof). TCRαV families and subfamilies are known in the art, e.g., as described in Yassai et al., (2009) Immunogenetics 61 (7) pp: 493-502. Different gene nomenclatures for any one of the TCRαV families and subfamilies described herein are also encompassed by the present disclosure. For example, the TCRαV nomenclature as described by Scaviner D. and Lefranc, M.-P., Exp. Clin. Immunogenet, 17, 83-96 (2000); Lefranc, M.-P. and Lefranc, G., The T Cell Receptor FactsBook, Academic Press, Harcourt, 398 pages (2001); Arden, B. et al., Immunogenetics, 42, 455-500 (1995); and Wilson, R. K. et al., Immunological Reviews 101, 149-172 (1988) are encompassed by the present invention, each of which are hereby incorporated by reference in their entirety.

In some embodiments, the TCRαV-binding domain specifically binds to any one of: TCRαV1, TCRαV2, TCRαV3, TCRαV4, TCRαV5, TCRαV6, TCRαV7, TCRαV8, TCRαV9, TCRαV10, TCRαV11, TCRαV12, TCRαV13, TCRαV14/DV4, TCRαV15, TCRαV16, TCRαV17, TCRαV18, TCRαV19, TCRαV20, TCRαV21, TCRαV22, TCRαV23/DV6, TCRαV24, TCRαV25, TCRαV26, TCRαV27, TCRαV28, TCRαV29/DV5, TCRαV30, TCRαV31, TCRαV32, TCRαV33, TCRαV34, TCRαV35, TCRαV36/DV7, TCRαV37, TCRαV38, TCRαV39, TCRαV40, TCRαV41, or a variant thereof. In some embodiments, the TCRαV-binding domain specifically binds to any one of: TCRαV1-1, TCRαV1-2, TCRαV8-1, TCRαV8-2, TCRαV8-3, TCRαV8-4, TCRαV8-5, TCRαV8-6, TCRαV8-7, TCRαV9-1, TCRαV9-2, TCRαV12-1, TCRαV12-2, TCRαV12-3, TCRαV13-1, TCRαV13-2, TCRαV26-1, TCRαV26-2, TCRαV38-1, TCRαV38-2/DV8, or a variant thereof. See, also, Scaviner D. and Lefranc, M.-P., Exp. Clin. Immunogenet, 17, 83-96 (2000).

Exemplary amino acid sequences for TCRβV family members are shown in Table 10 below and can also be found on the ImMunoGeneTics Information System website: http://www.imgt.org/, or in a similar resource.

TABLE 10
TCRBV

| | | | SEQ ID |
|---|---|---|---|
| Target | Species | Amino Acid Sequence | NO |
| TCRBV5-5 | Human | DAGVTQSPTHLIKTRGQQVTLRCSPISGH | 93 |
| | | KSVSWYQQVLGQGPQFIFQYYEKEERGRG | |
| | | NFPDRFSARQFPNYSSELNVNALLLGDSA | |
| | | LYLCASSL | |
| TCRBV5-5 | Cynomolgus | DGGVTQSPRHLIKTTGQQVTLRCSPISGH | 94 |
| | Monkey | TSVSWYQQALGQGPQFIFEYEEKERERGN | |
| | | FPDRFSGHQFSNYSSELNVNALEAGDSAL | |
| | | YLCASSL | |
| TCRBV5-6 | Human | DAGVTQSPTHLIKTRGQQVTLRCSPKSGH | 95 |
| | | DTVSWYQQALGQGPQFIFQYYEEEERQRG | |
| | | NFPDRFSGHQFPNYSSELNVNALLLGDSA | |
| | | LYLCASSL | |
| TCRBV5-6 | Cynomolgus | DGGVTQSPRHLIKTTGQQVTLRCSPISGH | 96 |
| | Monkey | TSVSWYQQALGQGPQFIFEYEEKERERGN | |
| | | FPDRFSGHQFSNYSSELNVNALEAGDSAL | |
| | | YLCASSL | |
| TCRBV5-6 | Rhesus | DTGVTQSPTHLIRKRGQQVTLRCSPISGH | 97 |
| | Monkey | NTVSWYQQALGQGPQFIFQYYEKEERERG | |
| | | NFPGRFSGHQFPNYSSELNVSALLLGDSA | |
| | | LYLCASSL | |
| TCRBV6-5 | Human | NAGVTQTPKFQVLKTGQSMTLQCAQDMNH | 98 |
| | | EYMSWYRQDPGMGLRLIHYSVGAGITDQG | |
| | | EVPNGYNVSRSTTEDFPLRLLSAAPSQTS | |
| | | VYFCASSY | |
| TCRBV6-5 | Cynomolgus | NVGVTQTPKFQVLKTGQSMTVQCAQDMNH | 99 |
| | Monkey | NFMYWYRQDPGMGLRLIHYSGAAGTTDKG | |
| | | EVPSGYNVSRLNTEDFPLRLESADPSQTS | |
| | | VYICASSY | |
| TCRBV6-5 | Rhesus | NVGVTQTPKFQVLKTGQSMTVQCAQDMNH | 100 |
| | Monkey | NFMYWYRQDPGMGLRLIHYSGAAGTTDKG | |
| | | EVPSGYNVSRLNTEDFPLRLESADPSQTS | |
| | | VYFCASSY | |
| TCRBV12-3 | Human | DAGVIQSPRHEVTEMGQEVTLRCKPISGH | 101 |
| | | NSLFWYRQTMMRGLELLIYFNNNVPIDDS | |
| | | GMPEDRFSAKMPNASFSTLKIQPSEPR | |
| TCRBV12-3 | Cynomolgus | DAGVIQSPRHEVTEMGKEVTLRCEPISGH | 102 |
| | Monkey | TYLFWYRQTMMRGLEFLIYFNNKSPIDDS | |
| | | GMPKDRFSAKMPDASFSTLKIQPSEPRDS | |
| | | AVYFCASSL | |
| TCRBV12-3 | Rhesus | DAGVIQSPQHEVTEMGKEVTLRCEPISGH | 103 |
| | Monkey | TYLFWYRQTMMRGLEFLIYFNNKSPIDDS | |
| | | GMPKDRFSATMPEVSFSTLKIQPSEPRDS | |
| | | AVYFCASSL | |
| TCRBV12-4 | Human | DAGVIQSPRHEVTEMGQEVTLRCKPISGH | 104 |
| | | DYLFWYRQTMMRGLELLIYFNNNVPIDDS | |
| | | GMPEDRFSAKMPNASFSTLKIQPSEPRDS | |
| | | AVYFCASSL | |
| TCRBV12-4 | Cynomolgus | DAGVTQTPRHKVTEMGQEVTMRCQPILGH | 105 |
| | Monkey | NTVFWYRQTVMQGLELLVYLRNKASLDDS | |
| | | GMPKDRFSAEMPDASLATLKIQPSEPRDS | |
| | | AVYLCASGL | |
| TCRBV12-4 | Rhesus | DAGVTQTPRHKVTEMGQEVTMRCQPILGH | 106 |
| | Monkey | NTVFWYRQTVMQGLELLVYLRNKASLDDS | |
| | | GMPKDRFSAEMPDASLATLKIQPSEPRDS | |
| | | AVYLCASGL | |
| TCRBV20-1 | Human | GAVVSQHPSWVICKSGTSVKIECRSLDFQ | 107 |
| | | ATTMFWYRQFPKQSLMLMATSNEGSKATY | |
| | | EQGVEKDKFLINHASLTLSTLTVTSAHPE | |
| | | DSSFYICSAR | |
| TCRBV20-1 | Cynomolgus | AAVVSQYPSRVICKRGTSVKIECRCLDFQ | 108 |
| | Monkey | ATTMFWYRQFQTQSLILMATSNEGSGVTY | |
| | | EQGVKQDKFPINHPNLTFSTLTVTNAHPE | |
| | | DSSFYICSAR | |
| TCRBV20-1 | Rhesus | AAVVSQYPSRVICKRGTSVKIECRCLDFQ | 109 |
| | Monkey | ATTMFWYRQFQTQSLILMATSNEGSGVTY | |
| | | EQGVKQDKFPINHPNLTFSTLTVTNAHPE | |
| | | DSSFYICSAR | |

In some embodiments, TCRβV comprises or consists of an amino acid sequence of any one of SEQ ID NOs: 93-109. In some embodiments, TCRβV comprises or consists of an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NOs: 93-109. In some embodiments, TCRβV comprises or consists of an amino acid sequence of any one of SEQ ID NOs: 93-109 with one or more mutations therein. In some embodiments, TCRβV comprises 1, 2, 3, 4, 5, 6, 7, 8, 9, 10, or more mutations in any one of SEQ ID NOs: 93-109. In some embodiments, TCRβV comprises 1-5, 5-10, 11-15, 15-20, 20-25, 25-30, or more than 30 mutations in any one of SEQ ID NOs: 93-109.

An exemplary amino acid sequence for TCRαV family members is shown in Table 11 below and can also be found on the ImMunoGeneTics Information System website: http://www.imgt.org/, or in a similar resource.

TABLE 11
TCRaV

| | | | SEQ |
|---|---|---|---|
| | | | ID |
| Target | Species | Amino Acid Sequence | NO |
| TCRaV1-2 | Human | WGVFLLYVSMKMGGTTGQNIDQP | 149 |
| | | TEMTATEGAIVQINCTYQTSGFN | |
| | | GLFWYQQHAGEAPTFLSYNVLDG | |
| | | LEEKGRFSSFLSRSKGYSYLLLK | |
| | | ELQMKDSASYLCA | |

In some embodiments, TCRαV comprises or consists of an amino acid sequence of SEQ ID NO: 149. In some embodiments, TCRαV comprises or consists of an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 149. In some embodiments, TCRαV comprises or consists of an amino acid sequence of SEQ ID NO: 149 with one or more mutations therein. In some embodiments, TCRαV comprises 1, 2, 3, 4, 5, 6, 7, 8, 9, 10, or more mutations in SEQ ID NO: 149. In some embodiments, TCRαV comprises 1-5, 5-10, 11-15, 15-20, 20-25, 25-30, or more than 30 mutations in SEQ ID NO: 149.

In some embodiments, the TCRβV-binding domain specifically binds to a TCRβV with high affinity. In some embodiments, the TCRβV-binding domain has a dissociation constant of about 10-5 to about 10-12 moles per liter or molar (M). For example, in some embodiments, the TCRβV-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including any ranges or subranges therebetween. In some embodiments, the TCRβV-binding domain has a dissociation constant of less than 200 nM.

In some embodiments, the TCRαV-binding domain specifically binds to a TCRαV with high affinity. In some embodiments, the TCRαV-binding domain has a dissociation constant of about 10-5 to about 10-12 moles per liter or molar (M). For example, in some embodiments, the TCRαV-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including any ranges or subranges therebetween. In some embodiments, the TCRαV-binding domain has a dissociation constant of less than 200 nM.

In some embodiments, the first binding domain of the multi-specific polypeptide specifically binds CD3 (i.e., a CD3-binding domain). In some embodiments, the CD3-binding domain comprises an immunoglobulin molecule. In some embodiments, the CD3-binding domain is an antibody (e.g., a polyclonal or monoclonal antibody). In some embodiments, the CD3-binding domain is a chimeric antibody. In some embodiments, the CD3-binding domain is a humanized antibody. In some embodiments, the CD3-binding domain is a human antibody.

In some embodiments, the CD3-binding domain is an antigen-binding fragment of an antibody. Non-limiting examples of functional fragments include single-chain Fvs (scFv), Fab fragments, CrossFab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In some embodiments, the CD3-binding binding domain is a Fab comprising a Fab heavy chain and Fab light chain. In some embodiments, the CD3-binding domain is a single chain variable fragment (scFv), Fab, Fab′, or F(ab′)₂.

In some embodiments, the CD3-binding domain comprises: (a) an immunoglobulin heavy chain variable (VH) region comprising a heavy chain complementarity determining region (CDR) 1 (HCDR1), a heavy chain CDR2 (HCDR2), and a heavy chain CDR3 (HCDR3); and (b) an immunoglobulin light chain variable (VL) region comprising a light chain complementarity determining region (CDR) 1 (LCDR1), a light chain CDR2 (LCDR2), and a light chain CDR3 (LCDR3).

In some embodiments, the VH region and the VL region are numbered with reference to the Kabat numbering scheme. In some embodiments, the VH region and the VL region are numbered with reference to the IMGT numbering scheme. In some embodiments, the VH region and the VL region are numbered with reference to the Chothia numbering scheme. In some embodiments, the VH region and the VL region are numbered with reference to the Martin (Enhanced Chothia or AbM) numbering scheme. In some embodiments, the VH region and the VL region are numbered with reference to the Honneger's (Aho) numbering scheme.

In some embodiments, the CD3-binding domain specifically binds to a CD3 with high affinity. In some embodiments, the CD3-binding domain has a dissociation constant of about 10-5 to about 10-12 moles per liter or molar (M). For example, in some embodiments, the CD3-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including any ranges or subranges therebetween. In some embodiments, the CD3-binding domain has a dissociation constant of less than 200 nM. In some embodiments, the CD3 is a human CD3.

In some embodiments, the CD3-binding domain is a Fab comprising the VH region, the VL region, a first heavy chain constant region (CH1), and a light chain constant region (CL), wherein the C-terminus of the VH region is connected to the N-terminus of the CH1 region (e.g., a polypeptide chain comprising N′-VH-CH1-C′), and the C-terminus of the VL region is connected to the N-terminus of the CL region (e.g., a polypeptide chain comprising N′-VL-CL-C′). In some embodiments, the CH1 region and the CL region of the Fab are connected by a disulfide bond. In some embodiments, the Fab is a charge steering Fab.

In some embodiments, the CD3-binding domain is a CrossFab comprising the VH region, the VL region, a first heavy chain constant region (CH1), and a light chain constant region (CL), wherein the C-terminus of the VH region is connected to the N-terminus of the CL region (e.g., a polypeptide chain comprising N′-VH-CL-C′), and the C-terminus of the VL region is connected to the N-terminus of the CH1 region (e.g., a polypeptide chain comprising N′-VL-CH1-C′). In some embodiments, the CH1 region and the CL region of the CrossFab are connected by a disulfide bond. In some embodiments, the CrossFab is a charge steering CrossFab.

In some embodiments, the CD3-binding domain is an scFv. In some embodiments, the CD3-binding domain comprises a VH and a VL, wherein the C-terminus of the VH region is linked to the N-terminus of the VL region. In some embodiments, the CD3-binding domain comprises a VH and a VL, wherein the C-terminus of the VL region is linked to the N-terminus of the VH region. In some embodiments, the VH and VL are linked by a peptide linker (PL1).

Exemplary anti-CD3 CDRs and VH/VL are provided in Table 12 below.

TABLE 12
CD3 CDRs and VH/VL

| | | SEQ |
|---|---|---|
| | | ID |
| Description | Amino Acid Sequence | NO |
| HCDR1 (Kabat) | KYAMN | 201 |
| HCDR2 (Kabat) | RIRSKYNNYATYYADSVKD | 202 |
| HCDR3 (Kabat) | HGNFGNSYISYWAY | 203 |
| LCDR1 (Kabat) | GSSTGAVTSGNYPN | 204 |
| LCDR2 (Kabat) | GTKFLAP | 205 |
| LCDR3 (Kabat) | VLWYSNRWV | 206 |
| HCDR1 (IMGT) | GFTFNKYA | 207 |
| HCDR2 (IMGT) | IRSKYNNYAT | 208 |
| HCDR3 (IMGT) | VRHGNFGNSYISYWAY | 209 |
| LCDR1 (IMGT) | TGAVTSGNY | 210 |
| LCDR2 (IMGT) | GTK | 211 |
| LCDR3 (IMGT) | VLWYSNRWV | 212 |
| VH | EVQLVESGGGLVQPGGSLK | 213 |
| | LSCAASGFTFNKYAMNWVR | |
| | QAPGKGLEWVARIRSKYNN | |
| | YATYYADSVKDRFTISRDD | |
| | SKNTAYLQMNNLKTEDTAV | |
| | YYCVRHGNFGNSYISYWAY | |
| | WGQGTLVTVSS | |
| VL | QTVVTQEPSLTVSPGGTVT | 214 |
| | LTCGSSTGAVTSGNYPNWV | |
| | QQKPGQAPRGLIGGTKFLA | |
| | PGTPARFSGSLLGGKAALT | |
| | LSGVQPEDEAEYYCVLWYS | |
| | NRWVFGGGTKLTVL | |

In some embodiments, the HCDR1 comprises SEQ ID NO: 201. In some embodiments, the HCDR2 comprises SEQ ID NO: 202. In some embodiments, the HCDR3 comprises SEQ ID NO: 203. In some embodiments, the LCDR1 comprises SEQ ID NO: 204. In some embodiments, the LCDR2 comprises SEQ ID NO: 205. In some embodiments, the LCDR3 comprises SEQ ID NO: 206.

In some embodiments, the HCDR1 comprises SEQ ID NO: 207. In some embodiments, the HCDR2 comprises SEQ ID NO: 208. In some embodiments, the HCDR3 comprises SEQ ID NO: 209. In some embodiments, the LCDR1 comprises SEQ ID NO: 210. In some embodiments, the LCDR2 comprises SEQ ID NO: 211. In some embodiments, the LCDR3 comprises SEQ ID NO: 212.

In some embodiments, the CD3-binding domain comprises: (a) an HCDR1 comprising SEQ ID NO: 201; (b) an HCDR2 comprising SEQ ID NO: 202; (c) an HCDR3 comprising SEQ ID NO: 203; (d) an LCDR1 comprising SEQ ID NO: 204; (e) an LCDR2 comprising SEQ ID NO: 205; and (f) an LCDR3 comprising SEQ ID NO: 206. In some embodiments, the CDRs comprise 1, 2, 3, or more amino acid substitutions.

In some embodiments, the CD3-binding domain comprises: (a) an HCDR1 comprising SEQ ID NO: 207; (b) an HCDR2 comprising SEQ ID NO: 208; (c) an HCDR3 comprising SEQ ID NO: 209; (d) an LCDR1 comprising SEQ ID NO: 210; (e) an LCDR2 comprising SEQ ID NO: 211; and (f) an LCDR3 comprising SEQ ID NO: 212. In some embodiments, the CDRs comprise 1, 2, 3, or more amino acid substitutions.

In some embodiments, the VH region of the CD3-binding domain is the VH region of an IgA, IgD, IgE, IgG, or IgM. In some embodiments, the VH region of the CD3-binding domain is the VH region of a subclass of an IgA, IgD, IgE, IgG, or IgM, such as, e.g., IgG1, IgG2, IgG3, IgG4, IgA1, and IgA2. In some embodiments, the VL region of the CD3-binding domain is the VL region of a kappa light chain. In some embodiments, the VL region of the CD3-binding domain is the VL region of a lambda light chain.

In some embodiments, the VH region of the CD3-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 213, and wherein the VL region of the CD3-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 214. In some embodiments, the VH region of the CD3-binding domain comprises SEQ ID NO: 213, and wherein the VL region of the CD3-binding domain comprises SEQ ID NO: 214.

In some embodiments, the CD3-binding domain comprises an scFv comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 215.

In some embodiments, the CD3-binding domain comprises an scFv comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 216.

In some embodiments, the CD3-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 217 and 219.

In some embodiments, the CD3-binding domain comprises a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 218 and 220.

In some embodiments, the CD3-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 217, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 218. In some embodiments, the CD3-binding domain comprises a heavy chain comprising SEQ ID NO: 217, and a light chain comprising SEQ ID NO: 218. In some embodiments, the CD3-binding domain comprises a heavy chain comprising SEQ ID NO: 217 with 1, 2, 3, 4, 5, 6, 7, 8, 9, 10, or more mutations therein. In some embodiments, the CD3-binding domain comprises a light chain comprising SEQ ID NO: 218 with 1, 2, 3, 4, 5, 6, 7, 8, 9, 10, or more mutations therein. In some embodiments, the CD3-binding domain is a Fab.

In some embodiments, the CD3-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 219, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 220. In some embodiments, the CD3-binding domain comprises a heavy chain comprising SEQ ID NO: 219, and a light chain comprising SEQ ID NO: 220. In some embodiments, the CD3-binding domain comprises a heavy chain comprising SEQ ID NO: 219 with 1, 2, 3, 4, 5, 6, 7, 8, 9, 10, or more mutations therein. In some embodiments, the CD3-binding domain comprises a light chain comprising SEQ ID NO: 220 with 1, 2, 3, 4, 5, 6, 7, 8, 9, 10, or more mutations therein. In some embodiments, the CD3-binding domain is a Cross-Fab.

In some embodiments, the CD3-binding domain is a blinatumomab antibody, or a fragment or variant thereof. In some embodiments, the CD3-binding domain comprises a VH region from blinatumomab or a fragment or variant thereof. In some embodiments, the CD3-binding domain comprises a VL region from blinatumomab or a fragment or variant thereof. In some embodiments, the CD3-binding domain comprises a VH region and a VL region from blinatumomab or a fragment or variant thereof. In some embodiments, the CD3-binding domain comprises CDRs from blinatumomab or a fragment or variant thereof. In some embodiments, the CDRs comprise 1, 2, 3, or more amino acid substitutions.

### 1.2.2. Second Binding Domain

In some embodiments, the multi-specific polypeptides of the disclosure comprise a second binding domain.

In some embodiments, the second binding domain comprises an immunoglobulin molecule. In some embodiments, the second binding domain is an antibody (e.g., a polyclonal or monoclonal antibody). In some embodiments, the second binding domain is a chimeric antibody. In some embodiments, the second binding domain is a humanized antibody. In some embodiments, the second binding domain is a human antibody.

In some embodiments, the second binding domain is an antigen-binding fragment of an antibody. Non-limiting examples of functional fragments include single-chain Fvs (scFv), Fab fragments, CrossFab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In some embodiments, the second binding domain is a Fab comprising a Fab heavy chain and Fab light chain.

In some embodiments, the second binding domain comprises: (a) an immunoglobulin heavy chain variable (VH) region comprising a heavy chain complementarity determining region (CDR) 1 (HCDR1), a heavy chain CDR2 (HCDR2), and a heavy chain CDR3 (HCDR3); and (b) an immunoglobulin light chain variable (VL) region comprising a light chain complementarity determining region (CDR) 1 (LCDR1), a light chain CDR2 (LCDR2), and a light chain CDR3 (LCDR3).

In some embodiments, the VH region and the VL region are numbered with reference to the Kabat numbering scheme. In some embodiments, the VH region and the VL region are numbered with reference to the IMGT numbering scheme. In some embodiments, the VH region and the VL region are numbered with reference to the Chothia numbering scheme. In some embodiments, the VH region and the VL region are numbered with reference to the Martin (Enhanced Chothia or AbM) numbering scheme. In some embodiments, the VH region and the VL region are numbered with reference to the Honneger's (Aho) numbering scheme.

In some embodiments, the second binding domain specifically binds to a protein expressed on a B cell. In some embodiments, the second binding domain specifically binds CD19 (i.e., a CD19-binding domain). In some embodiments, the CD19-binding domain specifically binds to a CD19 with high affinity. In some embodiments, the CD19-binding domain has a dissociation constant of about 10-5 to about 10-12 moles per liter or molar (M). For example, in some embodiments, the CD19-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including any ranges or subranges therebetween. In some embodiments, the CD19-binding domain has a dissociation constant of less than 200 nM.

CD19 is a type-I transmembrane glycoprotein that belongs to the immunoglobulin superfamily and is widely expressed on B cells throughout most stages of B-cell differentiation. In B cells, CD19 usually associates with CD21, CD81, and CD225, forming a signal transduction complex. Expression of CD19 is also found in the majority of B cell-derived malignancies, as well as on follicular dendritic cells. The canonical sequence of human CD19 protein can be found in Uniprot ID P15391.

Exemplary CD19 sequences are provided in Table 13 below.

TABLE 13
CD19

| | | SEQ ID |
|---|---|---|
| Description | Amino Acid Sequence | NO |
| Human CD19 | PEEPLVVKVEEGDNAVLQCLKGTSDGPTQQLTWSRE | 151 |
| | SPLKPFLKLSLGLPGLGIHMRPLAIWLFIFNVSQQM | |
| | GGFYLCQPGPPSEKAWQPGWTVNVEGSGELFRWNVS | |
| | DLGGLGCGLKNRSSEGPSSPSGKLMSPKLYVWAKDR | |
| | PEIWEGEPPCLPPRDSLNQSLSQDLTMAPGSTLWLS | |
| | CGVPPDSVSRGPLSWTHVHPKGPKSLLSLELKDDRP | |
| | ARDMWVMETGLLLPRATAQDAGKYYCHRGNLTMSFH | |
| | LEITARPVLWHWLLRTGGWKVSAVTLAYLIFCLCSL | |
| | VGILHLQRALVLRRKRKRMTDPTRRFFKVTPPPGSG | |
| | PQNQYGNVLSLPTPTSGLGRAQRWAAGLGGTAPSYG | |
| | NPSSDVQADGALGSRSPPGVGPEEEEGEGYEEPDSE | |
| | EDSEFYENDSNLGQDQLSQDGSGYENPEDEPLGPED | |
| | EDSFSNAESYENEDEELTQPVARTMDFLSPHGSAWD | |
| | PSREATSLGSQSYEDMRGILYAAPQLRSIRGQPGPN | |
| | HEEDADSYENMDNPDGPDPAWGGGGRMGTWSTR | |
| Cynomolgus | QEPLVVKVEEGDNAVLQCLEGTSDGPTQQLVWCRDS | 152 |
| Monkey | PFEPFLNLSLGLPGMGIRMGPLGIWLLIFNVSNQTG | |
| CD19 | GFYLCQPGLPSEKAWQPGWTVSVEGSGELFRWNVSD | |
| | LGGLGCGLKNRSSEGPSSPSGKLNSSQLYVWAKDRP | |
| | EMWEGEPVCGPPRDSLNQSLSQDLTMAPGSTLWLSC | |
| | GVPPDSVSRGPLSWTHVRPKGPKSSLLSLELKDDRP | |
| | DRDMWVVDTGLLLTRATAQDAGKYYCHRGNWTKSFY | |
| | LEITARPALWHWLLRIGGWKVPAVTLTYLIFCLCSL | |
| | VGILQLQRALVLRRKRKRMTDPTRRFFKVTPPPGSG | |
| | PQNQYGNVLSLPTPTSGLGRAQRWAAGLGGTAPSYG | |
| | NPSSDVQVDGAVGSRSPPGAGPEEEEGEGYEEPDSE | |
| | EGSEFYENDSNFGQDQLSQDGSGYENPEDEPLGPED | |
| | EDSFSNAESYENEDEELTQPVARTMDFLSPHGSAWD | |
| | PSREATSLGSQSYEDMRGLLYAAPQLRTIRGQPGPN | |
| | HEEDADSYENMDNPDGPDPAWGGGGRMGTWSAR | |
| Rhesus Monkey | QEPLVVKVEEGDNAVLQCLEGTSDGPTQQLVWCRDS | 153 |
| CD19 | PFEPFLNLSLGLPGMGIRMGPLGIWLLIFNVSNQTG | |
| | GFYLCQPGLPSEKAWQPGWTVSVEGSGELFRWNVSD | |
| | LGGLGCGLKNRSSEGPSSPSGKLNSSQLYVWAKDRP | |
| | EMWEGEPVCGPPRDSLNOSLSQDLTMAPGSTLWLSC | |
| | GVPPDSVSRGPLSWTHVRPKGPKSSLLSLELKDDRP | |
| | DRDMWVVDTGLLLTRATAQDAGKYYCHRGNWTKSFY | |
| | LEITARPALWHWLLRIGGWKVPAVTLTYLIFCLCSL | |
| | VGILQLQRALVLRRKRKRMTDPTRRFFKVTPPPGSG | |
| | PQNQYGNVLSLPTPTSGLGRAQRWAAGLGGTAPSYG | |
| | NPSSDVQVDGAVGSRSPPGAGPEEEEGEGYEEPDSE | |
| | EGSEFYENDSNFGQDQLSQDGSGYENPEDEPLGPED | |
| | EDSFSNAESYENEDEELTQPVARTMDFLSPHGSAWD | |
| | PSREATSLGSQSYEDMRGLLYAAPQLRTIRGQPGPN | |
| | HEEDADSYENMDNPDGPDPAWGGGGRMGTWSAR | |

In some embodiments, CD19 comprises or consists of an amino acid sequence of any one of SEQ ID NOs: 151-153. In some embodiments, CD19 comprises or consists of an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NOs: 151-153. In some embodiments, CD19 comprises or consists of an amino acid sequence of any one of SEQ ID NOs: 151-153 with one or more mutations therein. In some embodiments, CD19 comprises 1, 2, 3, 4, 5, 6, 7, 8, 9, 10, or more mutations in any one of SEQ ID NOs: 151-153. In some embodiments, CD19 comprises 1-5, 5-10, 11-15, 15-20, 20-25, 25-30, or more than 30 mutations in any one of SEQ ID NOs: 151-153. In some embodiments, the CD19 protein comprises an extracellular domain sequence having at least 70%, at least 75%, at least 80%, at least 85%, at least 90%, at least 95%, at least 96%, at least 97%, at least 98%, at least 99%, at least 99.5%, or 100% identity to amino acids 1-272 of SEQ ID NO: 151-153. In some embodiments, the CD19-binding domain specifically binds the extracellular domain of CD19.

In some embodiments, the CD19-binding domain comprises: (i) an immunoglobulin heavy chain variable (VH) region comprising a heavy chain CDR1 (HCDR1), a heavy chain CDR2 (HCDR2), and a heavy chain CDR3 (HCDR3), and (ii) an immunoglobulin light chain variable (VL) region comprising a light chain CDR1 (LCDR1), a light chain CDR2 (LCDR2), and a light chain CDR3 (LCDR3). In some embodiments, the CD19-binding domain is a single chain variable fragment (scFv), Fab, CrossFab, Fab′, or F(ab′)₂.

In some embodiments, the CD19-binding domain is a Fab comprising the VH region, the VL region, a first heavy chain constant region (CH1), and a light chain constant region (CL), wherein the C-terminus of the VH region is connected to the N-terminus of the CH1 region (e.g., a polypeptide chain comprising N′-VH-CH1-C′), and the C-terminus of the VL region is connected to the N-terminus of the CL region (e.g., a polypeptide chain comprising N′-VL-CL-C′). In some embodiments, the CH1 region and the CL region of the Fab are connected by a disulfide bond. In some embodiments, the Fab is a charge steering Fab.

In some embodiments, the CD19-binding domain is a CrossFab comprising the VH region, the VL region, a first heavy chain constant region (CH1), and a light chain constant region (CL), wherein the C-terminus of the VH region is connected to the N-terminus of the CL region (e.g., a polypeptide chain comprising N′-VH-CL-C′), and the C-terminus of the VL region is connected to the N-terminus of the CH1 region (e.g., a polypeptide chain comprising N′-VL-CH1-C′). In some embodiments, the CH1 region and the CL region of the CrossFab are connected by a disulfide bond. In some embodiments, the CrossFab is a charge steering CrossFab.

In some embodiments, the CD19-binding domain is an scFv. In some embodiments, the CD19-binding domain comprises a VH and a VL, wherein the C-terminus of the VH region is linked to the N-terminus of the VL region. In some embodiments, the CD19-binding domain comprises a VH and a VL, wherein the C-terminus of the VL region is linked to the N-terminus of the VH region. In some embodiments, the VH and VL are linked by a peptide linker (PL2).

In some embodiments, the peptide linker (PL2) is (G)n, n=1-30. In some embodiments, the peptide linker (PL2) is (G×S)n, x=1-5, n=1-5. In some embodiments, the peptide linker (PL2) is (S×G)n, x=1-5, n=1-5. In some embodiments, the peptide linker (PL2) is (GSGGS) n, n=1-5. In some embodiments, the peptide linker (PL2) is (GGGGS)n, n=1-5. In some embodiments, the peptide linker (PL2) is (GGGGS)5. In some embodiments, the peptide linker (PL2) is (GGGGS)4. In some embodiments, the peptide linker (PL2) is (GGGGS)3. In some embodiments, the peptide linker (PL2) is (GGGGS)2. In some embodiments, the peptide linker (PL2) is (GGGGS)1. Exemplary linkers are provided in Table 16 of the disclosure.

Exemplary anti-CD19 CDRs are provided in Table 14 (Kabat numbering scheme) and Table 15 (IMGT numbering scheme) below.

TABLE 14
CD19 CDR-Kabat

| Target | | | | | | |
|---|---|---|---|---|---|---|
| Description | HCDR1 | HCDR2 | HCDR3 | LCDR1 | LCDR2 | LCDR3 |
| CD19 | SYWMN | QIWPGD | RETTTV | KASQSVD | DASNLVS | QQSTEDP |
| | (SEQ ID | GDTNYN | GRYYYA | YDGDSYL | (SEQ ID | WT |
| | NO: 301) | GKFKG | MDY | N | NO: 327) | (SEQ ID |
| | | (SEQ ID | (SEQ ID | (SEQ ID | | NO: 332) |
| | | NO: 308) | NO: 315) | NO: 321) | | |
| CD19 | SNWMH | EIDPSD | GSNPYY | SASSGVN | DTSKLAS | HQRGSYT |
| | (SEQ ID | SYTNYN | YAMDY | YMH | (SEQ ID | (SEQ ID |
| | NO: 302) | QNFQG | (SEQ ID | (SEQ ID | NO: 328) | NO: 333) |
| | | (SEQ ID | NO: 316) | NO: 322) | | |
| | | NO: 309) | | | | |
| CD19 | SNWMH | EIDPSD | GSNPYY | SASSGVN | DTSKLAS | HQRGSYT |
| | (SEQ ID | SYTNYN | YAMDY | YMH | (SEQ ID | (SEQ ID |
| | NO: 302) | QKFKG | (SEQ ID | (SEQ ID | NO: 328) | NO: 333) |
| | | (SEQ ID | NO: 316) | NO: 322) | | |
| | | NO: 310) | | | | |
| CD19 | SYVMH | YINPYN | GTYYYG | RSSKSLQ | RMSNLNS | MQHLEYP |
| | (SEQ ID | DGTKYN | TRVFDY | NVNGNTY | (SEQ ID | IT |
| | NO: 303) | EKFQG | (SEQ ID | LY | NO: 329) | (SEQ ID |
| | | (SEQ ID | NO: 317) | (SEQ ID | | NO: 334) |
| | | NO: 311) | | NO: 323) | | |
| CD19 | SNWMH | EIDPSD | GSNPYY | SASSGVN | DTSKLAS | HQRGSYT |
| | (SEQ ID | SYTNYN | YAMDY | YMH | (SEQ ID | (SEQ ID |
| | NO: 302) | QNFQG | (SEQ ID | (SEQ ID | NO: 328) | NO: 333) |
| | | (SEQ ID | NO: 316) | NO: 322) | | |
| | | NO: 309) | | | | |
| CD19 | TSGMG | HIWWDD | MELWSY | SASSSVS | DTSKLAS | FQGSVYP |
| | VG | DKRYNP | YFDY | YMH | (SEQ ID | FT |
| | (SEQ ID | ALKS | (SEQ ID | (SEQ ID | NO: 328) | (SEQ ID |
| | NO: 304) | (SEQ ID | NO: 318) | NO: 324) | | NO: 335) |
| | | NO: 312) | | | | |
| CD19 | SSWMN | RIYPGD | SGFITT | RASESVD | EASNQGS | QQSKEVP |
| | (SEQ ID | GDTNYN | VRDFDY | TFGISFM | (SEQ ID | FT |
| | NO: 305) | VKFKG | (SEQ ID | N | NO: 330) | (SEQ ID |
| | | (SEQ ID | NO: 319) | (SEQ ID | | NO: 336) |
| | | NO: 313) | | NO: 325) | | |
| CD19 | TSTMG | FIWWDD | MELWSY | SASSSVG | DTSKLAS | FQGSVYP |
| | VG | DKRYNP | YFDY | YMH | (SEQ ID | FT |
| | (SEQ ID | NLKS | (SEQ ID | | NO: 328) | (SEQ ID |
| | NO: 306) | (SEQ ID | NO: 318) | (SEQ ID | | NO: 335) |
| | | NO: 314) | | NO: 326) | | |
| CD19 | SGMGV | HIWWDD | MELWSY | SASSSVG | DTSKLAS | FQGSVYP |
| | S | DKRYNP | YFDP | YMH | (SEQ ID | FT |
| | (SEQ ID | ALKS | (SEQ ID | (SEQ ID | NO: 328) | (SEQ ID |
| | NO: 307) | (SEQ ID | NO: 320) | NO: 326) | | NO: 335) |
| | | NO: 312) | | | | |
| CD19 | TSTMG | FIWWDD | MELWSY | SASSSVG | DTSKLAF | FQGSVYP |
| | VG | DKRYNP | YFDP | YMH | (SEQ ID | FT |
| | (SEQ ID | NLKS | (SEQ ID | (SEQ ID | NO: 331) | (SEQ ID |
| | NO: 306) | (SEQ ID | NO: 320) | NO: 326) | | NO: 335) |
| | | NO: 314) | | | | |

TABLE 15
CD19 CDR-IMGT

| Target | | | | | | |
|---|---|---|---|---|---|---|
| Description | HCDR1 | HCDR2 | HCDR3 | LCDR1 | LCDR2 | LCDR3 |
| CD19 | GYAFSSY | IWPGDGD | ARRETT | QSVDY | DAS | QQSTED |
| | W | T | TVGRYY | DGDSY | (SEQ ID | PWT |
| | (SEQ ID | (SEQ ID | YAMDY | (SEQ ID | NO: 361) | (SEQ ID |
| | NO: 337) | NO: 344) | (SEQ ID | NO: 355) | | NO: 332) |
| | | | NO: 349) | | | |
| CD19 | GYTFTSN | IDPSDSY | ARGSNP | SGVNY | DTS | HQRGSY |
| | W | T | YYYAMD | (SEQ ID | (SEQ ID | T |
| | (SEQ ID | (SEQ ID | Y | NO: 356) | NO: 362) | (SEQ ID |
| | NO: 338) | NO: 345) | (SEQ ID | | | NO: 333) |
| | | | NO: 350) | | | |
| CD19 | GYTFTSN | IDPSDSY | ARGSNP | SGVNY | DTS | HQRGSY |
| | W | T | YYYAMD | (SEQ ID | (SEQ ID | T |
| | (SEQ ID | (SEQ ID | Y | NO: 356) | NO: 362) | (SEQ ID |
| | NO: 338) | NO: 345) | (SEQ ID | | | NO: 333) |
| | | | NO: 350) | | | |
| CD19 | GYTFTSY | INPYNDG | ARGTYY | KSLQN | RMS | MQHLEY |
| | V | T | YGTRVF | VNGNT | (SEQ ID | PIT |
| | (SEQ ID | (SEQ ID | DY | Y | NO: 363) | (SEQ ID |
| | NO: 339) | NO: 346) | (SEQ ID | (SEQ ID | | NO: 334) |
| | | | NO: 351) | NO: 357) | | |
| CD19 | GYTFTSN | IDPSDSY | ARGSNP | SGVNY | DTS | HQRGSY |
| | W | T | YYYAMD | (SEQ ID | (SEQ ID | T |
| | (SEQ ID | (SEQ ID | Y | NO: 356) | NO: 362) | (SEQ ID |
| | NO: 338) | NO: 345) | (SEQ ID | | | NO: 333) |
| | | | NO: 350) | | | |
| CD19 | GGSISTS | IWWDDDK | ARMELW | SSVSY | DTS | FQGSVY |
| | GMG | (SEQ ID | SYYFDY | (SEQ ID | (SEQ ID | PFT |
| | (SEQ ID | NO: 347) | (SEQ ID | NO: 358) | NO: 362) | (SEQ ID |
| | NO: 340) | | NO: 352) | | | NO: 335) |
| CD19 | GFTFSSS | IYPGDGD | ARSGFI | ESVDT | EAS | QQSKEV |
| | W | T | TTVRDF | FGISF | (SEQ ID | PFT |
| | (SEQ ID | (SEQ ID | DY | (SEQ ID | NO: 364) | (SEQ ID |
| | NO: 341) | NO: 348) | (SEQ ID | NO: 359) | | NO: 336) |
| | | | NO: 353) | | | |
| CD19 | GGSISTS | IWWDDDK | ARMELW | SSVGY | DTS | FQGSVY |
| | TMG | (SEQ ID | SYYFDY | (SEQ ID | (SEQ ID | PFT |
| | (SEQ ID | NO: 347) | (SEQ ID | NO: 360) | NO: 362) | (SEQ ID |
| | NO: 342) | | NO: 352) | | | NO: 335) |
| CD19 | GGSIASG | IWWDDDK | ARMELW | SSVGY | DTS | FQGSVY |
| | MG | (SEQ ID | SYYFDP | (SEQ ID | (SEQ ID | PFT |
| | (SEQ ID | NO: 347) | (SEQ ID | NO: 360) | NO: 362) | (SEQ ID |
| | NO: 343) | | NO: 354) | | | NO: 335) |
| CD19 | GGSISTS | IWWDDDK | ARMELW | SSVGY | DTS | FQGSVY |
| | TMG | (SEQ ID | SYYFDP | (SEQ ID | (SEQ ID | PFT |
| | (SEQ ID | NO: 347) | (SEQ ID | NO: 360) | NO: 362) | (SEQ ID |
| | NO: 342) | | NO: 354) | | | NO: 335) |

In some embodiments, the HCDR1 comprises any one of SEQ ID NO: 301-307 and 337-343. In some embodiments, the HCDR2 comprises any one of SEQ ID NO: 308-314 and 344-348. In some embodiments, the HCDR3 comprises any one of SEQ ID NO: 315-320 and 349-354. In some embodiments, the LCDR1 comprises any one of SEQ ID NO: 321-326 and 355-360. In some embodiments, the LCDR2 comprises any one of SEQ ID NO: 327-331 and 361-364. In some embodiments, the LCDR3 comprises any one of SEQ ID NO: 332-336.

In some embodiments, the CD19-binding domain comprises: (a) an HCDR1 comprising SEQ ID NO: 301; (b) an HCDR2 comprising SEQ ID NO: 308; (c) an HCDR3 comprising SEQ ID NO: 315; (d) an LCDR1 comprising SEQ ID NO: 321; (e) an LCDR2 comprising SEQ ID NO: 327; and (f) an LCDR3 comprising SEQ ID NO: 332. In some embodiments, the CDRs comprise 1, 2, 3, or more amino acid substitutions.

In some embodiments, the CD19-binding domain comprises: (a) an HCDR1 comprising SEQ ID NO: 302; (b) an HCDR2 comprising SEQ ID NO: 309; (c) an HCDR3 comprising SEQ ID NO: 316; (d) an LCDR1 comprising SEQ ID NO: 322; (e) an LCDR2 comprising SEQ ID NO: 328; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 302; (b) an HCDR2 comprising SEQ ID NO: 310; (c) an HCDR3 comprising SEQ ID NO: 316; (d) an LCDR1 comprising SEQ ID NO: 322; (e) an LCDR2 comprising SEQ ID NO: 328; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 303; (b) an HCDR2 comprising SEQ ID NO: 311; (c) an HCDR3 comprising SEQ ID NO: 317; (d) an LCDR1 comprising SEQ ID NO: 323; (e) an LCDR2 comprising SEQ ID NO: 329; and (f) an LCDR3 comprising SEQ ID NO: 334. In some embodiments, the CDRs comprise 1, 2, 3, or more amino acid substitutions.

In some embodiments, the CD19-binding domain comprises: (a) an HCDR1 comprising SEQ ID NO: 302; (b) an HCDR2 comprising SEQ ID NO: 309; (c) an HCDR3 comprising SEQ ID NO: 316; (d) an LCDR1 comprising SEQ ID NO: 322; (e) an LCDR2 comprising SEQ ID NO: 328; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 304; (b) an HCDR2 comprising SEQ ID NO: 312; (c) an HCDR3 comprising SEQ ID NO: 318; (d) an LCDR1 comprising SEQ ID NO: 324; (e) an LCDR2 comprising SEQ ID NO: 328; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 305; (b) an HCDR2 comprising SEQ ID NO: 313; (c) an HCDR3 comprising SEQ ID NO: 319; (d) an LCDR1 comprising SEQ ID NO: 325; (e) an LCDR2 comprising SEQ ID NO: 330; and (f) an LCDR3 comprising SEQ ID NO: 336. In some embodiments, the CDRs comprise 1, 2, 3, or more amino acid substitutions.

In some embodiments, the CD19-binding domain comprises: (a) an HCDR1 comprising SEQ ID NO: 306; (b) an HCDR2 comprising SEQ ID NO: 314; (c) an HCDR3 comprising SEQ ID NO: 318; (d) an LCDR1 comprising SEQ ID NO: 326; (e) an LCDR2 comprising SEQ ID NO: 328; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 307; (b) an HCDR2 comprising SEQ ID NO: 312; (c) an HCDR3 comprising SEQ ID NO: 320; (d) an LCDR1 comprising SEQ ID NO: 326; (e) an LCDR2 comprising SEQ ID NO: 328; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 306; (b) an HCDR2 comprising SEQ ID NO: 314; (c) an HCDR3 comprising SEQ ID NO: 320; (d) an LCDR1 comprising SEQ ID NO: 326; (e) an LCDR2 comprising SEQ ID NO: 331; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 337; (b) an HCDR2 comprising SEQ ID NO: 344; (c) an HCDR3 comprising SEQ ID NO: 349; (d) an LCDR1 comprising SEQ ID NO: 355; (e) an LCDR2 comprising SEQ ID NO: 361; and (f) an LCDR3 comprising SEQ ID NO: 332. In some embodiments, the CDRs comprise 1, 2, 3, or more amino acid substitutions.

In some embodiments, the CD19-binding domain comprises: (a) an HCDR1 comprising SEQ ID NO: 338; (b) an HCDR2 comprising SEQ ID NO: 345; (c) an HCDR3 comprising SEQ ID NO: 350; (d) an LCDR1 comprising SEQ ID NO: 356; (e) an LCDR2 comprising SEQ ID NO: 362; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 338; (b) an HCDR2 comprising SEQ ID NO: 345; (c) an HCDR3 comprising SEQ ID NO: 350; (d) an LCDR1 comprising SEQ ID NO: 356; (e) an LCDR2 comprising SEQ ID NO: 362; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 339; (b) an HCDR2 comprising SEQ ID NO: 346; (c) an HCDR3 comprising SEQ ID NO: 351; (d) an LCDR1 comprising SEQ ID NO: 357; (e) an LCDR2 comprising SEQ ID NO: 363; and (f) an LCDR3 comprising SEQ ID NO: 334. In some embodiments, the CDRs comprise 1, 2, 3, or more amino acid substitutions.

In some embodiments, the CD19-binding domain comprises: (a) an HCDR1 comprising SEQ ID NO: 338; (b) an HCDR2 comprising SEQ ID NO: 345; (c) an HCDR3 comprising SEQ ID NO: 350; (d) an LCDR1 comprising SEQ ID NO: 356; (e) an LCDR2 comprising SEQ ID NO: 362; and (f) an LCDR3 comprising SEQ ID NO: 333. In some embodiments, the CDRs comprise 1, 2, 3, or more amino acid substitutions.

In some embodiments, the CD19-binding domain comprises: (a) an HCDR1 comprising SEQ ID NO: 340; (b) an HCDR2 comprising SEQ ID NO: 347; (c) an HCDR3 comprising SEQ ID NO: 352; (d) an LCDR1 comprising SEQ ID NO: 358; (e) an LCDR2 comprising SEQ ID NO: 362; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 341; (b) an HCDR2 comprising SEQ ID NO: 348; (c) an HCDR3 comprising SEQ ID NO: 353; (d) an LCDR1 comprising SEQ ID NO: 359; (e) an LCDR2 comprising SEQ ID NO: 364; and (f) an LCDR3 comprising SEQ ID NO: 336. In some embodiments, the CDRs comprise 1, 2, 3, or more amino acid substitutions.

In some embodiments, the CD19-binding domain comprises: (a) an HCDR1 comprising SEQ ID NO: 342; (b) an HCDR2 comprising SEQ ID NO: 347; (c) an HCDR3 comprising SEQ ID NO: 352; (d) an LCDR1 comprising SEQ ID NO: 360; (e) an LCDR2 comprising SEQ ID NO: 362; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 343; (b) an HCDR2 comprising SEQ ID NO: 347; (c) an HCDR3 comprising SEQ ID NO: 354; (d) an LCDR1 comprising SEQ ID NO: 360; (e) an LCDR2 comprising SEQ ID NO: 362; and (f) an LCDR3 comprising SEQ ID NO: 335. In some embodiments, the CDRs comprise 1, 2, 3, or more amino acid substitutions.

In some embodiments, the CD19-binding domain comprises: (a) an HCDR1 comprising SEQ ID NO: 342; (b) an HCDR2 comprising SEQ ID NO: 347; (c) an HCDR3 comprising SEQ ID NO: 354; (d) an LCDR1 comprising SEQ ID NO: 360; (e) an LCDR2 comprising SEQ ID NO: 362; and (f) an LCDR3 comprising SEQ ID NO: 335. In some embodiments, the CDRs comprise 1, 2, 3, or more amino acid substitutions.

In some embodiments, the VH region of the CD19-binding domain is the VH region of an IgA, IgD, IgE, IgG, or IgM. In some embodiments, the VH region of the CD19-binding domain is the VH region of a subclass of an IgA, IgD, IgE, IgG, or IgM, such as, e.g., IgG1, IgG2, IgG3, IgG4, IgA1, and IgA2. In some embodiments, the VL region of the CD19-binding domain is the VL region of a kappa light chain. In some embodiments, the VL region of the CD19-binding domain is the VL region of a lambda light chain.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 366, 368, 370, 372, 374, 376, 378, 380, 382, and 384. In some embodiments, the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 365, 367, 369, 371, 373, 375, 377, 379, 381, and 383.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 365, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 366. In some embodiments, the VH region of the CD19-binding domain comprises SEQ ID NO: 365, and wherein the VL region of the CD19-binding domain comprises SEQ ID NO: 366.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 367, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 368. In some embodiments, the VH region of the CD19-binding domain comprises SEQ ID NO: 367, and wherein the VL region of the CD19-binding domain comprises SEQ ID NO: 368.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 369, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 370. In some embodiments, the VH region of the CD19-binding domain comprises SEQ ID NO: 369, and wherein the VL region of the CD19-binding domain comprises SEQ ID NO: 370.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 371, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 372. In some embodiments, the VH region of the CD19-binding domain comprises SEQ ID NO: 371, and wherein the VL region of the CD19-binding domain comprises SEQ ID NO: 372.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 373, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 374. In some embodiments, the VH region of the CD19-binding domain comprises SEQ ID NO: 373, and wherein the VL region of the CD19-binding domain comprises SEQ ID NO: 374.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 375, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 376. In some embodiments, the VH region of the CD19-binding domain comprises SEQ ID NO: 375, and wherein the VL region of the CD19-binding domain comprises SEQ ID NO: 376.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 377, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 378. In some embodiments, the VH region of the CD19-binding domain comprises SEQ ID NO: 377, and wherein the VL region of the CD19-binding domain comprises SEQ ID NO: 378.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 379, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 380. In some embodiments, the VH region of the CD19-binding domain comprises SEQ ID NO: 379, and wherein the VL region of the CD19-binding domain comprises SEQ ID NO: 380.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 381, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 382. In some embodiments, the VH region of the CD19-binding domain comprises SEQ ID NO: 381, and wherein the VL region of the CD19-binding domain comprises SEQ ID NO: 382.

In some embodiments, the VH region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 383, and wherein the VL region of the CD19-binding domain comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 384. In some embodiments, the VH region of the CD19-binding domain comprises SEQ ID NO: 383, and wherein the VL region of the CD19-binding domain comprises SEQ ID NO: 384.

In some embodiments, the CD19-binding domain comprises an scFv comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 385.

In some embodiments, the CD19-binding domain comprises an scFv comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 386.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 387, 389, 391, 393, 395, 397, 399, 401, 403, and 405.

In some embodiments, the CD19-binding domain comprises a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO: 388, 390, 392, 394, 396, 398, 400, 402, 404, and 406.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 387, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 388. In some embodiments, the CD19-binding domain comprises a heavy chain comprising SEQ ID NO: 387, and a light chain comprising SEQ ID NO: 388. In some embodiments, the CD19-binding domain comprises a heavy chain comprising SEQ ID NO: 387 with 1, 2, 3, 4, 5, 6, 7, 8, 9, 10, or more mutations therein. In some embodiments, the CD19-binding domain comprises a light chain comprising SEQ ID NO: 388 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 389, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 390. In some embodiments, the CD19-binding domain comprises a heavy chain comprising SEQ ID NO: 389, and a light chain comprising SEQ ID NO: 390. In some embodiments, the CD19-binding domain comprises a heavy chain comprising SEQ ID NO: 389 with 1, 2, 3, 4, 5, 6, 7, 8, 9, 10, or more mutations therein. In some embodiments, the CD19-binding domain comprises a light chain comprising SEQ ID NO: 390 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 391, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 392. In some embodiments, the CD19-binding domain comprises a heavy chain comprising SEQ ID NO: 391, and a light chain comprising SEQ ID NO: 392. In some embodiments, the CD19-binding domain comprises a heavy chain comprising SEQ ID NO: 391 with 1, 2, 3, 4, 5, 6, 7, 8, 9, 10, or more mutations therein. In some embodiments, the CD19-binding domain comprises a light chain comprising SEQ ID NO: 392 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 393, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 394. In some embodiments, the CD19-binding domain comprises a heavy chain comprising SEQ ID NO: 393, and a light chain comprising SEQ ID NO: 394. In some embodiments, the CD19-binding domain comprises a heavy chain comprising SEQ ID NO: 393 with 1, 2, 3, 4, 5, 6, 7, 8, 9, 10, or more mutations therein. In some embodiments, the CD19-binding domain comprises a light chain comprising SEQ ID NO: 394 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 395, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 396. In some embodiments, the CD19-binding domain comprises a heavy chain comprising SEQ ID NO: 395, and a light chain comprising SEQ ID NO: 396. In some embodiments, the CD19-binding domain comprises a heavy chain comprising SEQ ID NO: 395 with 1, 2, 3, 4, 5, 6, 7, 8, 9, 10, or more mutations therein. In some embodiments, the CD19-binding domain comprises a light chain comprising SEQ ID NO: 396 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 397, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 398. In some embodiments, the CD19-binding domain comprises a heavy chain comprising SEQ ID NO: 397, and a light chain comprising SEQ ID NO: 398. In some embodiments, the CD19-binding domain comprises a heavy chain comprising SEQ ID NO: 397 with 1, 2, 3, 4, 5, 6, 7, 8, 9, 10, or more mutations therein. In some embodiments, the CD19-binding domain comprises a light chain comprising SEQ ID NO: 398 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 399, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 400. In some embodiments, the CD19-binding domain comprises a heavy chain comprising SEQ ID NO: 399, and a light chain comprising SEQ ID NO: 400. In some embodiments, the CD19-binding domain comprises a heavy chain comprising SEQ ID NO: 399 with 1, 2, 3, 4, 5, 6, 7, 8, 9, 10, or more mutations therein. In some embodiments, the CD19-binding domain comprises a light chain comprising SEQ ID NO: 400 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 401, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 402. In some embodiments, the CD19-binding domain comprises a heavy chain comprising SEQ ID NO: 401, and a light chain comprising SEQ ID NO: 402. In some embodiments, the CD19-binding domain comprises a heavy chain comprising SEQ ID NO: 401 with 1, 2, 3, 4, 5, 6, 7, 8, 9, 10, or more mutations therein. In some embodiments, the CD19-binding domain comprises a light chain comprising SEQ ID NO: 402 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 403, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 404. In some embodiments, the CD19-binding domain comprises a heavy chain comprising SEQ ID NO: 403, and a light chain comprising SEQ ID NO: 404. In some embodiments, the CD19-binding domain comprises a heavy chain comprising SEQ ID NO: 403 with 1, 2, 3, 4, 5, 6, 7, 8, 9, 10, or more mutations therein. In some embodiments, the CD19-binding domain comprises a light chain comprising SEQ ID NO: 404 with 1, 2, 3, 4, 5, 6, 7, 8, 9, 10, or more mutations therein. In some embodiments, the CD19-binding domain is a Fab.

In some embodiments, the CD19-binding domain comprises a heavy chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 405, and a light chain comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 406. In some embodiments, the CD19-binding domain comprises a heavy chain comprising SEQ ID NO: 405, and a light chain comprising SEQ ID NO: 406. In some embodiments, the CD19-binding domain comprises a heavy chain comprising SEQ ID NO: 405 with 1, 2, 3, 4, 5, 6, 7, 8, 9, 10, or more mutations therein. In some embodiments, the CD19-binding domain comprises a light chain comprising SEQ ID NO: 406 with 1, 2, 3, 4, 5, 6, 7, 8, 9, 10, or more mutations therein. In some embodiments, the CD19-binding domain is a Fab.

In some embodiments, the CD19-binding domain is an antibody selected from the group consisting of: blinatumomab, coltuximab, denintuzumab, duvortuxizumab, emfizatamab, ensomafusp, inebilizumab, loncastuximab, obexelimab, tafasitamab, and taplitumomab, or a fragment or variant thereof. In some embodiments, the CD19-binding domain comprises a VH region from an antibody selected from the group consisting of: blinatumomab, coltuximab, denintuzumab, duvortuxizumab, emfizatamab, ensomafusp, inebilizumab, loncastuximab, obexelimab, tafasitamab, and taplitumomab, or a fragment or variant thereof. In some embodiments, the CD19-binding domain comprises a VL region from an antibody selected from the group consisting of: blinatumomab, coltuximab, denintuzumab, duvortuxizumab, emfizatamab, ensomafusp, inebilizumab, loncastuximab, obexelimab, tafasitamab, and taplitumomab, or a fragment or variant thereof. In some embodiments, the CD19-binding domain comprises a VH region and a VL region from an antibody selected from the group consisting of: blinatumomab, coltuximab, denintuzumab, duvortuxizumab, emfizatamab, ensomafusp, inebilizumab, loncastuximab, obexelimab, tafasitamab, and taplitumomab, or a fragment or variant thereof. In some embodiments, the CD19-binding domain comprises CDRs from an antibody selected from the group consisting of: blinatumomab, coltuximab, denintuzumab, duvortuxizumab, emfizatamab, ensomafusp, inebilizumab, loncastuximab, obexelimab, tafasitamab, and taplitumomab, or a fragment or variant thereof. In some embodiments, the CDRs comprise 1, 2, 3, or more amino acid substitutions.

### 1.2.3. Half-Life Extension Moiety

In some embodiments, the multi-specific polypeptide of the disclosure comprises a half-life extension moiety. In some embodiments, the half-life extension moiety comprises a polypeptide. In some embodiments, the half-life extension moiety comprises a non-polypeptide moiety. In some embodiments, the half-life extension moiety comprises a polymer.

In some embodiments, the half-life extension moiety is selected from the group consisting of a fragment crystallizable (Fc) region (e.g., a single chain Fc), an albumin (e.g., human serum albumin), transferrin, a binding domain that specifically binds to transferrin or albumin (e.g., an albumin binding domain (ABD)), an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising Proline, Alanine and Serine (PAS), or a derivative or functional fragment thereof.

In some embodiments, the half-life extension moiety comprises an immunoglobulin constant region or a portion thereof. An immunoglobulin constant region is comprised of domains denoted CH (constant heavy) domains (CH1, CH2, etc.). Depending on the isotype, (i.e., IgG, IgM, IgA IgD, or IgE), the constant region can be comprised of three or four CH domains. Some isotypes (e.g., IgG) constant regions also contain a hinge region. See Janeway et al. 2001, Immunobiology, Garland Publishing, N.Y., N.Y.

The immunoglobulin constant region or a portion thereof of the disclosure may be obtained from a number of different sources. In some embodiments, an immunoglobulin constant region or a portion thereof is derived from a human immunoglobulin. In some embodiments, an immunoglobulin constant region or a portion thereof is derived from an immunoglobulin of another mammalian species, including for example, a rodent (e.g., a mouse, rat, rabbit, guinea pig) or non-human primate (e.g., chimpanzee, macaque) species. Moreover, the immunoglobulin constant region or a portion thereof may be derived from any immunoglobulin class, including IgM, IgG, IgD, IgA and IgE, and any immunoglobulin isotype, including IgG1, IgG2, IgG3 and IgG4. In some embodiments, the immunoglobulin constant region is derived from human isotype IgG1.

A variety of the immunoglobulin constant region gene sequences (e.g., human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g., hinge, CH2, and/or CH3 sequences, or portions thereof) can be derived from these sequences using art recognized techniques. The genetic material obtained using any of the foregoing methods may then be altered or synthesized to obtain polypeptides of the present disclosure. It will further be appreciated that the scope of this disclosure encompasses alleles, variants and mutations of constant region DNA sequences.

In some embodiments, an immunoglobulin constant region used herein can include all domains and the hinge region or portions thereof. In some embodiment, the immunoglobulin constant region or a portion thereof comprises CH2 domain, CH3 domain, and a hinge region, i.e., an Fc region or an FcRn binding partner.

In some embodiments, the half-life extension moiety comprises a fragment crystallizable (Fc) region. In some embodiments, the Fc region may comprise one or more of the CH2, CH3, and CH4 domains, as well as the hinge region. In some embodiments, fusion proteins comprising an Fc region of an immunoglobulin bestow several desirable properties on a fusion protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1).

In some embodiments, the Fc region comprises two polypeptide chains. In some embodiments, each polypeptide chain is derived from an antibody heavy chain. In some embodiments, both polypeptide chains of the Fc region have the same domain organization (e.g., comprising a CH2 domain and a CH3 domain).

In some embodiments, a polypeptide chain of the Fc region comprises a CH2 domain (about amino acids 231-340 of an antibody Fc region according to EU numbering), and a CH3 domain (about amino acids 341-438 of an antibody Fc region according to EU numbering). In some embodiment, a polypeptide chain of the Fc region comprises a hinge (e.g., upper, middle, and/or lower hinge region) domain (about amino acids 216-230 of an antibody Fc region according to EU numbering), a CH2 domain, and a CH3 domain. In some embodiments, a polypeptide chain of the Fc region comprises a hinge, a CH2 domain, a CH3 domain, and a CH4 domain. In some embodiments, a polypeptide chain of the Fc region comprises at least one of: a hinge, a CH2 domain, a CH3 domain, and a CH4 domain, or a variant, portion, or fragment thereof. In some embodiments, a polypeptide chain of the Fc region comprises a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), or a CH2 domain (or a portion thereof) fused to both a hinge domain (or a portion thereof) and a CH3 domain (or a portion thereof). In some embodiments, a polypeptide chain of the Fc region lacks at least a portion of a CH2 domain (e.g., all or part of a CH2 domain). In some embodiment, a polypeptide chain of the Fc region comprises or consists of amino acids corresponding to EU numbers 221 to 447.

In some embodiments, an Fc region of the disclosure is derived from a human immunoglobulin. In some embodiments, an Fc region is derived from an immunoglobulin of another mammalian species, including for example, a rodent (e.g., a mouse, rat, rabbit, guinea pig) or non-human primate (e.g., chimpanzee, macaque) species. In some embodiments, the Fc region is derived from any immunoglobulin class, including IgM, IgG, IgD, IgA and IgE, and any immunoglobulin isotype, including IgG1, IgG2, IgG3 and IgG4. In some embodiments, it is derived from the human isotype IgG1.

In some embodiments, the Fc region of the disclosure is an Fc variant. In some embodiments, each polypeptide chain of the Fc variant has the same amino acid mutation(s). In some embodiments, different polypeptides of the Fc variant have different amino acid mutation(s) —e.g., the knobs-into-holes format. Descriptions of the knobs-into-holes format can be found, for example, in WO 1996/027011, the content of which is incorporated by reference in its entirety.

In some embodiments, the first polypeptide of the Fc region comprises a hole mutation, and wherein second polypeptide of the Fc region comprises a knob mutation. In some embodiments, the first polypeptide of the Fc region comprises a hole mutation T366S, L368A, and/or Y407V according to EU numbering. In some embodiments, the first polypeptide of the Fc region comprises the hole mutations T366S, L368A, and Y407V according to EU numbering. In some embodiments, the second polypeptide of the Fc region comprises a knob mutation at the position T366 according to EU numbering. In some embodiments, the second polypeptide of the Fc region comprises the knob mutation T366W according to EU numbering.

In some embodiments, the Fc region having the knobs-into-holes format further comprises an engineered disulfide bond formed between an engineered cysteine on each of the polypeptides of the Fc region. In some embodiments, the first polypeptide of the Fc region comprising the hole mutation further comprises a mutation Y349C according to EU numbering, wherein the second polypeptide of the Fc region comprising the knob mutation further comprises a mutation S354C according to EU numbering, and wherein the Fc region comprises an engineered disulfide bond formed between these two cysteines.

In some embodiments, the Fc region is an Fc variant comprising one or more mutations that diminishes or removes an effector function. In some embodiments, the effector function is selected from the group consisting of antibody-dependent cellular cytotoxicity (ADCC), antibody-dependent cellular phagocytosis (ADCP), complement-dependent cytotoxicity (CDC), binding to a Fcγ receptor (FcγR), or any combination thereof. In some embodiments, the Fc region is an Fc variant lacking an endogenous glycan/glycosylation site. In some embodiments, one or both of the polypeptides of the Fc region comprise a mutation L234A, L235A, P329G, and/or N297G according to EU numbering. Further descriptions of such Fc variants can be found, for example, in US20120251531A1, the content of which is incorporated by reference in its entirety.

In some embodiments, one or both of the polypeptides of the Fc region comprise the mutation N297G according to EU numbering. In some embodiments, the N297G mutation removes an endogenous glycan/glycosylation site of the Fc region.

In some embodiments, one or both of the polypeptides of the Fc region comprise a mutation L234A, L235A, and/or P329G according to EU numbering. In some embodiments, one or both of the polypeptides of the Fc region comprise mutations L234A and L235A according to EU numbering. In some embodiments, one or both of the polypeptides of the Fc region comprise mutations L234A, L235A, and P329G according to EU numbering. In some embodiments, both of the polypeptides of the Fc region comprise mutations L234A, L235A, and P329G according to EU numbering. In some embodiments, one or both of the polypeptides of the Fc region further comprise a mutation N297G according to EU numbering. In some embodiments, both of the polypeptides of the Fc region further comprise a mutation N297G according to EU numbering. In some embodiments, the Fc region lacks one or more effector functions and/or lacks glycosylation.

In some embodiments, one or both of the polypeptides of the Fc region comprise the mutation H435R and/or Y436F according to EU numbering. In some embodiments, one or both of the polypeptides of the Fc region comprise the mutations H435R/Y436F according to EU numbering. In some embodiments, only the first polypeptide of the Fc region comprises the hole mutation(s) further comprise the mutations H435R/Y436F according to EU numbering. In some embodiments, the H435R and/or Y436F mutations in the CH3 domain diminishes or removes protein A binding capability of the Fc region.

In some embodiments, one or both of the polypeptides of the Fc region comprise the mutation the deletion of G446 and/or K447 according to EU numbering. In some embodiments, both of the polypeptides of the Fc region comprise the mutation corresponding to the deletion of G446 and K447 according to EU numbering.

In some embodiments, the Fc variant comprises the mutations L234A, L235A, P329G, N297G, and deletion of G446 and K447 according to EU numbering. In some embodiments, the Fc variant has the knobs-into-holes format. In some embodiments, the first polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 154, and wherein the second polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 155.

In some embodiments, the Fc variant comprises the mutations L234A, L235A, P329G, H435R, Y436F according to EU numbering. In some embodiments, the Fc variant has the knobs-into-holes format. In some embodiments, the first polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 156, and wherein the second polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 157.

In some embodiments, the Fc variant has a change in at least one effector function imparted by an Fc region comprising said wild-type Fc domain (e.g., an improvement or reduction in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In some embodiments, the Fc variant provides an engineered cysteine residue.

In some embodiments, the Fc region of the disclosure is an Fc variant known to impart a change (e.g., an enhancement or reduction) in effector function and/or FcR binding. Specifically, the Fc region may include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, U52007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, the content of each of which is incorporated by reference in its entirety. In some embodiments, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

The Fc region of IgG can be modified according to well recognized procedures such as site directed mutagenesis and the like to yield modified IgG or Fc fragments or portions thereof that will be bound by Fc receptors. Such modifications include modifications remote from the Fc receptor contact sites as well as modifications within the contact sites that preserve or even enhance binding to the Fc receptors. For example, the following single amino acid residues in human IgG1 Fc (Fc γ1) can be substituted without significant loss of Fc binding affinity for Fc receptors: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wild type proline substituted by alanine at position number 238. As an example, a specific embodiment incorporates the N297A mutation, removing a highly conserved N-glycosylation site. In addition to alanine other amino acids may be substituted for the wild-type amino acids at the positions specified above. Mutations may be introduced into Fc giving rise to more than one hundred Fc regions distinct from the native Fc. Additionally, combinations of two, three, or more of these individual mutations may be introduced together, giving rise to hundreds more Fc regions. Moreover, one polypeptide chain of the Fc region may be mutated and the other polypeptide chain of the Fc region not mutated at all, or they both may be mutated but with different mutations.

Certain of the above mutations may confer new functionality upon the Fc region. For example, the substitution N297A removes a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for Fc receptors may be increased beyond that of wild type in some instances. This increased affinity may reflect an increased “on” rate, a decreased “off” rate or both an increased “on” rate and a decreased “off” rate. Examples of mutations believed to impart an increased affinity for Fc receptors include, but not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity may arise from mutations of this region, as for example by replacing amino acids 233-236 of human IgGI “ELLG” to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In some embodiments, the Fc region is hemi-glycosylated (for example, containing a first, glycosylated CH2 region and a second, aglycosylated CH2 region. In some embodiments, the Fc region is fully glycosylated. In other embodiments, the Fc region is aglycosylated.

In some embodiments, the Fc region comprises an amino acid substitution that alters the antigen-independent effector functions of the Ig constant region, in particular the circulating half-life of the protein. Such proteins exhibit either increased or decreased binding to an Fc receptor when compared to proteins lacking these substitutions and, therefore, have an increased or decreased half-life in serum, respectively. Fc variants with improved affinity for an Fc receptor are anticipated to have longer serum half-lives, and such molecules have useful applications in methods of treating mammals where long half-life of the administered polypeptide is desired, e.g., to treat a chronic disease or disorder (see, e.g., U.S. Pat. Nos. 7,348,004, 7,404,956, and 7,862,820). In contrast, Fc variants with decreased Fc receptor binding affinity are expected to have shorter half-lives, and such molecules are also useful, for example, for administration to a mammal where a shortened circulation time may be advantageous, e.g., for in vivo diagnostic imaging or in situations where the starting polypeptide has toxic side effects when present in the circulation for prolonged periods. Fc variants with decreased Fc receptor binding affinity are also less likely to cross the placenta and, thus, are also useful in the treatment of diseases or disorders in pregnant women. In addition, other applications in which reduced Fc receptor binding affinity may be desired include those applications in which localization the brain, kidney, and/or liver is desired. In one exemplary embodiment, the polypeptide of the disclosure exhibits reduced transport across the epithelium of kidney glomeruli from the vasculature. In another embodiment, the polypeptide of the disclosure exhibits reduced transport across the blood brain barrier (BBB) from the brain, into the vascular space. In some embodiments, the Fc region comprises one or more amino acid substitutions within the “Fc receptor binding loop” of an Ig constant region. The Fc receptor binding loop is comprised of amino acid residues 280-299 (according to EU numbering) of a wild-type, full-length, Fc region. In other embodiments, the Fc region comprises one or more amino acid substitutions at an amino acid position corresponding to any one of the following EU positions: 256, 277-281, 283-288, 303-309, 313, 338, 342, 376, 381, 384, 385, 387, 434 (e.g., N434A or N434K), and 438, which results in altered Fc receptor binding affinity. Exemplary amino acid substitutions which altered Fc receptor binding activity are disclosed in International PCT Publication No. WO05/047327.

In some embodiments, the half-life extension moiety comprises a single chain Fc (scFc). In some embodiments, the scFc molecule comprises a first and second Fc polypeptide sequences each comprising a hinge region, a CH2 domain, and a CH3 domain, and wherein the Fc polypeptide sequences are joined by a polypeptide linker so as to allow the formation of a functional Fc region from a single polypeptide unit having the amino to carboxyl order: Hinge-CH2-CH3-linker-Hinge-CH2-CH3. Further descriptions of scFc can be found, for example, in US20110081345, the content of which is incorporated by reference in its entirety.

In some embodiments, the half-life extension moiety comprises a serum protein or a fragment thereof. In some embodiments, the serum protein comprises a thyroxine-binding protein, a transthyretin, a 1-acid glycoprotein, a transferrin, transferrin receptor or a transferrin-binding portion thereof, a fibrinogen, or an albumin.

In some embodiments, the half-life extension moiety comprises an albumin or a functional fragment thereof. Human serum albumin (I, or HA), a protein of about 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. In some embodiments, the albumin or a fragment or variant thereof extends (or is capable of extending) the half-life of the polypeptide. Further examples of albumin or the fragments or variants thereof are disclosed in US Pat. Publ. Nos. 2008/0194481 A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, the content of each of which is incorporated by reference in its entirety.

In some embodiments, the half-life extension moiety comprises a transferrin or a fragment thereof. Any transferrin may be used to make the multi-specific polypeptides of the disclosure. As an example, wild-type human Tf (Tf) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936.

Transferrin comprises two domains, N domain and C domain. N domain comprises two subdomains, NI domain and N2 domain, and C domain comprises two subdomains, C1 domain and C2 domain. In some embodiments, the transferrin portion of the multi-specific polypeptide includes a transferrin splice variant. In one example, a transferrin splice variant can be a splice variant of human transferrin, e.g., Genbank Accession AAA61140. In some embodiments, the transferrin portion of the multi-specific polypeptide includes one or more domains of the transferrin sequence, e.g., N domain, C domain, N1 domain, N2 domain, C1 domain, C2 domain or any combinations thereof.

In some embodiments, the half-life extension moiety comprises a binding moiety binds serum protein or a fragment thereof. In some embodiments, the binding moiety is a polypeptide, a ligand, or a small molecule. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the half-life extension moiety comprises an albumin binding moiety, which comprises an albumin binding peptide, a bacterial albumin binding domain, an albumin-binding antibody fragment, or any combinations thereof. In some embodiments, the albumin binding moiety is an antibody. In some embodiments, the antibody comprises a single domain antibody, a single chain variable fragment, or a Fab. In some embodiments, the single domain antibody comprises a single domain antibody that binds to albumin. In some embodiments, the antibody is a human or humanized antibody. In some embodiments, the antibody is selected from the group consisting of 645gHlgL1, 645dsgH5gL4, 23-13-A01-sc02. In some embodiments, the albumin binding moiety is a bacterial albumin binding protein, an antibody or an antibody fragment thereof including domain antibodies (see U.S. Pat. No. 6,696,245), or one of streptococcal protein G (Konig, T. and Skerra, A. (1998) J. Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, His, Ile, Leu, or Lys; Xaa₃ is Ala, Asp, Phe, Trp, or Tyr, and Xaa₄ is Asp, Gly, Leu, Phe, Ser, or Thr as described in US patent application 2003/0069395 or Dennis et al. (Dennis et al. (2002) J. Biol. Chem. 277, 35035-35043), the content of each of which is incorporated by reference in its entirety.

In some embodiments, the half-life extension moiety comprises a PAS sequence. A PAS sequence, as refers to an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the multi-specific polypeptide. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline may be selected from the group consisting of Arg, Asn, Asp, Cys, Gln, Glu, Gly, His, Ile, Leu, Lys, Met, Phe, Thr, Trp, Tyr, and Val. Under physiological conditions, the PAS sequence stretch forms a random coil conformation and thereby can mediate an increased in vivo and/or in vitro stability to the polypeptide. In some embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable. Non-limiting examples of the PAS sequences forming random coil conformation comprise an amino acid sequence selected from the group consisting of ASPAAPAPASPAAPAPSAPA (SEQ ID NO: 1131), AAPASPAPAAPSAPAPAAPS (SEQ ID NO: 1132), APSSPSPSAPSSPSPASPSS (SEQ ID NO: 1133), APSSPSPSAPSSPSPASPS (SEQ ID NO: 1134), SSPSAPSPSSPASPSPSSPA (SEQ ID NO: 1135), AASPAAPSAPPAAASPAAPSAPPA (SEQ ID NO: 1136) and ASAAAPAAASAAASAPSAAA (SEQ ID NO: 1137) or any combinations thereof. Additional examples of PAS sequences are known from, e.g., US Pat. Publ. No. 2010/0292130 A1 and PCT Appl. Publ. No. WO 2008/155134 A1, the content of each of which is incorporated by reference in its entirety.

In some embodiments, the half-life extension moiety comprises a glycine-rich homo-amino-acid polymer (HAP). The HAP sequence can comprise a repetitive sequence of glycine, which has at least 50 amino acids, 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In some embodiments, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is about 20, 21, 22, 23, 24, 25, 26, 26, 28, 29, 30, 31, 32, 33, 34, 35, 36, 37, 38, 39, or 40. In another embodiment, n is about 50, 60, 70, 80, 90, 100, 110, 120, 130, 140, 150, 160, 170, 180, 190, or 200.

In some embodiments, the half-life extension moiety comprises a polyethylene glycol, ethylene glycol/propylene glycol copolymer, carboxymethylcellulose, dextran, polyvinyl alcohol, poly(alkylene oxide), poly(vinyl pyrrolidone), poly(vinyl alcohol), polyoxazoline, or poly(acryloylmorpholine). Also provided by the disclosure are chemically modified derivatives of the multi-specific polypeptide of the disclosure which may provide additional advantages such as increased solubility, stability and circulating time of the polypeptide, or decreased immunogenicity (see U.S. Pat. No. 4,179,337). The chemical moieties for modification can be selected from the group consisting of water soluble polymers including, but not limited to, polyethylene glycol, ethylene glycol/propylene glycol copolymers, carboxymethylcellulose, dextran, and polyvinyl alcohol. The multi-specific polypeptide may be modified at random positions within the molecule or at the N- or C-terminus, or at predetermined positions within the molecule and may include one, two, three or more attached chemical moieties. The polymer can be of any molecular weight and can be branched or unbranched. For polyethylene glycol, in some embodiments, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa. In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The number of polyethylene glycol moieties attached to each multi-specific polypeptide of the disclosure (i.e., the degree of substitution) may also vary. For example, the pegylated proteins of the disclosure may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In some embodiments, the half-life extension moiety comprises a hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8 (8), 271-278 (1987); and Weidler et al., Arzneim.-Forschung/Drug Res., 41, 494-498 (1991)). Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions, it is possible to exploit the different reactivity of the respective hydroxy group in the unsubstituted glucose monomer with respect to a hydroxyethylation. Owing to this fact, the skilled person is able to influence the substitution pattern to a limited extent. HES is mainly characterized by the molecular weight distribution and the degree of substitution. The degree of substitution, denoted as DS, relates to the molar substitution, is known to the skilled people. See Sommermeyer et al., Krankenhauspharmazie, 8 (8), 271-278 (1987), as cited above, in particular p. 273. In one embodiment, hydroxyethyl starch has a mean molecular weight (weight mean) of from 1 to 300 kD, from 2 to 200 kD, from 3 to 100 kD, or from 4 to 70 kD. Hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolaemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD, including any ranges or subranges therebetween. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD, including any ranges or subranges therebetween. In certain embodiments, the heterologous moiety can be mixtures of hydroxyethyl starches having different mean molecular weights and/or different degrees of substitution and/or different ratios of C2:C6 substitution. Therefore, mixtures of hydroxyethyl starches may be employed having different mean molecular weights and different degrees of substitution and different ratios of C2:C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2:C6 substitution, or having about the same mean molecular weight and about the same degree of substitution and about the same ratio of C2:C6 substitution.

In some embodiments, the half-life extension moiety comprises a polysialic acid (PSA) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp 335-348. They can be produced in various degrees of polymerization (e.g., from n=about 80 or more sialic acid residues down to n=2) by limited acid hydrolysis or by digestion with neuraminidases, or by fractionation of the natural, bacterially derived forms of the polymer. The composition of different polysialic acids also varies such that there are homopolymeric forms i.e., the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist-such as the alternating alpha-2,8 alpha-2,9 polysialic acid of E. coli strain K92 and group C polysaccharides of N. meningitidis. Sialic acid may also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during fetal development (wherein the polymer has an anti-adhesive function) Cho and Troy, P.N.A.S., USA, 91 (1994) 11427-11431, which is incorporated by reference herein in its entirety. The alpha-2,8-linked polysialic acid of E. coli strain K1 is also known as ‘colominic acid’ and is used (in various lengths) to exemplify the present disclosure. Various methods of attaching or conjugating polysialic acids to a polypeptide have been described, for example, in U.S. Pat. No. 5,846,951 and US 2007/0191597 A1.

In some embodiments, the half-life extension moiety comprises an XTEN Sequence. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a fusion protein partner, XTENs can serve as a carrier, conferring certain desirable pharmacokinetic, physicochemical and pharmaceutical properties when linked to the binding domains of the disclosure. Such desirable properties include but are not limited to enhanced pharmacokinetic parameters and solubility characteristics. In some embodiments, the XTEN sequence of the disclosure is a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In some embodiments, XTEN is a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In some embodiments, the XTEN sequence of the disclosure comprises one or more sequence motif of 9 to 14 amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids selected from the group consisting of glycine (G), alanine (A), serine(S), threonine (T), glutamate I and proline (P). In some embodiments, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology. In other embodiments, the XTEN comprises multiple units of motif sequences from two or more of the motif families. These sequences can be selected to achieve desired physical/chemical characteristics, including such properties as net charge, hydrophilicity, lack of secondary structure, or lack of repetitiveness that are conferred by the amino acid composition of the motifs, described more fully below. In other embodiments, the motifs incorporated into the XTEN can be selected and assembled using the methods described herein to achieve an XTEN of about 36 to about 3000 amino acid residues. In some embodiments, the XTEN sequence of the disclosure affects the physical or chemical property, e.g., pharmacokinetics, of the polypeptide of the present disclosure. The XTEN sequence used in the present disclosure can exhibit one or more of the following advantageous properties: conformational flexibility, enhanced aqueous solubility, high degree of protease resistance, low immunogenicity, low binding to mammalian receptors, or increased hydrodynamic (or Stokes) radii. In a specific embodiment, the XTEN sequence linked to the binding domain(s) of the disclosure increases pharmacokinetic properties such as longer terminal half-life or increased area under the curve (AUC), so that the multi-specific polypeptide stays in vivo for an increased period of time compared to a control polypeptide lacking the XTEN sequence. In some embodiments, the XTEN sequence of the disclosure increases pharmacokinetic properties such as longer terminal half-life or increased area under the curve (AUC), so that the multi-specific polypeptide stays in vivo for an increased period of time compared to a control polypeptide lacking the XTEN sequence. A variety of methods and assays can be employed to determine the physical/chemical properties of proteins comprising the XTEN sequence. Such methods include, but are not limited to analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Amau et al., Prot Expr and Purif 48, 1-13 (2006). More description of XTEN can be found, for example, in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2, the content of each of which is incorporated herein by its entirety.

In some embodiments, the half-life extension moiety comprises an immunoglobulin binding polypeptide. In some embodiments, the binding polypeptide is a polypeptide, a ligand, or a small molecule. In some embodiments, the immunoglobulin molecule comprises IgG1, IgG2, IgG3, IgG4, sIgA, IgM or IgD. In some embodiments, the immunoglobulin binding polypeptide can bind to an Fc region and can improve a half-life of the multi-specific polypeptide of the disclosure. In some embodiments, the immunoglobulin binding polypeptide has greater than about 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, or 500 amino acid residues. In some embodiments, the immunoglobulin binding polypeptide of the disclosure comprises a 13-mer IgG-Fc domain binding peptide (IgGBP). More description of the immunoglobulin binding polypeptide useful for the disclosure can be found, for example, in DeLano W L, et al. (2000) Science 287:1279-1283. US Patent Publication No. 20170334954, US Patent Publication No. 20170210777, or PCT Publication No. WO120171069158, the content of each of which is incorporated herein by its entirety.

### 1.2.4. Domain Organization of the Multi-Specific Polypeptides

In some embodiments, the first binding domain and/or the second binding domain comprises a regular Fab.

In some embodiments, the first binding domain and/or the second binding domain comprises a CrossFab. The term “CrossFab” refers to a Fab fragment, wherein the constant regions of the heavy and light chain (i.e., the CH1 and CL regions) are exchanged. Accordingly, the CrossFab comprises a first polypeptide chain comprising, from N-terminus to C-terminus, a VH and a CL region, and a second polypeptide chain comprising, from N-terminus to C-terminus, a VL and a CH1 region. Further descriptions of the CrossFab can be found, for example, in US20200199234 and US20240019424, the content of each of which is incorporated by reference in its entirety.

In some embodiments, the Fab is a charge steering Fab. In some embodiments, the CrossFab is a charge steering CrossFab. In some embodiments, electrostatics are used to skew the formation of target Fab with CH1 and CL regions comprising charge steering mutation(s).

In some embodiments, the charge steering Fab or CrossFab comprises a CH1 region comprising a mutation K147E and/or K213E according to EU numbering, and a CL region comprising a mutation E123R and/or Q124K according to EU numbering. In some embodiments, the charge steering Fab or CrossFab comprises a CH1 region comprising mutations K147E/K213E according to EU numbering, and a CL region comprising mutations E123R/Q124K according to EU numbering.

In some embodiments, the multi-specific polypeptide comprises, from N-terminus to C-terminus, a first binding domain and a second binding domain. In some embodiments, the multi-specific polypeptide comprises only one first binding domain and only one second binding domain. In some embodiments, the multi-specific polypeptide further comprises a half-life extension moiety (e.g., a Fc region). In some embodiments, the multi-specific polypeptide comprises, from N-terminus to C-terminus, a first binding domain, a second binding domain, and a half-life extension moiety (e.g., a Fc region). In some embodiments, the multi-specific polypeptide comprises a first binding domain connected to a second binding domain, and a half-life extension moiety (e.g., a Fc region) connected to the second binding domain, but no connection between the first binding domain and the half-life extension moiety other than through the second binding domain. In some embodiments, the Fc region has a knobs-into-holes format (a first polypeptide of the Fc region comprising a hole mutation, and a second polypeptide of the Fc region comprises a knob mutation). In some embodiments, the second binding domain is connected to the first polypeptide of the Fc region comprising a hole mutation, and optionally, the second polypeptide of the Fc region comprising a knob mutation is not connected to the first or the second binding domain. In some embodiments, the second binding domain is connected to the second polypeptide of the Fc region comprising a knob mutation, and optionally, the first polypeptide of the Fc region comprising a hole mutation is not connected to the first or the second binding domain.

In some embodiments, the multi-specific polypeptide comprises a first and a second binding domain, wherein the C-terminus of the VH region of the first binding domain is connected to the N-terminus of the VH region of the second binding domain. In some embodiments, the first binding domain is an scFv and the second binding domain is an scFv. In some embodiments, the first binding domain is an scFv and the second binding domain is a Fab. In some embodiments, the first binding domain is an scFv and the second binding domain is a CrossFab. In some embodiments, the C-terminus of the VL region of the second binding domain (e.g., an scFv) is connected to a half-life extension moiety. In some embodiments, the C-terminus of the VL region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the VH region of the first binding domain is connected to the N-terminus of the VL region of the second binding domain. In some embodiments, the first binding domain is an scFv and the second binding domain is an scFv. In some embodiments, the first binding domain is an scFv and the second binding domain is a Fab. In some embodiments, the first binding domain is an scFv and the second binding domain is a CrossFab. In some embodiments, the C-terminus of the VH region of the second binding domain (e.g., an scFv) is connected to a half-life extension moiety. In some embodiments, the C-terminus of the VH region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the VL region of the first binding domain is connected to the N-terminus of the VH region of the second binding domain. In some embodiments, the first binding domain is an scFv and the second binding domain is an scFv. In some embodiments, the first binding domain is an scFv and the second binding domain is a Fab. In some embodiments, the first binding domain is an scFv and the second binding domain is a CrossFab. In some embodiments, the C-terminus of the VL region of the second binding domain (e.g., an scFv) is connected to a half-life extension moiety. In some embodiments, the C-terminus of the VL region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the VL region of the first binding domain is connected to the N-terminus of the VL region of the second binding domain. In some embodiments, the first binding domain is an scFv and the second binding domain is an scFv. In some embodiments, the first binding domain is an scFv and the second binding domain is a Fab. In some embodiments, the first binding domain is an scFv and the second binding domain is a CrossFab. In some embodiments, the C-terminus of the VH region of the second binding domain (e.g., an scFv) is connected to a half-life extension moiety. In some embodiments, the C-terminus of the VH region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the CH1 region of the first binding domain is connected to the N-terminus of the VH region of the second binding domain. In some embodiments, the first binding domain is a Fab and the second binding domain is an scFv. In some embodiments, the first binding domain is a Fab and the second binding domain is a Fab. In some embodiments, the first binding domain is a Fab and the second binding domain is a CrossFab. In some embodiments, the first binding domain is a CrossFab and the second binding domain is an scFv. In some embodiments, the first binding domain is a CrossFab and the second binding domain is a Fab. In some embodiments, the first binding domain is a CrossFab and the second binding domain is a CrossFab. In some embodiments, the C-terminus of the VL region of the second binding domain (e.g., an scFv) is connected to a half-life extension moiety. In some embodiments, the C-terminus of the VL region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the CH1 region of the first binding domain is connected to the N-terminus of the VL region of the second binding domain. In some embodiments, the first binding domain is a Fab and the second binding domain is an scFv. In some embodiments, the first binding domain is a Fab and the second binding domain is a Fab. In some embodiments, the first binding domain is a Fab and the second binding domain is a CrossFab. In some embodiments, the first binding domain is a CrossFab and the second binding domain is an scFv. In some embodiments, the first binding domain is a CrossFab and the second binding domain is a Fab. In some embodiments, the first binding domain is a CrossFab and the second binding domain is a CrossFab. In some embodiments, the C-terminus of the VH region of the second binding domain (e.g., an scFv) is connected to a half-life extension moiety. In some embodiments, the C-terminus of the VH region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the CL region of the first binding domain is connected to the N-terminus of the VH region of the second binding domain. In some embodiments, the first binding domain is a Fab and the second binding domain is an scFv. In some embodiments, the first binding domain is a Fab and the second binding domain is a Fab. In some embodiments, the first binding domain is a Fab and the second binding domain is a CrossFab. In some embodiments, the first binding domain is a CrossFab and the second binding domain is an scFv. In some embodiments, the first binding domain is a CrossFab and the second binding domain is a Fab. In some embodiments, the first binding domain is a CrossFab and the second binding domain is a CrossFab. In some embodiments, the C-terminus of the VL region of the second binding domain (e.g., an scFv) is connected to a half-life extension moiety. In some embodiments, the C-terminus of the VL region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises a first and a second binding domain, wherein the C-terminus of the CL region of the first binding domain is connected to the N-terminus of the VL region of the second binding domain. In some embodiments, the first binding domain is a Fab and the second binding domain is an scFv. In some embodiments, the first binding domain is a Fab and the second binding domain is a Fab. In some embodiments, the first binding domain is a Fab and the second binding domain is a CrossFab. In some embodiments, the first binding domain is a CrossFab and the second binding domain is an scFv. In some embodiments, the first binding domain is a CrossFab and the second binding domain is a Fab. In some embodiments, the first binding domain is a CrossFab and the second binding domain is a CrossFab. In some embodiments, the C-terminus of the VH region of the second binding domain (e.g., an scFv) is connected to a half-life extension moiety. In some embodiments, the C-terminus of the VH region of the second binding domain (e.g., an scFv) is connected to the N-terminus of a polypeptide of the Fc region. In some embodiments, the C-terminus of the CH1 region of the second binding domain (e.g., a Fab or CrossFab) is connected to a half-life extension moiety. In some embodiments, the C-terminus of the CH1 region of the second binding domain (e.g., a Fab or CrossFab) is connected to the N-terminus of a polypeptide of the Fc region. In some embodiments, the C-terminus of the CL region of the second binding domain (e.g., a Fab or CrossFab) is connected to a half-life extension moiety. In some embodiments, the C-terminus of the CL region of the second binding domain (e.g., a Fab or CrossFab) is connected to the N-terminus of a polypeptide of the Fc region.

In some embodiments, the multi-specific polypeptide comprises, from N-terminus to C-terminus, a second binding domain and a first binding domain. In some embodiments, the multi-specific polypeptide comprises only one first binding domain and only one second binding domain. In some embodiments, the multi-specific polypeptide further comprises a half-life extension moiety (e.g., a Fc region). In some embodiments, the multi-specific polypeptide comprises, from N-terminus to C-terminus, a second binding domain, a first binding domain, and a half-life extension moiety (e.g., a Fc region). In some embodiments, the multi-specific polypeptide comprises a second binding domain connected to a first binding domain, and a half-life extension moiety (e.g., a Fc region) connected to the first binding domain, but no connection between the second binding domain and the half-life extension moiety other than through the first binding domain. In some embodiments, the Fc region has a knobs-into-holes format (a first polypeptide of the Fc region comprising a hole mutation, and a second polypeptide of the Fc region comprising a knob mutation). In some embodiments, the first binding domain is connected to the first polypeptide of the Fc region comprising a hole mutation, and optionally, the second polypeptide of the Fc region comprising a knob mutation is not connected to the first or the second binding domain. In some embodiments, the first binding domain is connected to the second polypeptide of the Fc region comprising a knob mutation, and optionally, the first polypeptide of the Fc region comprising a hole mutation is not connected to the first or the second binding domain.

In some embodiments, the multi-specific polypeptide comprises a first and a second binding domain, wherein the C-terminus of the VH region of the second binding domain is connected to the N-terminus of the VH region of the first binding domain. In some embodiments, the first binding domain is an scFv and the second binding domain is an scFv. In some embodiments, the first binding domain is a Fab and the second binding domain is an scFv. In some embodiments, the first binding domain is a CrossFab and the second binding domain is an scFv. In some embodiments, the C-terminus of the VL region of the first binding domain (e.g., an scFv) is connected to a half-life extension moiety. In some embodiments, the C-terminus of the VL region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domain, wherein the C-terminus of the VH region of the second binding domain is connected to the N-terminus of the VL region of the first binding domain. In some embodiments, the first binding domain is an scFv and the second binding domain is an scFv. In some embodiments, the first binding domain is a Fab and the second binding domain is an scFv. In some embodiments, the first binding domain is a CrossFab and the second binding domain is an scFv. In some embodiments, the C-terminus of the VH region of the first binding domain (e.g., an scFv) is connected to a half-life extension moiety. In some embodiments, the C-terminus of the VH region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domain, wherein the C-terminus of the VL region of the second binding domain is connected to the N-terminus of the VH region of the first binding domain. In some embodiments, the first binding domain is an scFv and the second binding domain is an scFv. In some embodiments, the first binding domain is a Fab and the second binding domain is an scFv. In some embodiments, the first binding domain is a CrossFab and the second binding domain is an scFv. In some embodiments, the C-terminus of the VL region of the first binding domain (e.g., an scFv) is connected to a half-life extension moiety. In some embodiments, the C-terminus of the VL region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domains, and wherein the C-terminus of the VL region of the second binding domain is connected to the N-terminus of the VL region of the first binding domain. In some embodiments, the first binding domain is an scFv and the second binding domain is an scFv. In some embodiments, the first binding domain is a Fab and the second binding domain is an scFv. In some embodiments, the first binding domain is a CrossFab and the second binding domain is an scFv. In some embodiments, the C-terminus of the VH region of the first binding domain (e.g., an scFv) is connected to a half-life extension moiety. In some embodiments, the C-terminus of the VH region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domains, and wherein the C-terminus of the CH1 region of the second binding domain is connected to the N-terminus of the VH region of the first binding domain. In some embodiments, the first binding domain is an scFv and the second binding domain is a Fab. In some embodiments, the first binding domain is a Fab and the second binding domain is a Fab. In some embodiments, the first binding domain is a CrossFab and the second binding domain is a Fab. In some embodiments, the first binding domain is an scFv and the second binding domain is a CrossFab. In some embodiments, the first binding domain is a Fab and the second binding domain is a CrossFab. In some embodiments, the first binding domain is a CrossFab and the second binding domain is a CrossFab. In some embodiments, the C-terminus of the VL region of the first binding domain (e.g., an scFv) is connected to a half-life extension moiety. In some embodiments, the C-terminus of the VL region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domain, wherein the C-terminus of the CH1 region of the second binding domain is connected to the N-terminus of the VL region of the first binding domain. In some embodiments, the first binding domain is an scFv and the second binding domain is a Fab. In some embodiments, the first binding domain is a Fab and the second binding domain is a Fab. In some embodiments, the first binding domain is a CrossFab and the second binding domain is a Fab. In some embodiments, the first binding domain is an scFv and the second binding domain is a CrossFab. In some embodiments, the first binding domain is a Fab and the second binding domain is a CrossFab. In some embodiments, the first binding domain is a CrossFab and the second binding domain is a CrossFab. In some embodiments, the C-terminus of the VH region of the first binding domain (e.g., an scFv) is connected to a half-life extension moiety. In some embodiments, the C-terminus of the VH region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domain, wherein the C-terminus of the CL region of the second binding domain is connected to the N-terminus of the VH region of the first binding domain. In some embodiments, the first binding domain is an scFv and the second binding domain is a Fab. In some embodiments, the first binding domain is a Fab and the second binding domain is a Fab. In some embodiments, the first binding domain is a CrossFab and the second binding domain is a Fab. In some embodiments, the first binding domain is an scFv and the second binding domain is a CrossFab. In some embodiments, the first binding domain is a Fab and the second binding domain is a CrossFab. In some embodiments, the first binding domain is a CrossFab and the second binding domain is a CrossFab. In some embodiments, the C-terminus of the VL region of the first binding domain (e.g., an scFv) is connected to a half-life extension moiety. In some embodiments, the C-terminus of the VL region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the multi-specific polypeptide comprises a first and a second binding domain, wherein the C-terminus of the CL region of the second binding domain is connected to the N-terminus of the VL region of the first binding domain. In some embodiments, the first binding domain is an scFv and the second binding domain is a Fab. In some embodiments, the first binding domain is a Fab and the second binding domain is a Fab. In some embodiments, the first binding domain is a CrossFab and the second binding domain is a Fab. In some embodiments, the first binding domain is an scFv and the second binding domain is a CrossFab. In some embodiments, the first binding domain is a Fab and the second binding domain is a CrossFab. In some embodiments, the first binding domain is a CrossFab and the second binding domain is a CrossFab. In some embodiments, the C-terminus of the VH region of the first binding domain (e.g., an scFv) is connected to a half-life extension moiety. In some embodiments, the C-terminus of the VH region of the first binding domain (e.g., an scFv) is connected to the N-terminus of a polypeptide of the Fc region. In some embodiments, the C-terminus of the CH1 region of the first binding domain (e.g., a Fab or CrossFab) is connected to a half-life extension moiety. In some embodiments, the C-terminus of the CH1 region of the first binding domain (e.g., a Fab or CrossFab) is connected to the N-terminus of a polypeptide of the Fc region. In some embodiments, the C-terminus of the CL region of the first binding domain (e.g., a Fab or CrossFab) is connected to a half-life extension moiety. In some embodiments, the C-terminus of the CL region of the first binding domain (e.g., a Fab or CrossFab) is connected to the N-terminus of a polypeptide of the Fc region.

In some embodiments, the first binding domain is an scFv that connects via its C-terminus to the N-terminus of the VH region of the second binding domain via a peptide linker (PLB). In some embodiments, the scFv has a VH at the N-terminus and a VL at the C-terminus. In some embodiments, the scFv has a VL at the N-terminus and a VH at the C-terminus. In some embodiments, the second binding domain is a Fab. In some embodiments, the C-terminus of the CH1 region of the second binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the first binding domain is an scFv that connects via its C-terminus to the N-terminus of the VL region of the second binding domain via a peptide linker (PLB). In some embodiments, the scFv has a VH at the N-terminus and a VL at the C-terminus. In some embodiments, the scFv has a VL at the N-terminus and a VH at the C-terminus. In some embodiments, the second binding domain is a Fab. In some embodiments, the C-terminus of the CH1 region of the second binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the second binding domain is a Fab that connects via the C-terminus of its CH1 to a N-terminus of the first binding domain via a peptide linker (PLB). In some embodiments, the first binding domain is an scFv. In some embodiments, the scFv has a VH at the N-terminus and a VL at the C-terminus. In some embodiments, the scFv has a VL at the N-terminus and a VH at the C-terminus. In some embodiments, the C-terminus of the first binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the first binding domain is a Fab that connects via the C-terminus of its CH1 region to a N-terminus of the second binding domain via a peptide linker (PLB). In some embodiments, the second binding domain is an scFv. In some embodiments, the scFv has a VH at the N-terminus and a VL at the C-terminus. In some embodiments, the scFv has a VL at the N-terminus and a VH at the C-terminus. In some embodiments, the C-terminus of the second binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the second binding domain is an scFv that connects via the C-terminus of its CH1 to a N-terminus of the first binding domain via a peptide linker (PLB). In some embodiments, the scFv has a VH at the N-terminus and a VL at the C-terminus. In some embodiments, the scFv has a VL at the N-terminus and a VH at the C-terminus. In some embodiments, the first binding domain is a Fab that connects via the N-terminus of its VH region to the C-terminus of the second binding domain via the peptide linker (PLB). In some embodiments, the C-terminus of the CH1 region of the first binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the second binding domain is an scFv that connects via the C-terminus of its CH1 to a N-terminus of the first binding domain via a peptide linker (PLB). In some embodiments, the scFv has a VH at the N-terminus and a VL at the C-terminus. In some embodiments, the scFv has a VL at the N-terminus and a VH at the C-terminus. In some embodiments, the first binding domain is a Fab that connects via the N-terminus of its VL region to the C-terminus of the second binding domain via the peptide linker (PLB). In some embodiments, the C-terminus of the CH1 region of the first binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the second binding domain is a Fab that connects via the C-terminus of its CH1 to a N-terminus of the first binding domain via a peptide linker (PLB). In some embodiments, the second binding domain is a charge steering Fab. In some embodiments, the first binding domain is a CrossFab that connects via the N-terminus of its VL region to the C-terminus of the CH1 of the second binding domain via the peptide linker PLB. In some embodiments, the C-terminus of the CH1 region of the first binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the first binding domain is a CrossFab that connects via the C-terminus of its CH1 region to a N-terminus of the second binding domain via a peptide linker (PLB). In some embodiments, the second binding domain is a Fab that connects via the N-terminus of its VL region to the C-terminus of the CH1 region of the first binding domain via the peptide linker (PLB). In some embodiments, the second binding domain is a charge steering Fab. In some embodiments, the C-terminus of the CH1 region of the second binding domain is connected to the N-terminus of the second polypeptide of the Fc region comprising a knob mutation via a peptide linker (PLE). In some embodiments, the first polypeptide of the Fc region comprising a hole mutation does not connect to any binding domain via its N-terminus.

In some embodiments, the multi-specific polypeptide comprises a first binding domain and a second binding domain that are connected to each other only through a half-life extension moiety (e.g., a Fc region). In some embodiments, the first binding domain of the multi-specific polypeptide is linked to one polypeptide chain of the Fc region, and the second binding domain of the multi-specific polypeptide is linked to the other polypeptide chain of the Fc region.

In some embodiments, a C-terminus of the first binding domain is connected to a N-terminus of one polypeptide chain of the Fc region, and a C-terminus of the second binding domain is connected to a N-terminus of the other polypeptide chain of the Fc region. In some embodiments, at least one of the binding domains is an scFv that connects to the polypeptide of the Fc region via its C-terminus. The scFv may have a VH at the N-terminus and a VL at the C-terminus, and connects to the polypeptide of the Fc region via the C-terminus of the VL. Alternatively, the scFv may have a VL at the N-terminus and a VH at the C-terminus, and connects to the polypeptide of the Fc region via the C-terminus of the VH. In some embodiments, at least one of the binding domains is a Fab or Cross-Fab that connects to the polypeptide of the Fc region via the C-terminus of its CH1 region. In some embodiments, at least one of the binding domains is a Fab or Cross-Fab that connects to the polypeptide of the Fc region via the C-terminus of its CL region.

In some embodiments, the Fc region has a knobs-in-holes format. In some embodiments, the first binding domain is linked to the second polypeptide of the Fc region comprising a knob mutation, and wherein the second binding domain is linked to the first polypeptide of the Fc region comprising a hole mutation.

In some embodiments, the Fc region has a knobs-in-holes format. In some embodiments, the second binding domain is linked to the second polypeptide of the Fc region comprising a knob mutation, and wherein the first binding domain is linked to the first polypeptide of the Fc region comprising a hole mutation.

In some embodiments, the first binding domain is a Fab connected to the second polypeptide of the Fc region comprising a knob mutation, and the second binding domain is an scFv connected to the first polypeptide of the Fc region comprising a hole mutation. In some embodiments, the first binding domain is a CrossFab connected to the second polypeptide of the Fc region comprising a knob mutation, and the second binding domain is an scFv connected to the first polypeptide of the Fc region comprising a hole mutation. In some embodiments, the scFv has a VH at the N-terminus and a VL at the C-terminus. In some embodiments, the scFv has a VL at the N-terminus and a VH at the C-terminus.

In some embodiments, the first binding domain is a Fab connected to the second polypeptide of the Fc region comprising a knob mutation, and the second binding domain is an scFv connected to the first polypeptide of the Fc region comprising a hole mutation. In some embodiments, the first binding domain is a CrossFab connected to the second polypeptide of the Fc region comprising a knob mutation, and the second binding domain is an scFv connected to the first polypeptide of the Fc region comprising a hole mutation. In some embodiments, the scFv has a VH at the N-terminus and a VL at the C-terminus. In some embodiments, the scFv has a VL at the N-terminus and a VH at the C-terminus.

In some embodiments, the first binding domain is a CrossFab connected to the second polypeptide of the Fc region comprising a knob mutation, and the second binding domain is a Fab connected to the first polypeptide of the Fc region comprising a hole mutation. In some embodiments, the first binding domain connects to the second polypeptide of the Fc region via the C-terminus of its CH1 domain. Alternatively, in some embodiments, the first binding domain connects to the second polypeptide of the Fc region via the C-terminus of its CL domain. In some embodiments, the second binding domain connects to the first polypeptide of the Fc region via the C-terminus of its CH1 domain.

In some embodiments, the first binding domain is a CrossFab connected to the second polypeptide of the Fc region comprising a knob mutation, and the second binding domain is a charge steering Fab connected to the first polypeptide of the Fc region comprising a hole mutation. In some embodiments, the first binding domain connects to the second polypeptide of the Fc region via the C-terminus of its CH1 domain. In some embodiments, the second binding domain connects to the first polypeptide of the Fc region via the C-terminus of its CH1 domain.

In some embodiments, the multi-specific polypeptide comprises three non-identical polypeptide chains. In some embodiments, the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab light chain; and the third polypeptide comprises: (i) an anti-CD19 Fab heavy chain; (ii) a PLE linker; and (v) an Fc heavy chain comprising a knob mutation. In some embodiments, the anti-TCRβ scFv is an anti-TCRβV6-5 scFv.

In some embodiments, the multi-specific polypeptide comprises three non-identical polypeptide chains. In some embodiments, the first polypeptide comprises: (i) a PLE linker; and (ii) an Fc heavy chain comprising a hole mutation; the second polypeptide comprises: (i) an anti-CD19 Fab light chain; and the third polypeptide comprises: (i) an anti-TCRβ scFv; (ii) a PLB linker; (iii) an anti-CD19 Fab heavy chain; (iv) a PLE linker; and (iii) an Fc heavy chain comprising a knob mutation. In some embodiments, the anti-TCRβ scFv is an anti-TCRβV6-5 scFv.

In some embodiments, the multi-specific polypeptide comprises three non-identical polypeptide chains. In some embodiments, the first polypeptide comprises: (i) a PLE linker; and (ii) an Fc heavy chain comprising a hole mutation; the second polypeptide comprises: (i) an anti-CD19 Fab heavy chain; (ii) a PLE linker; and (iii) an Fc heavy chain comprising a knob mutation; and the third polypeptide comprises: (i) an anti-TCRβ scFv; (ii) a PLB linker; (iii) an anti-CD19 Fab light chain. In some embodiments, the anti-TCRβ scFv is an anti-TCRβV6-5 scFv.

In some embodiments, the multi-specific polypeptide comprises three non-identical polypeptide chains. In some embodiments, the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab heavy chain; (iv) a PLE linker; and (v) an Fc heavy chain comprising a knob mutation; and the third polypeptide comprises an anti-CD19 Fab light chain. In some embodiments, the anti-TCRβ scFv is an anti-TCRβV6-5 scFv.

### 1.2.5. Linkers

In some embodiments, the multi-specific polypeptide of the disclosure comprises one or more linkers. In some embodiments, a linker connects the VH region and the VL region of a binding domain (e.g., the first binding domain and/or the second binding domain). In some embodiments, a linker connects the first binding domain and the second binding domain. In some embodiments, a linker connects the first or the second binding domain with a half-life extension moiety. In some embodiments, the linker is or comprises a single amino acid, a peptide, or a polypeptide. In some embodiments, the linker is a non-peptide linker. In some embodiments, the non-peptide linker is a non-proteinaceous polymer such as polyethylene glycol (PEG), polypropylene glycol, polyoxyalkylenes, or a copolymer of polyethylene glycol and polypropylene glycol. In some embodiments, the non-peptide linker is a chemical linker.

In some embodiments, the first binding domain comprises a VH region and a VL region linked by a peptide linker (“PL1”). Suitable linkers are generally those which allow both the VH region and the VL region to fold with a three-dimensional structure very similar to the VH region and the VL region produced individually without any linker or other component. In some embodiments, the C-terminus of the VH region of the first binding domain is linked to the N-terminus of the VL region of the first binding domain via the peptide linker PL1. In some embodiments, the C-terminus of the VL region of the first binding domain is linked to the N-terminus of the VH region of the first binding domain via the peptide linker PL1. In some embodiments, the first binding domain is an scFv (that is, the peptide linker PL1 may be an scFv linker).

In some embodiments, the second binding domain comprises a VH region and a VL region linked by a peptide linker (“PL2”). Suitable linkers are generally those which allow both the VH region and the VL region to fold with a three-dimensional structure very similar to the VH region and the VL region produced individually without any linker or other component. In some embodiments, the C-terminus of the VH region of the second binding domain is linked to the N-terminus of the VL region of the second binding domain via the peptide linker PL2. In some embodiments, the C-terminus of the VL region of the second binding domain is linked to the N-terminus of the VH region of the second binding domain via the peptide linker PL2. In some embodiments, the second binding domain is an scFv (that is, the peptide linker PL2 may be an scFv linker).

In some embodiments, the first binding domain is linked (connected) to the second binding domain via a peptide linker (“PLB”). In some embodiments, a C-terminus of the first binding domain is linked to a N-terminus of the second binding domain via the peptide linker PLB. In some embodiments, a C-terminus of the second binding domain is linked to a N-terminus of the first binding domain via the peptide linker PLB.

In some embodiments, the first binding domain is linked (connected) to the half-life extension moiety (e.g., a Fc region) via a peptide linker (“PLE”). In some embodiments, the second binding domain is linked (connected) to the half-life extension moiety (e.g., a Fc region) via a peptide linker (“PLE”). In some embodiments, a C-terminus of the first binding domain is linked (connected) to the N-terminus of the Fc region via the peptide linker PLE. In some embodiments, a C-terminus of the second binding domain is linked (connected) to the N-terminus of the Fc region via the peptide linker PLE.

In some embodiments, the peptide linker of the disclosure (e.g., PL1, PL2, PLB, and/or PLE) can predominantly include the following amino acid residues: Gly, Ser, Ala, Pro, or Thr. Exemplary linkers include, but are not limited to, glycine-serine linkers, glycine-alanine linkers, proline-threonine, and alanine-serine linkers.

In some embodiments, the linker is between about 5 amino acids to about 10 amino acids in length. In some embodiments, the linker is between about 5 amino acids to about 15 amino acids in length. In some embodiments, the linker is between about 5 amino acids to about 20 amino acids in length. In some embodiments, the linker is between about 5 amino acids to about 30 amino acids in length. In some embodiments, the linker is between about 10 amino acids to about 15 amino acids in length. In some embodiments, the linker is between about 10 amino acids to about 20 amino acids in length. In some embodiments, the linker is between about 10 amino acids to about 30 amino acids in length. In some embodiments, the linker is between about 15 amino acids to about 30 amino acids in length. In some embodiments, the linker is between about 15 amino acids to about 45 amino acids in length. In some embodiments, the linker is 5 amino acids in length.

In some embodiments, the linker is 10 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments, the linker is (G)n, n=1-30. In some embodiments, the linker is (G×S)n, x=1-5, n=15. In some embodiments, the linker is (S×G)n, x=1-5, n=15. In some embodiments, the linker is (GSGGS)n, n=1-5. In some embodiments, the linker is (GGGGS)n, n=1-5. Exemplary linkers are provided in Table 16 below.

TABLE 16
Exemplary Linkers

| | SEQ ID |
|---|---|
| Amino Acid Sequence | NO |
| GSGSG | 110 |
| GGSGGGG | 111 |
| GGGGS | 112 |
| GGGGSGGGGS | 113 |
| GGGGSGGGGSGGGGS | 114 |
| GGGGSGGGGSGGGGSGGGGS | 115 |
| GGGGSGGGGSGGGGSGGGGSGGGGS | 116 |

In some embodiments, peptide linker PL1 and/or PL2 has a length that is adequate to link the VH region and the VL region in such a way that they assume the correct conformation relative to one another so that they retain the desired activity.

In some embodiments, the peptide linker PLE has at least 5 amino acids in length. In some embodiments, the peptide linker PLE is between 5-10 amino acids, between 11-15 amino acids, between 16-20 amino acids, between 21-25 amino acids, or between 26-30 amino acids, in length. In some embodiments, the peptide linker PLE comprises one of the amino acid sequences in Table 16 above. In some embodiments, the peptide linker PLE comprises the amino acid sequence KTHTCP (SEQ ID NO: 158). In some embodiments, the amino acid immediately before the lysine of the amino acid sequence KTHTCP (SEQ ID NO: 158) is not an aspartate. In some embodiments, the peptide linker PLE comprises a (GGGGS) n, n=1-5 sequence and the amino acid sequence KTHTCP (SEQ ID NO: 158). In some embodiments, the peptide linker (PLE) comprises or consists of the amino acid sequence GGGGSGGGGSKTHTCP (SEQ ID NO: 159). In some embodiments, the peptide linker PLE comprises or consists of the amino acid sequence DKTHTCP (SEQ ID NO: 160). In some embodiments, the peptide linker PLE comprises or consists of the amino acid sequence DKTHTCPPCP (SEQ ID NO: 161). In some embodiments, the peptide linker PLE comprises or consists of the amino acid sequence EPKSCDKTHTCPPCP (SEQ ID NO: 162). In some embodiments, the peptide linker PLE comprises or consists of the amino acid sequence ERKCCVECPPCP (SEQ ID NO: 163). In some embodiments, the peptide linker PLE comprises or consists of the amino acid sequence ELKTPLGDTTHTCPRCP (SEQ ID NO: 164). In some embodiments, the peptide linker PLE comprises or consists of the amino acid sequence EPKSCDTPPPCPRCP (SEQ ID NO: 165). In some embodiments, the peptide linker PLE comprises or consists of the amino acid sequence ESKYGPPCPSCP (SEQ ID NO: 166).

In some embodiments, the peptide linker PLB connects the first binding domain and the second binding domain. In some embodiments, the peptide linker PLB has at least 5 amino acids in length. In some embodiments, the peptide linker PLB is between 5-10 amino acids, between 11-15 amino acids, between 16-20 amino acids, between 21-25 amino acids, or between 26-30 amino acids, in length. In some embodiments, the peptide linker PLB comprises one of the amino acid sequences in Table 16 above. In some embodiments, the peptide linker (PLB) comprises the formula of (Gly₄Ser)ₙ, wherein n=1-5. In some embodiments, the peptide linker (PLB) is (Gly₄Ser)₃. In some embodiments, the peptide linker (PLB) is (Gly₄Ser)₂. In some embodiments, the peptide linker (PLB) is (Gly₄Ser)₁.

### 1.2.6. Non-Limiting Examples of Multi-Specific Polypeptides

None-limiting examples of multi-specific polypeptides of the disclosure (e.g., those used in the Examples section below) are provided in Table 17 below.

In some embodiments, the multi-specific polypeptide comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 407-991 and 1159-1179. In some embodiments, the multi-specific polypeptide comprises polypeptide chains having at least 80%, at least 85%, at least 90%, at least 95%, at least 97%, at least 98%, at least 99%, or 100%, identity to the polypeptides of any of the binders listed in Table 17 below—for example, in some embodiments, the multi-specific polypeptide specifically binds to the antigens of TCE-1 binder and comprises four polypeptide chains, which comprise amino acid sequences having at least 80%, at least 85%, at least 90%, at least 91%, at least 92%, at least 93%, at least 94%, at least 95%, at least 96%, at least 97%, at least 98%, at least 99%, or 100%, identity to SEQ ID NO: 407, 502, 688, and 840, respectively.

TABLE 17
Multi-specific Polypeptides

| | SEQ | SEQ ID | SEQ ID | SEQ |
|---|---|---|---|---|
| | ID NO | NO for | NO for | ID NO |
| | for Chain 1 | Chain 2 | Chain 3 | for Chain 4 |
| ID | (LC1) | (HC1) | (LC2) | (HC2) |
| TCE-1 | 407 | 502 | 688 | 840 |
| TCE-2 | 408 | 503 | 689 | 841 |
| TCE-3 | 409 | 504 | 690 | 842 |
| TCE-4 | 410 | 505 | 691 | 843 |
| TCE-5 | 411 | 506 | 692 | 844 |
| TCE-6 | 412 | 507 | 693 | 845 |
| TCE-7 | 413 | 508 | 694 | 846 |
| TCE-8 | 414 | 509 | 695 | 847 |
| TCE-9 | 415 | 510 | | |
| TCE-10 | 416 | 511 | | |
| TCE-11 | 417 | 512 | | |
| TCE-12 | 418 | 513 | | |
| TCE-13 | 419 | 514 | | |
| TCE-14 | 420 | 515 | | |
| TCE-15 | 421 | 516 | | |
| TCE-16 | 422 | 517 | | |
| TCE-17 | 423 | 518 | | |
| TCE-18 | 424 | 519 | | |
| TCE-19 | 425 | 520 | | |
| TCE-20 | 426 | 521 | | |
| TCE-21 | 427 | 522 | | |
| TCE-22 | 428 | 523 | | |
| TCE-23 | 429 | 524 | | |
| TCE-24 | 430 | 525 | | |
| TCE-25 | 431 | 526 | 696 | 848 |
| TCE-26 | 432 | 527 | | |
| TCE-27 | 433 | 528 | | |
| TCE-28 | 434 | 529 | 697 | 849 |
| TCE-29 | 435 | 530 | 698 | 850 |
| TCE-30 | 436 | 531 | 699 | 851 |
| TCE-31 | 437 | 532 | 700 | 852 |
| TCE-32 | 438 | 533 | 701 | 853 |
| TCE-33 | 439 | 534 | 702 | 854 |
| TCE-34 | 440 | 535 | 703 | 855 |
| TCE-35 | 441 | 536 | 704 | 856 |
| TCE-36 | 442 | 537 | 705 | 857 |
| TCE-37 | 443 | 538 | 706 | 858 |
| TCE-38 | 444 | 539 | 707 | 859 |
| TCE-39 | 445 | 540 | 708 | 860 |
| TCE-40 | 446 | 541 | 709 | 861 |
| TCE-41 | 447 | 542 | 710 | 862 |
| TCE-42 | 448 | 543 | 711 | 863 |
| TCE-43 | 449 | 544 | 712 | 864 |
| TCE-44 | 450 | 545 | 713 | 865 |
| TCE-45 | 451 | 546 | 714 | 866 |
| TCE-46 | 452 | 547 | 715 | 867 |
| TCE-47 | 453 | 548 | 716 | 868 |
| TCE-48 | 454 | 549 | 717 | 869 |
| TCE-49 | 455 | 550 | 718 | 870 |
| TCE-50 | 456 | 551 | 719 | 871 |
| TCE-51 | 457 | 552 | 720 | 872 |
| TCE-52 | 458 | | | |
| TCE-53 | 459 | 553 | 721 | 873 |
| TCE-54 | 460 | 554 | 722 | 874 |
| TCE-55 | 461 | 555 | 723 | 875 |
| TCE-56 | 462 | 556 | 724 | 876 |
| TCE-57 | 463 | 557 | 725 | 877 |
| TCE-58 | 464 | 558 | 726 | 878 |
| TCE-59 | 465 | 559 | 727 | 879 |
| TCE-60 | 466 | 560 | 728 | 880 |
| TCE-61 | 467 | 561 | 729 | 881 |
| TCE-62 | 468 | 562 | 730 | 882 |
| TCE-63 | 469 | 563 | 731 | 883 |
| TCE-64 | 470 | 564 | 732 | 884 |
| TCE-65 | 471 | 565 | 733 | 885 |
| TCE-66 | 472 | 566 | 734 | 886 |
| TCE-67 | 473 | 567 | 735 | 887 |
| TCE-68 | 474 | 568 | 736 | 888 |
| TCE-69 | 475 | 569 | 737 | 889 |
| TCE-70 | 476 | 570 | 738 | 890 |
| TCE-71 | 477 | 571 | 739 | 891 |
| TCE-72 | 478 | 572 | 740 | 892 |
| TCE-73 | 479 | 573 | 741 | 893 |
| TCE-74 | 480 | 574 | 742 | 894 |
| TCE-75 | 481 | 575 | 743 | 895 |
| TCE-76 | 482 | 576 | 744 | 896 |
| TCE-77 | 483 | 577 | 745 | 897 |
| TCE-78 | 484 | 578 | 746 | 898 |
| TCE-79 | 485 | 579 | 747 | 899 |
| TCE-80 | | 580 | 748 | 900 |
| TCE-81 | | 581 | 749 | 901 |
| TCE-82 | | 582 | 750 | 902 |
| TCE-83 | | 583 | 751 | 903 |
| TCE-84 | | 584 | 752 | 904 |
| TCE-85 | | 585 | 753 | 905 |
| TCE-86 | | 586 | 754 | 906 |
| TCE-87 | | 587 | 755 | 907 |
| TCE-88 | | 588 | 756 | 908 |
| TCE-89 | | 589 | 757 | 909 |
| TCE-90 | | 590 | 758 | 910 |
| TCE-91 | | 591 | 759 | 911 |
| TCE-92 | | 592 | 760 | 912 |
| TCE-93 | | 593 | 761 | 913 |
| TCE-94 | | 594 | 762 | 914 |
| TCE-95 | | 595 | 763 | 915 |
| TCE-96 | | 596 | 764 | 916 |
| TCE-97 | | 597 | 765 | 917 |
| TCE-98 | | 598 | 766 | 918 |
| TCE-99 | | 599 | 76 | 919 |
| TCE-100 | | 600 | 768 | 920 |
| TCE-101 | | 601 | 769 | 921 |
| TCE-102 | | 602 | 770 | 922 |
| TCE-103 | | 603 | 771 | 923 |
| TCE-104 | | 604 | 772 | 924 |
| TCE-105 | | 605 | 773 | 925 |
| TCE-106 | | 606 | 774 | 926 |
| TCE-107 | | 607 | 775 | 927 |
| TCE-108 | | 608 | 776 | 928 |
| TCE-109 | | 609 | 777 | 929 |
| TCE-110 | | 610 | 778 | 930 |
| TCE-111 | | 611 | 779 | 931 |
| TCE-112 | | 612 | 780 | 932 |
| TCE-113 | | 613 | 781 | 933 |
| TCE-114 | | 614 | 782 | 934 |
| TCE-115 | | 615 | 783 | 935 |
| TCE-116 | | 616 | 784 | 936 |
| TCE-117 | | 617 | 785 | 937 |
| TCE-118 | | 618 | 786 | 938 |
| TCE-119 | | 619 | 787 | 939 |
| TCE-120 | | 620 | 788 | 940 |
| TCE-121 | | 621 | 789 | 941 |
| TCE-122 | | 622 | 790 | 942 |
| TCE-123 | | 623 | 791 | 943 |
| TCE-124 | | 624 | 792 | 944 |
| TCE-125 | | 625 | 793 | 945 |
| TCE-126 | 486 | 626 | | |
| TCE-127 | 487 | 627 | | |
| TCE-128 | 488 | 628 | | |
| TCE-129 | 489 | 629 | | |
| TCE-130 | 490 | 630 | | |
| TCE-131 | 491 | 631 | | |
| TCE-132 | 492 | 632 | | |
| TCE-133 | 493 | 633 | | |
| TCE-134 | 494 | 634 | | |
| TCE-135 | 495 | 635 | | |
| TCE-136 | 496 | 636 | | |
| TCE-137 | 497 | 637 | | |
| TCE-138 | 498 | 638 | | |
| TCE-139 | 499 | 639 | | |
| TCE-140 | 500 | 640 | | |
| TCE-141 | 501 | 641 | | |
| TCE-142 | | 642 | 794 | 946 |
| TCE-143 | | 643 | 795 | 947 |
| TCE-144 | | 644 | 796 | 948 |
| TCE-145 | | 645 | 797 | 949 |
| TCE-146 | | 646 | 798 | 950 |
| TCE-147 | | 647 | 799 | 951 |
| TCE-148 | | 648 | 800 | 952 |
| TCE-149 | | 649 | 801 | 953 |
| TCE-150 | | 650 | 802 | 954 |
| TCE-151 | | 651 | 803 | 955 |
| TCE-152 | | 652 | 804 | 956 |
| TCE-153 | | 653 | 805 | 957 |
| TCE-154 | | 654 | 806 | 958 |
| TCE-155 | | 655 | 807 | 959 |
| TCE-156 | | 656 | 808 | 960 |
| TCE-157 | | 657 | 809 | 961 |
| TCE-158 | | 658 | 810 | 962 |
| TCE-159 | | 659 | 811 | 963 |
| TCE-160 | | 660 | 812 | 964 |
| TCE-161 | | 661 | 813 | 965 |
| TCE-162 | | 662 | 814 | 966 |
| TCE-163 | | 663 | 815 | 967 |
| TCE-164 | | 664 | 816 | 968 |
| TCE-165 | | 665 | 817 | 969 |
| TCE-166 | | 666 | 818 | 970 |
| TCE-167 | | 667 | 819 | 971 |
| TCE-168 | | 668 | 820 | 972 |
| TCE-169 | | 669 | 821 | 973 |
| TCE-170 | | 670 | 822 | 974 |
| TCE-171 | | 671 | 823 | 975 |
| TCE-172 | | 672 | 824 | 976 |
| TCE-173 | | 673 | 825 | 977 |
| TCE-174 | | 674 | 826 | 978 |
| TCE-175 | | 675 | 827 | 979 |
| TCE-176 | | 676 | 828 | 980 |
| TCE-177 | | 677 | 829 | 981 |
| TCE-178 | | 678 | 830 | 982 |
| TCE-179 | | 679 | 831 | 983 |
| TCE-180 | | 680 | 832 | 984 |
| TCE-181 | | 681 | 833 | 985 |
| TCE-182 | | 682 | 834 | 986 |
| TCE-183 | | 683 | 835 | 987 |
| TCE-184 | | 684 | 836 | 988 |
| TCE-185 | | 685 | 837 | 989 |
| TCE-186 | | 686 | 838 | 990 |
| TCE-187 | | 687 | 839 | 991 |
| CLY-018 | | 1159 | 1166 | 1173 |
| CLY-019 | | 1160 | 1167 | 1174 |
| CLY-020 | | 1161 | 1168 | 1175 |
| CLY-021 | | 1162 | 1169 | 1176 |
| CLY-022 | | 1163 | 1170 | 1177 |
| CLY-023 | | 1164 | 1171 | 1178 |
| CLY-024 | | 1165 | 1172 | 1179 |

In some embodiments, the multi-specific polypeptide comprises: (i) an anti-TCRβV6-5 binding domain comprising: (a) an HCDR1 comprising SEQ ID NO: 1138 or SEQ ID NO: 1143; (b) an HCDR2 comprising SEQ ID NO: 2 or SEQ ID NO: 18; (c) an HCDR3 comprising SEQ ID NO: 1139 or SEQ ID NO: 1144; (d) an LCDR1 comprising SEQ ID NO: 1140 or SEQ ID NO: 1145; (e) an LCDR2 comprising SEQ ID NO: 5 or SSS; and (f) an LCDR3 comprising SEQ ID NO: 1141 or SEQ ID NO: 1146; and (ii) an anti-CD19 binding domain comprising: (a) an HCDR1 comprising SEQ ID NO: 303 or SEQ ID NO: 339; (b) an HCDR2 comprising SEQ ID NO: 311 or SEQ ID NO: 346; (c) an HCDR3 comprising SEQ ID NO: 317 or SEQ ID NO: 351; (d) an LCDR1 comprising SEQ ID NO: 323 or SEQ ID NO: 357; (e) an LCDR2 comprising SEQ ID NO: 329 or SEQ ID NO: 363; and (f) an LCDR3 comprising SEQ ID NO: 334. In some embodiments, the CDRs comprise 1, 2, 3, or more amino acid modifications. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) an HCDR1 comprising SEQ ID NO: 1 or SEQ ID NO: 17; (b) an HCDR2 comprising SEQ ID NO: 1142 or SEQ ID NO: 1147; (c) an HCDR3 comprising SEQ ID NO: 1139 or SEQ ID NO: 1144; (d) an LCDR1 comprising SEQ ID NO: 1140 or SEQ ID NO: 1145; (e) an LCDR2 comprising SEQ ID NO: 5 or SSS; and (f) an LCDR3 comprising SEQ ID NO: 1141 or SEQ ID NO: 1146; and (ii) an anti-CD19 binding domain comprising: (a) an HCDR1 comprising SEQ ID NO: 306 or SEQ ID NO: 342; (b) an HCDR2 comprising SEQ ID NO: 314 or SEQ ID NO: 347; (c) an HCDR3 comprising SEQ ID NO: 318 or SEQ ID NO: 352; (d) an LCDR1 comprising SEQ ID NO: 326 or SEQ ID NO: 360; (e) an LCDR2 comprising SEQ ID NO: 328 or SEQ ID NO: 362; and (f) an LCDR3 comprising SEQ ID NO: 335. In some embodiments, the CDRs comprise 1, 2, 3, or more amino acid modifications. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) an HCDR1 comprising SEQ ID NO: 1138 or SEQ ID NO: 1143; (b) an HCDR2 comprising SEQ ID NO: 2 or SEQ ID NO: 18; (c) an HCDR3 comprising SEQ ID NO: 1139 or SEQ ID NO: 1144; (d) an LCDR1 comprising SEQ ID NO: 1140 or SEQ ID NO: 1145; (e) an LCDR2 comprising SEQ ID NO: 5 or SSS; and (f) an LCDR3 comprising SEQ ID NO: 1141 or SEQ ID NO: 1146; and (ii) an anti-CD19 binding domain comprising: (a) an HCDR1 comprising SEQ ID NO: 306 or SEQ ID NO: 342; (b) an HCDR2 comprising SEQ ID NO: 314 or SEQ ID NO: 347; (c) an HCDR3 comprising SEQ ID NO: 318 or SEQ ID NO: 352; (d) an LCDR1 comprising SEQ ID NO: 326 or SEQ ID NO: 360; (e) an LCDR2 comprising SEQ ID NO: 328 or SEQ ID NO: 362; and (f) an LCDR3 comprising SEQ ID NO: 335. In some embodiments, the CDRs comprise 1, 2, 3, or more amino acid modifications. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) an HCDR1 comprising SEQ ID NO: 1 or SEQ ID NO: 17; (b) an HCDR2 comprising SEQ ID NO: 1142 or SEQ ID NO: 1147; (c) an HCDR3 comprising SEQ ID NO: 1139 or SEQ ID NO: 1144; (d) an LCDR1 comprising SEQ ID NO: 1140 or SEQ ID NO: 1145; (e) an LCDR2 comprising SEQ ID NO: 5 or SSS; and (f) an LCDR3 comprising SEQ ID NO: 1141 or SEQ ID NO: 1146; and (ii) an anti-CD19 binding domain comprising: (a) an HCDR1 comprising SEQ ID NO: 303 or SEQ ID NO: 339; (b) an HCDR2 comprising SEQ ID NO: 311 or SEQ ID NO: 346; (c) an HCDR3 comprising SEQ ID NO: 317 or SEQ ID NO: 351; (d) an LCDR1 comprising SEQ ID NO: 323 or SEQ ID NO: 357; (e) an LCDR2 comprising SEQ ID NO: 329 or SEQ ID NO: 363; and (f) an LCDR3 comprising SEQ ID NO: 334. In some embodiments, the CDRs comprise 1, 2, 3, or more amino acid modifications. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1148; and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1149; and (ii) an anti-CD19 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 371, and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 372. In some embodiments, the VH region of the anti-TCRβV6-5 binding domain comprises a cysteine residue at amino acid position 44; and the VL region of the anti-TCRβV6-5 binding domain comprises a cysteine residue at amino acid position 100. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1150; and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1149; and (ii) an anti-CD19 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 383, and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 384. In some embodiments, the VH region of the anti-TCRβV6-5 binding domain comprises a cysteine residue at amino acid position 44; and the VL region of the anti-TCRβV6-5 binding domain comprises a cysteine residue at amino acid position 100. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1148; and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1149; and (ii) an anti-CD19 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 383, and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 384. In some embodiments, the VH region of the anti-TCRβV6-5 binding domain comprises a cysteine residue at amino acid position 44; and the VL region of the anti-TCRβV6-5 binding domain comprises a cysteine residue at amino acid position 100. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide comprises: (i) an anti-TCRβV6-5 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1150; and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1149; and (ii) an anti-CD19 binding domain comprising: (a) a VH region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 371, and (b) a VL region comprising an amino acid sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 372. In some embodiments, the VH region of the anti-TCRβV6-5 binding domain comprises a cysteine residue at amino acid position 44; and the VL region of the anti-TCRβV6-5 binding domain comprises a cysteine residue at amino acid position 100. In some embodiments, the anti-TCRβV6-5 binding domain is an scFv and the anti-CD19 binding domain is a Fab. In some embodiments, the multi-specific polypeptide further comprises a half-life extension moiety.

In some embodiments, the multi-specific polypeptide complex provided herein comprises a first polypeptide, a second polypeptide, and a third polypeptide. In some embodiments, the first polypeptide comprises or consists of an amino acid sequence of SEQ ID NO: 1159; the second polypeptide comprises or consists of an amino acid sequence of SEQ ID NO: 1166; and the third polypeptide comprises or consists of an amino acid sequence of SEQ ID NO: 1173. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1159; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1166; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1173. In some embodiments, the first polypeptide comprises an amino acid sequence of SEQ ID NO: 1159 with one or more mutations therein; the second polypeptide comprises an amino acid sequence of SEQ ID NO: 1166 with one or more mutations therein; and/or the third polypeptide comprises an amino acid sequence of SEQ ID NO: 1173 with one or more mutations therein. For example, in some embodiments, the first polypeptide comprises 1, 2, 3, 4, 5, 6, 7, 8, 9, 10, or more mutations in SEQ ID NO: 1159; the second polypeptide comprises 1, 2, 3, 4, 5, 6, 7, 8, 9, 10, or more mutations in SEQ ID NO: 1166; and/or the third polypeptide comprises 1, 2, 3, 4, 5, 6, 7, 8, 9, 10, or more mutations in SEQ ID NO: 1173. In some embodiments, the first polypeptide comprises the amino acid sequence of SEQ ID NO: 1159 with 1-5, 5-10, 11-15, 15-20, 20-25, 25-30, or more than 30 mutations; the second polypeptide comprises the amino acid sequence of SEQ ID NO: 1166 with 1-5, 5-10, 11-15, 15-20, 20-25, 25-30, or more than 30 mutations; and/or the third polypeptide comprises the amino acid sequence of SEQ ID NO: 1173 with 1-5, 5-10, 11-15, 15-20, 20-25, 25-30, or more than 30 mutations.

In some embodiments, the multi-specific polypeptide complex provided herein comprises a first polypeptide, a second polypeptide, and a third polypeptide. In some embodiments, the first polypeptide comprises or consists of an amino acid sequence of SEQ ID NO: 1160; the second polypeptide comprises or consists of an amino acid sequence of SEQ ID NO: 1167; and the third polypeptide comprises or consists of an amino acid sequence of SEQ ID NO: 1174. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1160; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1167; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1174. In some embodiments, the first polypeptide comprises an amino acid sequence of SEQ ID NO: 1160 with one or more mutations therein; the second polypeptide comprises an amino acid sequence of SEQ ID NO: 1167 with one or more mutations therein; and/or the third polypeptide comprises an amino acid sequence of SEQ ID NO: 1174 with one or more mutations therein. For example, in some embodiments, the first polypeptide comprises 1, 2, 3, 4, 5, 6, 7, 8, 9, 10, or more mutations in SEQ ID NO: 1160; the second polypeptide comprises 1, 2, 3, 4, 5, 6, 7, 8, 9, 10, or more mutations in SEQ ID NO: 1167; and/or the third polypeptide comprises 1, 2, 3, 4, 5, 6, 7, 8, 9, 10, or more mutations in SEQ ID NO: 1174. In some embodiments, the first polypeptide comprises the amino acid sequence of SEQ ID NO: 1160 with 1-5, 5-10, 11-15, 15-20, 20-25, 25-30, or more than 30 mutations; the second polypeptide comprises the amino acid sequence of SEQ ID NO: 1167 with 1-5, 5-10, 11-15, 15-20, 20-25, 25-30, or more than 30 mutations; and/or the third polypeptide comprises the amino acid sequence of SEQ ID NO: 1174 with 1-5, 5-10, 11-15, 15-20, 20-25, 25-30, or more than 30 mutations.

In some embodiments, the multi-specific polypeptide complex provided herein comprises a first polypeptide and a second polypeptide. In some embodiments, the first polypeptide comprises or consists of an amino acid sequence of SEQ ID NO: 1161; the second polypeptide comprises or consists of an amino acid sequence of SEQ ID NO: 1168; and the third polypeptide comprises or consists of an amino acid sequence of SEQ ID NO: 1175. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1161; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1168; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1175. In some embodiments, the first polypeptide comprises an amino acid sequence of SEQ ID NO: 1161 with one or more mutations therein; the second polypeptide comprises an amino acid sequence of SEQ ID NO: 1168 with one or more mutations therein; and/or the third polypeptide comprises an amino acid sequence of SEQ ID NO: 1175 with one or more mutations therein. For example, in some embodiments, the first polypeptide comprises 1, 2, 3, 4, 5, 6, 7, 8, 9, 10, or more mutations in SEQ ID NO: 1161; the second polypeptide comprises 1, 2, 3, 4, 5, 6, 7, 8, 9, 10, or more mutations in SEQ ID NO: 1168; and/or the third polypeptide comprises 1, 2, 3, 4, 5, 6, 7, 8, 9, 10, or more mutations in SEQ ID NO: 1175. In some embodiments, the first polypeptide comprises the amino acid sequence of SEQ ID NO: 1161 with 1-5, 5-10, 11-15, 15-20, 20-25, 25-30, or more than 30 mutations; the second polypeptide comprises the amino acid sequence of SEQ ID NO: 1168 with 1-5, 5-10, 11-15, 15-20, 20-25, 25-30, or more than 30 mutations; and/or the third polypeptide comprises the amino acid sequence of SEQ ID NO: 1175 with 1-5, 5-10, 11-15, 15-20, 20-25, 25-30, or more than 30 mutations.

In some embodiments, the multi-specific polypeptide complex provided herein comprises a first polypeptide and a second polypeptide. In some embodiments, the first polypeptide comprises or consists of an amino acid sequence of SEQ ID NO: 1162; the second polypeptide comprises or consists of an amino acid sequence of SEQ ID NO: 1169; and the third polypeptide comprises or consists of an amino acid sequence of SEQ ID NO: 1176. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1162; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1169; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1176. In some embodiments, the first polypeptide comprises an amino acid sequence of SEQ ID NO: 1162 with one or more mutations therein; the second polypeptide comprises an amino acid sequence of SEQ ID NO: 1169 with one or more mutations therein; and/or the third polypeptide comprises an amino acid sequence of SEQ ID NO: 1176 with one or more mutations therein. For example, in some embodiments, the first polypeptide comprises 1, 2, 3, 4, 5, 6, 7, 8, 9, 10, or more mutations in SEQ ID NO: 1162; the second polypeptide comprises 1, 2, 3, 4, 5, 6, 7, 8, 9, 10, or more mutations in SEQ ID NO: 1169; and/or the third polypeptide comprises 1, 2, 3, 4, 5, 6, 7, 8, 9, 10, or more mutations in SEQ ID NO: 1176. In some embodiments, the first polypeptide comprises the amino acid sequence of SEQ ID NO: 1162 with 1-5, 5-10, 11-15, 15-20, 20-25, 25-30, or more than 30 mutations; the second polypeptide comprises the amino acid sequence of SEQ ID NO: 1169 with 1-5, 5-10, 11-15, 15-20, 20-25, 25-30, or more than 30 mutations; and/or the third polypeptide comprises the amino acid sequence of SEQ ID NO: 1176 with 1-5, 5-10, 11-15, 15-20, 20-25, 25-30, or more than 30 mutations.

In some embodiments, the multi-specific polypeptide complex provided herein comprises a first polypeptide and a second polypeptide. In some embodiments, the first polypeptide comprises or consists of an amino acid sequence of SEQ ID NO: 1163; the second polypeptide comprises or consists of an amino acid sequence of SEQ ID NO: 1170; and the third polypeptide comprises or consists of an amino acid sequence of SEQ ID NO: 1177. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1163; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1170; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1177. In some embodiments, the first polypeptide comprises an amino acid sequence of SEQ ID NO: 1163 with one or more mutations therein; the second polypeptide comprises an amino acid sequence of SEQ ID NO: 1170 with one or more mutations therein; and/or the third polypeptide comprises an amino acid sequence of SEQ ID NO: 1177 with one or more mutations therein. For example, in some embodiments, the first polypeptide comprises 1, 2, 3, 4, 5, 6, 7, 8, 9, 10, or more mutations in SEQ ID NO: 1163; the second polypeptide comprises 1, 2, 3, 4, 5, 6, 7, 8, 9, 10, or more mutations in SEQ ID NO: 1170; and/or the third polypeptide comprises 1, 2, 3, 4, 5, 6, 7, 8, 9, 10, or more mutations in SEQ ID NO: 1177. In some embodiments, the first polypeptide comprises the amino acid sequence of SEQ ID NO: 1163 with 1-5, 5-10, 11-15, 15-20, 20-25, 25-30, or more than 30 mutations; the second polypeptide comprises the amino acid sequence of SEQ ID NO: 1170 with 1-5, 5-10, 11-15, 15-20, 20-25, 25-30, or more than 30 mutations; and/or the third polypeptide comprises the amino acid sequence of SEQ ID NO: 1177 with 1-5, 5-10, 11-15, 15-20, 20-25, 25-30, or more than 30 mutations.

In some embodiments, the multi-specific polypeptide complex provided herein comprises a first polypeptide and a second polypeptide. In some embodiments, the first polypeptide comprises or consists of an amino acid sequence of SEQ ID NO: 1164; the second polypeptide comprises or consists of an amino acid sequence of SEQ ID NO: 1171; and the third polypeptide comprises or consists of an amino acid sequence of SEQ ID NO: 1178. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1164; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1171; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1178. In some embodiments, the first polypeptide comprises an amino acid sequence of SEQ ID NO: 1164 with one or more mutations therein; the second polypeptide comprises an amino acid sequence of SEQ ID NO: 1171 with one or more mutations therein; and/or the third polypeptide comprises an amino acid sequence of SEQ ID NO: 1178 with one or more mutations therein. For example, in some embodiments, the first polypeptide comprises 1, 2, 3, 4, 5, 6, 7, 8, 9, 10, or more mutations in SEQ ID NO: 1164; the second polypeptide comprises 1, 2, 3, 4, 5, 6, 7, 8, 9, 10, or more mutations in SEQ ID NO: 1171; and/or the third polypeptide comprises 1, 2, 3, 4, 5, 6, 7, 8, 9, 10, or more mutations in SEQ ID NO: 1178. In some embodiments, the first polypeptide comprises the amino acid sequence of SEQ ID NO: 1164 with 1-5, 5-10, 11-15, 15-20, 20-25, 25-30, or more than 30 mutations; the second polypeptide comprises the amino acid sequence of SEQ ID NO: 1171 with 1-5, 5-10, 11-15, 15-20, 20-25, 25-30, or more than 30 mutations; and/or the third polypeptide comprises the amino acid sequence of SEQ ID NO: 1178 with 1-5, 5-10, 11-15, 15-20, 20-25, 25-30, or more than 30 mutations.

In some embodiments, the multi-specific polypeptide complex provided herein comprises a first polypeptide and a second polypeptide. In some embodiments, the first polypeptide comprises or consists of an amino acid sequence of SEQ ID NO: 1165; the second polypeptide comprises or consists of an amino acid sequence of SEQ ID NO: 1172; and the third polypeptide comprises or consists of an amino acid sequence of SEQ ID NO: 1179. In some embodiments, the first polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1165; the second polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1172; and the third polypeptide comprises an amino acid sequence with at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO: 1179. In some embodiments, the first polypeptide comprises an amino acid sequence of SEQ ID NO: 1165 with one or more mutations therein; the second polypeptide comprises an amino acid sequence of SEQ ID NO: 1172 with one or more mutations therein; and/or the third polypeptide comprises an amino acid sequence of SEQ ID NO: 1179 with one or more mutations therein. For example, in some embodiments, the first polypeptide comprises 1, 2, 3, 4, 5, 6, 7, 8, 9, 10, or more mutations in SEQ ID NO: 1165; the second polypeptide comprises 1, 2, 3, 4, 5, 6, 7, 8, 9, 10, or more mutations in SEQ ID NO: 1172; and/or the third polypeptide comprises 1, 2, 3, 4, 5, 6, 7, 8, 9, 10, or more mutations in SEQ ID NO: 1179. In some embodiments, the first polypeptide comprises the amino acid sequence of SEQ ID NO: 1165 with 1-5, 5-10, 11-15, 15-20, 20-25, 25-30, or more than 30 mutations; the second polypeptide comprises the amino acid sequence of SEQ ID NO: 1172 with 1-5, 5-10, 11-15, 15-20, 20-25, 25-30, or more than 30 mutations; and/or the third polypeptide comprises the amino acid sequence of SEQ ID NO: 1179 with 1-5, 5-10, 11-15, 15-20, 20-25, 25-30, or more than 30 mutations.

### 1.2.7. Modifications and Variations

Amino acid sequence modification(s) of the multi-specific polypeptides, the first binding domains, the second binding domains, or the half-life extension moieties provided herein are contemplated. These modifications can provide for some additional property, such as to increase its bio-longevity, to alter its secretory characteristics, etc. In addition, it may be desirable to improve the binding affinity of binding domains to their corresponding binding targets; it may also be desirable to improve other biological properties, including but not limited to specificity, thermostability, expression level, or solubility. Thus, in addition to the first binding domains, the second binding domains, and the half-life extension moieties described herein, it is contemplated that variants can be prepared.

In some embodiments, the multi-specific polypeptides, the first binding domains, the second binding domains, or the half-life extension moieties provided herein are chemically modified, for example, by the covalent attachment of any type of molecule to the multi-specific polypeptides, the first binding domains, the second binding domains, or the half-life extension moieties. Exemplary non-limiting modifications include glycosylation, acetylation, pegylation, phosphorylation, amidation, derivatization by known protecting/blocking groups, proteolytic cleavage, linkage to a cellular ligand or other protein, etc. Additionally, the multi-specific polypeptides, the first binding domains, the second binding domains, or the half-life extension moieties may contain one or more non-classical amino acids.

In some embodiments, variations may be a substitution, deletion, or insertion of one or more amino acids that results in a change in the amino acid sequence as compared with the disclosed sequences. The variation allowed may be determined by systematically making insertions, deletions, or substitutions of amino acids in the sequence and testing the resulting variants for activity exhibited by the full-length or mature native sequence.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Table 18 below lists exemplary conservative amino acid substitutions. Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

TABLE 18
Conservative Substitutions

| Original AA | Conservative Substitution |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Substitution of one or more CDR residues or omission of one or more CDRs is also contemplated. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

Substitutions may be in the range of about 1 to 100 amino acids. In some embodiments, the substitution includes fewer than about 100 amino acid substitutions, fewer than about 95 amino acid substitutions, fewer than about 90 amino acid substitutions, fewer than about 85 amino acid substitutions, fewer than about 80 amino acid substitutions, fewer than about 75 amino acid substitutions, fewer than about 70 amino acid substitutions, fewer than about 65 amino acid substitutions, fewer than about 50 amino acid substitutions, fewer than about 45 amino acid substitutions, fewer than about 40 amino acid substitutions, fewer than about 35 amino acid substitutions, fewer than about 30 amino acid substitutions, fewer than about 25 amino acid substitutions, fewer than about 20 amino acid substitutions, fewer than about 15 amino acid substitutions, fewer than about 10 amino acid substitutions, fewer than about 5 amino acid substitutions, fewer than about 4 amino acid substitutions, fewer than about 3 amino acid substitutions, or fewer than about 2 amino acid substitutions relative to the original molecule.

Amino acid sequence insertions include amino- and/or carboxyl-terminal fusions ranging in length from 1 residue to polypeptides containing 100 or more residues, as well as intrasequence insertions of single or multiple amino acid residues. Examples of terminal insertions include an antibody with an N-terminal methionyl residue. Other insertional variants of the antibody molecule include the fusion to the N or C terminus of the antibody to an enzyme (e.g., for antibody-directed enzyme prodrug therapy) or a polypeptide which increases the serum half-life of the antibody.

In some embodiments, the insertion is about 1 amino acid to about 100 amino acids. In some embodiments, the insertion is at least about 1 amino acid. In some embodiments, the insertion is at most about 100 amino acids. In some embodiments, the insertion is about 1 amino acid to about 5 amino acids, about 1 amino acid to about 10 amino acids, about 1 amino acid to about 20 amino acids, about 1 amino acid to about 30 amino acids, about 1 amino acid to about 40 amino acids, about 1 amino acid to about 50 amino acids, about 1 amino acid to about 60 amino acids, about 1 amino acid to about 70 amino acids, about 1 amino acid to about 80 amino acids, about 1 amino acid to about 90 amino acids, about 1 amino acid to about 100 amino acids, about 5 amino acids to about 10 amino acids, about 5 amino acids to about 20 amino acids, about 5 amino acids to about 30 amino acids, about 5 amino acids to about 40 amino acids, about 5 amino acids to about 50 amino acids, about 5 amino acids to about 60 amino acids, about 5 amino acids to about 70 amino acids, about 5 amino acids to about 80 amino acids, about 5 amino acids to about 90 amino acids, about 5 amino acids to about 100 amino acids, about 10 amino acids to about 20 amino acids, about 10 amino acids to about 30 amino acids, about 10 amino acids to about 40 amino acids, about 10 amino acids to about 50 amino acids, about 10 amino acids to about 60 amino acids, about 10 amino acids to about 70 amino acids, about 10 amino acids to about 80 amino acids, about 10 amino acids to about 90 amino acids, about 10 amino acids to about 100 amino acids, about 20 amino acids to about 30 amino acids, about 20 amino acids to about 40 amino acids, about 20 amino acids to about 50 amino acids, about 20 amino acids to about 60 amino acids, about 20 amino acids to about 70 amino acids, about 20 amino acids to about 80 amino acids, about 20 amino acids to about 90 amino acids, about 20 amino acids to about 100 amino acids, about 30 amino acids to about 40 amino acids, about 30 amino acids to about 50 amino acids, about 30 amino acids to about 60 amino acids, about 30 amino acids to about 70 amino acids, about 30 amino acids to about 80 amino acids, about 30 amino acids to about 90 amino acids, about 30 amino acids to about 100 amino acids, about 40 amino acids to about 50 amino acids, about 40 amino acids to about 60 amino acids, about 40 amino acids to about 70 amino acids, about 40 amino acids to about 80 amino acids, about 40 amino acids to about 90 amino acids, about 40 amino acids to about 100 amino acids, about 50 amino acids to about 60 amino acids, about 50 amino acids to about 70 amino acids, about 50 amino acids to about 80 amino acids, about 50 amino acids to about 90 amino acids, about 50 amino acids to about 100 amino acids, about 60 amino acids to about 70 amino acids, about 60 amino acids to about 80 amino acids, about 60 amino acids to about 90 amino acids, about 60 amino acids to about 100 amino acids, about 70 amino acids to about 80 amino acids, about 70 amino acids to about 90 amino acids, about 70 amino acids to about 100 amino acids, about 80 amino acids to about 90 amino acids, about 80 amino acids to about 100 amino acids, or about 90 amino acids to about 100 amino acids, including any ranges or subranges therebetween. In some embodiments, the insertion is about 1 amino acid, about 5 amino acids, about 10 amino acids, about 20 amino acids, about 30 amino acids, about 40 amino acids, about 50 amino acids, about 60 amino acids, about 70 amino acids, about 80 amino acids, about 90 amino acids, or about 100 amino acids, including any ranges or subranges therebetween.

Amino acid sequence deletions include amino- and/or carboxyl-terminal deletions ranging in length from one residue to polypeptides containing a hundred or more residues, as well as intrasequence deletions of single or multiple amino acid residues.

In some embodiments, the deletion is about 1 amino acid to about 100 amino acids. In some embodiments, the deletion is at least about 1 amino acid. In some embodiments, the deletion is at most about 100 amino acids. In some embodiments, the deletion is about 1 amino acid to about 5 amino acids, about 1 amino acid to about 10 amino acids, about 1 amino acid to about 20 amino acids, about 1 amino acid to about 30 amino acids, about 1 amino acid to about 40 amino acids, about 1 amino acid to about 50 amino acids, about 1 amino acid to about 60 amino acids, about 1 amino acid to about 70 amino acids, about 1 amino acid to about 80 amino acids, about 1 amino acid to about 90 amino acids, about 1 amino acid to about 100 amino acids, about 5 amino acids to about 10 amino acids, about 5 amino acids to about 20 amino acids, about 5 amino acids to about 30 amino acids, about 5 amino acids to about 40 amino acids, about 5 amino acids to about 50 amino acids, about 5 amino acids to about 60 amino acids, about 5 amino acids to about 70 amino acids, about 5 amino acids to about 80 amino acids, about 5 amino acids to about 90 amino acids, about 5 amino acids to about 100 amino acids, about 10 amino acids to about 20 amino acids, about 10 amino acids to about 30 amino acids, about 10 amino acids to about 40 amino acids, about 10 amino acids to about 50 amino acids, about 10 amino acids to about 60 amino acids, about 10 amino acids to about 70 amino acids, about 10 amino acids to about 80 amino acids, about 10 amino acids to about 90 amino acids, about 10 amino acids to about 100 amino acids, about 20 amino acids to about 30 amino acids, about 20 amino acids to about 40 amino acids, about 20 amino acids to about 50 amino acids, about 20 amino acids to about 60 amino acids, about 20 amino acids to about 70 amino acids, about 20 amino acids to about 80 amino acids, about 20 amino acids to about 90 amino acids, about 20 amino acids to about 100 amino acids, about 30 amino acids to about 40 amino acids, about 30 amino acids to about 50 amino acids, about 30 amino acids to about 60 amino acids, about 30 amino acids to about 70 amino acids, about 30 amino acids to about 80 amino acids, about 30 amino acids to about 90 amino acids, about 30 amino acids to about 100 amino acids, about 40 amino acids to about 50 amino acids, about 40 amino acids to about 60 amino acids, about 40 amino acids to about 70 amino acids, about 40 amino acids to about 80 amino acids, about 40 amino acids to about 90 amino acids, about 40 amino acids to about 100 amino acids, about 50 amino acids to about 60 amino acids, about 50 amino acids to about 70 amino acids, about 50 amino acids to about 80 amino acids, about 50 amino acids to about 90 amino acids, about 50 amino acids to about 100 amino acids, about 60 amino acids to about 70 amino acids, about 60 amino acids to about 80 amino acids, about 60 amino acids to about 90 amino acids, about 60 amino acids to about 100 amino acids, about 70 amino acids to about 80 amino acids, about 70 amino acids to about 90 amino acids, about 70 amino acids to about 100 amino acids, about 80 amino acids to about 90 amino acids, about 80 amino acids to about 100 amino acids, or about 90 amino acids to about 100 amino acids, including any ranges or subranges therebetween. In some embodiments, the deletion is about 1 amino acid, about 5 amino acids, about 10 amino acids, about 20 amino acids, about 30 amino acids, about 40 amino acids, about 50 amino acids, about 60 amino acids, about 70 amino acids, about 80 amino acids, about 90 amino acids, or about 100 amino acids, including any ranges or subranges therebetween.

### 1.3. Polynucleotides and Vectors

In some embodiments, the disclosure provides recombinant nucleic acid molecules (i.e., polynucleotides) encoding the multi-specific polypeptide of the disclosure. Polynucleotides disclosed herein can be at least about 5, at least about 10, at least about 15, at least about 20, at least about 25, at least about 30, at least about 40, at least about 50, at least about 100, at least about 200, at least about 300, at least about 400, at least about 500, at least about 1000, at least about 5000, at least about 10000, or at least about 15000 or more nucleotides in length, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

The present disclosure further relates to variants of the polynucleotides disclosed herein. The polynucleotide variants can contain alterations in the coding regions, non-coding regions, or both.

In some embodiments, a polynucleotide variant comprising a nucleotide sequence that is at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the nucleotide sequence of a polynucleotide disclosed herein.

In some embodiments, a polynucleotide variant contains substitutions, additions, or deletions that alter the properties or activities of the encoded polypeptide. In some embodiments, a polynucleotide variant contains silent substitutions, additions, or deletions that does not alter the properties or activities of the encoded polypeptide. In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, polynucleotides are codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, (xi) isolated removal of spurious translation initiation sites, (xii) elimination of fortuitous polyadenylation sites otherwise leading to truncated RNA transcripts, (xiii) elimination of fortuitous predicted splice sites, and/or (xiv) disruption of CpG dinucleotides.

It will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere or as known in the art, such that their overall length may vary considerably. It is therefore contemplated that a polynucleotide fragment of almost any length may be employed in particular embodiments. In some embodiments, the total length is limited by the ease of preparation and use in the intended recombinant DNA protocol.

Polynucleotides can be prepared, isolated, purified, manipulated, and/or expressed using any of a variety of well-established techniques known and available in the art.

In some embodiments, the disclosure provides vectors comprising the recombinant nucleic acid molecules (i.e., polynucleotides) of the disclosure.

In order to express the multi-specific polypeptides described herein in a cell, an expression cassette encoding the multi-specific polypeptides can be inserted into a nucleic acid vector. The “expression cassette” contains the gene of interest. The cassette is positionally and sequentially oriented within the vector such that the nucleic acid in the cassette can be transcribed into RNA, and when necessary, translated into a protein or a polypeptide, undergo appropriate post-translational modifications required for activity in the host cell, and be translocated to the appropriate compartment for biological activity by targeting to appropriate intracellular compartments or secretion into extracellular compartments. In some embodiments, the cassette has its 3′ and 5′ ends adapted for ready insertion into a vector, e.g., it has restriction endonuclease sites at each end. The cassette can be removed and inserted into a plasmid or viral vector as a single unit.

The host cell may be co-transfected with two vectors provided herein, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 1986, 322:52; and Kohler, Proc. Natl. Acad. Sci. USA, 1980, 77:2197-9).

In some embodiments, vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. In some embodiments, the coding sequences of the multi-specific polypeptides disclosed herein can be ligated into such vectors for expression in mammalian cells.

In some embodiments, non-viral vectors are used to deliver one or more polynucleotides contemplated herein. In some embodiments, the recombinant vector comprising a polynucleotide encoding the multi-specific polypeptides described herein is a plasmid. Numerous suitable plasmid expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other plasmid vector may be used so long as it is compatible with the host cell.

In some embodiments, viral vectors are used to deliver one or more polynucleotides contemplated herein. Suitable viral vectors include, but are not limited to, viral vectors based on adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al, PNAS 94:6916 6921, 1997; Bennett et al., Invest Opthalmol Vis Sci 38:2857 2863, 1997; Jomary et al., Gene Ther 4:683 690, 1997, Rolling et al.,

Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al, Virol. (1988) 166:154-165; and Flotte et al., PNAS (1993) 90:10613-10617); alphaviruses; arenaviruses; baculovirus; herpes simplex virus; human immunodeficiency virus (see, e.g., Miyoshi et al., PNAS 94:10319 23, 1997; Takahashi et al., J Virol 73:7812 7816, 1999); poliovirus; poxvirus; retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); SV40; vaccinia virus; and the like. Examples of vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. A viral vector delivered by such viruses or viral particles may be referred to by the type of virus to deliver the viral vector (e.g., a lentiviral vector is a viral vector that is to be delivered by a lentivirus). A viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

In some embodiments, the vector is an integrating vector. In some embodiments, a polynucleotide is introduced into a target or host cell using a transposon vector system. In some embodiments, the transposon vector system comprises a vector comprising transposable elements and a polynucleotide contemplated herein; and a transposase. In some embodiments, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and variants thereof. The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302:93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

In some embodiments, a polynucleotide sequence encoding the multi-specific polypeptides disclosed herein is operably linked to one or more control elements that allow expression of the polynucleotide in both prokaryotic and eukaryotic cells. “Control elements” refer those non-translated regions of the vector which interact with host cellular proteins to carry out transcription and translation. Non-limiting examples of control elements include origin of replication, selection cassettes, constitutive and inducible promoters, enhancers, translation initiation signals (Shine Dalgarno sequence or Kozak sequence) introns, transcription terminators, 5′ and 3′ untranslated regions. See e.g., Bitter et al. (1987) Methods in Enzymology, 153:516-544) Such elements may vary in their strength and specificity. The transcriptional control element may be functional in either a eukaryotic cell (e.g., a mammalian cell) or a prokaryotic cell (e.g., bacterial or archaeal cell).

In some embodiments, polynucleotides encoding the multi-specific polypeptides described herein are operably linked to a promoter and/or an enhancer. The term “promoter” as used herein refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. The term “enhancer” refers to a segment of DNA which contains sequences capable of providing enhanced transcription and in some instances can function independent of their orientation relative to another control sequence. An enhancer can function cooperatively or additively with promoters and/or other enhancer elements.

Non-limiting examples of suitable eukaryotic promoters (promoters functional in a eukaryotic cell) include those from cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter, and mouse metallothionein-1. Selection of the appropriate vector and promoter is well within the level of ordinary skill in the art.

In some embodiments, a polynucleotide sequence encoding the multi-specific polypeptides described herein is operably linked to a constitutive promoter. In such embodiments, the polynucleotides encoding the multi-specific polypeptides described herein are constitutively and/or ubiquitously expressed in a cell.

In some embodiments, a polynucleotide sequence encoding the multi-specific polypeptides described herein is operably linked to an inducible promoter. In such embodiments, polynucleotides encoding the multi-specific polypeptides described herein are conditionally expressed. As used herein, “conditional expression” may refer to any type of conditional expression including, but not limited to, inducible expression; repressible expression; expression in cells or tissues having a particular physiological, biological, or disease state (e.g., cell type or tissue specific expression) etc. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, the vectors described herein further comprise a transcription termination signal. Elements directing the efficient termination and polyadenylation of the heterologous nucleic acid transcripts increases heterologous gene expression. Transcription termination signals are generally found downstream of the polyadenylation signal. In some embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In some embodiments, the core poly(A) sequence is an ideal polyA sequence (e.g., AATAAA, ATTAAA, AGTAAA). In some embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art.

In some embodiments, a vector may also comprise a sequence encoding a signal peptide (e.g., for nuclear localization, nucleolar localization, mitochondrial localization), fused to the polynucleotide encoding the multi-specific polypeptides. For example, a vector may comprise a nuclear localization sequence (e.g., from SV40) fused to the polynucleotide encoding the multi-specific polypeptides. In some embodiments, the signal peptide is an Igk signal peptide. In some embodiments, the signal peptide is a CD8 signal peptide.

The expression vector may also include nucleotide sequences encoding protein tags (e.g., 6×His tag, hemagglutinin tag, green fluorescent protein, etc.) that are fused to the site-directed modifying polypeptide, thus resulting in a chimeric polypeptide.

Methods of introducing polynucleotides and recombinant vectors into a host cell are known in the art. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: S0169-409X (12) 00283-9), microfluidics delivery methods (See e.g., International PCT Publication No. WO 2013/059343), and the like.

In some embodiments, delivery via electroporation comprises mixing the cells with the polynucleotides encoding the multi-specific polypeptide in a cartridge, chamber, or cuvette and applying one or more electrical impulses of defined duration and amplitude. In some embodiments, cells are mixed with polynucleotides encoding the multi-specific polypeptides in a vessel connected to a device (e.g., a pump) which feeds the mixture into a cartridge, chamber, or cuvette wherein one or more electrical impulses of defined duration and amplitude are applied, after which the cells are delivered to a second vessel. Illustrative examples of polynucleotide delivery systems suitable for use in particular embodiments contemplated include, but are not limited to, those provided by Amaxa Biosystems, Maxcyte, Inc., BTX Molecular Delivery Systems, Neon™ Transfection Systems, and Copernicus Therapeutics Inc. Lipofection reagents are sold commercially (e.g., Transfectam™ and Lipofectin™). Cationic and neutral lipids that are suitable for efficient lipofection of polynucleotides have been described in the literature. See e.g., Liu et al. (2003) Gene Therapy. 10:180-187; and Balazs et al. (2011) Journal of Drug Delivery. 2011:1-12.

In some embodiments, polynucleotides encoding the multi-specific polypeptides described herein are introduced to a cell in a non-viral delivery vehicle, such as a transposon, a nanoparticle (e.g., a lipid nanoparticle), a liposome, an exosome, an attenuated bacterium, or a virus-like particle. In some embodiments, the vehicle is an attenuated bacterium (e.g., naturally or artificially engineered to be invasive but attenuated to prevent pathogenesis including Listeria monocytogenes, certain Salmonella strains, Bifidobacterium longum, and modified Escherichia coli), bacteria having nutritional and tissue-specific tropism to target specific cells, and bacteria having modified surface proteins to alter target cell specificity. In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a biological liposome. For example, the biological liposome is a phospholipid-based particle derived from human cells (e.g., erythrocyte ghosts, which are red blood cells broken down into spherical structures derived from the subject and wherein tissue targeting can be achieved by attachment of various tissue or cell-specific ligands), secretory exosomes, or subject derived membrane-bound nanovesicles (30-100 nm) of endocytic origin (e.g., can be produced from various cell types and can therefore be taken up by cells without the need for targeting ligands).

In some embodiments, vectors comprising polynucleotides encoding multi-specific polypeptides described herein are introduced to cells by viral delivery methods, e.g., by viral transduction. A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce nonproliferating cells.

In some embodiments, the vehicle is a mammalian virus-like particle. For example, modified viral particles can be generated (e.g., by purification of the “empty” particles followed by ex vivo assembly of the virus with the desired cargo).

The vehicle can also be engineered to incorporate targeting ligands to alter target tissue specificity.

### 1.4. Pharmaceutical Compositions

In some embodiments, the disclosure provides a pharmaceutical composition. In some embodiments, the pharmaceutical composition comprises the multi-specific polypeptide of the disclosure and a pharmaceutically acceptable carrier, diluent, or excipient. In some embodiments, the pharmaceutical composition comprises the recombinant nucleic acid molecule of the disclosure and a pharmaceutically acceptable carrier, diluent, or excipient. In some embodiments, the pharmaceutical composition comprises the vector of the disclosure and a pharmaceutically acceptable carrier, diluent, or excipient.

In some embodiments, the multi-specific polypeptides disclosed herein are formulated according to known methods to prepare pharmaceutically useful compositions. In some embodiments, the multi-specific polypeptide is combined in a mixture with a pharmaceutically acceptable carrier, diluent or excipient.

Pharmaceutically acceptable carrier, diluent or excipient includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

Pharmaceutically acceptable salt includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In some embodiments, organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions disclosed herein can be provided as a controlled release or sustained release system. In some embodiments, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In some embodiments, polymeric materials can be used to achieve controlled or sustained release of a prophylactic or therapeutic agent or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly (acrylic acid), poly(ethylene-co-vinyl acetate), poly (methacrylic acid), polyglycolides (PLG), polyanhydrides, poly (N-vinyl pyrrolidone), poly(vinyl alcohol), polyacrylamide, poly (ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of a particular target tissue, for example, the kidney, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy &Oncology 39:179-89 (1996); Song et al., PDA J. of Pharma. Sci. &Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The multi-specific polypeptides may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

In some embodiments, the pharmaceutical compositions disclosed herein to are sterile. The pharmaceutical composition may be rendered sterile by filtration through sterile filtration membranes. The pharmaceutical compositions disclosed herein generally can be placed into a container having a sterile access port, for example, an intravenous solution bag or vial having a stopper pierceable by a hypodermic injection needle.

### 1.5. Methods

Provided herein are methods of expanding T cells, comprising contacting a T cell with a multi-specific polypeptide, thereby inducing a production of a population of T cells, wherein the multi-specific polypeptide comprises a first binding domain that specifically binds to a T cell receptor beta variable region (TCRβV) or a T cell receptor alpha variable region (TCRαV) expressed on the T cell, and wherein the population of T cells comprises a plurality of memory T cells and effector T cells. In some embodiments, the method expands T cells in vivo. In some embodiments, the method expands T cells in vitro. In some embodiments, the method expands T cells ex vivo.

Also provided herein are methods of expanding T cells, comprising contacting a T cell with a multi-specific polypeptide, thereby inducing a production of a population of T cells, wherein the multi-specific polypeptide comprises a first binding domain that specifically binds to CD3, and wherein the population of T cells comprises a plurality of memory T cells and effector T cells. In some embodiments, the method expands T cells in vivo. In some embodiments, the method expands T cells in vitro. In some embodiments, the method expands T cells ex vivo.

Also provided herein are methods of depleting B cells in a subject having a disease or disorder related to the B cells, comprising administering to the subject an effective amount of the multi-specific polypeptide of the disclosure. In some embodiments, the method decreases the number of B cells by at least 50%, at least 55%, at least 60%, at least 65%, at least 70%, at least 75%, at least 80%, at least 85%, at least 90%, at least 95%, at least 98%, at least 99%, at least 99.5%, at least 99.8%, at least 99.9%, or 100%, in the subject. In some embodiments, the method decreases the number of B cells by about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 98%, about 98% to about 99%, about 99% to about 99.5%, about 99.5% to about 99.8%, about 99.8% to about 99.9%, or about 99.9% to about 100%, in the subject, including all ranges and subranges therebetween. In some embodiments, the B cells are autoreactive B cells. In some embodiments, the B cells are autoreactive memory B cells. In some embodiments, the method reduces autoantibodies.

In some embodiments, the present disclosure provides a method of repopulating naive B cells in a subject, comprising administering to the subject an effective amount of the multi-specific polypeptide of the disclosure. In some embodiments, the subject has a disease or disorder related to B cells. In some embodiments, the method increases the number of naive B cells by at least 50%, at least 55%, at least 60%, at least 65%, at least 70%, at least 75%, at least 80%, at least 85%, at least 90%, at least 95%, at least 98%, at least 99%, or 100% in the subject. In some embodiments, the method increases the number of naive B cells by about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 98%, about 98% to about 99%, or about 99% to about 100% in the subject.

Also provided herein are methods of treating a disease or disorder in a subject in need thereof, comprising administering a therapeutically effective amount of a pharmaceutical composition comprising a multi-specific polypeptide to the subject. In some embodiments, the multi-specific polypeptide contacts a T cell of the subject, thereby inducing a production of a population of T cells, wherein the multi-specific polypeptide comprises a first binding domain that specifically binds to a T cell receptor beta variable region (TCRβV) or a T cell receptor alpha variable region (TCRαV) expressed on the T cell, and wherein the population of T cells comprises a plurality of memory T cells and effector T cells. In some embodiments, the multi-specific polypeptide contacts a T cell of the subject, thereby inducing a production of a population of T cells, wherein the multi-specific polypeptide comprises a first binding domain that specifically binds to CD3, and wherein the population of T cells comprises a plurality of memory T cells and effector T cells.

In some embodiments, the population of T cells has an increased durability than that of a CD3 binder-expanded population of T cells. The population of T cells disclosed herein refer to the population of T cells produced as a result of the methods of expanding T cells disclosed herein using the multi-specific polypeptide disclosed herein, or the population of T cells induced as a result of the methods of treatment disclosed herein using the multi-specific polypeptide disclosed herein. A variety of features showing increased durability are disclosed in detail below. In some embodiments, the measurement of the population of T cells are performed at day 1, 2, 3, 4, 5, 6, 7, 8, 9, 10, or 11 after an initial contact of the T cell. In some embodiments, the comparison between the population of T cells expanded by the multi-specific polypeptide using the methods disclosed herein (i.e., the methods of expanding T cells disclosed herein or the methods of treatment disclosed herein) and the CD3 binder-expanded population of T cells are performed under the same condition. In some embodiments, the comparison between the population of T cells expanded by the multi-specific polypeptide using the methods disclosed herein (i.e., the methods of expanding T cells disclosed herein or the methods of treatment disclosed herein) and the CD3 binder-expanded population of T cells are performed at day 1, 2, 3, 4, 5, 6, 7, 8, 9, 10, or 11 after an initial contact of the T cell.

In some embodiments, the population of T cells has an increased percentage of effector T cells than that of a CD3 binder-expanded population of T cells. In some embodiments, the percentage of effector T cells of the population of T cells is at least about 10%. In some embodiments, the percentage of effector T cells of the population of T cells is about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40%, including any ranges or subranges therebetween. In some embodiments, the percentage of effector T cells of the population of T cells is about 10%, about 15%, about 20%, about 25%, about 30%, about 35%, or about 40%, including any ranges or subranges therebetween. In some embodiments, the percentage of effector T cells of the population of T cells is at least about 30%, at least about 40%, at least about 50%, at least about 60%, at least about 70%, at least about 80%, at least about 90%, or at least about 98% higher, including any ranges or subranges therebetween, than that of a CD3 binder-expanded population of T cells. In some embodiments, the percentage of effector T cells of the population of T cells is at least about 2 times, at least about 3 times, at least about 4 times, at least about 5 times, or at least about 6 times that of a CD3 binder-expanded population of T cells. In some embodiments, the effector T cells are effector memory T cells (TEM).

In some embodiments, the population of T cells has an increased expansion of effector T cells than that of a CD3 binder-expanded population of T cells. In some embodiments, the number of effector T cells of the population of T cells expands at least about 2 times. In some embodiments, the number of effector T cells of the population of T cells expands about 2 times to about 4 times, about 2 times to about 8 times, about 2 times to about 16 times, about 2 times to about 32 times, about 2 times to about 64 times, about 2 times to about 128 times, about 2 times to about 256 times, about 2 times to about 512 times, about 2 times to about 1,024 times, about 2 times to about 2,048 times, about 4 times to about 8 times, about 4 times to about 16 times, about 4 times to about 32 times, about 4 times to about 64 times, about 4 times to about 128 times, about 4 times to about 256 times, about 4 times to about 512 times, about 4 times to about 1,024 times, about 4 times to about 2,048 times, about 8 times to about 16 times, about 8 times to about 32 times, about 8 times to about 64 times, about 8 times to about 128 times, about 8 times to about 256 times, about 8 times to about 512 times, about 8 times to about 1,024 times, about 8 times to about 2,048 times, about 16 times to about 32 times, about 16 times to about 64 times, about 16 times to about 128 times, about 16 times to about 256 times, about 16 times to about 512 times, about 16 times to about 1,024 times, about 16 times to about 2,048 times, about 32 times to about 64 times, about 32 times to about 128 times, about 32 times to about 256 times, about 32 times to about 512 times, about 32 times to about 1,024 times, about 32 times to about 2,048 times, about 64 times to about 128 times, about 64 times to about 256 times, about 64 times to about 512 times, about 64 times to about 1,024 times, about 64 times to about 2,048 times, about 128 times to about 256 times, about 128 times to about 512 times, about 128 times to about 1,024 times, about 128 times to about 2,048 times, about 256 times to about 512 times, about 256 times to about 1,024 times, about 256 times to about 2,048 times, about 512 times to about 1,024 times, about 512 times to about 2,048 times, or about 1,024 times to about 2,048 times, including any ranges or subranges therebetween. In some embodiments, the number of effector T cells of the population of T cells expands about 2 times, about 4 times, about 8 times, about 16 times, about 32 times, about 64 times, about 128 times, about 256 times, about 512 times, about 1,024 times, or about 2,048 times, including any ranges or subranges therebetween. In some embodiments, the number of effector T cells of the population of T cells is at least about 2 times, at least about 4 times, at least about 8 times, at least about 16 times, at least about 32 times, at least about 64 times, at least about 128 times, or at least about 256 times that of a CD3 binder-expanded population of T cells. In some embodiments, the effector T cells are selected from the group consisting of effector memory T cells (TEM), T effector cells (TEFF), T effector cells re-expressing CD45RA (TEMRA), and a combination thereof.

In some embodiments, the population of T cells has an increased percentage of memory T cells than that of a CD3 binder-expanded population of T cells. In some embodiments, the percentage of memory T cells of the population of T cells is at least about 10%. In some embodiments, the percentage of memory T cells of the population of T cells is about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%, including any ranges or subranges therebetween. In some embodiments, the percentage of memory T cells of the population of T cells is about 10%, about 15%, about 20%, about 25%, about 30%, about 35%, about 40%, about 45%, or about 50%, including any ranges or subranges therebetween. In some embodiments, the percentage of memory T cells of the population of T cells is at least about 30%, at least about 40%, at least about 50%, at least about 60%, at least about 70%, at least about 80%, at least about 90%, or at least about 98% higher, including any ranges or subranges therebetween, than that of a CD3 binder-expanded population of T cells. In some embodiments, the percentage of memory T cells of the population of T cells is at least about 2 times, at least about 3 times, at least about 4 times, at least about 5 times, or at least about 6 times that of a CD3 binder-expanded population of T cells. In some embodiments, the memory T cells are central memory T cells (TCM).

In some embodiments, the population of T cells has an increased expansion of memory T cells than that of a CD3 binder-expanded population of T cells. In some embodiments, the number of memory T cells of the population of T cells expands at least about 8 times. In some embodiments, the number of memory T cells of the population of T cells expands about 8 times to about 16 times, about 8 times to about 32 times, about 8 times to about 64 times, about 8 times to about 128 times, about 8 times to about 256 times, about 8 times to about 512 times, about 8 times to about 1,024 times, about 8 times to about 2,048 times, about 8 times to about 4,096 times, about 16 times to about 32 times, about 16 times to about 64 times, about 16 times to about 128 times, about 16 times to about 256 times, about 16 times to about 512 times, about 16 times to about 1,024 times, about 16 times to about 2,048 times, about 16 times to about 4,096 times, about 32 times to about 64 times, about 32 times to about 128 times, about 32 times to about 256 times, about 32 times to about 512 times, about 32 times to about 1,024 times, about 32 times to about 2,048 times, about 32 times to about 4,096 times, about 64 times to about 128 times, about 64 times to about 256 times, about 64 times to about 512 times, about 64 times to about 1,024 times, about 64 times to about 2,048 times, about 64 times to about 4,096 times, about 128 times to about 256 times, about 128 times to about 512 times, about 128 times to about 1,024 times, about 128 times to about 2,048 times, about 128 times to about 4,096 times, about 256 times to about 512 times, about 256 times to about 1,024 times, about 256 times to about 2,048 times, about 256 times to about 4,096 times, about 512 times to about 1,024 times, about 512 times to about 2,048 times, about 512 times to about 4,096 times, about 1,024 times to about 2,048 times, about 1,024 times to about 4,096 times, or about 2,048 times to about 4,096 times, including any ranges or subranges therebetween. In some embodiments, the number of memory T cells of the population of T cells expands about 8 times, about 16 times, about 32 times, about 64 times, about 128 times, about 256 times, about 512 times, about 1,024 times, about 2,048 times, or about 4,096 times. In some embodiments, the number of memory T cells of the population of T cells expands about 2 times, about 4 times, about 8 times, about 16 times, about 32 times, about 64 times, about 128 times, about 256 times, about 512 times, about 1,024 times, or about 2,048 times, including any ranges or subranges therebetween. In some embodiments, the number of memory T cells of the population of T cells is at least about 2 times, at least about 4 times, at least about 8 times, at least about 16 times, at least about 32 times, at least about 64 times, at least about 128 times, at least about 256 times, at least about 512 times, at least about 1,024 times, or at least about 2,048 times that of a CD3 binder-expanded population of T cells. In some embodiments, the memory T cells are selected from the group consisting of central memory T cells (TCM), T memory stem cells (TSCM), and a combination thereof.

In some embodiments, the population of T cells has an increased expansion of CD8+ T cells than that of a CD3 binder-expanded population of T cells. In some embodiments, the number of CD8+ T cells of the population of T cells is at least about 2 times, at least about 4 times, at least about 8 times, at least about 16 times, at least about 32 times, at least about 64 times, at least about 128 times, at least about 256 times, or at least about 512 times that of a CD3 binder-expanded population of T cells.

In some embodiments, the population of T cells has an increased expansion of CD4+ (non-Treg) T cells than that of a CD3 binder-expanded population of T cells. In some embodiments, the number of CD4+ (non-Treg) T cells of the population of T cells is at least about 2 times, at least about 4 times, at least about 8 times, at least about 16 times, at least about 32 times, at least about 64 times, at least about 128 times, at least about 256 times, or at least about 512 times that of a CD3 binder-expanded population of T cells.

In some embodiments, the population of T cells has an increased expansion of regulatory T cells than that of a CD3 binder-expanded population of T cells. In some embodiments, the number of regulatory T cells of the population of T cells is at least about 2 times, at least about 3 times, at least about 4 times, at least about 5 times, at least about 10 times, or at least about 15 times that of a CD3 binder-expanded population of T cells.

In some embodiments, the population of T cells has a decreased percentage of regulatory T cells than that of a CD3 binder-expanded population of T cells.

In some embodiments, the population of T cells has an increased ratio of CD8+ T cells to regulatory T cells than that of a CD3 binder-expanded population of T cells. In some embodiments, the ratio of CD8+ T cells to regulatory T cells of the population of T cells is at least about 2. In some embodiments, the ratio of CD8+ T cells to regulatory T cells of the population of T cells is about 2 to about 4, about 2 to about 6, about 2 to about 10, about 2 to about 14, about 2 to about 18, about 2 to about 22, about 2 to about 26, about 2 to about 30, about 4 to about 6, about 4 to about 10, about 4 to about 14, about 4 to about 18, about 4 to about 22, about 4 to about 26, about 4 to about 30, about 6 to about 10, about 6 to about 14, about 6 to about 18, about 6 to about 22, about 6 to about 26, about 6 to about 30, about 10 to about 14, about 10 to about 18, about 10 to about 22, about 10 to about 26, about 10 to about 30, about 14 to about 18, about 14 to about 22, about 14 to about 26, about 14 to about 30, about 18 to about 22, about 18 to about 26, about 18 to about 30, about 22 to about 26, about 22 to about 30, or about 26 to about 30, including any ranges or subranges therebetween. In some embodiments, the ratio of CD8+ T cells to regulatory T cells of the population of T cells is about 2, about 4, about 6, about 10, about 14, about 18, about 22, about 26, or about 30, including any ranges or subranges therebetween. In some embodiments, the ratio of CD8+ T cells to regulatory T cells of the population of T cells is at least about 2 times, at least about 3 times, at least about 4 times, at least about 5 times, at least about 8 times, at least about 10 times, at least about 12 times, at least about 15 times, at least about 18 times, at least about 20 times, at least about 22 times, at least about 25 times, or at least about 30 times that of a CD3 binder-expanded population of T cells.

In some embodiments, the population of T cells has an increased ratio of CD4+ (non-Treg) T cells to regulatory T cells than that of a CD3 binder-expanded population of T cells. In some embodiments, the ratio of CD4+ (non-Treg) T cells to regulatory T cells of the population of T cells is at least about 6. In some embodiments, the ratio of CD4+ (non-Treg) T cells to regulatory T cells of the population of T cells is about 6 to about 15, about 6 to about 20, about 6 to about 25, about 6 to about 30, about 6 to about 35, about 6 to about 40, about 6 to about 45, about 6 to about 50, about 6 to about 60, about 6 to about 70, about 6 to about 80, about 15 to about 20, about 15 to about 25, about 15 to about 30, about 15 to about 35, about 15 to about 40, about 15 to about 45, about 15 to about 50, about 15 to about 60, about 15 to about 70, about 15 to about 80, about 20 to about 25, about 20 to about 30, about 20 to about 35, about 20 to about 40, about 20 to about 45, about 20 to about 50, about 20 to about 60, about 20 to about 70, about 20 to about 80, about 25 to about 30, about 25 to about 35, about 25 to about 40, about 25 to about 45, about 25 to about 50, about 25 to about 60, about 25 to about 70, about 25 to about 80, about 30 to about 35, about 30 to about 40, about 30 to about 45, about 30 to about 50, about 30 to about 60, about 30 to about 70, about 30 to about 80, about 35 to about 40, about 35 to about 45, about 35 to about 50, about 35 to about 60, about 35 to about 70, about 35 to about 80, about 40 to about 45, about 40 to about 50, about 40 to about 60, about 40 to about 70, about 40 to about 80, about 45 to about 50, about 45 to about 60, about 45 to about 70, about 45 to about 80, about 50 to about 60, about 50 to about 70, about 50 to about 80, about 60 to about 70, about 60 to about 80, or about 70 to about 80, including any ranges or subranges therebetween. In some embodiments, the ratio of CD4+ (non-Treg) T cells to regulatory T cells of the population of T cells is about 6, about 15, about 20, about 25, about 30, about 35, about 40, about 45, about 50, about 60, about 70, or about 80, including any ranges or subranges therebetween. In some embodiments, the ratio of CD4+ (non-Treg) T cells to regulatory T cells of the population of T cells is at least about 2 times, at least about 3 times, at least about 4 times, at least about 5 times, at least about 8 times, at least about 10 times, at least about 12 times, at least about 15 times, at least about 18 times, at least about 20 times, at least about 22 times, at least about 25 times, or at least about 30 times that of a CD3 binder-expanded population of T cells.

In some embodiments, the population of T cells has an increased cytotoxicity than that of a CD3 binder-expanded population of T cells. In some embodiments, the cytotoxicity is increased by at least about 20%, at least about 30%, at least about 40%, at least about 50%, at least about 60%, at least about 70%, at least about 80%, at least about 90%, or at least about 100%. In some embodiments, the cytotoxicity is increased at least about 2 times, at least about 3 times, at least about 4 time, at least about 5 times, or at least about 6 times.

In some embodiments, the population of T cells has a prolonged cytotoxicity than that of a CD3 binder-expanded population of T cells. In some embodiments, the cytotoxicity is prolonged for at least about 1 day. In some embodiments, the cytotoxicity is prolonged for about 1 day to about 3 days, about 1 day to about 5 days, about 1 day to about 7 days, about 1 day to about 10 days, about 1 day to about 15 days, about 1 day to about 20 days, about 1 day to about 25 days, about 1 day to about 30 days, about 1 day to about 40 days, about 1 day to about 50 days, about 1 day to about 70 days, about 1 day to about 90 days, about 3 days to about 5 days, about 3 days to about 7 days, about 3 days to about 10 days, about 3 days to about 15 days, about 3 days to about 20 days, about 3 days to about 25 days, about 3 days to about 30 days, about 3 days to about 40 days, about 3 days to about 50 days, about 3 days to about 70 days, about 3 days to about 90 days, about 5 days to about 7 days, about 5 days to about 10 days, about 5 days to about 15 days, about 5 days to about 20 days, about 5 days to about 25 days, about 5 days to about 30 days, about 5 days to about 40 days, about 5 days to about 50 days, about 5 days to about 70 days, about 5 days to about 90 days, about 7 days to about 10 days, about 7 days to about 15 days, about 7 days to about 20 days, about 7 days to about 25 days, about 7 days to about 30 days, about 7 days to about 40 days, about 7 days to about 50 days, about 7 days to about 70 days, about 7 days to about 90 days, about 10 days to about 15 days, about 10 days to about 20 days, about 10 days to about 25 days, about 10 days to about 30 days, about 10 days to about 40 days, about 10 days to about 50 days, about 10 days to about 70 days, about 10 days to about 90 days, about 15 days to about 20 days, about 15 days to about 25 days, about 15 days to about 30 days, about 15 days to about 40 days, about 15 days to about 50 days, about 15 days to about 70 days, about 15 days to about 90 days, about 20 days to about 25 days, about 20 days to about 30 days, about 20 days to about 40 days, about 20 days to about 50 days, about 20 days to about 70 days, about 20 days to about 90 days, about 25 days to about 30 days, about 25 days to about 40 days, about 25 days to about 50 days, about 25 days to about 70 days, about 25 days to about 90 days, about 30 days to about 40 days, about 30 days to about 50 days, about 30 days to about 70 days, about 30 days to about 90 days, about 40 days to about 50 days, about 40 days to about 70 days, about 40 days to about 90 days, about 50 days to about 70 days, about 50 days to about 90 days, or about 70 days to about 90 days. In some embodiments, the cytotoxicity is prolonged for about 1 day, about 3 days, about 5 days, about 7 days, about 10 days, about 15 days, about 20 days, about 25 days, about 30 days, about 40 days, or about 50 days, including any ranges or subranges therebetween.

In some embodiments, the population of T cells has reduced cytokine release than that of a CD3 binder-expanded population of T cells. In some embodiments, the cytokine release is reduced by at least about 20%, at least about 30%, at least about 40%, at least about 50%, at least about 60%, at least about 70%, at least about 80%, at least about 90%, or at least about 100%. In some embodiments, the cytokine release is reduced at least about 2 times, at least about 3 times, at least about 4 time, at least about 5 times, at least about 6 times, at least about 8 times, at least about 10 times, at least about 12 time, at least about 14 times, at least about 16 times, at least about 18 times, at least about 20 times, at least about 22 time, at least about 24 times, or at least about 26 times. In some embodiments, the cytokine is interferon-gamma (IFNγ). In some embodiments, the cytokine is tumor necrosis factor (TNF). In some embodiments, the cytokine is interleukin-6 (IL-6). In some embodiments, the cytokine is interleukin-2 (IL-2). In some embodiments, the cytokine is interleukin-10 (IL-10).

In some embodiments, the disease or disorder treated by the methods disclosed herein is a cancer. In some embodiments, the cancer is a blood cancer. In some embodiments, the blood cancer is leukemia. In some embodiments, the leukemia is acute myeloid leukemia (AML), treatment-related AML, secondary AML, B and T cell acute lymphoblastic leukemia (ALL), chronic lymphocytic leukemia (CLL), B chronic lymphocytic leukemia (B-CLL), hairy cell leukemia (HCL) chronic lymphocytic leukemia, or chronic myelogenous leukemia (CML). In some embodiments, the blood cancer is lymphoma. In some embodiments, the lymphoma is Hodgkin lymphoma, non-Hodgkin lymphoma, primary mediastinal large B-cell lymphoma, Burkitt's lymphoma (BL), T cell lymphoma, mantle cell lymphoma, follicular lymphoma, or diffuse large B-cell lymphoma. In some embodiments, the blood cancer is myelodysplastic syndrome (MDS). In some embodiments, the blood cancer is Myeloproliferative Disorder (MPD). In some embodiments, the blood cancer is myeloma. In some embodiments, the myeloma is multiple myeloma. In some embodiments, the blood cancer is chronic lymphocytic leukemia. In some embodiments, the blood cancer is non-Hodgkin lymphoma.

In some embodiments, the disease or disorder treated by the methods disclosed herein is an autoimmune disease. In some embodiments, the autoimmune disease is rheumatoid arthritis. In some embodiments, the autoimmune disease is Systemic lupus erythematosus. In some embodiments, the autoimmune disease is Multiple Sclerosis (MS), Type 1 Diabetes, Inflammatory Bowel Disease (IBD), Psoriasis, Hashimoto's Thyroiditis, Graves' Disease, Celiac Disease, Ankylosing Spondylitis, Sjögren's Syndrome, Myasthenia Gravis, Scleroderma, Autoimmune Hepatitis, Addison's Disease, Vasculitis, Pernicious anemia, immune thrombocytopenia purpura (ITP), ulcerative colitis (UC), Felty's syndrome, autoimmune hemolytic anemia, bullous pemphigoid, autoimmune neutropenia, autoimmune polyglandular syndrome 1, Goodpasture's disease, pemphigus vulgaris, polychondritis, epidermolysis bullosa acquisita, or pemphigus foliaceus.

In some embodiments, the subject is human. In some embodiments, the subject is non-human primates (e.g., monkeys, baboons, and chimpanzees), mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks). In some embodiments, the subject may be a neonate, a juvenile, or an adult.

In some embodiments, the subject has previously been treated with a multi-specific polypeptide comprising a first binding domain specifically binds to CD3 instead of TCRβV or TCRαV (e.g., a bispecific polypeptide binding CD3 and CD19). In some embodiments, the subject is relapsing to a treatment comprising a multi-specific polypeptide comprising a first binding domain specifically binds to CD3 instead of TCRβV or TCRαV (e.g., a bispecific polypeptide binding CD3 and CD19). In some embodiments, the subject is refractory to a treatment comprising a multi-specific polypeptide comprising a first binding domain specifically binds to CD3 instead of TCRβV or TCRαV (e.g., a bispecific polypeptide binding CD3 and CD19).

In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment results in an improvement or remediation of the symptoms of the disease. In some embodiments, treatment may refer to a short-term (e.g., temporary and/or acute) and/or a long-term (e.g., sustained) improvement or remediation in one or more disease symptoms. In some embodiments, the improvement is an observable or measurable improvement. In some embodiments, the improvement is an improvement in the general feeling of well-being of the subject.

Administration of the pharmaceutical compositions can occur by infusion (e.g., continuous or bolus), injection, irrigation, inhalation, consumption, electro-osmosis, hemodialysis, iontophoresis, and other methods known in the art.

In some embodiments administration route is intraarterial, intracranial, intradermal, intraduodenal, intramammary, intrameningeal, intraperitoneal, intrathecal, intratumoral, intravenous, intravitreal, ophthalmic, parenteral, spinal, subcutaneous, ureteral, urethral, vaginal, or intrauterine. In some embodiments, administration route is direct, local, or systemic.

The effective amount of the pharmaceutical compositions administered to a particular subject will depend on a variety of factors, several of which will differ from patient to patient including the disorder being treated and the severity of the disorder; activity of the specific agent(s) employed; the age, body weight, general health, sex and diet of the patient; the timing of administration, route of administration; the duration of the treatment; drugs used in combination; the judgment of the prescribing physician; and like factors known in the medical arts. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Dosage amounts of the multi-specific polypeptide can typically be in the range of from about 0.0001 mg/kg/day to about 1000 mg/kg/day, but can be higher or lower, depending upon, among other factors, the activity of the compound, its bioavailability, and various factors discussed above. In some embodiments, the dose is from about 0.0001 mg/kg to about 1000 mg/kg of body weight per day. In some embodiments, the dose is from about 0.001 mg/kg to about 1000 mg/kg of body weight per day. In some embodiments, the dose is from about 0.01 mg/kg to about 1000 mg/kg of body weight per day. In some embodiments, the dose is from about 0.1 mg/kg to about 100 mg/kg of body weight per day. In some embodiments, the dose is from about 0.5 mg/kg to about 50 mg/kg of body weight per day. In some embodiments, the dose is from about 1 mg/kg to about 25 mg/kg of body weight per day. In some embodiments, the dose is from about 5 mg/kg to about 15 mg/kg of body weight per day. In some embodiments, the dose is about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg, including any ranges or subranges therebetween.

In some embodiments, the multi-specific polypeptides of the present disclosure are dosed at about 0.0001 mg/kg, 0.0002 mg/kg, 0.0003 mg/kg, 0.0004 mg/kg, 0.0005 mg/kg, 0.0006 mg/kg, 0.0007 mg/kg, 0.0008 mg/kg, 0.0009 mg/kg, 0.001 mg/kg, 0.0011 mg/kg, 0.0012 mg/kg, 0.0013 mg/kg, 0.0014 mg/kg, 0.0015 mg/kg, 0.0016 mg/kg, 0.0017 mg/kg, 0.0018 mg/kg, 0.0019 mg/kg, 0.002 mg/kg, 0.0021 mg/kg, 0.0022 mg/kg, 0.0023 mg/kg, 0.0024 mg/kg, 0.0025 mg/kg, 0.0026 mg/kg, 0.0027 mg/kg, 0.0028 mg/kg, 0.0029 mg/kg, 0.003 mg/kg, 0.0031 mg/kg, 0.0032 mg/kg, 0.0033 mg/kg, 0.0034 mg/kg, 0.0035 mg/kg, 0.0036 mg/kg, 0.0037 mg/kg, 0.0038 mg/kg, 0.0039 mg/kg, 0.004 mg/kg, 0.0041 mg/kg, 0.0042 mg/kg, 0.0043 mg/kg, 0.0044 mg/kg, 0.0045 mg/kg, 0.0046 mg/kg, 0.0047 mg/kg, 0.0048 mg/kg, 0.0049 mg/kg, 0.005 mg/kg, 0.0051 mg/kg, 0.0052 mg/kg, 0.0053 mg/kg, 0.0054 mg/kg, 0.0055 mg/kg, 0.0056 mg/kg, 0.0057 mg/kg, 0.0058 mg/kg, 0.0059 mg/kg, 0.006 mg/kg, 0.0061 mg/kg, 0.0062 mg/kg, 0.0063 mg/kg, 0.0064 mg/kg, 0.0065 mg/kg, 0.0066 mg/kg, 0.0067 mg/kg, 0.0068 mg/kg, 0.0069 mg/kg, 0.007 mg/kg, 0.0071 mg/kg, 0.0072 mg/kg, 0.0073 mg/kg, 0.0074 mg/kg, 0.0075 mg/kg, 0.0076 mg/kg, 0.0077 mg/kg, 0.0078 mg/kg, 0.0079 mg/kg, 0.008 mg/kg, 0.0081 mg/kg, 0.0082 mg/kg, 0.0083 mg/kg, 0.0084 mg/kg, 0.0085 mg/kg, 0.0086 mg/kg, 0.0087 mg/kg, 0.0088 mg/kg, 0.0089 mg/kg, 0.009 mg/kg, 0.0091 mg/kg, 0.0092 mg/kg, 0.0093 mg/kg, 0.0094 mg/kg, 0.0095 mg/kg, 0.0096 mg/kg, 0.0097 mg/kg, 0.0098 mg/kg, 0.0099 mg/kg, 0.01 mg/kg, including all ranges and subranges therebetween.

In some embodiments, the multi-specific polypeptides of the present disclosure are dosed at about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, 1 mg/kg, 1.01 mg/kg, 1.02 mg/kg, 1.03 mg/kg, 1.04 mg/kg, 1.05 mg/kg, 1.06 mg/kg, 1.07 mg/kg, 1.08 mg/kg, 1.09 mg/kg, 1.1 mg/kg, 1.11 mg/kg, 1.12 mg/kg, 1.13 mg/kg, 1.14 mg/kg, 1.15 mg/kg, 1.16 mg/kg, 1.17 mg/kg, 1.18 mg/kg, 1.19 mg/kg, 1.2 mg/kg, 1.21 mg/kg, 1.22 mg/kg, 1.23 mg/kg, 1.24 mg/kg, 1.25 mg/kg, 1.26 mg/kg, 1.27 mg/kg, 1.28 mg/kg, 1.29 mg/kg, 1.3 mg/kg, 1.31 mg/kg, 1.32 mg/kg, 1.33 mg/kg, 1.34 mg/kg, 1.35 mg/kg, 1.36 mg/kg, 1.37 mg/kg, 1.38 mg/kg, 1.39 mg/kg, 1.4 mg/kg, 1.41 mg/kg, 1.42 mg/kg, 1.43 mg/kg, 1.44 mg/kg, 1.45 mg/kg, 1.46 mg/kg, 1.47 mg/kg, 1.48 mg/kg, 1.49 mg/kg, 1.5 mg/kg, 1.51 mg/kg, 1.52 mg/kg, 1.53 mg/kg, 1.54 mg/kg, 1.55 mg/kg, 1.56 mg/kg, 1.57 mg/kg, 1.58 mg/kg, 1.59 mg/kg, 1.6 mg/kg, 1.61 mg/kg, 1.62 mg/kg, 1.63 mg/kg, 1.64 mg/kg, 1.65 mg/kg, 1.66 mg/kg, 1.67 mg/kg, 1.68 mg/kg, 1.69 mg/kg, 1.7 mg/kg, 1.71 mg/kg, 1.72 mg/kg, 1.73 mg/kg, 1.74 mg/kg, 1.75 mg/kg, 1.76 mg/kg, 1.77 mg/kg, 1.78 mg/kg, 1.79 mg/kg, 1.8 mg/kg, 1.81 mg/kg, 1.82 mg/kg, 1.83 mg/kg, 1.84 mg/kg, 1.85 mg/kg, 1.86 mg/kg, 1.87 mg/kg, 1.88 mg/kg, 1.89 mg/kg, 1.9 mg/kg, 1.91 mg/kg, 1.92 mg/kg, 1.93 mg/kg, 1.94 mg/kg, 1.95 mg/kg, 1.96 mg/kg, 1.97 mg/kg, 1.98 mg/kg, 1.99 mg/kg, 2 mg/kg, 2.01 mg/kg, 2.02 mg/kg, 2.03 mg/kg, 2.04 mg/kg, 2.05 mg/kg, 2.06 mg/kg, 2.07 mg/kg, 2.08 mg/kg, 2.09 mg/kg, 2.1 mg/kg, 2.11 mg/kg, 2.12 mg/kg, 2.13 mg/kg, 2.14 mg/kg, 2.15 mg/kg, 2.16 mg/kg, 2.17 mg/kg, 2.18 mg/kg, 2.19 mg/kg, 2.2 mg/kg, 2.21 mg/kg, 2.22 mg/kg, 2.23 mg/kg, 2.24 mg/kg, 2.25 mg/kg, 2.26 mg/kg, 2.27 mg/kg, 2.28 mg/kg, 2.29 mg/kg, 2.3 mg/kg, 2.31 mg/kg, 2.32 mg/kg, 2.33 mg/kg, 2.34 mg/kg, 2.35 mg/kg, 2.36 mg/kg, 2.37 mg/kg, 2.38 mg/kg, 2.39 mg/kg, 2.4 mg/kg, 2.41 mg/kg, 2.42 mg/kg, 2.43 mg/kg, 2.44 mg/kg, 2.45 mg/kg, 2.46 mg/kg, 2.47 mg/kg, 2.48 mg/kg, 2.49 mg/kg, 2.5 mg/kg, 2.51 mg/kg, 2.52 mg/kg, 2.53 mg/kg, 2.54 mg/kg, 2.55 mg/kg, 2.56 mg/kg, 2.57 mg/kg, 2.58 mg/kg, 2.59 mg/kg, 2.6 mg/kg, 2.61 mg/kg, 2.62 mg/kg, 2.63 mg/kg, 2.64 mg/kg, 2.65 mg/kg, 2.66 mg/kg, 2.67 mg/kg, 2.68 mg/kg, 2.69 mg/kg, 2.7 mg/kg, 2.71 mg/kg, 2.72 mg/kg, 2.73 mg/kg, 2.74 mg/kg, 2.75 mg/kg, 2.76 mg/kg, 2.77 mg/kg, 2.78 mg/kg, 2.79 mg/kg, 2.8 mg/kg, 2.81 mg/kg, 2.82 mg/kg, 2.83 mg/kg, 2.84 mg/kg, 2.85 mg/kg, 2.86 mg/kg, 2.87 mg/kg, 2.88 mg/kg, 2.89 mg/kg, 2.9 mg/kg, 2.91 mg/kg, 2.92 mg/kg, 2.93 mg/kg, 2.94 mg/kg, 2.95 mg/kg, 2.96 mg/kg, 2.97 mg/kg, 2.98 mg/kg, 2.99 mg/kg, or 3.00 mg/kg, including all ranges and subranges therebetween. In some embodiments, the multi-specific polypeptide is administered subcutaneously. In some embodiments, the multi-specific polypeptide is dosed between about 0.07. to about 0.17 mg/kg. In some embodiments, the multi-specific polypeptide of the present disclosure is more potent in human PBMCs compared to cyno monkey PBMCs used to assess MABEL. The number of administrations of treatment to a subject may vary. In some embodiments, introducing the pharmaceutical compositions into the subject may be a one-time event. In some embodiments, such treatment may require an on-going series of repeated treatments (e.g., once per day, once per week, or multiple times per day or week). In some embodiments, multiple administrations of the pharmaceutical compositions may be required before an effect is observed. The exact protocols depend upon the disease or condition, the stage of the disease and parameters of the individual subject being treated.

### 7.6 Numbered Embodiments

Notwithstanding the appended claims, the disclosure sets forth the following numbered embodiments:

Embodiment 1. A multi-specific polypeptide comprising: (a) a first binding domain that specifically binds T cell receptor alpha variable region (TCRαV), or T cell receptor beta variable region (TCRβV); and (b) a second binding domain that specifically binds CD19.

Embodiment 2. A multi-specific polypeptide comprising: (a) a first binding domain that specifically binds CD3; and (b) a second binding domain that specifically binds CD19.

Embodiment 3. The multi-specific polypeptide of embodiment 1 or 2, further comprising: (c) a half-life extension moiety.

Embodiment 4. The multi-specific polypeptide of any one of embodiments 1-3, wherein the CD19 comprises an amino acid sequence that is at least 80%, at least 85%, at least 90%, at least 95%, at least 96%, at least 97%, at least 98%, at least 99%, or 100% identical to SEQ ID NO: 151.

Embodiment 5. The multi-specific polypeptide of any one of embodiments 1-4, wherein the second binding domain comprises:
(i) an immunoglobulin heavy chain variable (VH) region comprising a heavy chain CDR1 (HCDR1), a heavy chain CDR2 (HCDR2), and a heavy chain CDR3 (HCDR3), and
(ii) an immunoglobulin light chain variable (VL) region comprising a light chain CDR1 (LCDR1), a light chain CDR2 (LCDR2), and a light chain CDR3 (LCDR3).

Embodiment 6. The multi-specific polypeptide of embodiment 5, wherein the second binding domain is a single chain variable fragment (scFv), a Fab, a CrossFab, a Fab′, or a F(ab′)₂.

Embodiment 7. The multi-specific polypeptide of embodiment 6, wherein the Fab comprises the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

Embodiment 8. The multi-specific polypeptide of embodiment 6, wherein the CrossFab comprises the VH region, the VL region, a CH1 region, and a CL region, wherein the C-terminus of the VH region is connected to the N-terminus of the CL region, and the C-terminus of the VL region is connected to the N-terminus of the CH1 region.

Embodiment 9. The multi-specific polypeptide of embodiment 7 or 8, wherein the CH1 region and the CL region are connected by a disulfide bond.

Embodiment 10. The multi-specific polypeptide of any one of embodiments 6-9, wherein the Fab or the CrossFab is a charge steering Fab or a charge steering CrossFab.

Embodiment 11. The multi-specific polypeptide of embodiment 10, wherein a CH1 region of the charge steering Fab or the charge steering CrossFab comprises mutations K147E/K213E according to EU numbering, and a CL region of the charge steering Fab or the charge steering CrossFab comprises mutations E123R/Q124K according to EU numbering.

Embodiment 12. The multi-specific polypeptide of embodiment 6, wherein the scFv comprises the VH region and the VL region.

Embodiment 13. The multi-specific polypeptide of embodiment 12, wherein the C-terminus of the VH region is linked to the N-terminus of the VL region.

Embodiment 14. The multi-specific polypeptide of embodiment 12, wherein the C-terminus of the VL region is linked to the N-terminus of the VH region.

Embodiment 15. The multi-specific polypeptide of any one of embodiments 12-14, wherein the VH region and the VL region are linked by a peptide linker (PL2).

Embodiment 16. The multi-specific polypeptide of embodiment 15, wherein the peptide linker (PL2) comprises the formula of (Gly₄Ser)ₙ, wherein n=1-5.

Embodiment 17. The multi-specific polypeptide of embodiment 16, wherein the peptide linker (PL2) is (Gly₄Ser)₃.

Embodiment 18. The multi-specific polypeptide of embodiment 16, wherein the peptide linker (PL2) is (Gly₄Ser)₁.

Embodiment 19. The multi-specific polypeptide of any one of embodiments 5-18, wherein the HCDR1, the HCDR2, the HCDR3, the LCDR1, the LCDR2, and the LCDR3 of the second binding domain are according to one of the combinations of complementarity determining regions (CDRs) of Table 14 or Table 15.

Embodiment 20. The multi-specific polypeptide of embodiment 19, wherein the HCDR1 comprises an amino acid sequence of SYVMH (SEQ ID NO: 303), the HCDR2 comprises an amino acid sequence of YINPYNDGTKYNEKFQG (SEQ ID NO: 311), the HCDR3 comprises an amino acid sequence of GTYYYGTRVFDY (SEQ ID NO: 317), the LCDR1 comprises an amino acid sequence of RSSKSLQNVNGNTYLY (SEQ ID NO: 323), the LCDR2 comprises an amino acid sequence of RMSNLNS (SEQ ID NO: 329), and the LCDR3 comprises an amino acid sequence of MQHLEYPIT (SEQ ID NO: 334), wherein the CDRs correspond to the second binding domain and are numbered with reference to the Kabat numbering scheme.

Embodiment 21. The multi-specific polypeptide of embodiment 19, wherein the HCDR1 comprises an amino acid sequence of TSGMGVG (SEQ ID NO: 304), the HCDR2 comprises an amino acid sequence of HIWWDDDKRYNPALKS (SEQ ID NO: 312), the HCDR3 comprises an amino acid sequence of MELWSYYFDY (SEQ ID NO: 318), the LCDR1 comprises an amino acid sequence of SASSSVSYMH (SEQ ID NO: 324), the LCDR2 comprises an amino acid sequence of DTSKLAS (SEQ ID NO: 328), and the LCDR3 comprises an amino acid sequence of FQGSVYPFT (SEQ ID NO: 335), wherein the CDRs correspond to the second binding domain and are numbered with reference to the Kabat numbering scheme.

Embodiment 22. The multi-specific polypeptide of embodiment 19, wherein the HCDR1 comprises an amino acid sequence of TSTMGVG (SEQ ID NO: 306), the HCDR2 comprises an amino acid sequence of FIWWDDDKRYNPNLKS (SEQ ID NO: 314), the HCDR3 comprises an amino acid sequence of MELWSYYFDY (SEQ ID NO: 318), the LCDR1 comprises an amino acid sequence of SASSSVGYMH (SEQ ID NO: 326), the LCDR2 comprises an amino acid sequence of DTSKLAS (SEQ ID NO: 328), and the LCDR3 comprises an amino acid sequence of FQGSVYPFT (SEQ ID NO: 335), wherein the CDRs correspond to the second binding domain and are numbered with reference to the Kabat numbering scheme.

Embodiment 23. The multi-specific polypeptide of embodiment 19, wherein the HCDR1 comprises an amino acid sequence of TSTMGVG (SEQ ID NO: 306), the HCDR2 comprises an amino acid sequence of FIWWDDDKRYNPNLKS (SEQ ID NO: 314), the HCDR3 comprises an amino acid sequence of MELWSYYFDP (SEQ ID NO: 320), the LCDR1 comprises an amino acid sequence of SASSSVGYMH (SEQ ID NO: 326), the LCDR2 comprises an amino acid sequence of DTSKLAF (SEQ ID NO: 331), and the LCDR3 comprises an amino acid sequence of FQGSVYPFT (SEQ ID NO: 335), wherein the CDRs correspond to the second binding domain and are numbered with reference to the Kabat numbering scheme.

Embodiment 24. The multi-specific polypeptide of any one of embodiments 5-23, wherein the VH region of the second binding domain comprises an amino acid sequence that is at least 80%, at least 85%, at least 90%, at least 95%, at least 96%, at least 97%, at least 98%, at least 99%, or 100% identical to any one of SEQ ID NOs: 365, 367, 369, 371, 373, 375, 377, 379, 381, and 383, and wherein the VL region of the second binding domain comprises an amino acid sequence that is at least 80%, at least 85%, at least 90%, at least 95%, at least 97%, at least 98%, at least 99%, or 100% identical to SEQ ID NO: 366, 368, 370, 372, 374, 376, 378, 380, 382, and 384.

Embodiment 25. The multi-specific polypeptide of any one of embodiments 1-6 and 12-24, wherein the second binding domain comprises an scFv comprising an amino acid sequence that is at least 80%, at least 85%, at least 90%, at least 95%, at least 96%, at least 97%, at least 98%, at least 99%, or 100% identical to any one of SEQ ID NOs: 385-386.

Embodiment 26. The multi-specific polypeptide of any one of embodiments 1-11 and 19-24, wherein the second binding domain comprises a Fab or CrossFab comprising (i) a heavy chain comprising an amino acid sequence that is at least 80%, at least 85%, at least 90%, at least 95%, at least 96%, at least 97%, at least 98%, at least 99%, or 100% identical to any one of SEQ ID NOs: 387, 389, 391, 393, 395, 397, 399, 401, 403, and 405; and (ii) a light chain comprising an amino acid sequence that is at least 80%, at least 85%, at least 90%, at least 95%, at least 97%, at least 98%, at least 99%, or 100% identical to any one of SEQ ID NOs: 388, 390, 392, 394, 396, 398, 400, 402, 404, and 406.

Embodiment 27. The multi-specific polypeptide of any one of embodiments 1 and 3-26, wherein the first binding domain specifically binds TCRβV.

Embodiment 28. The multi-specific polypeptide of any one of embodiments 1 and 3-27, wherein the TCRβV is selected from the group consisting of TCRβV-5-5, TCRβV-5-6, TCRβV6-5, TCRβV-12-3, TCRβV-12-4, and TCRβV-20-1.

Embodiment 29. The multi-specific polypeptide of any one of embodiments 1 and 3-28, wherein the TCRβV comprises an amino acid sequence that is at least 80%, at least 85%, at least 90%, at least 95%, at least 96%, at least 97%, at least 98%, at least 99%, or 100% identical to any one of SEQ ID NOs: 100-116.

Embodiment 30. The multi-specific polypeptide of any one of embodiments 1 and 3-29, wherein the first binding domain comprises complementarity determining regions (CDRs) from a binding molecule selected from the group consisting of: IMMU 546, FIN9, AMKB1-2, ZOE, 3G5, ZIZOU4, IMMU 157, LC4, 3D11, 1C1, W112, MH3-2, 4H11, 36213, BAM13, H131, A.H-53, H132, IMMU 222, JU-74, JU74.3, OT145, BL37.2, S511, VER2.32.1, IG125, 56C5, 56C5.2, 16G8, MX-6, JR2, AF-23, AF23, AHUT7, TAMAYA1.2, BA62, BA62.6, C1, E17.5F3, E17.5F3.15.13, MPB2D5, C21, CAS1.1.3, CH92, 8F10, JOVI-3, WJF24, ELL1.4, JOVI-1, SP305, LG.3A10, 5A8A10, 5D9F1, and KFN; wherein the CDRs are each determined according to the Kabat numbering scheme, the Chothia numbering scheme, or the ImMunoGeneTics Information System (IMGT).

Embodiment 31. The multi-specific polypeptide of any one of embodiments 1 and 3-26, wherein the first binding domain specifically binds TCRαV.

Embodiment 32. The multi-specific polypeptide of any one of embodiments 1, 3-26, and 31, wherein the TCRαV is TCRαV 1-2.

Embodiment 33. The multi-specific polypeptide of any one of embodiments 1, 3-26, 31, and 32, wherein the TCRαV comprises an amino acid sequence that is at least 80%, at least 85%, at least 90%, at least 95%, at least 96%, at least 97%, at least 98%, at least 99%, or 100% identical to SEQ ID NO: 149.

Embodiment 34. The multi-specific polypeptide of any one of embodiments 1, 3-26, and 31-33, wherein the first binding domain comprises CDRs from a binding molecule selected from the group consisting of: C15, 6B11, SFB280, 3C10, F1, 6D6.6, and clone 34; wherein the CDRs are each determined according to the Kabat numbering scheme, the Chothia numbering scheme, or the ImMunoGeneTics Information System (IMGT).

Embodiment 35. The multi-specific polypeptide of any one of embodiments 1-34, wherein the first binding domain comprises: (i) a VH region comprising an HCDR1, an HCDR2, and an HCDR3, and (ii) a VL region comprising an LCDR1, an LCDR2, and an LCDR3.

Embodiment 36. The multi-specific polypeptide of embodiment 35, wherein the first binding domain is a single chain variable fragment (scFv), a Fab, a CrossFab, a Fab′, or a F(ab′)₂.

Embodiment 37. The multi-specific polypeptide of embodiment 36, wherein the Fab comprises the VH region, the VL region, a CH1 region, and a CL region, wherein the C-terminus of the VH region is connected to the N-terminus of the CH1 region, and the C-terminus of the VL region is connected to the N-terminus of the CL region.

Embodiment 38. The multi-specific polypeptide of embodiment 36, wherein the CrossFab comprises the VH region, the VL region, a CH1 region, and a CL region, wherein the C-terminus of the VH region is connected to the N-terminus of the CL region, and the C-terminus of the VL region is connected to the N-terminus of the CH1 region.

Embodiment 39. The multi-specific polypeptide of embodiment 37 or 38, wherein the CH1 region and the CL region are connected by a disulfide bond.

Embodiment 40. The multi-specific polypeptide of any one of embodiments 36-39, wherein the Fab or the CrossFab is a charge steering Fab or a charge steering CrossFab.

Embodiment 41. The multi-specific polypeptide of embodiment 40, wherein a CH1 region of the charge steering Fab or the charge steering CrossFab comprises mutations K147E/K213E according to EU numbering, and a CL region of the charge steering Fab or charge steering CrossFab comprises mutations E123R/Q124K according to EU numbering.

Embodiment 42. The multi-specific polypeptide of embodiment 36, wherein the scFv comprises the VH region and the VL region.

Embodiment 43. The multi-specific polypeptide of embodiment 42, wherein the C-terminus of the VH region is linked to the N-terminus of the VL region.

Embodiment 44. The multi-specific polypeptide of embodiment 42, wherein the C-terminus of the VL region is linked to the N-terminus of the VH region.

Embodiment 45. The multi-specific polypeptide of any one of embodiments 42-44, wherein the VH region and the VL region are linked by one or more engineered disulfide bond(s).

Embodiment 46. The multi-specific polypeptide of embodiment 45, wherein one cysteine that forms the engineered disulfide bond is located in the VH region, and wherein the other cysteine that forms the engineered disulfide bond is located in the VL region.

Embodiment 47. The multi-specific polypeptide of embodiment 45 or 46, wherein the positions of the one or more engineered disulfide bond(s) are selected from the group consisting of VH44-VL100, VH100B-VL49, VH100-VL50, VH101-VL46, and VH105-VL43.

Embodiment 48. The multi-specific polypeptide of any one of embodiments 42-47, wherein the VH region and the VL region are linked by a peptide linker (PL1).

Embodiment 49. The multi-specific polypeptide of embodiment 48, wherein the peptide linker (PL1) comprises the formula of (Gly₄Ser)ₙ, wherein n=1-5.

Embodiment 50. The multi-specific polypeptide of embodiment 49, wherein the peptide linker (PL1) is (Gly₄Ser)₃.

Embodiment 51. The multi-specific polypeptide of embodiment 49, wherein the peptide linker (PL1) is (Gly₄Ser)₁.

Embodiment 52. The multi-specific polypeptide of any one of embodiments 35-51, wherein the HCDR1, the HCDR2, the HCDR3, the LCDR1, the LCDR2, and the LCDR3 of the first binding domain are according to one of the combinations of complementarity determining regions (CDRs) of Table 1, Table 2, Table 6, or Table 7.

Embodiment 53. The multi-specific polypeptide of any one of embodiments 35-52, wherein the HCDR1 comprises an amino acid sequence of LYYIH (SEQ ID NO: 1138), the HCDR2 comprises an amino acid sequence of WFFPGSGNIKYNEKFKG (SEQ ID NO: 2), the HCDR3 comprises an amino acid sequence of SYYSFDVLDY (SEQ ID NO: 1139), the LCDR1 comprises an amino acid sequence of KASQNVGIRVV (SEQ ID NO: 1140), the LCDR2 comprises an amino acid sequence of SSSHRYS (SEQ ID NO: 5), and the LCDR3 comprises an amino acid sequence of QQFKSYPIT (SEQ ID NO: 1141), wherein the CDRs correspond to the first binding domain and are numbered with reference to the Kabat numbering scheme.

Embodiment 54. The multi-specific polypeptide of any one of embodiments 35-52, wherein the HCDR1 comprises an amino acid sequence of TYYIH (SEQ ID NO: 1), the HCDR2 comprises an amino acid sequence of WFFPRSGNIKYNEKFKG (SEQ ID NO: 1142), the HCDR3 comprises an amino acid sequence of SYYSFDVLDY (SEQ ID NO: 1139), the LCDR1 comprises an amino acid sequence of KASQNVGIRVV (SEQ ID NO: 1140), the LCDR2 comprises an amino acid sequence of SSSHRYS (SEQ ID NO: 5), and the LCDR3 comprises an amino acid sequence of QQFKSYPIT (SEQ ID NO: 1141), wherein the CDRs correspond to the first binding domain and are numbered with reference to the Kabat numbering scheme.

Embodiment 55. The multi-specific polypeptide of any one of embodiments 35-52, wherein the HCDR1 comprises an amino acid sequence of GYSFTLYY (SEQ ID NO: 1143), the HCDR2 comprises an amino acid sequence of FFPGSGNI (SEQ ID NO: 18), the HCDR3 comprises an amino acid sequence of AASYYSFDVLDY (SEQ ID NO: 1144), the LCDR1 comprises an amino acid sequence of QNVGIR (SEQ ID NO: 1145), the LCDR2 comprises an amino acid sequence of SSS, and the LCDR3 comprises an amino acid sequence of QQFKSYPIT (SEQ ID NO: 1146), wherein the CDRs correspond to the first binding domain and are numbered with reference to the IMGT numbering scheme.

Embodiment 56. The multi-specific polypeptide of any one of embodiments 35-52, wherein the HCDR1 comprises an amino acid sequence of GYSFTTYY (SEQ ID NO: 17), the HCDR2 comprises an amino acid sequence of FFPRSGNI (SEQ ID NO: 1147), the HCDR3 comprises an amino acid sequence of AASYYSFDVLDY (SEQ ID NO: 1144), the LCDR1 comprises an amino acid sequence of QNVGIR (SEQ ID NO: 1145), the LCDR2 comprises an amino acid sequence of SSS, and the LCDR3 comprises an amino acid sequence of QQFKSYPIT (SEQ ID NO: 1146), wherein the CDRs correspond to the first binding domain and are numbered with reference to the IMGT numbering scheme.

Embodiment 57. The multi-specific polypeptide of any one of embodiments 35-56, wherein the VH region of the first binding domain comprises an amino acid sequence that is at least 80%, at least 85%, at least 90%, at least 95%, at least 96%, at least 97%, at least 98%, at least 99%, or 100% identical to one of SEQ ID NOs listed in the VH column of Table 3 or Table 8, and wherein the VL region of the first binding domain comprises an amino acid sequence that is at least 80%, at least 85%, at least 90%, at least 95%, at least 96%, at least 97%, at least 98%, at least 99%, or 100% identical to the corresponding VL SEQ ID NO listed in the same row of Table 3 or Table 8.

Embodiment 58. The multi-specific polypeptide of any one of embodiments 35-56, wherein the VH region of the first binding domain comprises an amino acid sequence that is at least 80%, at least 85%, at least 90%, at least 95%, at least 96%, at least 97%, at least 98%, at least 99%, or 100% identical to SEQ ID NO: 1148 or SEQ ID NO: 1190, and wherein the VL region of the first binding domain comprises an amino acid sequence that is at least 80%, at least 85%, at least 90%, at least 95%, at least 96%, at least 97%, at least 98%, at least 99%, or 100% identical to SEQ ID NO: 1149 or SEQ ID NO: 1192.

Embodiment 59. The multi-specific polypeptide of any one of embodiments 35-56, wherein the VH region of the first binding domain comprises an amino acid sequence that is at least 80%, at least 85%, at least 90%, at least 95%, at least 96%, at least 97%, at least 98%, at least 99%, or 100% identical to SEQ ID NO: 1150 or SEQ ID NO: 1191, and wherein the VL region of the first binding domain comprises an amino acid sequence that is at least 80%, at least 85%, at least 90%, at least 95%, at least 96%, at least 97%, at least 98%, at least 99%, or 100% identical to SEQ ID NO: 1149 or SEQ ID NO: 1192.

Embodiment 60. The multi-specific polypeptide of any one of embodiments 35-51, wherein the HCDR1 comprises an amino acid sequence of SEQ ID NO: 201, the HCDR2 comprises an amino acid sequence of SEQ ID NO: 202, the HCDR3 comprises an amino acid sequence of SEQ ID NO: 203, the LCDR1 comprises an amino acid sequence of SEQ ID NO: 204, the LCDR2 comprises an amino acid sequence of SEQ ID NO: 205, and the LCDR3 comprises an amino acid sequence of SEQ ID NO: 206, wherein the CDRs correspond to the first binding domain and are numbered with reference to the Kabat numbering scheme.

Embodiment 61. The multi-specific polypeptide of embodiment 60, wherein the VH region of the first binding domain comprises an amino acid sequence that is at least 80%, at least 85%, at least 90%, at least 95%, at least 96%, at least 97%, at least 98%, at least 99%, or 100% identical to one of SEQ ID NO: 213, and wherein the VL region of the first binding domain comprises an amino acid sequence that is at least 80%, at least 85%, at least 90%, at least 95%, at least 96%, at least 97%, at least 98%, at least 99%, or 100% identical to the corresponding VL SEQ ID NO: 214.

Embodiment 62. The multi-specific polypeptide of any one of embodiments 1, 3-36, and 42-59, wherein the first binding domain comprises an scFv comprising an amino acid sequence that is at least 80%, at least 85%, at least 90%, at least 95%, at least 96%, at least 97%, at least 98%, at least 99%, or 100% identical to any one of SEQ ID NOs in Table 4 or Table 9.

Embodiment 63. The multi-specific polypeptide of any one of embodiments 1, 3-36, and 42-59, wherein the first binding domain comprises an scFv comprising an amino acid sequence that is at least 80%, at least 85%, at least 90%, at least 95%, at least 96%, at least 97%, at least 98%, at least 99%, or 100% identical to SEQ ID NO: 1151.

Embodiment 64. The multi-specific polypeptide of any one of embodiments 1, 3-36, and 42-59, wherein the first binding domain comprises an scFv comprising an amino acid sequence that is at least 80%, at least 85%, at least 90%, at least 95%, at least 96%, at least 97%, at least 98%, at least 99%, or 100% identical to SEQ ID NO: 1152.

Embodiment 65. The multi-specific polypeptide of any one of embodiments 1, 3-36, and 42-59, wherein the first binding domain comprises an scFv comprising an amino acid sequence that is at least 80%, at least 85%, at least 90%, at least 95%, at least 96%, at least 97%, at least 98%, at least 99%, or 100% identical to SEQ ID NO: 1153.

Embodiment 66. The multi-specific polypeptide of any one of embodiments 1, 3-36, and 42-59, wherein the first binding domain comprises an scFv comprising an amino acid sequence that is at least 80%, at least 85%, at least 90%, at least 95%, at least 96%, at least 97%, at least 98%, at least 99%, or 100% identical to SEQ ID NO: 1154.

Embodiment 67. The multi-specific polypeptide of any one of embodiments 1, 3-41, 56, and 57, wherein the first binding domain comprises a Fab or CrossFab comprising (i) a heavy chain comprising an amino acid sequence that is at least 80%, at least 85%, at least 90%, at least 95%, at least 96%, at least 97%, at least 98%, at least 99%, or 100% identical to any one of SEQ ID NOs: 243, 245, 1071-1086, 1112-1123, 1129,1130, 1155, and 1157; and (ii) a light chain comprising an amino acid sequence that is at least 80%, at least 85%, at least 90%, at least 95%, at least 96%, at least 97%, at least 98%, at least 99%, or 100% identical to any one of SEQ ID NOs: 244, 246, 1055-1070, 1100-1111, 1127,1128, 1156, and 1158.

Embodiment 68. The multi-specific polypeptide of any one of embodiments 3-67, wherein the half-life extension moiety is selected from the group consisting of a fragment crystallizable (Fc) region (e.g., a single chain Fc), an albumin (e.g., human serum albumin), transferrin, a binding domain that specifically binds to transferrin or albumin (e.g., an albumin binding domain (ABD)), an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising Proline, Alanine and Serine (PAS), or a derivative or functional fragment thereof.

Embodiment 69. The multi-specific polypeptide of any one of embodiments 3-68, wherein the half-life extension moiety is a fragment crystallizable (Fc) region.

Embodiment 70. The multi-specific polypeptide of embodiment 69, wherein the Fc region comprises two polypeptides.

Embodiment 71. The multi-specific polypeptide of embodiment 70, wherein the Fc region has a knobs-into-holes format, wherein the first polypeptide of the Fc region comprises a hole mutation, and wherein second polypeptide of the Fc region comprises a knob mutation.

Embodiment 72. The multi-specific polypeptide of embodiment 71, wherein the first polypeptide of the Fc region comprises mutations T366S, L368A and Y407V according to EU numbering, and wherein the second polypeptide of the Fc region comprises a mutation T366W according to EU numbering.

Embodiment 73. The multi-specific polypeptide of embodiment 71 or 72, wherein the first polypeptide of the Fc region comprising the hole mutation further comprises a mutation Y349C according to EU numbering, wherein the second polypeptide of the Fc region comprising the knob mutation further comprises a mutation S354C according to EU numbering, and wherein the Fc region comprises an engineered disulfide bond formed between these two cysteines.

Embodiment 74. The multi-specific polypeptide of any one of embodiments 69-73, wherein one or both of the polypeptides of the Fc region comprise mutations L234A, L235A, and P329G according to EU numbering.

Embodiment 75. The multi-specific polypeptide of any one of embodiments 69-74, wherein one or both of the polypeptides of the Fc region comprise the mutation N297G according to EU numbering.

Embodiment 76. The multi-specific polypeptide of any one of embodiments 69-75, wherein one or both of the polypeptides of the Fc region comprise mutations H435R and Y436F according to EU numbering.

Embodiment 77. The multi-specific polypeptide of any one of embodiments 69-76, wherein the Fc region lacks one or more effector functions and/or lacks glycosylation.

Embodiment 78. The multi-specific polypeptide of embodiment 77, wherein the effector function is selected from the group consisting of antibody-dependent cellular cytotoxicity (ADCC), antibody-dependent cellular phagocytosis (ADCP), complement-dependent cytotoxicity (CDC), binding to a Fcγ receptor (FcγR), or any combination thereof.

Embodiment 79. The multi-specific polypeptide of any one of embodiments 71-78, wherein the first polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 154, and wherein the second polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 155.

Embodiment 80. The multi-specific polypeptide of any one of embodiments 71-78, wherein the first polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 156, and wherein the second polypeptide of the Fc region with a peptide linker PLE of DKTHTCPPCP (SEQ ID NO: 161) comprises an amino acid sequence that is at least 80%, at least 85%, at least 90%, at least 95%, at least 97%, at least 98%, at least 99%, or 100% identical to SEQ ID NO: 157.

Embodiment 81. The multi-specific polypeptide of any one of embodiments 35-80, wherein the C-terminus of the VH region of the first binding domain is connected to the N-terminus of the VH region of the second binding domain.

Embodiment 82. The multi-specific polypeptide of any one of embodiments 35-80, wherein the C-terminus of the VH region of the first binding domain is connected to the N-terminus of the VL region of the second binding domain.

Embodiment 83. The multi-specific polypeptide of any one of embodiments 35-80, wherein the C-terminus of the VL region of the first binding domain is connected to the N-terminus of the VH region of the second binding domain.

Embodiment 84. The multi-specific polypeptide of any one of embodiments 35-80, wherein the C-terminus of the VL region of the first binding domain is connected to the N-terminus of the VL region of the second binding domain.

Embodiment 85. The multi-specific polypeptide of any one of embodiments 35-80, wherein the C-terminus of the CH1 region of the first binding domain is connected to the N-terminus of the VH region of the second binding domain.

Embodiment 86. The multi-specific polypeptide of any one of embodiments 35-80, wherein the C-terminus of the CH1 region of the first binding domain is connected to the N-terminus of the VL region of the second binding domain.

Embodiment 87. The multi-specific polypeptide of any one of embodiments 35-80, wherein the C-terminus of the CL region of the first binding domain is connected to the N-terminus of the VH region of the second binding domain.

Embodiment 88. The multi-specific polypeptide of any one of embodiments 35-80, wherein the C-terminus of the CL region of the first binding domain is connected to the N-terminus of the VL region of the second binding domain.

Embodiment 89. The multi-specific polypeptide of any one of embodiments 82, 84, 86, and 88, wherein the C-terminus of the VH region of the second binding domain is connected to the N-terminus of either the first polypeptide or the second polypeptide of the Fc region.

Embodiment 90. The multi-specific polypeptide of any one of embodiments 81, 83, 85, and 87, wherein the C-terminus of the VL region of the second binding domain is connected to the N-terminus of either the first polypeptide or the second polypeptide of the Fc region.

Embodiment 91. The multi-specific polypeptide of any one of embodiments 81-88, wherein the C-terminus of the CH1 region of the second binding domain is connected to the N-terminus of either the first polypeptide or the second polypeptide of the Fc region.

Embodiment 92. The multi-specific polypeptide of any one of embodiments 81-88, wherein the C-terminus of the CL region of the second binding domain is connected to the N-terminus of either the first polypeptide or the second polypeptide of the Fc region.

Embodiment 93. The multi-specific polypeptide of any one of embodiments 35-80, wherein the C-terminus of the VH region of the second binding domain is connected to the N-terminus of the VH region of the first binding domain.

Embodiment 94. The multi-specific polypeptide of any one of embodiments 35-80, wherein the C-terminus of the VH region of the second binding domain is connected to the N-terminus of the VL region of the first binding domain.

Embodiment 95. The multi-specific polypeptide of any one of embodiments 35-80, wherein the C-terminus of the VL region of the second binding domain is connected to the N-terminus of the VH region of the first binding domain.

Embodiment 96. The multi-specific polypeptide of any one of embodiments 35-80, wherein the C-terminus of the VL region of the second binding domain is connected to the N-terminus of the VL region of the first binding domain.

Embodiment 97. The multi-specific polypeptide of any one of embodiments 35-80, wherein the C-terminus of the CH1 region of the second binding domain is connected to the N-terminus of the VH region of the first binding domain.

Embodiment 98. The multi-specific polypeptide of any one of embodiments 35-80, wherein the C-terminus of the CH1 region of the second binding domain is connected to the N-terminus of the VL region of the first binding domain.

Embodiment 99. The multi-specific polypeptide of any one of embodiments 35-80, wherein the C-terminus of the CL region of the second binding domain is connected to the N-terminus of the VH region of the first binding domain.

Embodiment 100. The multi-specific polypeptide of any one of embodiments 35-80, wherein the C-terminus of the CL region of the second binding domain is connected to the N-terminus of the VL region of the first binding domain.

Embodiment 101. The multi-specific polypeptide of any one of embodiments 94, 96, 98, and 100, wherein the C-terminus of the VH region of the first binding domain is connected to the N-terminus of either the first polypeptide or the second polypeptide of the Fc region.

Embodiment 102. The multi-specific polypeptide of any one of embodiments 93, 95, 97, and 99, wherein the C-terminus of the VL region of the first binding domain is connected to the N-terminus of either the first polypeptide or the second polypeptide of the Fc region.

Embodiment 103. The multi-specific polypeptide of any one of embodiments 93-100, wherein the C-terminus of the CH1 region of the first binding domain is connected to the N-terminus of either the first polypeptide or the second polypeptide of the Fc region.

Embodiment 104. The multi-specific polypeptide of any one of embodiments 93-100, wherein the C-terminus of the CL region of the first binding domain is connected to the N-terminus of either the first polypeptide or the second polypeptide of the Fc region.

Embodiment 105. The multi-specific polypeptide of any one of embodiments 81-104, wherein the second binding domain is connected to the second polypeptide of the Fc region comprising the knob mutation.

Embodiment 106. The multi-specific polypeptide of any one of embodiments 81-104, wherein the second binding domain is connected to the first polypeptide of the Fc region comprising the hole mutation.

Embodiment 107. The multi-specific polypeptide of any one of embodiments 81-104, wherein the first binding domain is connected to the second polypeptide of the Fc region comprising the knob mutation.

Embodiment 108. The multi-specific polypeptide of any one of embodiments 81-104, wherein the first binding domain is connected to the first polypeptide of the Fc region comprising the hole mutation.

Embodiment 109. The multi-specific polypeptide of any one of embodiments 105-108, wherein the other polypeptide of the Fc region is not connected to any binding domain.

Embodiment 110. The multi-specific polypeptide of any one of embodiments 3-80, wherein the first binding domain is an scFv, wherein the second binding domain is a Fab, wherein the C-terminus of the first binding domain is connected to the N-terminus of the VH region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

Embodiment 111. The multi-specific polypeptide of any one of embodiments 3-80, wherein the first binding domain is an scFv, wherein the second binding domain is a Fab, wherein the C-terminus of the first binding domain is connected to the N-terminus of the VL region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

Embodiment 112. The multi-specific polypeptide of any one of embodiments 3-80, wherein the first binding domain is an scFv, wherein the second binding domain is a Fab, wherein the N-terminus of the first binding domain is connected to the C-terminus of the CH1 region of the second binding domain via a peptide linker (PLB), and wherein the C-terminus of the first binding domain is connected to the N-terminus of the second polypeptide of the Fc region comprising a knob mutation via a peptide linker (PLE).

Embodiment 113. The multi-specific polypeptide of any one of embodiments 3-80, wherein the first binding domain is a Fab, wherein the second binding domain is an scFv, wherein the C-terminus of the CH1 region of the first binding domain is connected to the N-terminus of the second binding domain via a peptide linker (PLB), and wherein the C-terminus of the second binding domain is connected to the N-terminus of the second polypeptide of the Fc region comprising a knob mutation via a peptide linker (PLE).

Embodiment 114. The multi-specific polypeptide of any one of embodiments 3-80, wherein the first binding domain is a Fab, wherein the second binding domain is an scFv, wherein the N-terminus of the VH region of the first binding domain is connected to the C-terminus of the second binding domain via a peptide linker (PLB), and wherein the C-terminus of the CH1 region of the first binding domain is connected to the N-terminus of the second polypeptide of the Fc region comprising a knob mutation via a peptide linker (PLE).

Embodiment 115. The multi-specific polypeptide of any one of embodiments 3-80, wherein the first binding domain is a Fab, wherein the second binding domain is an scFv, wherein the N-terminus of the VL region of the first binding domain is connected to the C-terminus of the second binding domain via a peptide linker (PLB), and wherein the C-terminus of the CH1 region of the first binding domain is connected to the N-terminus of the second polypeptide of the Fc region comprising a knob mutation via a peptide linker (PLE).

Embodiment 116. The multi-specific polypeptide of any one of embodiments 3-80, wherein the first binding domain is a CrossFab, wherein the second binding domain is a charge steering Fab, wherein the N-terminus of the VL region of the first binding domain is connected to the C-terminus of the CH1 region of the second binding domain via a peptide linker (PLB), and wherein the C-terminus of the CH1 region of the first binding domain is connected to the N-terminus of the second polypeptide of the Fc region comprising a knob mutation via a peptide linker (PLE).

Embodiment 117. The multi-specific polypeptide of any one of embodiments 3-80, wherein the first binding domain is a CrossFab, wherein the second binding domain is a charge steering Fab, wherein the C-terminus of the CH1 region of the first binding domain is connected to the N-terminus of the VL region of the second binding domain via a peptide linker (PLB), and wherein the C-terminus of the CH1 region of the second binding domain is connected to the N-terminus of the second polypeptide of the Fc region comprising a knob mutation via a peptide linker (PLE).

Embodiment 118. The multi-specific polypeptide of any one of embodiments 1-109, wherein the first binding domain and the second binding domain are connected via a peptide linker (PLB).

Embodiment 119. The multi-specific polypeptide of any one of embodiments 110-118, wherein the peptide linker (PLB) is at least 5 amino acids in length.

Embodiment 120. The multi-specific polypeptide of embodiment 119, wherein the peptide linker (PLB) comprises the formula of (Gly₄Ser)ₙ, wherein n=1-5.

Embodiment 121. The multi-specific polypeptide of embodiment 120, wherein the peptide linker (PLB) is (Gly₄Ser)₃.

Embodiment 122. The multi-specific polypeptide of embodiment 120, wherein the peptide linker (PLB) is (Gly₄Ser)₂.

Embodiment 123. The multi-specific polypeptide of embodiment 120, wherein the peptide linker (PLB) is (Gly₄Ser)₁.

Embodiment 124. The multi-specific polypeptide of any one of embodiments 70-80, wherein the first binding domain is linked to either the first polypeptide or the second polypeptide of the Fc region, and wherein the second binding domain is linked to the other polypeptide of the Fc region.

Embodiment 125. The multi-specific polypeptide of embodiment 124, wherein the first binding domain is linked to the second polypeptide of the Fc region comprising a knob mutation, and wherein the second binding domain is linked to the first polypeptide of the Fc region comprising a hole mutation.

Embodiment 126. The multi-specific polypeptide of embodiment 124 or 125, wherein the first binding domain is a Fab or CrossFab, and wherein the second binding domain is an scFv.

Embodiment 127. The multi-specific polypeptide of embodiment 124 or 125, wherein the first binding domain is a CrossFab connecting to the Fc region via the C-terminus of its CH1 region, and wherein the second binding domain is a Fab connecting to the Fc region via the C-terminus of its CH1 region.

Embodiment 128. The multi-specific polypeptide of embodiment 124 or 125, wherein the first binding domain is a CrossFab connecting to the Fc region via the C-terminus of its CH1 region, and wherein the second binding domain is a charge steering Fab connecting to the Fc region via the C-terminus of its CH1 region.

Embodiment 129. The multi-specific polypeptide of embodiment 124 or 125, wherein the first binding domain is a CrossFab connecting to the Fc region via the C-terminus of its CL region, and wherein the second binding domain is a Fab connecting to the Fc region via the C-terminus of its CH1 region.

Embodiment 130. The multi-specific polypeptide of any one of embodiments 124-129, wherein the C-terminus of the scFv or the CH1 region of the binding domain is linked to the N-terminus of the first or the second polypeptide of the Fc region.

Embodiment 131. The multi-specific polypeptide of any one of embodiments 124-129, wherein the CL region of the binding domain is linked to the N-terminus of the first or the second polypeptide of the Fc region.

Embodiment 132. The multi-specific polypeptide of any one of embodiments 3-131, wherein the binding domain and the half-life extension moiety are connected via a peptide linker (PLE).

Embodiment 133. The multi-specific polypeptide of embodiment 132, wherein the peptide linker (PLE) is at least 5 amino acids in length.

Embodiment 134. The multi-specific polypeptide of embodiment 133, wherein the peptide linker (PLE) is between 5-10 amino acids, between 11-15 amino acids, between 16-20 amino acids, between 21-25 amino acids, or between 26-30 amino acids, in length.

Embodiment 135. The multi-specific polypeptide of any one of embodiments 132-134, wherein the peptide linker (PLE) comprises an amino acid sequence having the formula of (Gly₄Ser)ₙ, wherein n=1-5.

Embodiment 136. The multi-specific polypeptide of embodiment 135, wherein the peptide linker (PLE) comprises (Gly₄Ser)₂.

Embodiment 137. The multi-specific polypeptide of any one of embodiments 132-134, wherein the peptide linker (PLE) comprises or consists of the amino acid sequence KTHTCP (SEQ ID NO: 158).

Embodiment 138. The multi-specific polypeptide of any one of embodiments 132-134, wherein the peptide linker (PLE) comprises or consists of the amino acid sequence DKTHTCP (SEQ ID NO: 160).

Embodiment 139. The multi-specific polypeptide of any one of embodiments 132-134, wherein the peptide linker (PLE) comprises or consists of the amino acid sequence GGGGSGGGGSKTHTCP (SEQ ID NO: 159).

Embodiment 140. The multi-specific polypeptide of any one of embodiments 1-139, wherein the multi-specific polypeptide comprises an amino acid sequence that is at least 80%, at least 85%, at least 90%, at least 95%, at least 97%, at least 98%, at least 99%, or 100% identical to any one of SEQ ID NOs: 407-991 and 1159-1179.

Embodiment 141. The multi-specific polypeptide of any one of embodiments 1-139, wherein the multi-specific polypeptide comprises three non-identical polypeptides.

Embodiment 142. The multi-specific polypeptide of embodiment 141, wherein the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab light chain; and the third polypeptide comprises: (i) an anti-CD19 Fab heavy chain; (ii) a PLE linker; and (iii) an Fc heavy chain comprising a knob mutation.

Embodiment 143. The multi-specific polypeptide of embodiment 141, wherein the first polypeptide comprises: (i) a PLE linker; and (ii) an Fc heavy chain comprising a hole mutation; the second polypeptide comprises: (i) an anti-CD19 Fab light chain; and the third polypeptide comprises: (i) an anti-TCRβ scFv; (ii) a PLB linker; (iii) an anti-CD19 Fab heavy chain; (iv) a PLE linker; and (iii) an Fc heavy chain comprising a knob mutation.

Embodiment 144. The multi-specific polypeptide of embodiment 141, wherein the first polypeptide comprises: (i) a PLE linker; and (ii) an Fc heavy chain comprising a hole mutation;

the second polypeptide comprises: (i) an anti-CD19 Fab heavy chain; (ii) a PLE linker; and (iii) an Fc heavy chain comprising a knob mutation; and the third polypeptide comprises: (i) an anti-TCRβ scFv; (ii) a PLB linker; (iii) an anti-CD19 Fab light chain.

Embodiment 145. The multi-specific polypeptide of embodiment 141, wherein the first polypeptide comprises: (i) a PLE linker; and (ii) an Fc heavy chain comprising a hole mutation; the second polypeptide comprises: (i) an anti-TCRβ scFv; (ii) a PLB linker; (iii) an anti-CD19 Fab heavy chain; (iv) a PLE linker; and (v) an Fc heavy chain comprising a knob mutation; and the third polypeptide comprises an anti-CD19 Fab light chain.

Embodiment 146. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1159; the second polypeptide comprises an amino acid sequence that is at least 80%, at least 85%, at least 90%, at least 95%, at least 97%, at least 98%, at least 99%, or 100% identical to SEQ ID NO: 1166; and the third polypeptide comprises an amino acid sequence that is at least 80%, at least 85%, at least 90%, at least 95%, at least 97%, at least 98%, at least 99%, or 100% identical to SEQ ID NO: 1173.

Embodiment 147. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1160; the second polypeptide comprises an amino acid sequence that is at least 80%, at least 85%, at least 90%, at least 95%, at least 97%, at least 98%, at least 99%, or 100% identical to SEQ ID NO: 1167; and the third polypeptide comprises an amino acid sequence that is at least 80%, at least 85%, at least 90%, at least 95%, at least 97%, at least 98%, at least 99%, or 100% identical to SEQ ID NO: 1174.

Embodiment 148. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1161; the second polypeptide comprises an amino acid sequence that is at least 80%, at least 85%, at least 90%, at least 95%, at least 97%, at least 98%, at least 99%, or 100% identical to SEQ ID NO: 1168; and the third polypeptide comprises an amino acid sequence that is at least 80%, at least 85%, at least 90%, at least 95%, at least 97%, at least 98%, at least 99%, or 100% identical to SEQ ID NO: 1175.

Embodiment 149. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1162; the second polypeptide comprises an amino acid sequence that is at least 80%, at least 85%, at least 90%, at least 95%, at least 97%, at least 98%, at least 99%, or 100% identical to SEQ ID NO: 1169; and the third polypeptide comprises an amino acid sequence that is at least 80%, at least 85%, at least 90%, at least 95%, at least 97%, at least 98%, at least 99%, or 100% identical to SEQ ID NO: 1176.

Embodiment 150. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1163; the second polypeptide comprises an amino acid sequence that is at least 80%, at least 85%, at least 90%, at least 95%, at least 97%, at least 98%, at least 99%, or 100% identical to SEQ ID NO: 1170; and the third polypeptide comprises an amino acid sequence that is at least 80%, at least 85%, at least 90%, at least 95%, at least 97%, at least 98%, at least 99%, or 100% identical to SEQ ID NO: 1177.

Embodiment 151. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1164; the second polypeptide comprises an amino acid sequence that is at least 80%, at least 85%, at least 90%, at least 95%, at least 97%, at least 98%, at least 99%, or 100% identical to SEQ ID NO: 1171; and the third polypeptide comprises an amino acid sequence that is at least 80%, at least 85%, at least 90%, at least 95%, at least 97%, at least 98%, at least 99%, or 100% identical to SEQ ID NO: 1178.

Embodiment 152. The multi-specific polypeptide of embodiment 141, wherein the first polypeptide comprises an amino acid sequence that is at least 80%, at least 85%, at least 90%, at least 95%, at least 97%, at least 98%, at least 99%, or 100% identical to SEQ ID NO: 1165; the second polypeptide comprises an amino acid sequence that is at least 80%, at least 85%, at least 90%, at least 95%, at least 97%, at least 98%, at least 99%, or 100% identical to SEQ ID NO: 1172; and the third polypeptide comprises an amino acid sequence that is at least 80%, at least 85%, at least 90%, at least 95%, at least 97%, at least 98%, at least 99%, or 100% identical to SEQ ID NO: 1179.

Embodiment 153. The multi-specific polypeptide of any one of embodiments 5-152, wherein the VH and VL regions are numbered with reference to the Kabat numbering scheme.

Embodiment 154. A recombinant nucleic acid molecule encoding the multi-specific polypeptide of any one of embodiments 1-153.

Embodiment 155. A vector comprising the recombinant nucleic acid molecule of embodiment 154.

Embodiment 156. A pharmaceutical composition comprising (i) the multi-specific polypeptide of any one of embodiments 1-153, the recombinant nucleic acid molecule of embodiment 154, or the vector of embodiment 155, and (ii) a pharmaceutically acceptable carrier, diluent, or excipient.

Embodiment 157. A method of producing a multi-specific polypeptide, comprising introducing the recombinant nucleic acid molecule of embodiment 154, or the vector of embodiment 155 into a host cell under conditions to express the multi-specific polypeptide complex in the cell, wherein the method is ex vivo.

Embodiment 158. A method of expanding a T cell population, wherein the method comprises contacting an initial T cell population with the multi-specific polypeptide of any one of embodiments 1-153 or the pharmaceutical composition of embodiment 156, thereby producing an expanded population of T cells; wherein the initial T cell population expresses TCRβV or TCRαV on the cell surface, and wherein the first binding domain of the multi-specific polypeptide specifically binds the TCRβV or TCRαV.

Embodiment 159. The method of embodiment 158, wherein the method expands T cells in vitro or ex vivo.

Embodiment 160. The method of embodiment 158, wherein the method expands T cells in vivo in a subject.

Embodiment 161. The method of any one of embodiments 158-160, wherein the initial population of T cells comprises a plurality of effector T cells and/or memory T cells.

Embodiment 162. The method of any one of embodiments 158-161, wherein the expanded population of T cells has an increased expansion of the effector T cells and/or memory T cells compared to a control T cell population that was contacted with a control multi-specific polypeptide comprising a first binding domain that specifically binds to CD3 instead of TCRβV or TCRαV.

Embodiment 163. The method of embodiment 162, wherein the effector T cells are selected from the group consisting of effector memory T cells (TEM), T effector cells (TEFF), T effector cells re-expressing CD45RA (TEMRA), and a combination thereof.

Embodiment 164. The method of embodiment 162, wherein the memory T cells are selected from the group consisting of central memory T cells (Tcm), T memory stem cells (Tscm), and a combination thereof.

Embodiment 165. The method of any one of embodiments 158-164, wherein the expanded population of T cells has an increased percentage of effector T cells and/or memory T cells compared to that of a control T cell population contacted with a control multi-specific polypeptide comprising a first binding domain that specifically binds to CD3 instead of TCRβV or TCRαV.

Embodiment 166. The method of embodiment 165, wherein the effector T cells are effector memory T cells (TEM).

Embodiment 167. The method of embodiment 165, wherein the memory T cells are central memory T cells (Tcm).

Embodiment 168. The method of any one of embodiments 158-167, wherein the expanded population of T cells has an increased and/or prolonged cytotoxicity compared to that of a control T cell population contacted with a control multi-specific polypeptide comprising a first binding domain that specifically binds to CD3 instead of TCRβV or TCRαV.

Embodiment 169. The method of any one of embodiments 158-168, wherein the comparison between the expanded population of T cells expanded by the multi-specific polypeptide and control T cell population contacted with the control multi-specific polypeptide is performed 1, 2, 3, 4, 5, 6, 7, 8, 9, 10, or 11 days after an initial contact of the T cells.

Embodiment 170. The method of any one of embodiments 158-169, wherein the method depletes B cells.

Embodiment 171. A method of depleting B cells in a subject having a disease or disorder related to the B cells, comprising administering to the subject an effective amount of the multi-specific polypeptide of any one of embodiments 1-153 or the pharmaceutical composition of embodiment 156.

Embodiment 172. The method of embodiment 171, wherein the method decreases the number of B cells by at least 50%, at least 55%, at least 60%, at least 65%, at least 70%, at least 75%, at least 80%, at least 85%, at least 90%, at least 95%, at least 98%, at least 99%, at least 99.5%, at least 99.8%, at least 99.9%, or 100%, in the subject.

Embodiment 173. The method of embodiment 171 or 172, wherein the B cells are autoreactive B cells.

Embodiment 174. The method of embodiment 173, wherein the B cells are autoreactive B memory cells.

Embodiment 175. The method of any one of embodiments 171-174, wherein the method reduces the production of autoantibodies.

Embodiment 176. A method of repopulating naive B cells in a subject, comprising administering to the subject an effective amount of the multi-specific polypeptide of any one of embodiments 1-153 or the pharmaceutical composition of embodiment 156.

Embodiment 177. The method of embodiment 176, wherein the subject has a disease or disorder related to B cells.

Embodiment 178. The method of embodiment 176 or 177, wherein the method increases the number of naive B cells by at least 50%, at least 55%, at least 60%, at least 65%, at least 70%, at least 75%, at least 80%, at least 85%, at least 90%, at least 95%, at least 98%, at least 99%, or 100%, in the subject.

Embodiment 179. A method of treating a disease or disorder in a subject in need thereof, comprising administering to the subject an effective amount of the multi-specific polypeptide of any one of embodiments 1-153 or the pharmaceutical composition of embodiment 156.

Embodiment 180. The method of embodiment 179, wherein the disease or disorder is a cancer.

Embodiment 181. The method of embodiment 180, wherein the cancer is a blood cancer.

Embodiment 182. The method of embodiment 181, wherein the blood cancer is chronic lymphocytic leukemia or non-Hodgkin lymphoma.

Embodiment 183. The method of embodiment 179, wherein the disease or disorder is an autoimmune disease.

Embodiment 184. The method of embodiment 183, wherein the autoimmune disease is rheumatoid arthritis or Systemic lupus erythematosus.

Embodiment 185. The method of any one of embodiments 179-184, wherein the subject has previously been treated with a multi-specific polypeptide comprising a first binding domain specifically binds to CD3 instead of TCRβV or TCRαV.

Embodiment 186. The method of any one of embodiments 179-185, wherein the subject is relapsing or refractory to a treatment comprising a multi-specific polypeptide comprising a first binding domain specifically binds to CD3 instead of TCRβV or TCRαV.

Embodiment 187. The method of any one of embodiments 179-186, wherein the subject is administered between about 0.07 mg/kg and 0.17 mg/kg of the multi-specific polypeptide.

Embodiment 188. The method of any one of embodiments 179-187, wherein the administration is intravenous, intraperitoneal, or subcutaneous.

### 2. Examples

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 2.1. Example 1: ELISA Binding Studies of T Cell Engagers (TCEs)

Methods: Various T cell engagers were evaluated for their ability to bind human or cynomolgus monkey cognate antigens, CD19, CD3, or TRBV in a standard enzyme linked immunosorbent assay (ELISA) format. First, soluble antigen proteins were either directly coated on high binding ELISA plates or biotinylated antigens were captured on neutravidin coated plates. T cell engagers diluted in buffer were then added to the antigen coated plates. Bound T cell engager was detected using a standard horse radish peroxidase conjugate secondary antibody. The concentration of each T cell engager required to achieve 50% maximal signal (EC50) was calculated using GraphPad Prism software. The results are shown in Table 19 below:

TABLE 19
EC50s of ELISA Binding Assays

| | Human | Human CD3 |
|---|---|---|
| T cell | CD19 | or TRBV |
| engager | ELISA EC50 | ELISA EC50 |
| # | (nM) | (nM) |
| TCE-1 | 0.06 | 0.04 |
| TCE-2 | 0.05 | 0.77 |
| TCE-3 | 0.04 | 1.29 |
| TCE-4 | 0.11 | |
| TCE-5 | 0.56 | 0.04 |
| TCE-6 | 0.62 | 0.55 |
| TCE-7 | 1.62 | 0.69 |
| TCE-8 | 2.5 | |
| TCE-9 | 0.22 | 0.07 |
| TCE-10 | 0.34 | 0.1 |
| TCE-11 | 0.3 | 0.73 |
| TCE-12 | 0.46 | 1 |
| TCE-13 | 0.26 | 4.39 |
| TCE-14 | 0.29 | 11.89 |
| TCE-15 | 0.18 | |
| TCE-16 | 0.88 | |
| TCE-17 | 7.29 | 0.19 |
| TCE-18 | 38.53 | 0.32 |
| TCE-19 | 7.16 | 1.66 |
| TCE-20 | 18.5 | 3.55 |
| TCE-21 | 9.13 | 6.87 |
| TCE-22 | 58.22 | 5.77 |
| TCE-23 | | |
| TCE-24 | | |
| TCE-25 | | |
| TCE-26 | | |
| TCE-27 | | |
| TCE-28 | 0.04 | 3.72 |
| TCE-29 | 0.07 | 1979 |
| TCE-30 | 0.05 | 1695 |
| TCE-31 | 0.05 | 1063 |
| TCE-32 | 0.04 | 1284 |
| TCE-33 | 0.05 | 6374 |
| TCE-34 | 1.42 | 1.67 |
| TCE-35 | 1.97 | 7.2 |
| TCE-36 | 1.63 | 8.16 |
| TCE-37 | 3.33 | 6.01 |
| TCE-38 | 1.81 | 291.1 |
| TCE-39 | 1.29 | 288.5 |
| TCE-40 | 0.04 | 17.42 |
| TCE-41 | 0.07 | 20.74 |
| TCE-42 | 0.14 | 25.17 |
| TCE-43 | 0.14 | 19.27 |
| TCE-44 | 0.06 | 25.93 |
| TCE-45 | 0.05 | 30.63 |
| TCE-46 | 2.03 | 0.94 |
| TCE-47 | 1.9 | 2.2 |
| TCE-48 | 1.64 | 2.37 |
| TCE-49 | 1.99 | 3.4 |
| TCE-50 | 2.09 | 2.53 |
| TCE-51 | 1.39 | 1.53 |
| TCE-52 | | |
| TCE-53 | 0.12 | 3.49 |
| TCE-54 | | |
| TCE-55 | 0.08 | 0.22 |
| TCE-56 | 0.06 | 0.89 |
| TCE-57 | 0.05 | 0.08 |
| TCE-58 | | |
| TCE-59 | 0.02 | 0.04 |
| TCE-60 | 0.03 | 0.6 |
| TCE-61 | 0.03 | 0.07 |
| TCE-62 | | |
| TCE-63 | 0.03 | 0.05 |
| TCE-64 | 0.2 | 0.78 |
| TCE-65 | 0.11 | 0.06 |
| TCE-66 | | |
| TCE-67 | 0.08 | 0.05 |
| TCE-68 | 0.08 | 0.3 |
| TCE-69 | 0.08 | 0.04 |
| TCE-70 | | |
| TCE-71 | 0.07 | 0.04 |
| TCE-72 | 0.06 | 0.55 |
| TCE-73 | 0.07 | 0.1 |
| TCE-74 | | |
| TCE-75 | 0.02 | 0.03 |
| TCE-76 | 0.07 | 0.59 |
| TCE-77 | 0.07 | 0.12 |
| TCE-78 | | |
| TCE-79 | 0.08 | 0.09 |
| TCE-80 | 0.15 | 2.27 |
| TCE-81 | 4.13 | 419.9 |
| TCE-82 | 0.29 | 1.45 |
| TCE-83 | 0.46 | 4.56 |
| TCE-84 | 0.12 | 0.25 |
| TCE-85 | 0.35 | 1.97 |
| TCE-86 | 0.34 | 0.22 |
| TCE-87 | 0.38 | 0.4 |
| TCE-88 | 0.08 | 0.14 |
| TCE-89 | 0.07 | 0.08 |
| TCE-90 | 0.18 | 0.16 |
| TCE-91 | 0.2 | 0.24 |
| TCE-92 | 3.09 | 8.99 |
| TCE-93 | 0.24 | 1.11 |
| TCE-94 | 0.15 | 0.4 |
| TCE-95 | 0.22 | 0.41 |
| TCE-96 | 0.1 | 15.97 |
| TCE-97 | 0.14 | 5.84 |
| TCE-98 | 0.27 | 0.96 |
| TCE-99 | 0.13 | 0.75 |
| TCE-100 | 0.32 | 0.56 |
| TCE-101 | | |
| TCE-102 | 0.12 | 13.45 |
| TCE-103 | 0.16 | 8.47 |
| TCE-104 | 0.17 | 1.84 |
| TCE-105 | | |
| TCE-106 | 0.17 | 3062 |
| TCE-107 | 0.14 | 2343 |
| TCE-108 | | |
| TCE-109 | | |
| TCE-110 | 0.14 | 0.38 |
| TCE-111 | 0.12 | 0.89 |
| TCE-112 | 0.18 | 10640 |
| TCE-113 | 0.15 | 433.4 |
| TCE-114 | 0.14 | 0.47 |
| TCE-115 | 0.14 | 0.29 |
| TCE-116 | 0.21 | 2.12 |
| TCE-117 | 0.65 | 10.45 |
| TCE-118 | 0.19 | 1.42 |
| TCE-119 | 0.22 | 1.62 |
| TCE-120 | 0.1 | 6.49 |
| TCE-121 | 0.12 | 2.75 |

### 2.2. Example 2: T Cell Engagers Binding to TRBV or TRAV Induce More Robust Expansion of Target T Cells Compared to T-Cell Engagers Binding to CD3 in Healthy Donor PBMCs

Methods: T cell engagers were evaluated in flow cytometry based functional ex vivo assays using human or cynomolgus monkey PBMCs that comprise unmanipulated physiologic ratios of T cells and B cells. Titrated T cell engagers were incubated with freshly thawed 250,000 human or cynomolgus monkey PBMCs per well for up to 7 days. Readouts were taken on days 1, 3, 5, and/or 7 as well as pre-treatment (day 0). At the indicated timepoints supernatants were harvested for cytokine measurements and cells were harvested for flow cytometry measurements. Supernatants from human PBMCs were analyzed for cytokines using a Meso Scale Device kit following the manufacturer's instructions. Supernatants from cynomolgus monkey PBMCs were analyzed using a cytometric bead array following the manufacturer's instructions. Flow cytometry measurements included live/dead, CD3, CD8, CD4, Foxp3, TRBV, CD25, Granzyme B, Ki-67, CCR7, CD45RA, CD95, CD20, and/or CD19 in the panel. Counting beads were included to accurately quantify absolute number of cells present in the samples. Flow cytometric readouts were plotted against the logarithmic concentration of the T cell engager. Where indicated the concentration of the T cell engager required to reach half maximum readout was calculated in GraphPad Prism and reported as an EC50. Readouts included T cell expansion (change in absolute number of viable T cells or T cell subsets), T cell activation, as well as killing of B cells (change in viable B cell counts). Fold change in T cell counts was calculated as T cell engager target positive T cells (CD3+ or TRBV+) post T cell engager treatment divided by the analogous T cell counts without treatment. Flow cytometry gating was used to define cell populations as follows, and all viable cells were gated with live/dead stain:

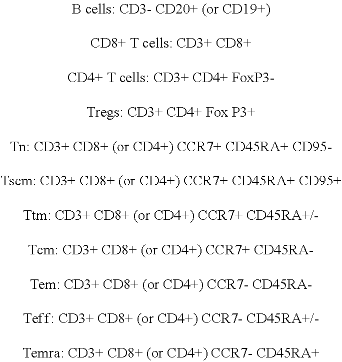

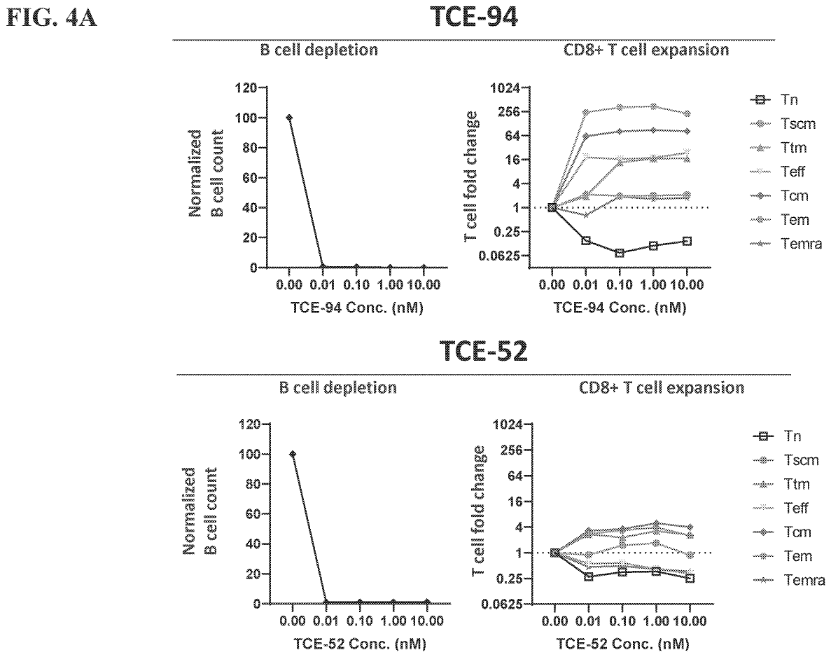

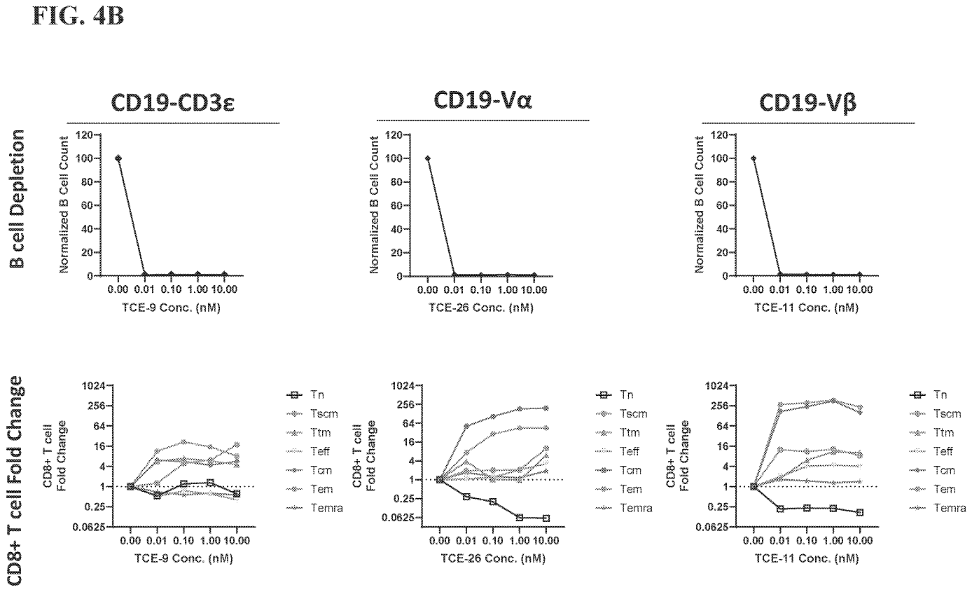

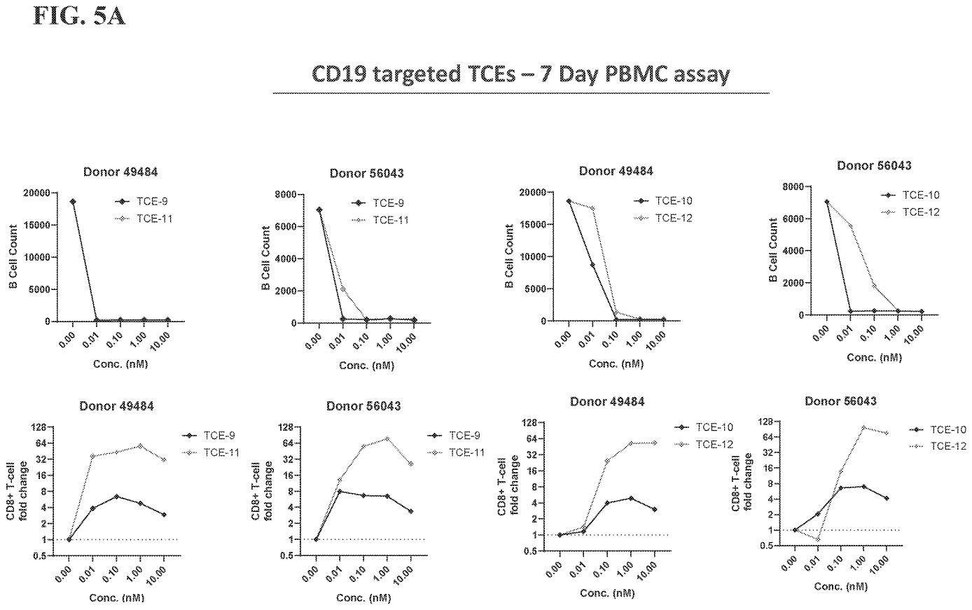

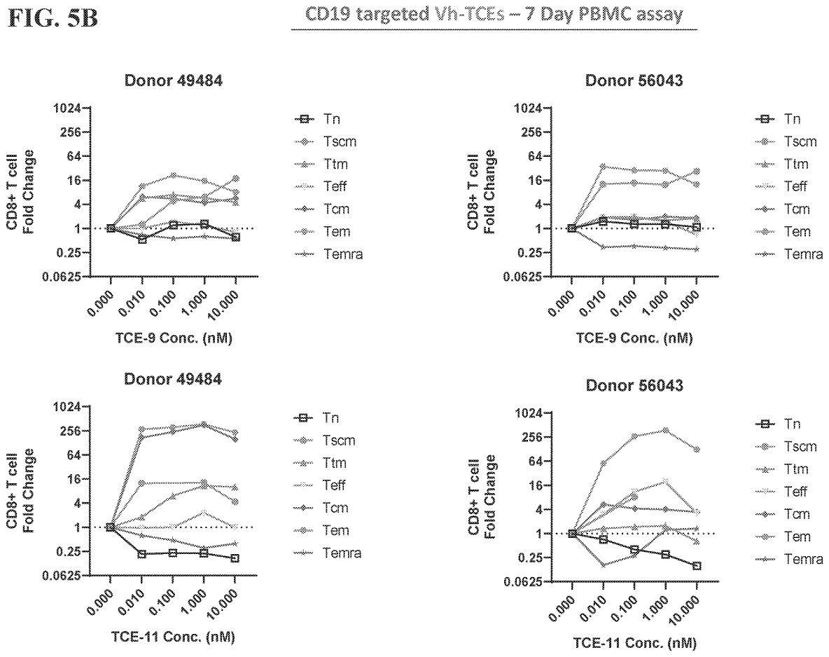

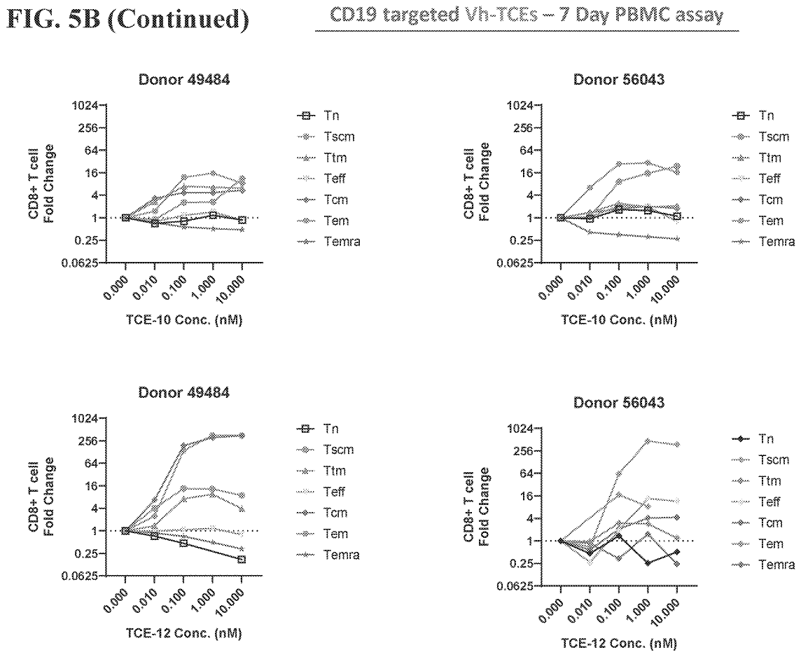

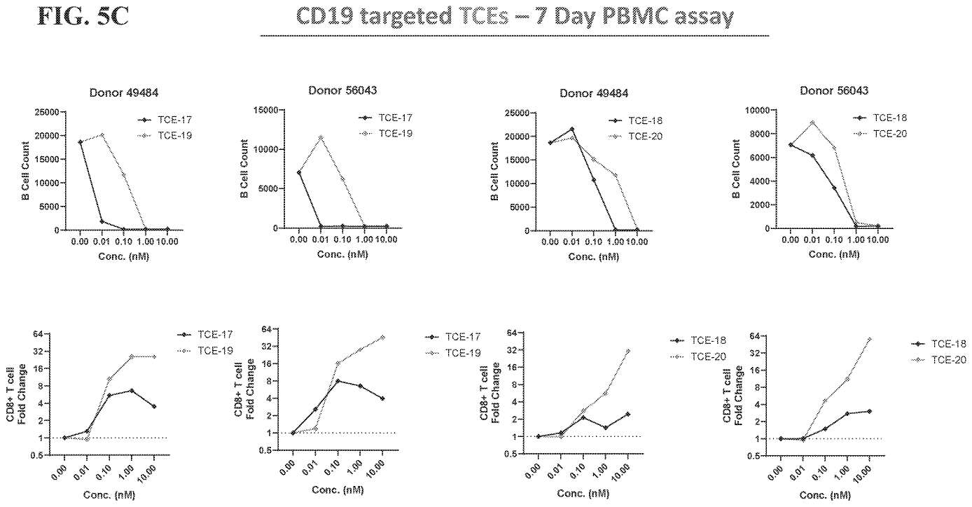

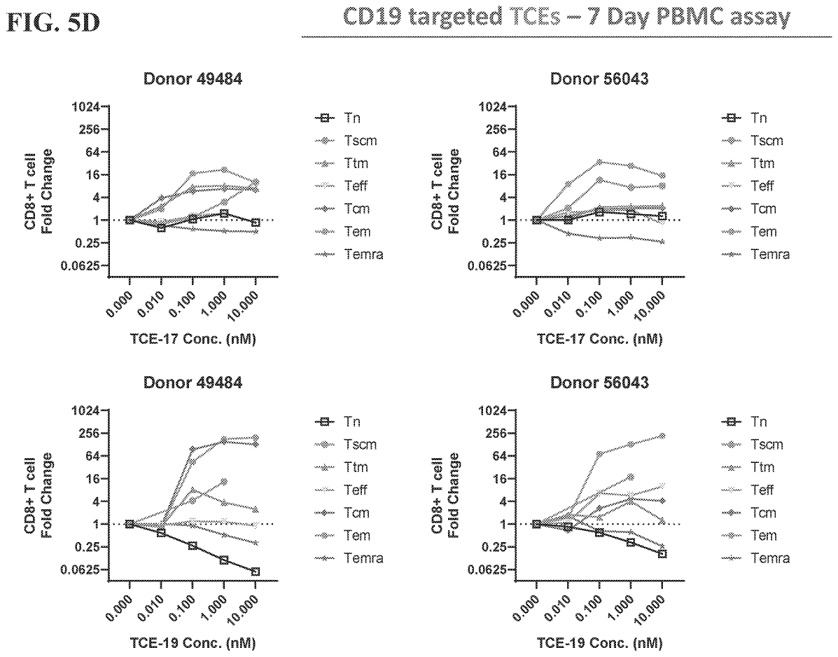

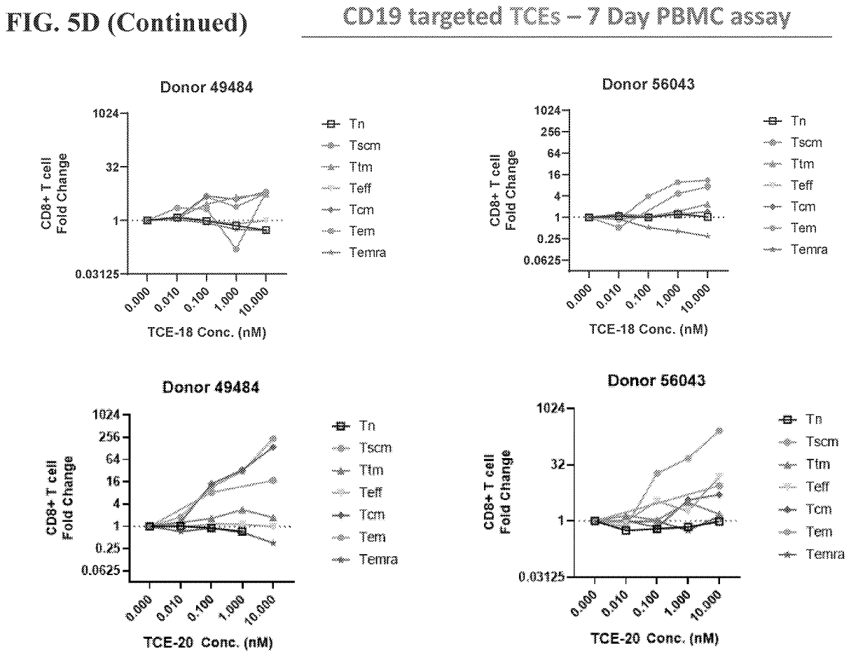

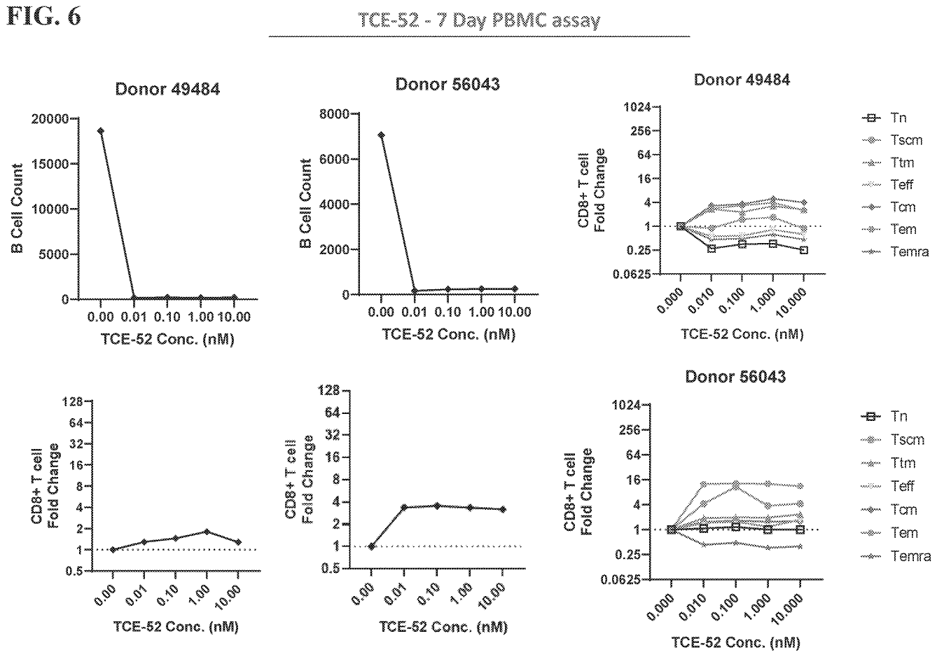

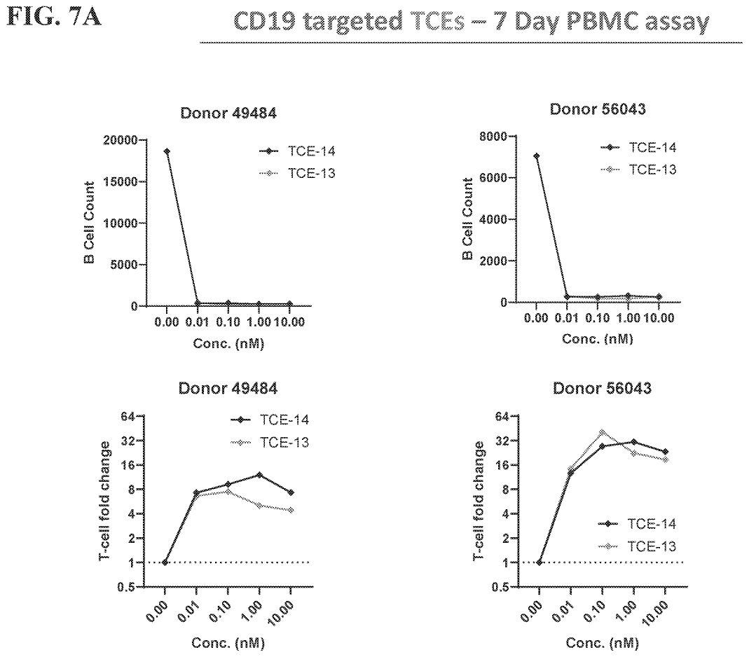

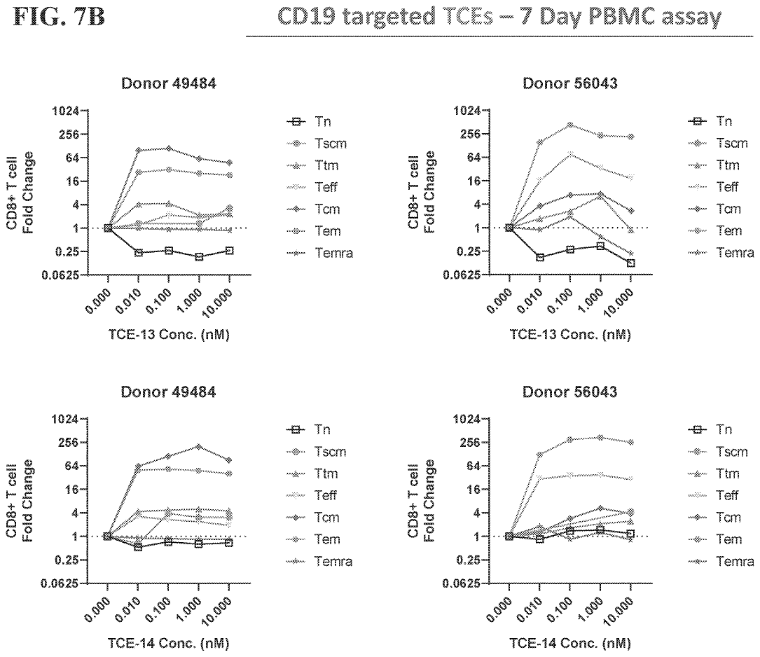

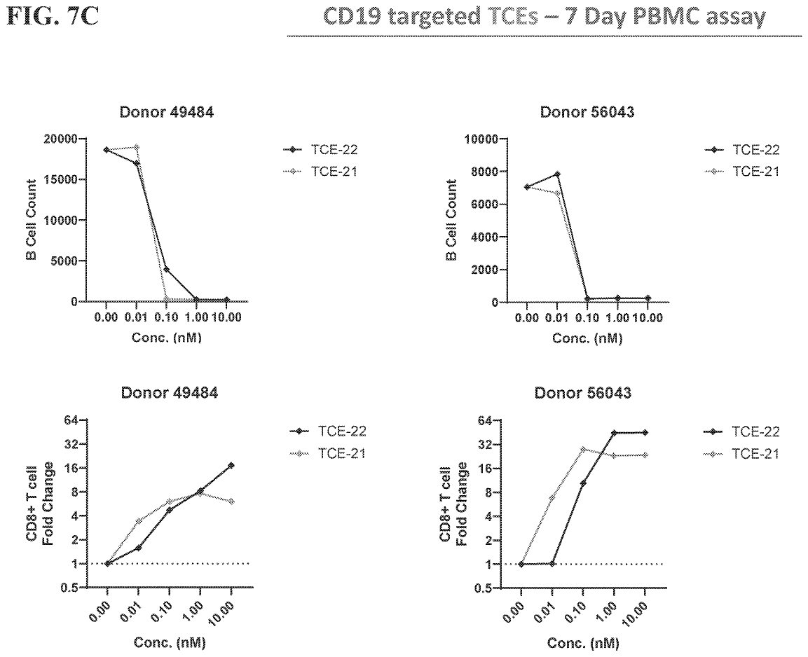

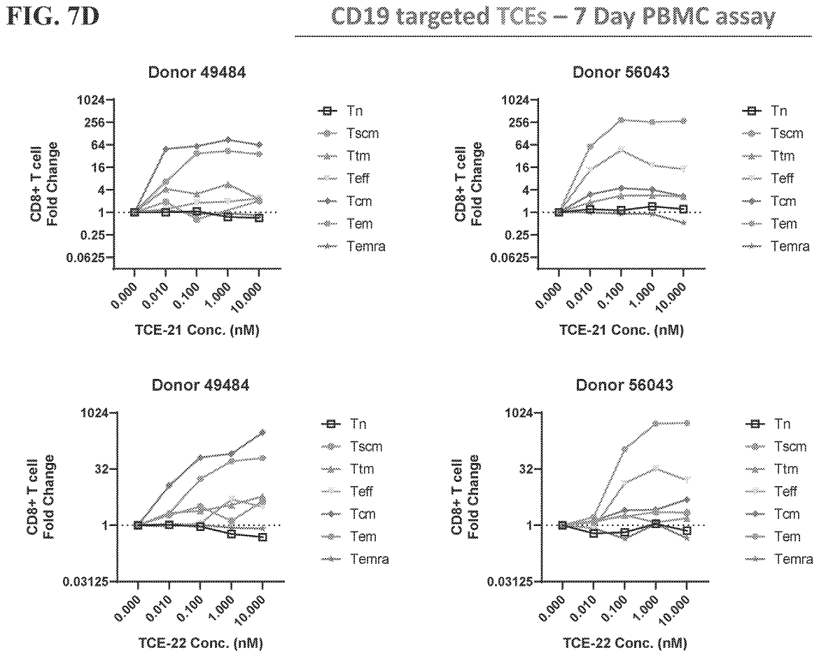

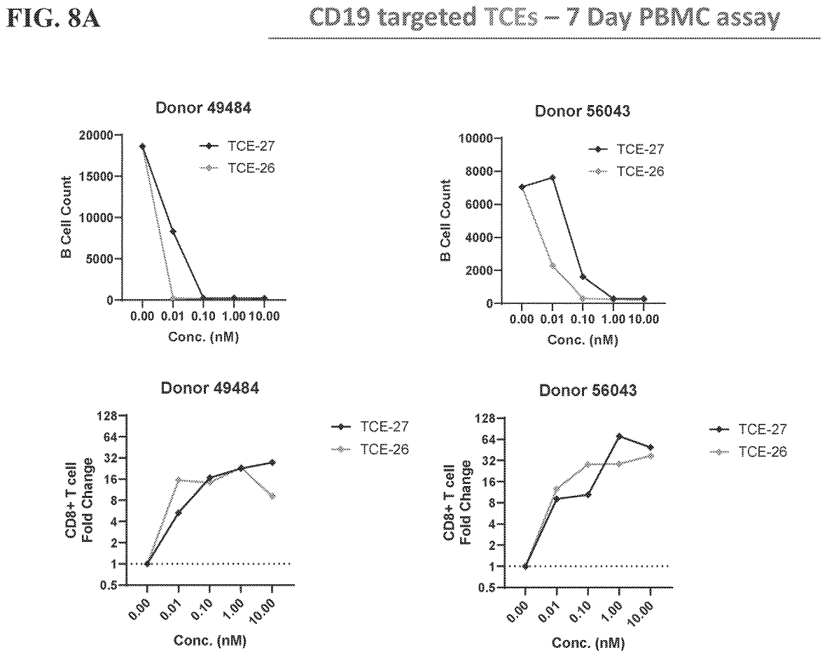

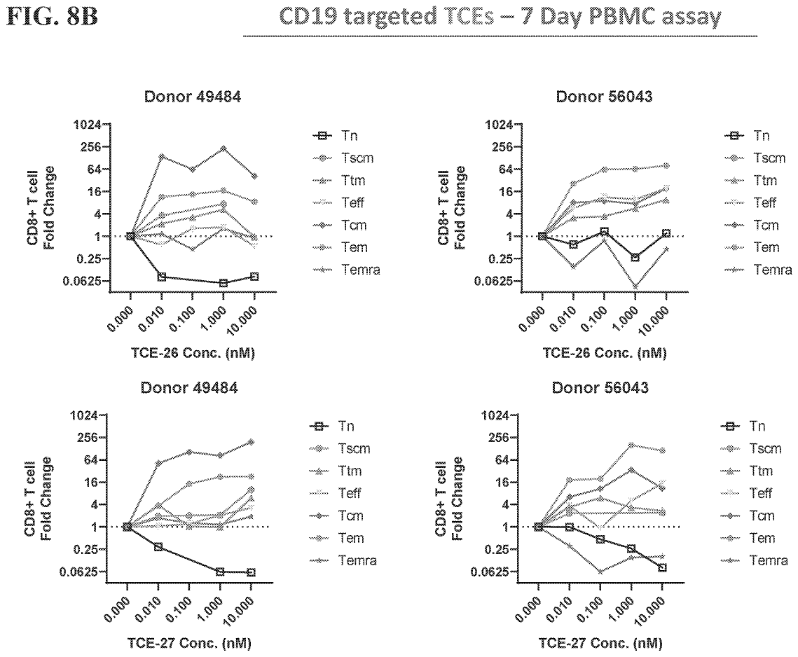

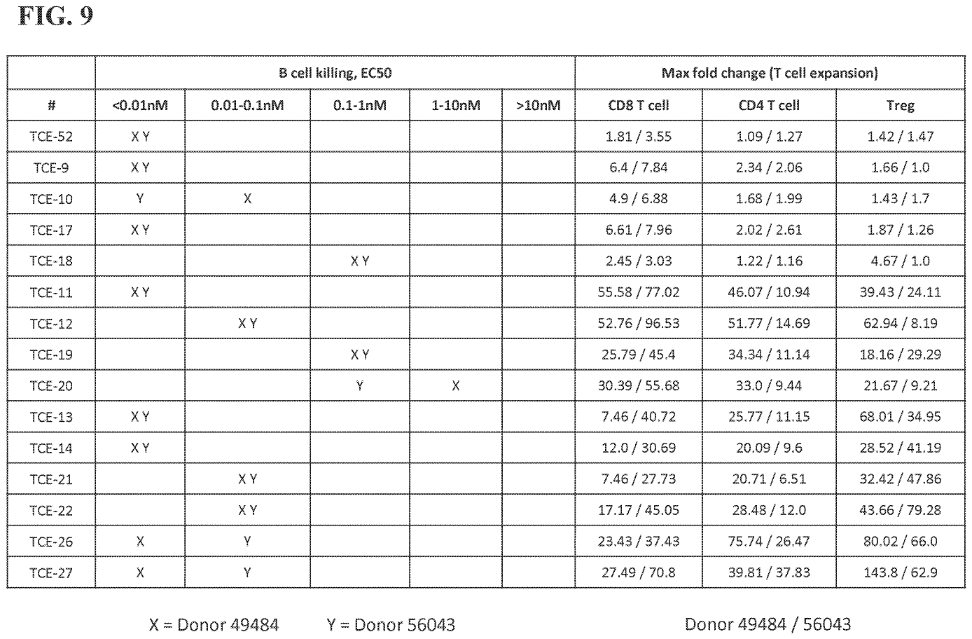

A series of experiments demonstrate that anti-CD19 T-cell engagers binding to TRBV or TRAV can induce more robust expansion of memory and effector T cells compared to T-cell engagers binding to CD3, without compromising the ability to deplete B cells. As shown in FIG. 4A, a T cell engager TCE-94, which specifically binds CD19 and TRBV, expanded memory populations and maintained effector populations with complete B cell depletion. In contrast, Blinatumomab (TCE-52), which specifically binds CD19 and CD3, induced minimal T cell expansion with reduction in effector populations. Likewise, in FIG. 4B, three T cell engagers having an identical CD19 binding domain, but different T cell binding domains, were tested for their abilities to deplete B cells and expand T cells of healthy donor PBMCs. TCE-9, which specifically binds CD38, induced nominal memory population expansion and a reduction in effector populations. In contrast, the other two molecules TCE-26 and TCE-11, which bind the alpha variable domain or the beta variable domain of TCR, respectively, both induced significant expansion of memory cell population while completely depleting B cells. These results indicate that the activities of such T cell engagers depend on the target epitope on the T cells: those with specific binding to the TCR receptor beta or alpha variable domain, instead of CD3, are more effective at inducing memory population expansion that provides a reservoir for effector cell replenishment. Such results are reproduced for alternative formats of T cell engagers. As shown in FIGS. 5A-5D and 6, T cell engagers targeting CD19×CD3 (i.e., TCE-9, TCE-10, TCE-17, TCE-18, and TCE-52) generally induced minimal T cell expansion with reduction in effector population, whereas T cell engagers targeting CD19×TRBV6-5 (i.e., TCE-11, TCE-12, TCE-19, and TCE-20) induced robust expansion of memory and effector T cells. In addition to T cell engagers targeting TRBV6-5, other T cell engagers targeting TRBV12-3/4 (e.g., TCE-13, TCE-14, TCE-21, and TCE-22) or TRAV1-2 (e.g., TCE-26 and TCE-27) also induced B cell depletion and robust memory and effector T cell expansion in healthy human PBMCs, as shown in FIG. 7A-8B. These results are summarized in FIG. 9. Overall, these results demonstrate the superior abilities of TRAV or TRBV targeting T cell engagers to expand memory and effector T cells while depleting B cells.

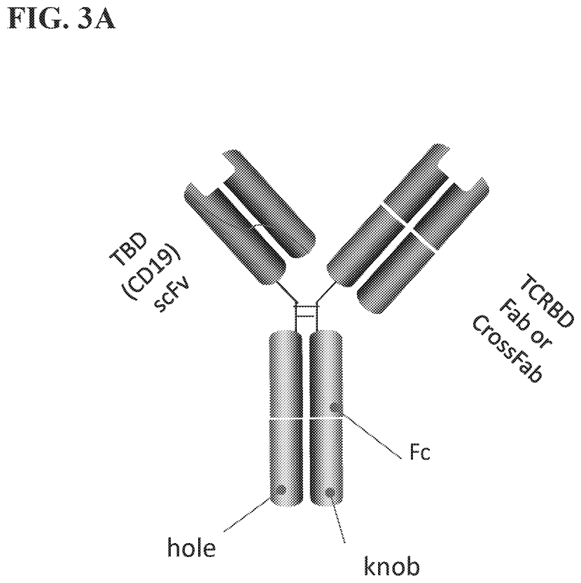

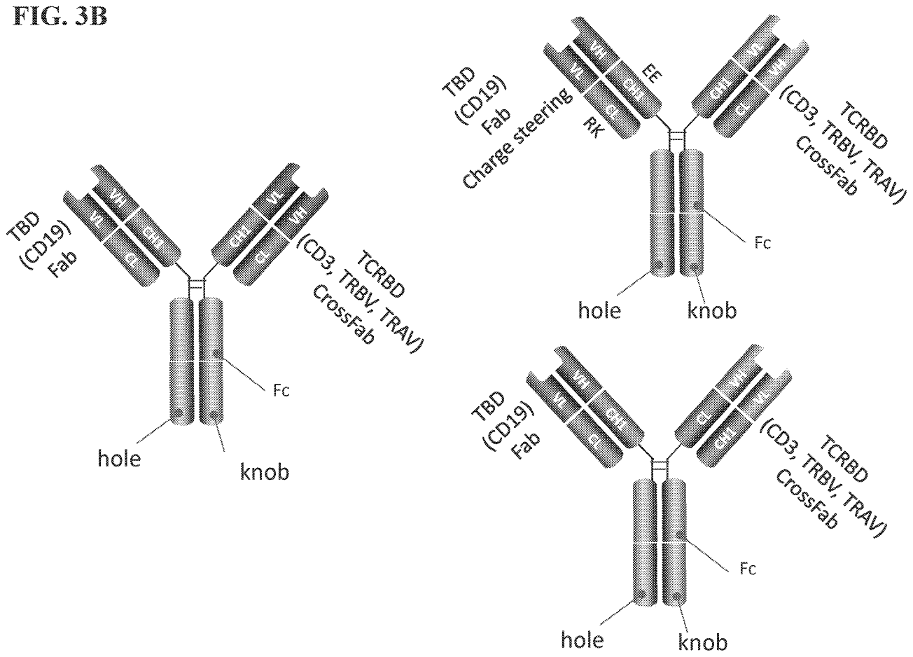

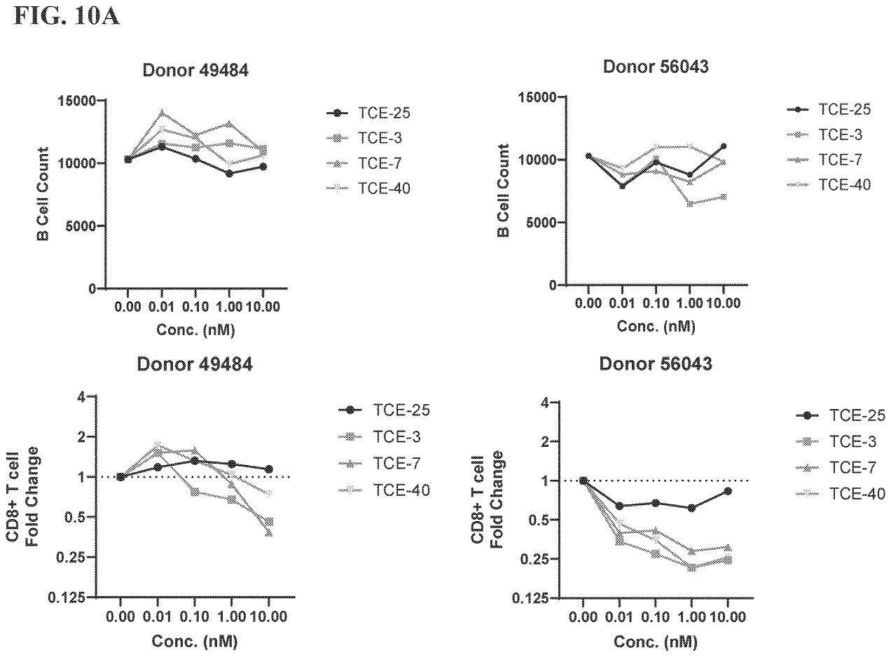

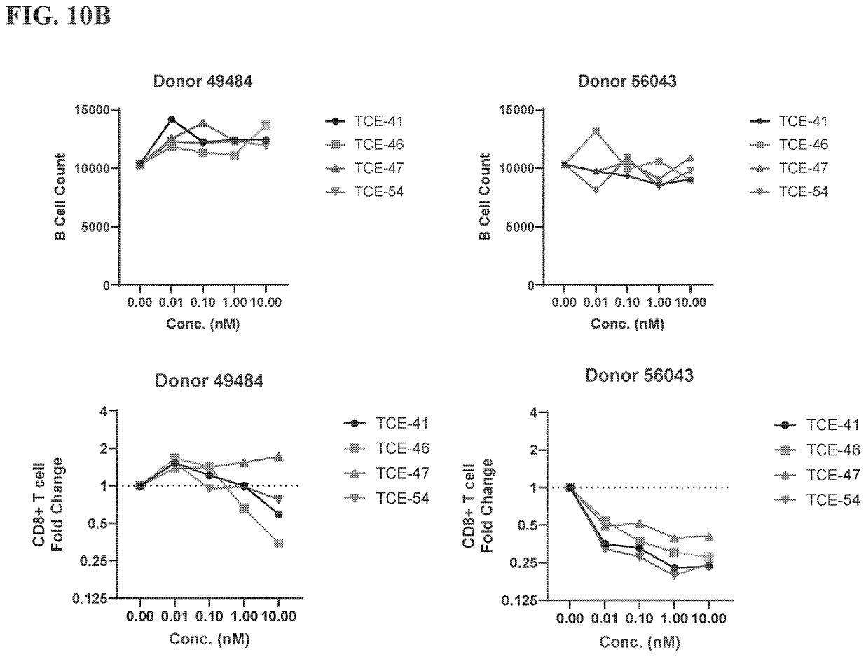

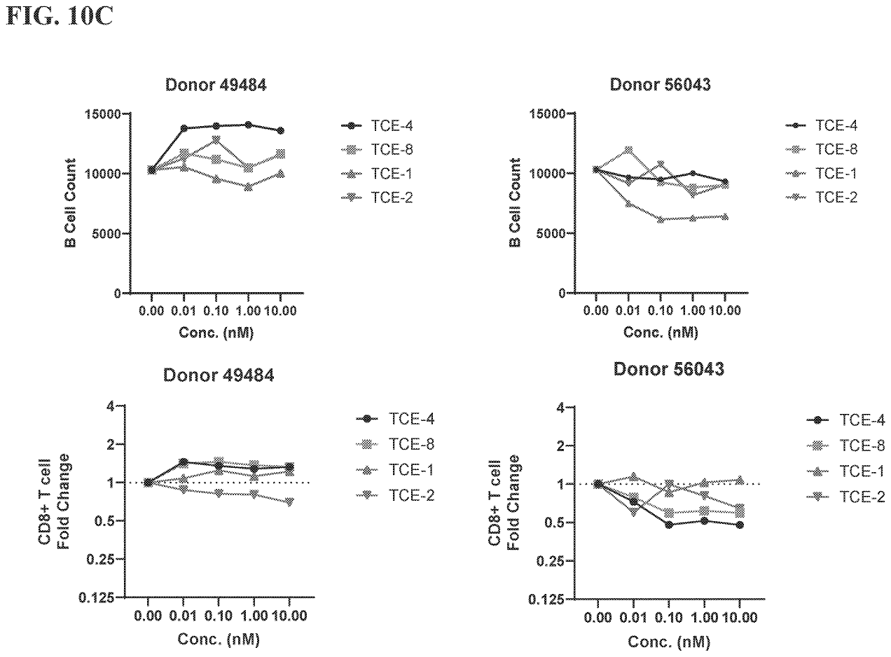

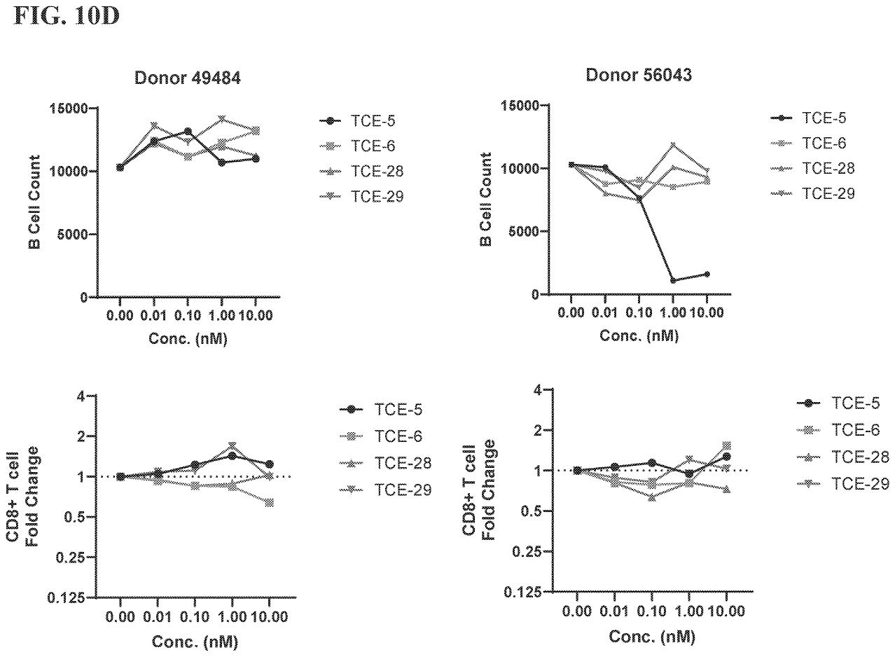

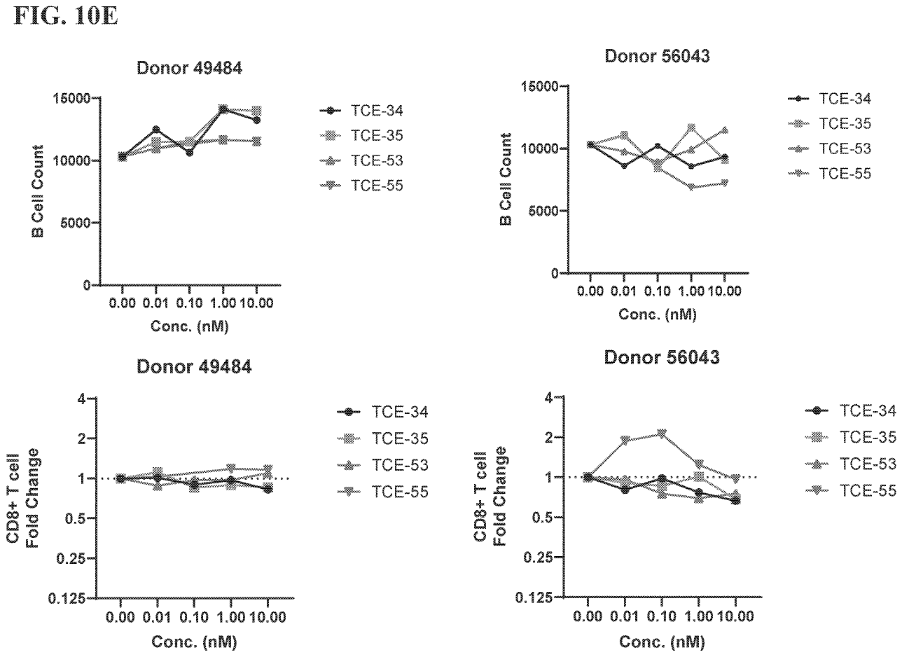

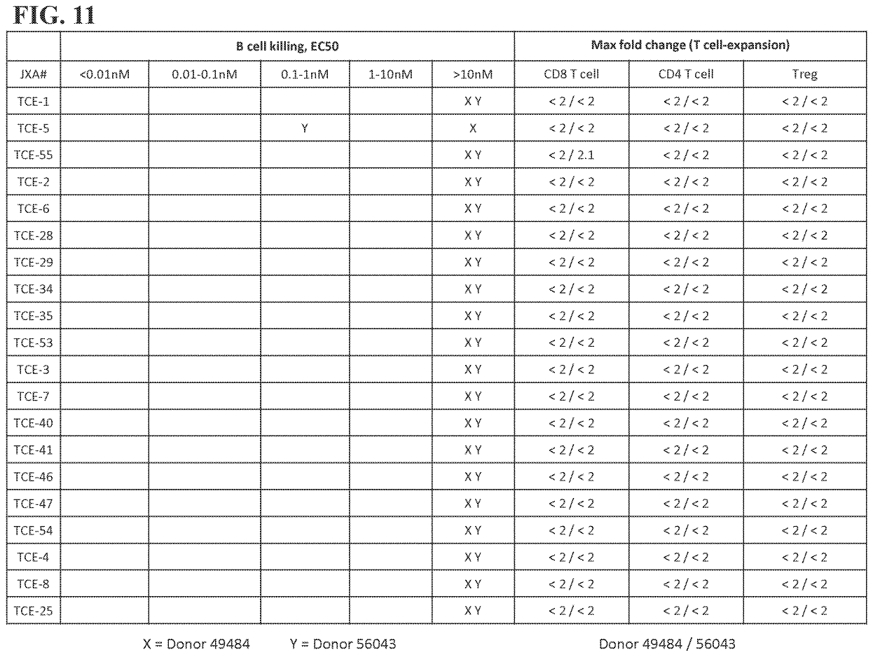

Next, different formats of T cell engagers comprising a half-life extension moiety-a Fc region-were tested for their abilities to deplete B cells and expand T cells. Surprisingly, some formats of “symmetric” T cell engagers (i.e., Y shaped IgG polypeptides comprising a CD19 Fab or CrossFab linked to one Fc chain and a TRBV or TRAV Fab or CrossFab linked to the other Fc chain of FIG. 3B) displayed lower B-cell depletion, as shown in FIG. 10A-10E, despite their abilities to bind to both CD19 and TRBV/TRAV antigens in ELISA. These results are summarized in FIG. 11. Thus, activities of such T cell engagers can be improved by changing how the binding domains are connected to the Fc region.

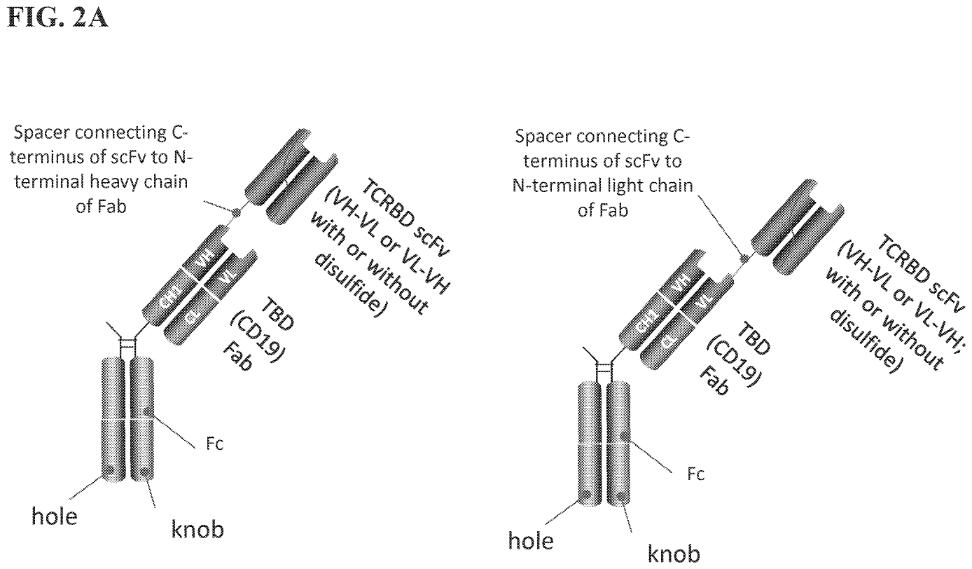

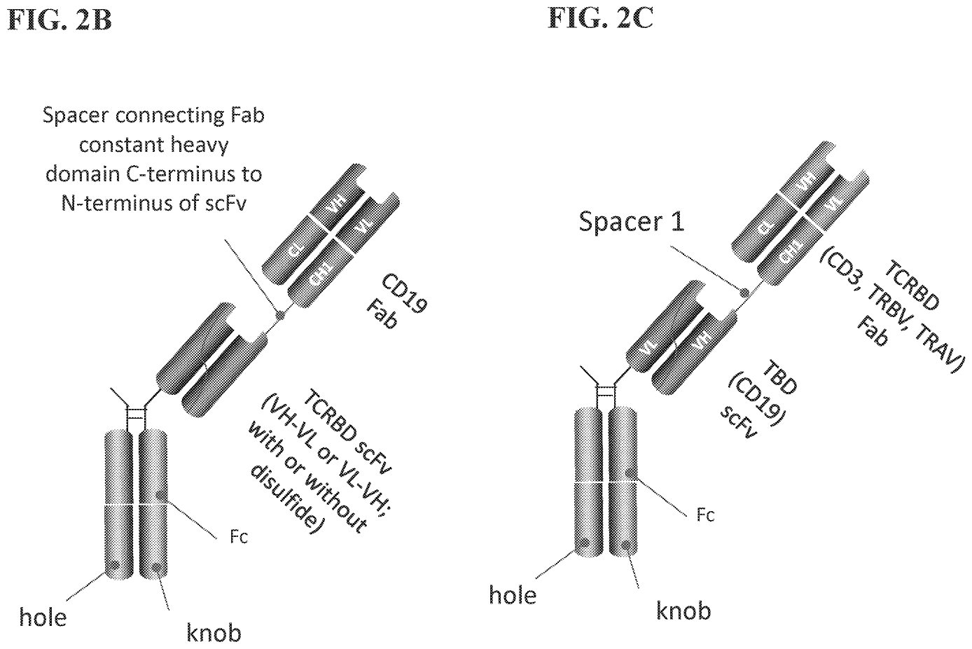

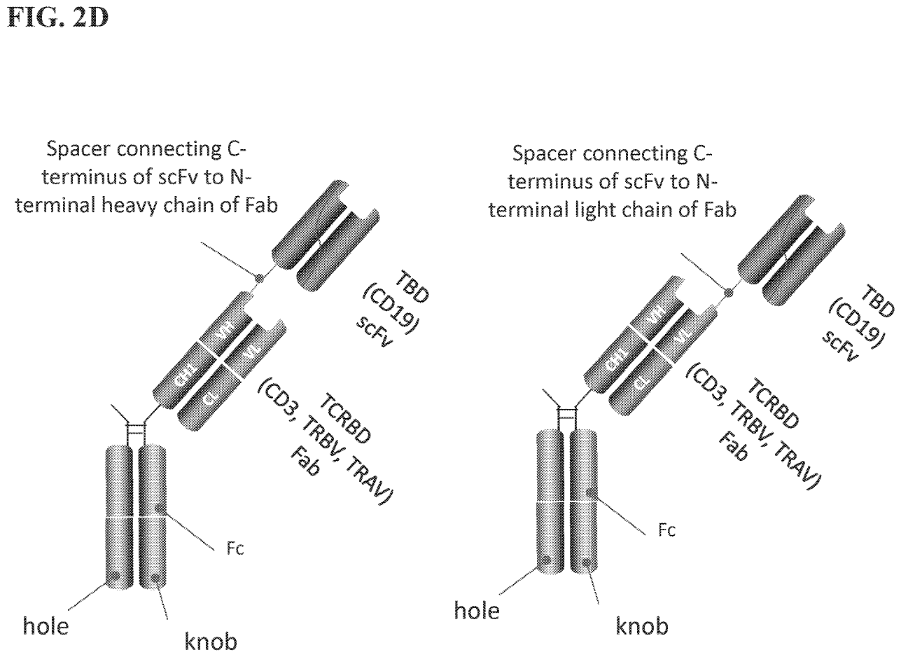

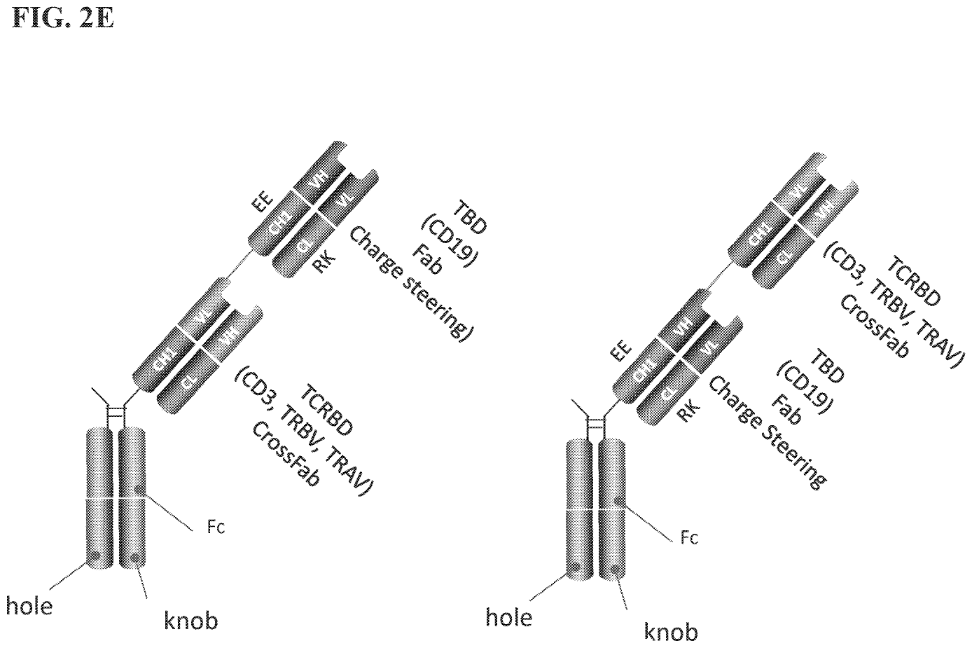

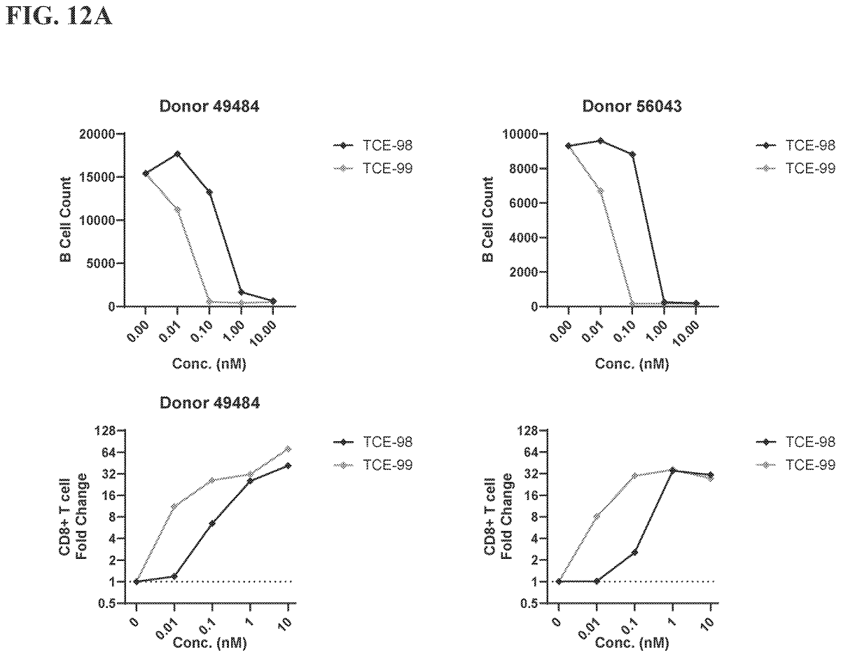

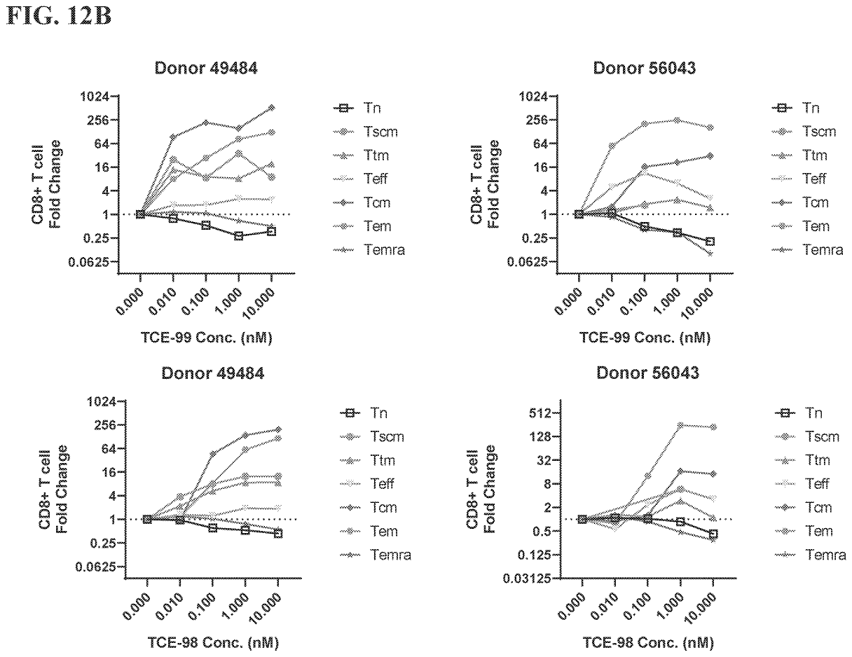

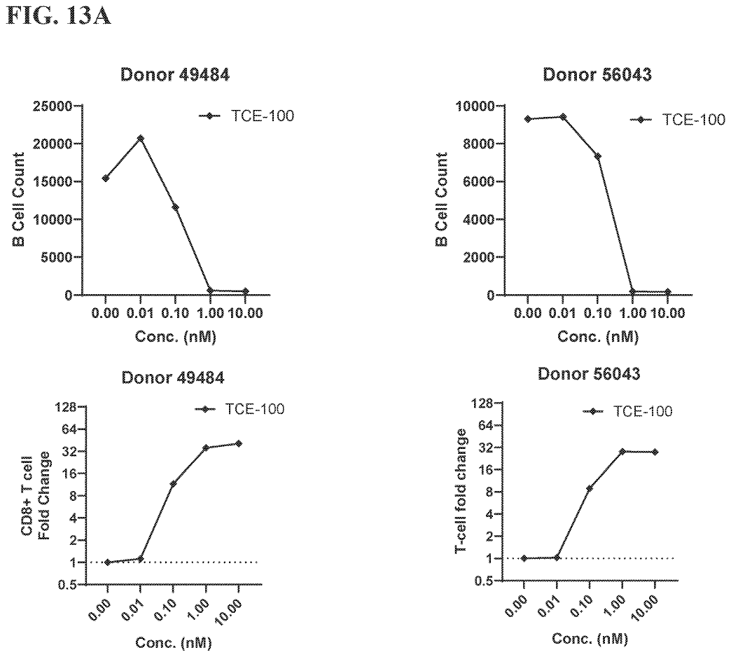

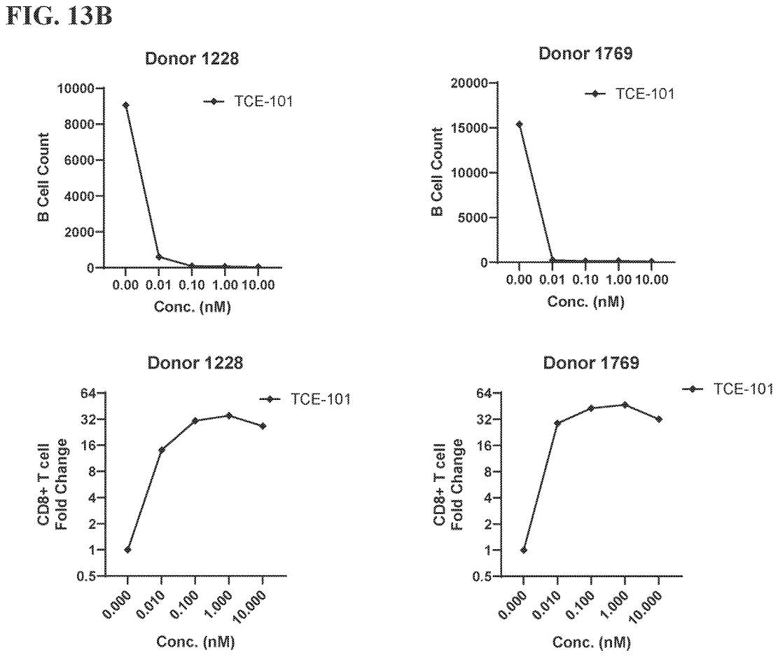

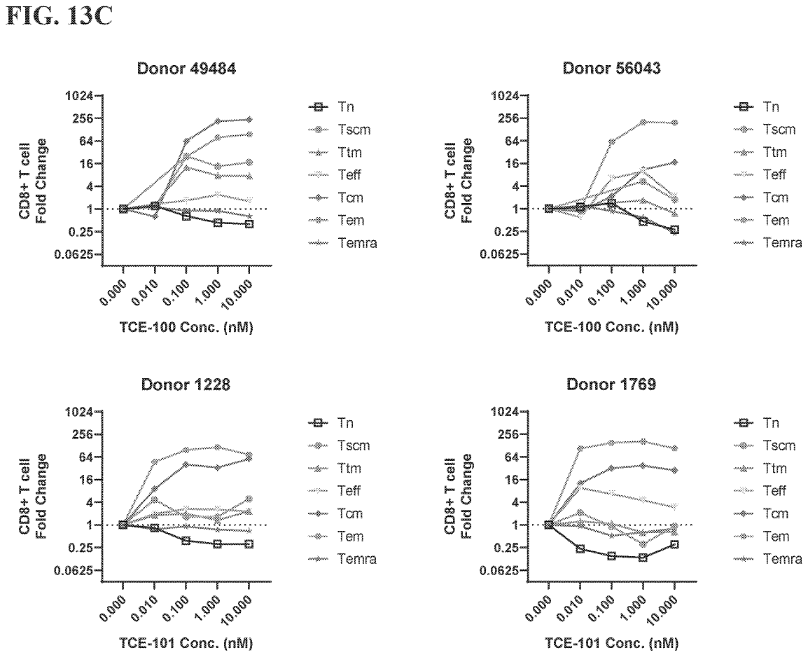

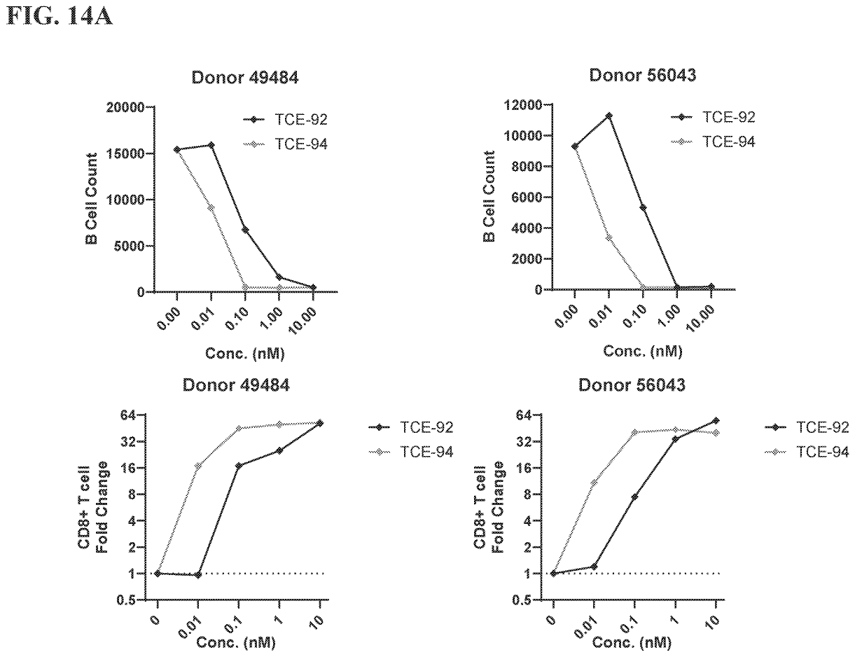

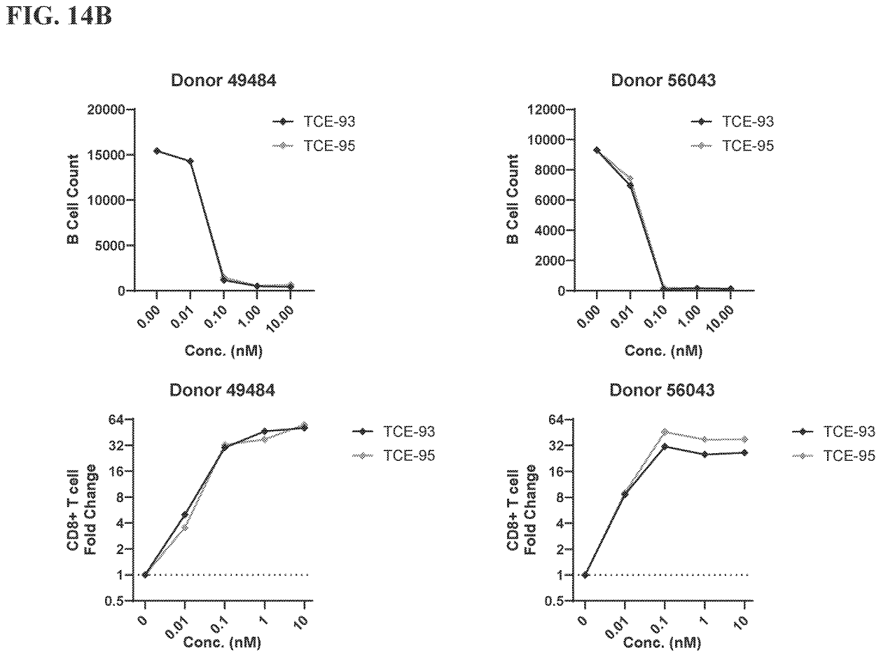

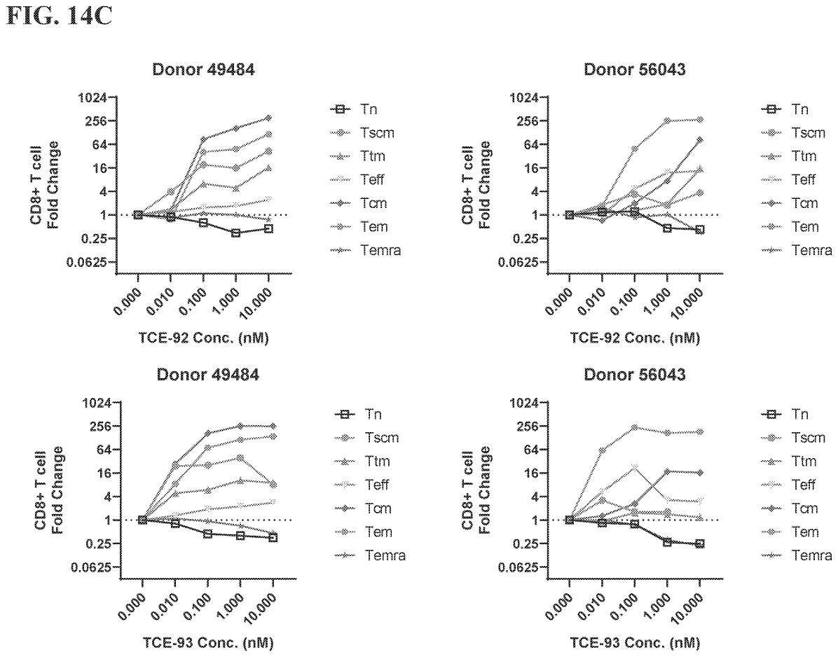

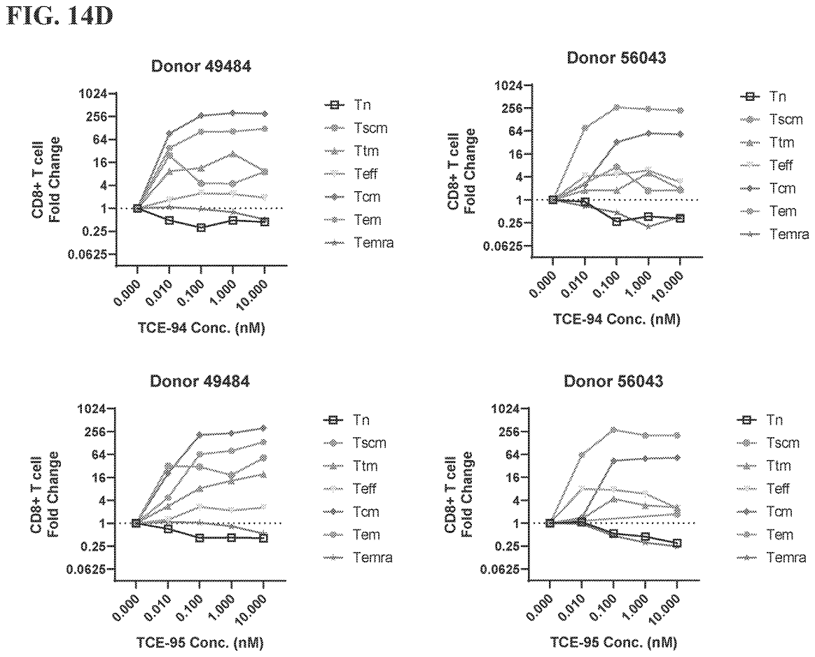

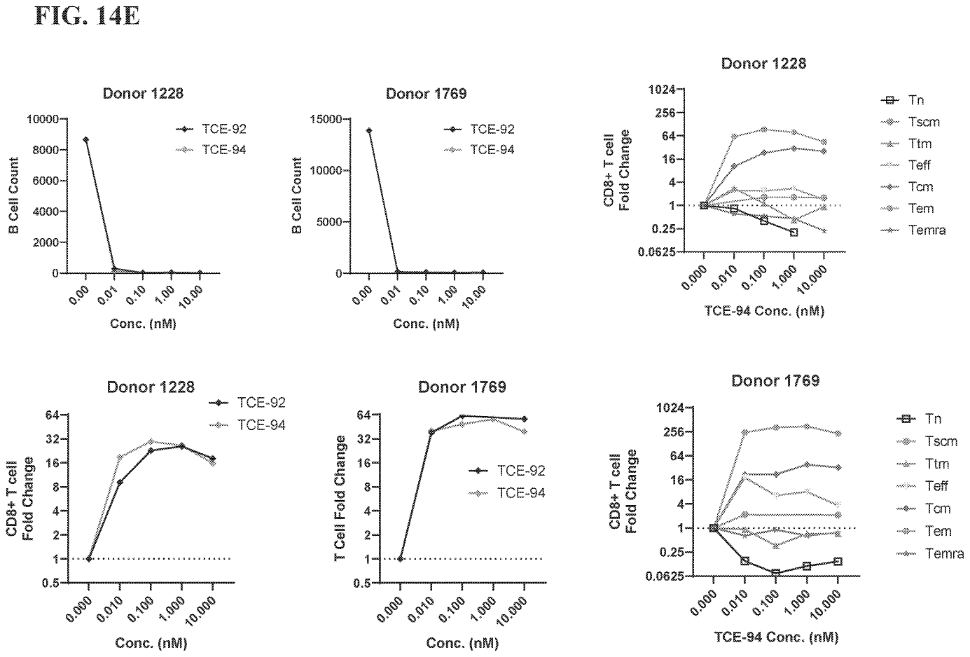

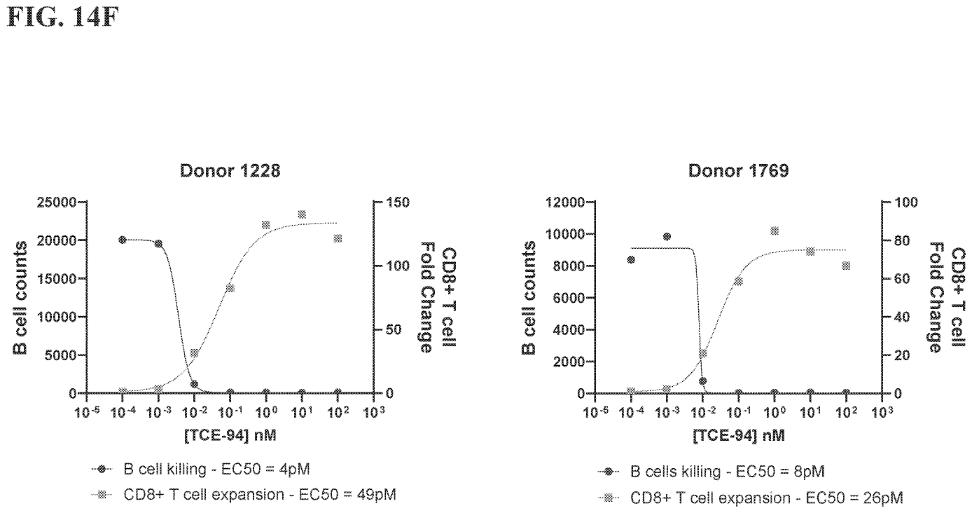

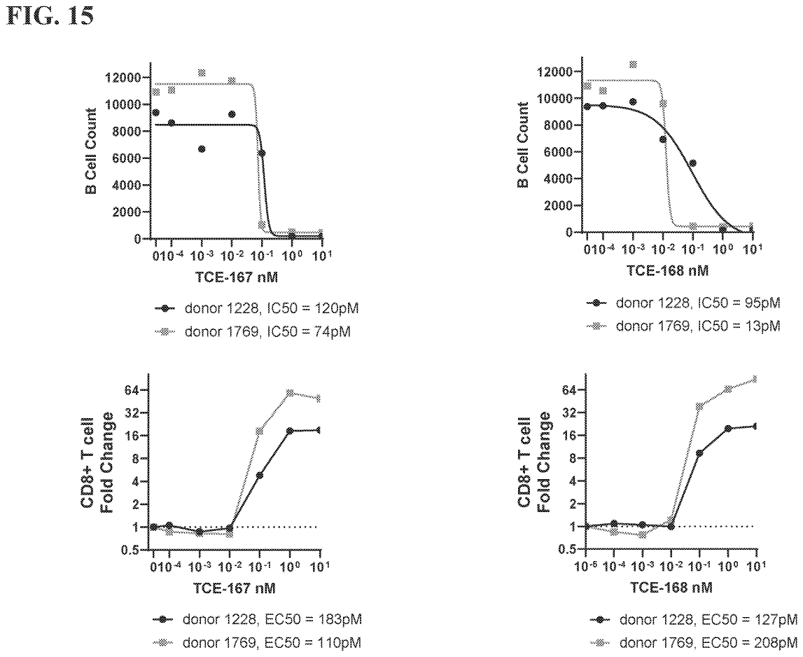

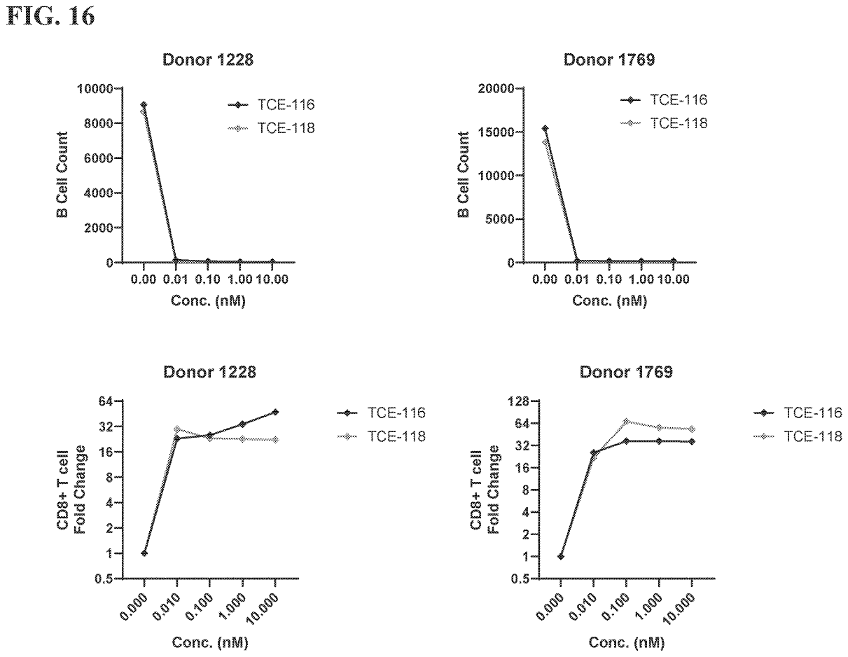

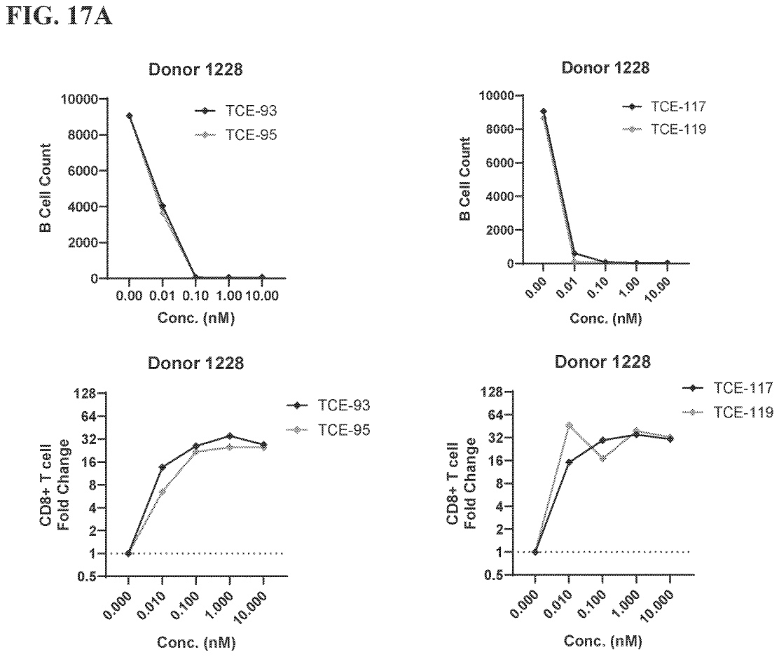

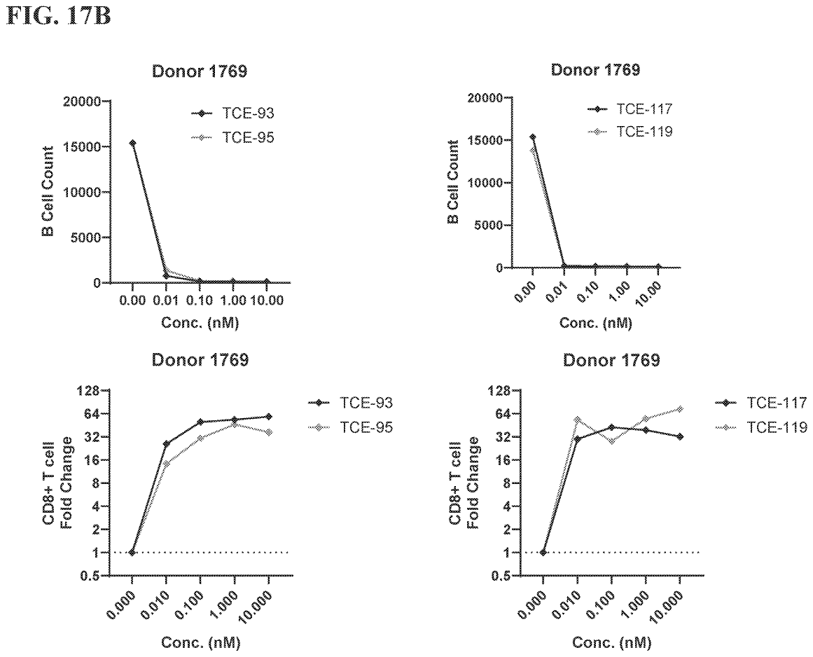

In contrast to the symmetric, Y-shaped T cell engagers above, which are mostly inactive, T cell engagers having both binding domains on the same Fc chain displayed robust activities. In particular, as shown in FIG. 12A-12B, CD19×TRBV6-5 T cell engagers TCE-98 and TCE-99, which adopt the asymmetric formats shown in FIG. 2A, induced potent B-cell depletion and T cell expansion in healthy donor PBMCs. Introducing an engineered disulfide bond (C44:C100) in the anti-TRBV scFv of TCE-98 or TCE-99 further improved the potency of the T cell engagers, as demonstrated by TCE-100 and TCE-101 in FIG. 13A-13C. These results were confirmed with alternative CD19×TRBV6-5 T cell engagers TCE-92 to TCE-95, all of which contain an alternative set of CD19 binding VH/VL that were derived from Tafasitamab, as shown in FIG. 14A-14F. Likewise, TCE-167 and TCE-468, which contain a set of CD19 binding VH/VL derived from denintuzumab, also demonstrated potent B cell depletion and T cell expansion as shown in FIG. 15. The order of VH/VL in the anti-TRBV scFv can be reversed and the resultant T cell engagers retained their potency in B cell depletion and T cell expansion, as demonstrated by TCE-116 and TCE-118 in FIG. 16 (compared with TCE-92 and TCE-94 data, supra), as well as the data comparison between TCE-117/TCE-119 and TCE-93/TCE-95 in FIG. 17A-17B.

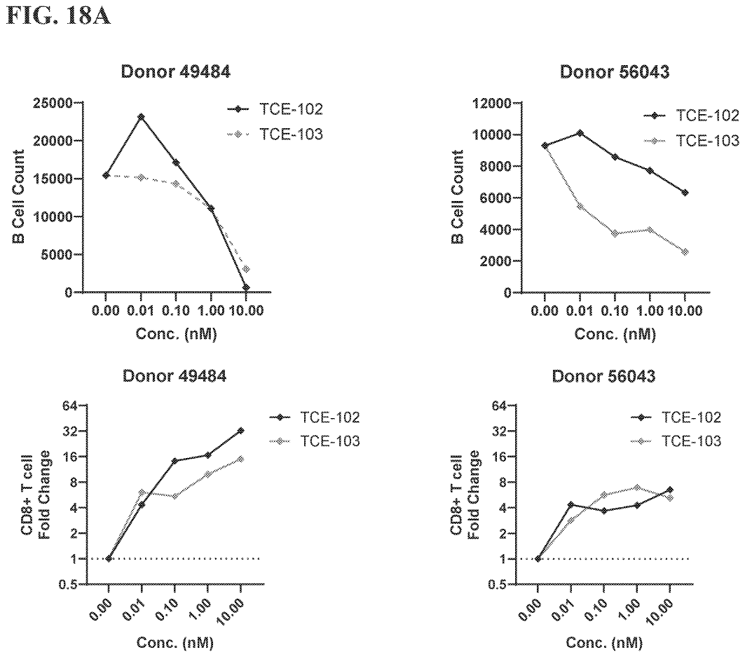

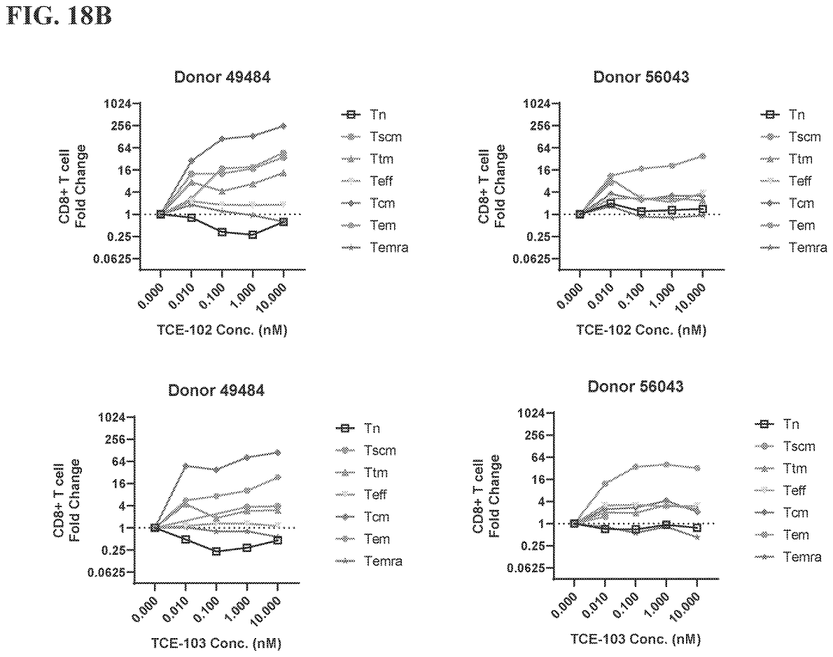

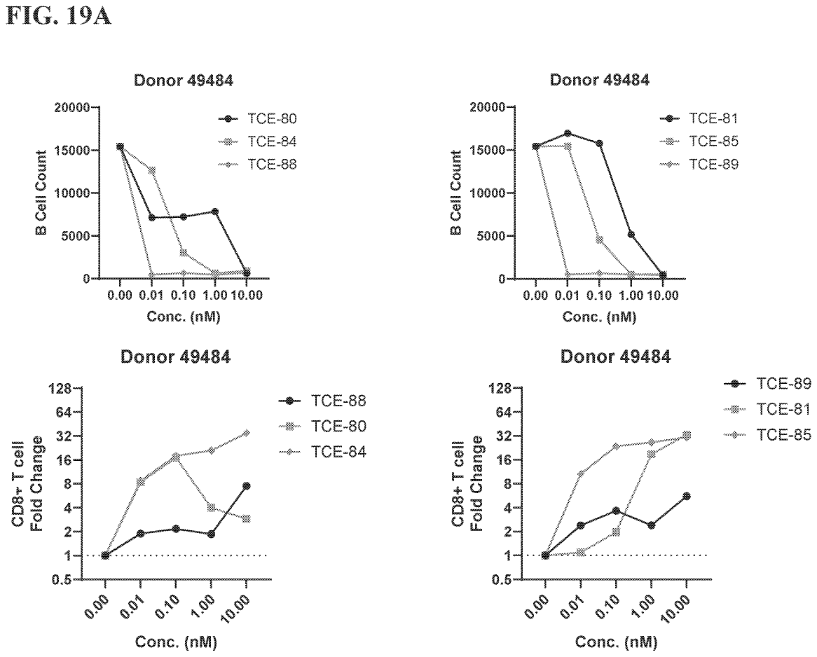

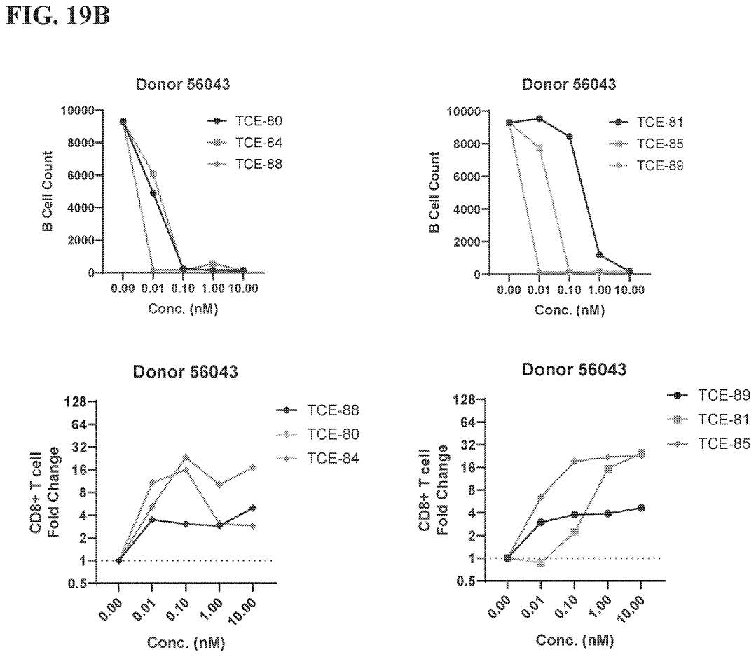

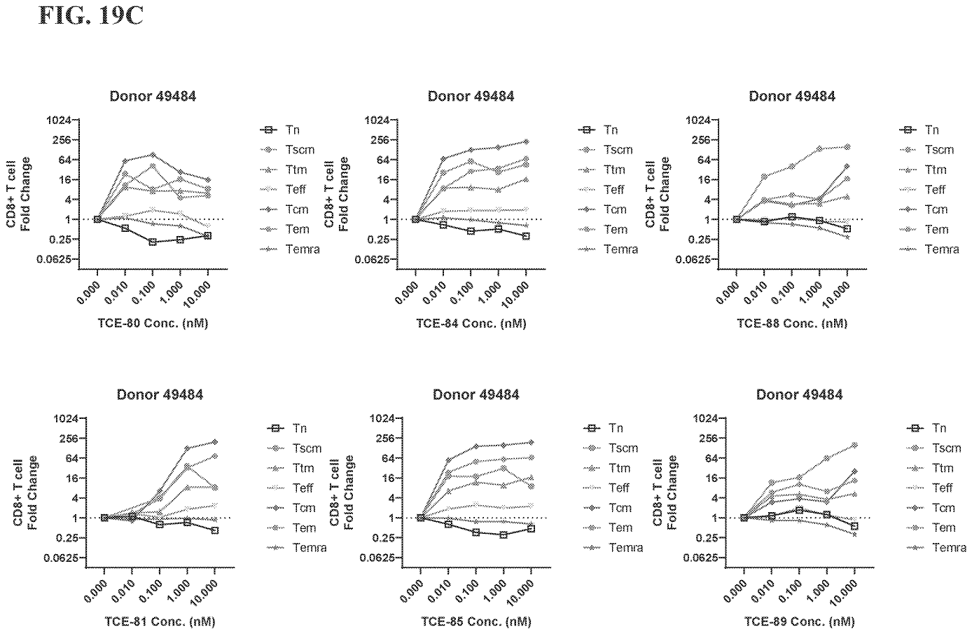

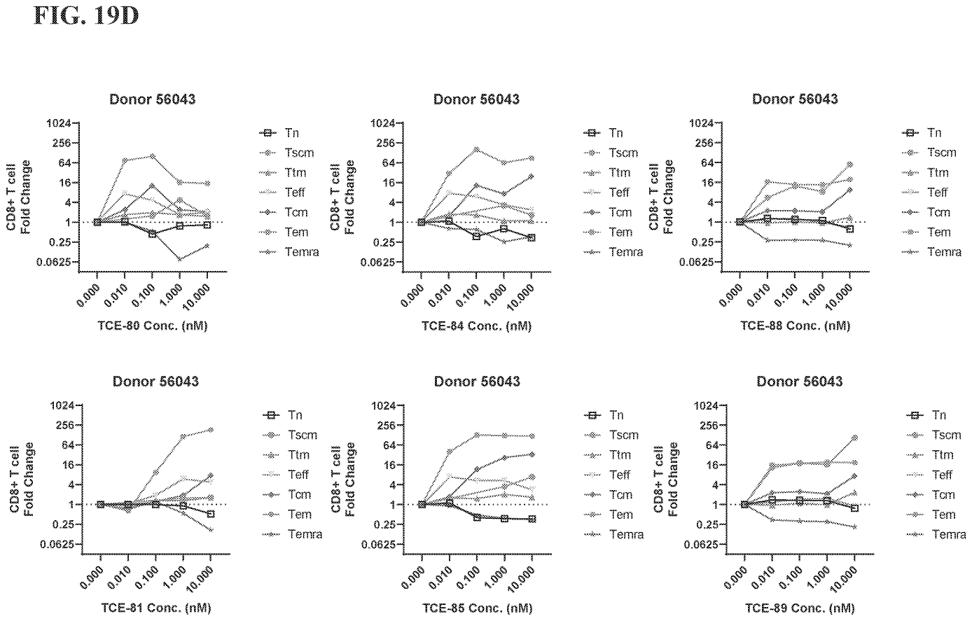

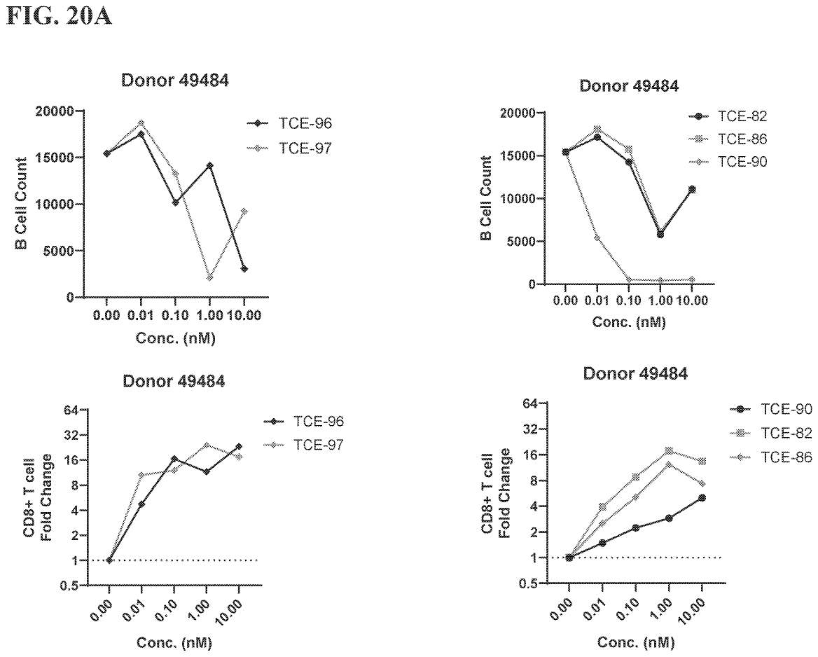

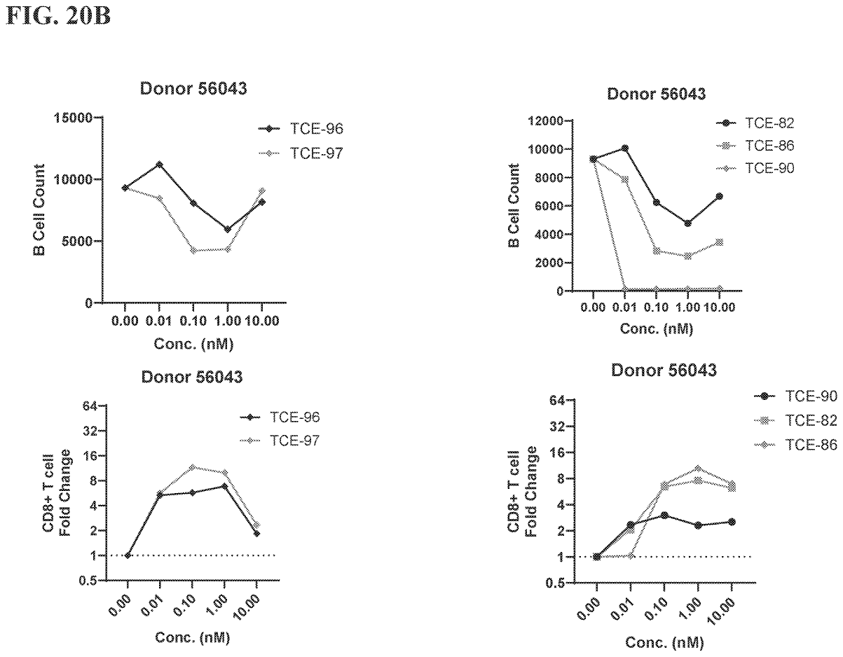

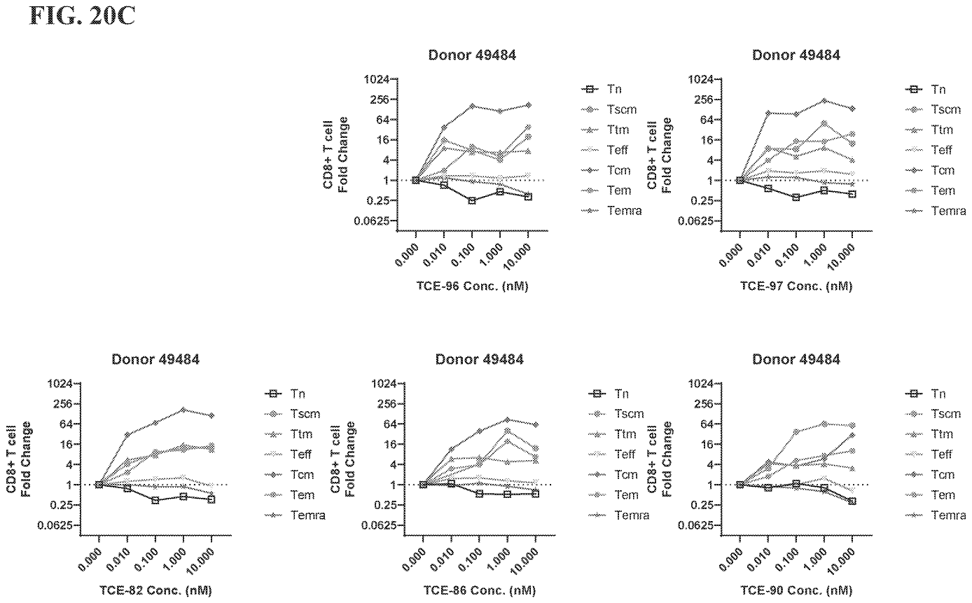

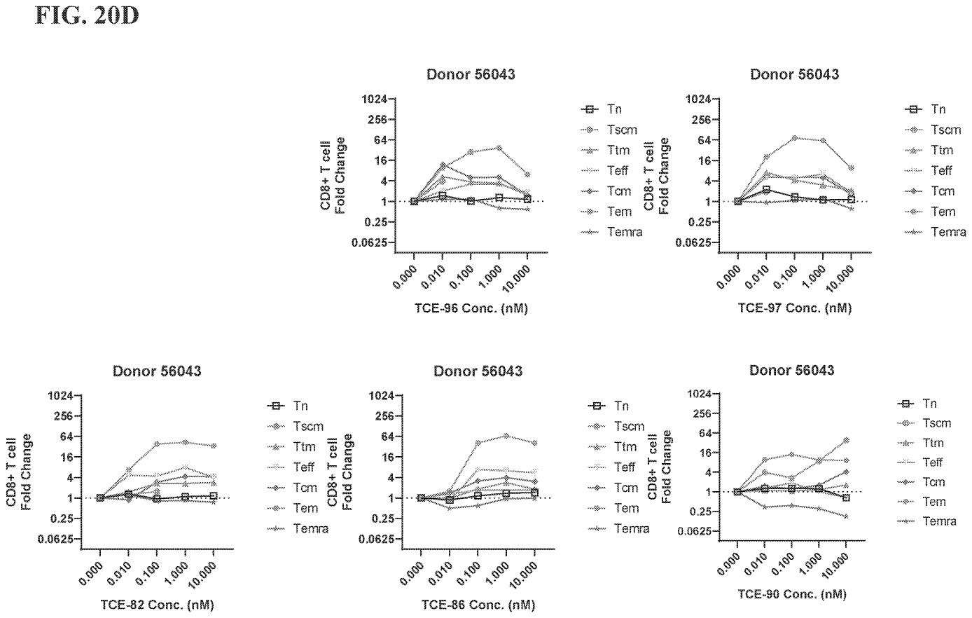

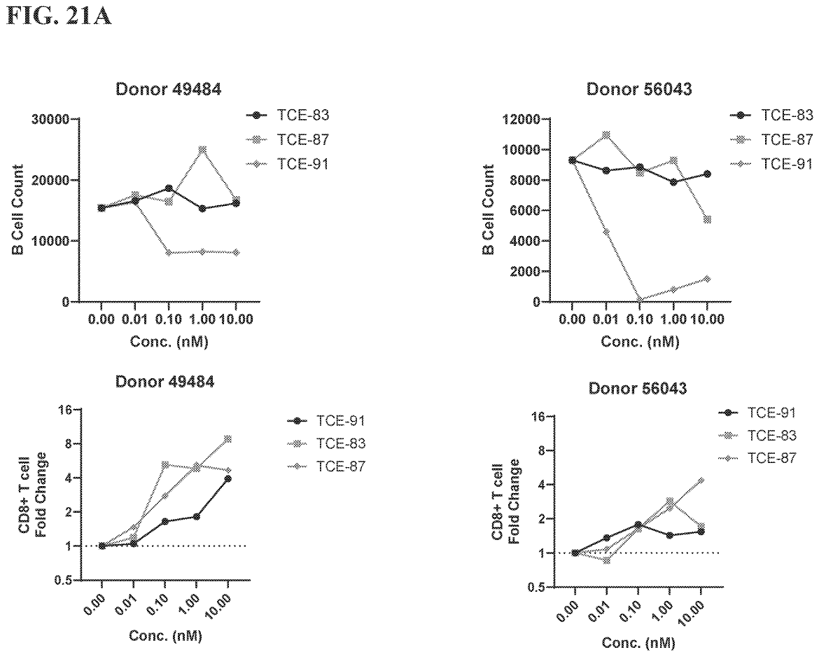

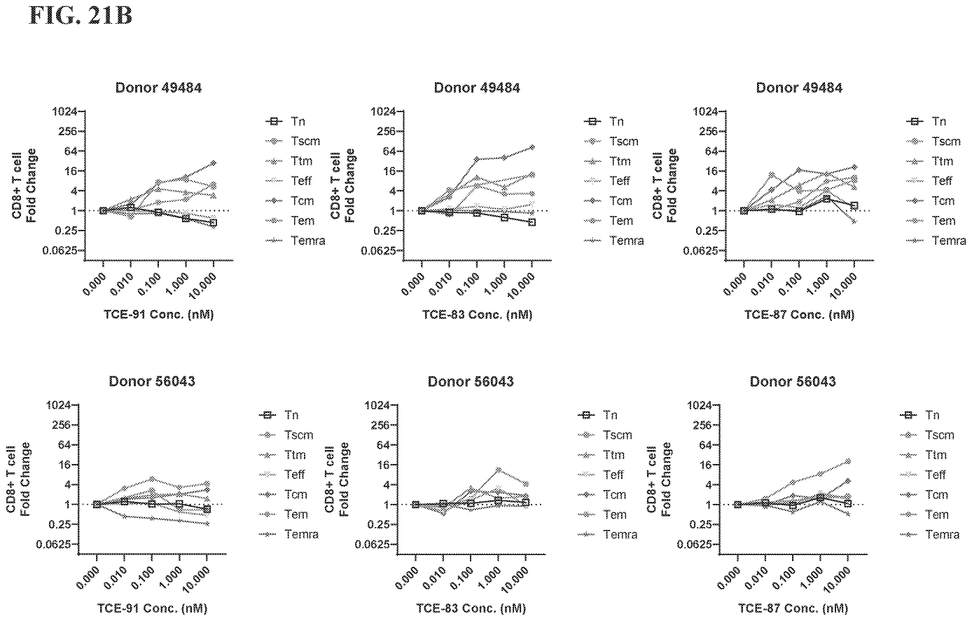

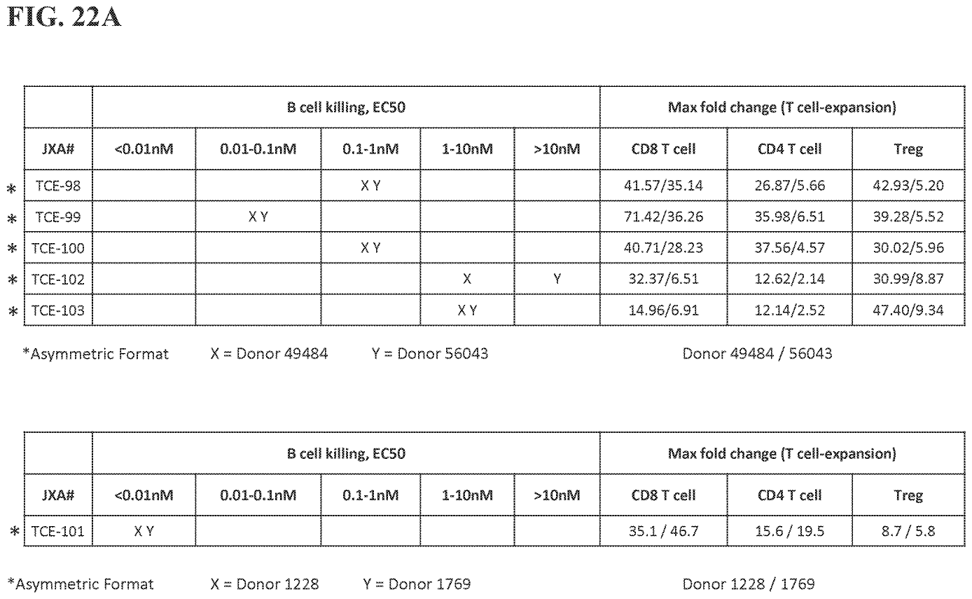

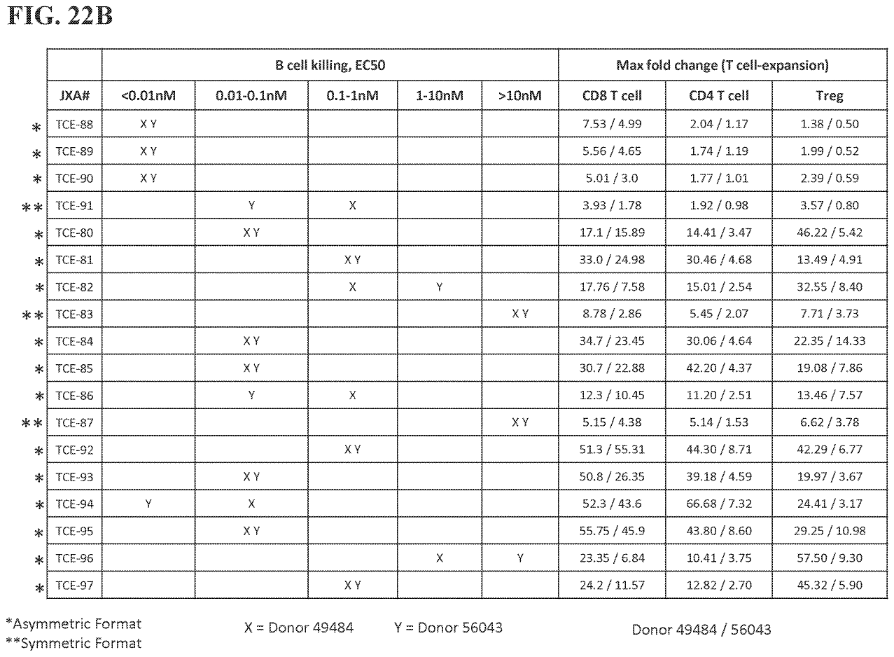

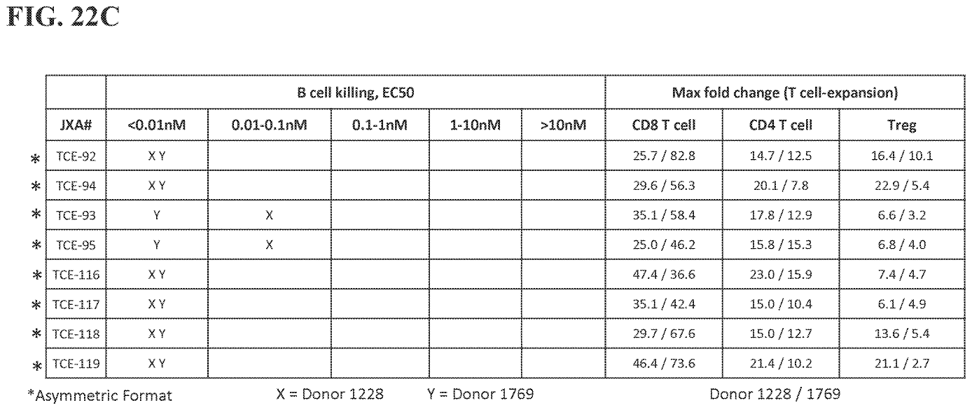

Other asymmetric and symmetric formats shown in FIG. 2B-3A also yielded active T cell engagers with an Fc region. As shown in FIG. 18-21B, despite the differences in the overall formats, anti-CD19 T cell engagers binding to TRBV generally possessed more potent capabilities of B cell depletion and T cell expansion (i.e., TCE-80 to TCE-87; TCE-92 to TCE-103; and TCE-116-119), compared to anti-CD19 T cell engagers binding to CD3 (i.e., TCE-88 to TCE-91). These results are summarized in FIG. 22A-22C.

### 2.3. Example 3: T Cell Engagers Binding to TRBV or TRAV Induce More Robust Expansion of Target T Cells Compared to T Cell Engagers Binding to CD3 in Disease State PBMCs Obtained from Autoimmune Disease Patients

Methods: T cell engagers were evaluated in flow cytometry based functional ex vivo assays using human disease state PBMCs collected from patients with autoimmune disease, systemic Lupus erythematosus (SLE) or Rheumatoid arthritis (RA). The assay was run using disease state PBMCs that comprise unmanipulated physiologic ratios of T cells and B cells. Titrated T cell engagers were incubated with freshly thawed 250,000 disease state PBMCs per well for up to 7 days. Readouts were taken on days 1, 3, 5, and/or 7 as well as pre-treatment (day 0). At the indicated timepoints supernatants were harvested for cytokine measurements and cells were harvested for flow cytometry measurements. Supernatants from PBMCs were analyzed for cytokines using a Meso Scale Device kit following the manufacturer's instructions. Flow cytometry measurements included live/dead, CD3, CD8, CD4, Foxp3, TRBV, CD25, CCR7, CD45RA, CD95, CD20, and/or CD19 in the panel. Counting beads were included to accurately quantify absolute number of cells present in the samples. Flow cytometric readouts were plotted against the logarithmic concentration of the T cell engagers. Where indicated the concentration of T cell engagers required to reach half maximum readout was calculated in GraphPad Prism and reported as an EC50. Readouts included T cell expansion (increase in absolute number of T cells or T cell subsets), T cell activation (CD25) as well as killing of B cells (decrease in CD3-CD20+ cells). Fold change in T cell counts was calculated as T cell engager target positive T cells (CD3+ or TRBV+) post T cell engager treatment divided by the analogous T cell counts without treatment. The maximum B cell depletion at any T cell engager concentration, Emax, was calculated and reported as a percent depletion relative to the untreated samples. Flow cytometry gating was used to define cell populations as in the Examples above.

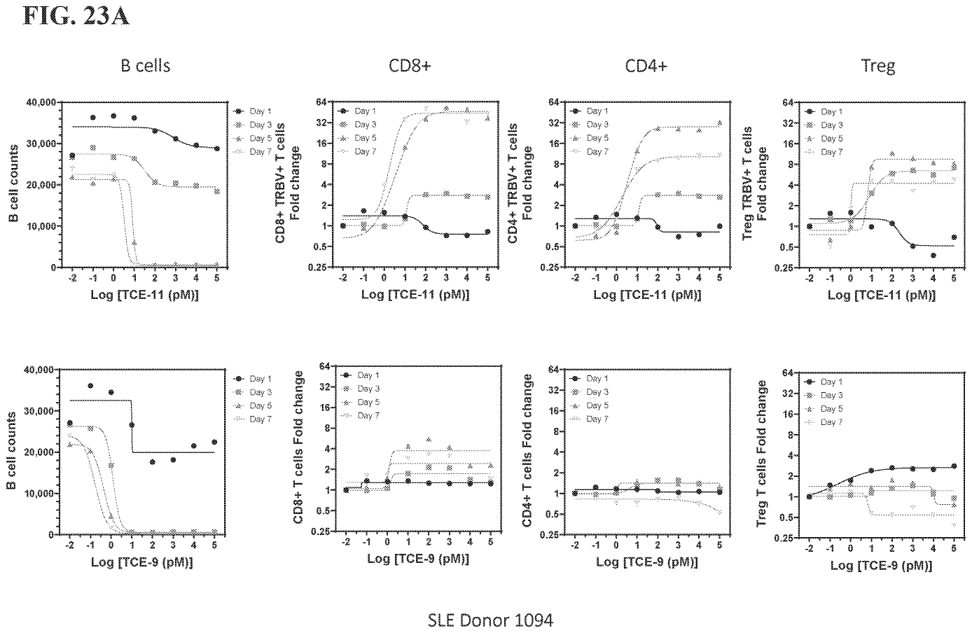

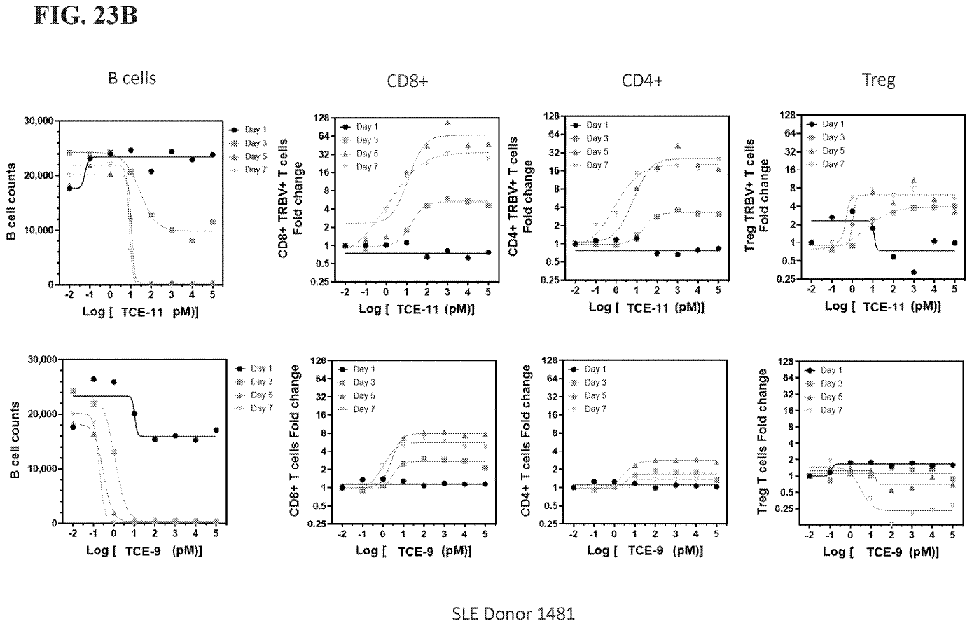

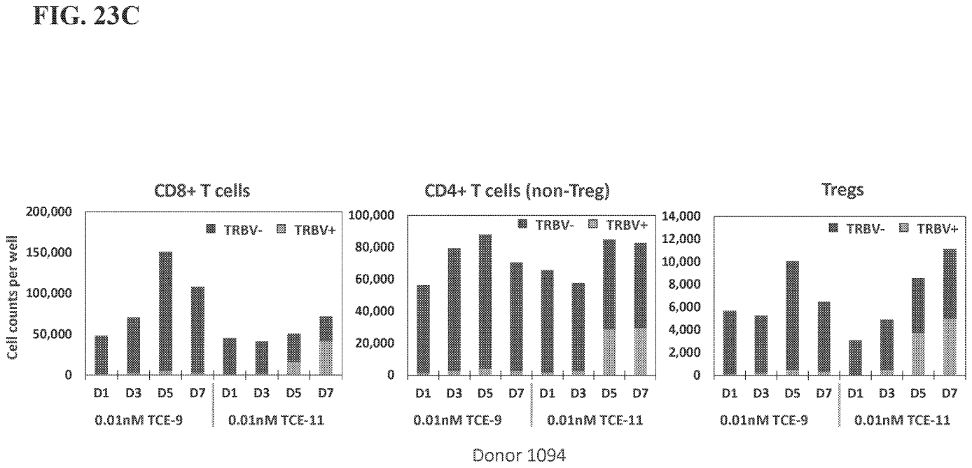

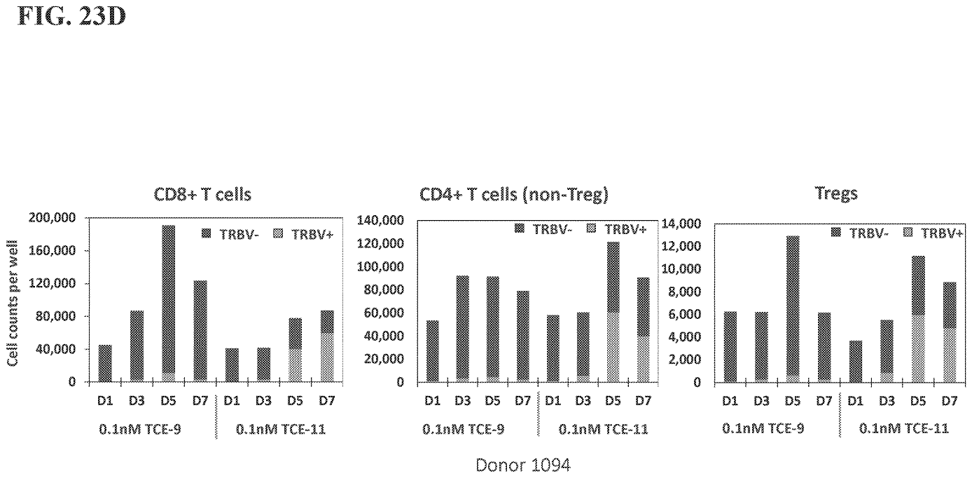

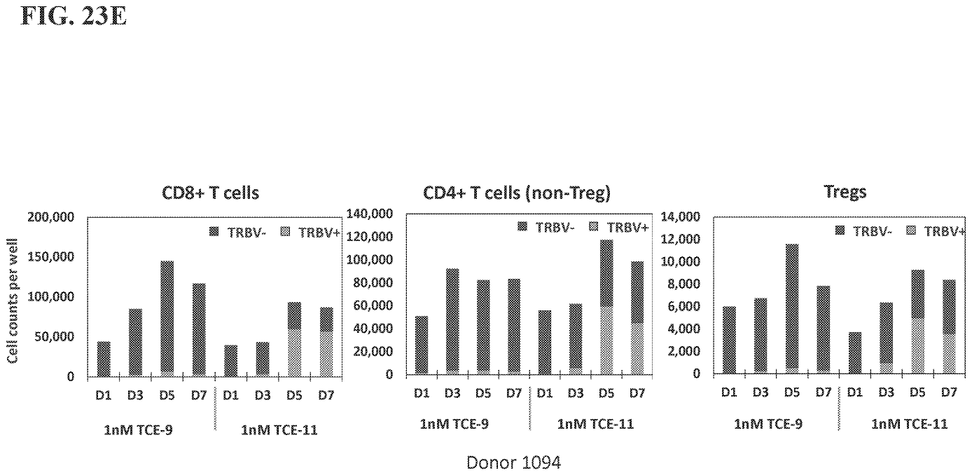

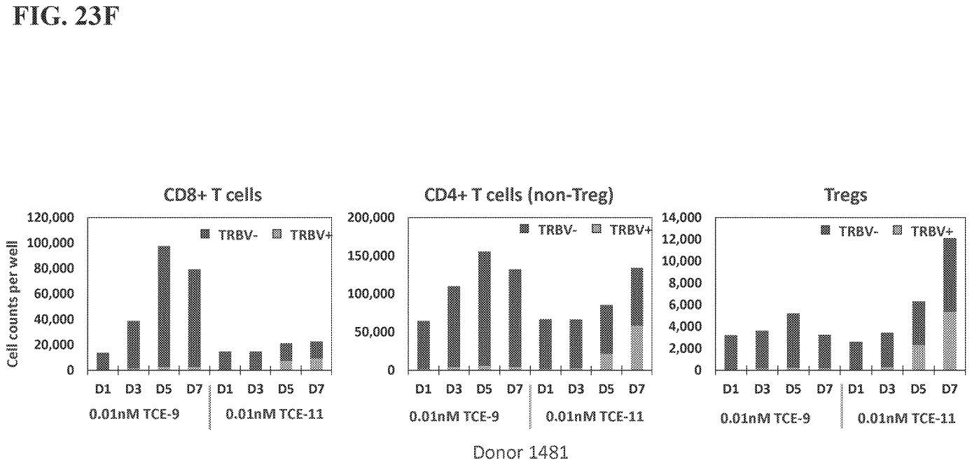

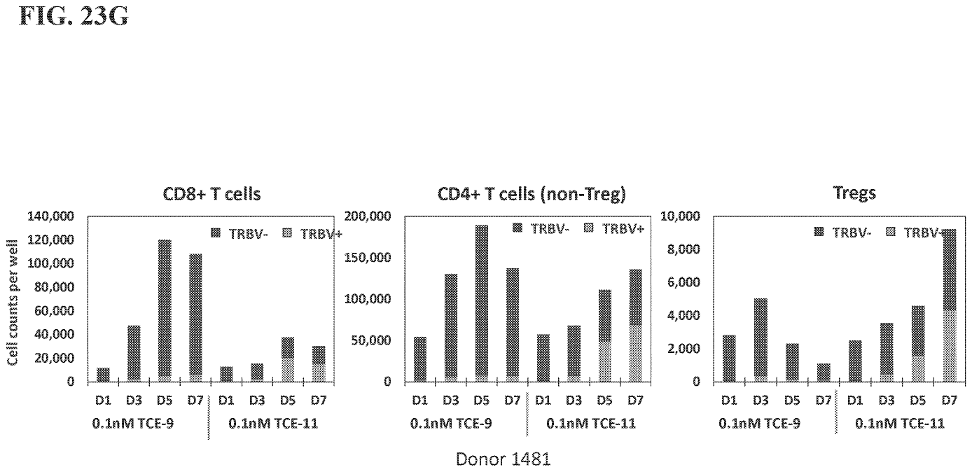

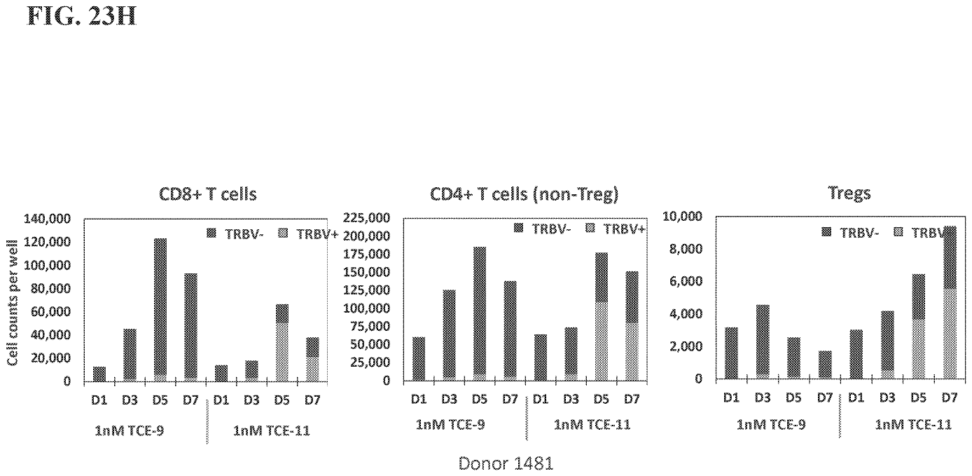

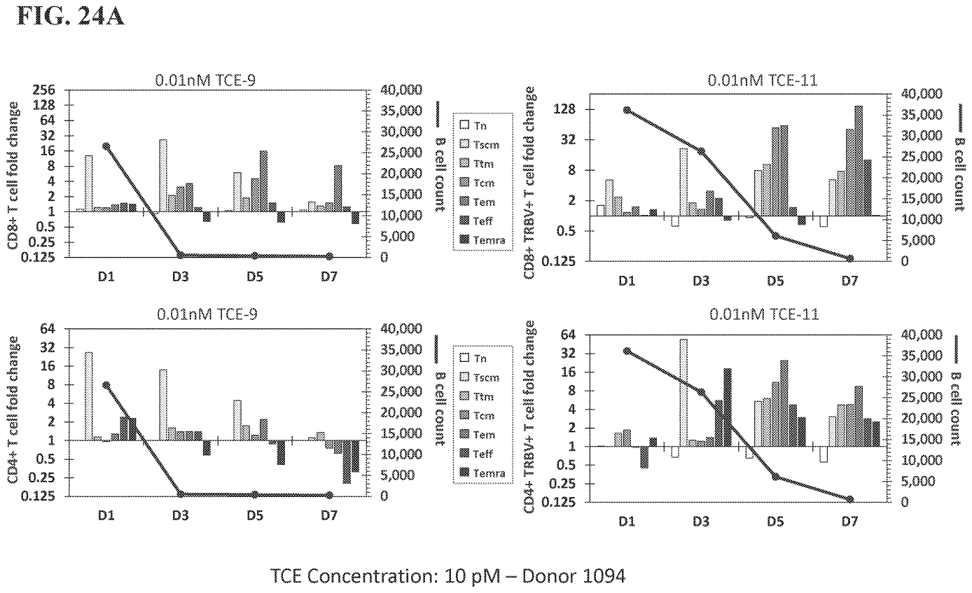

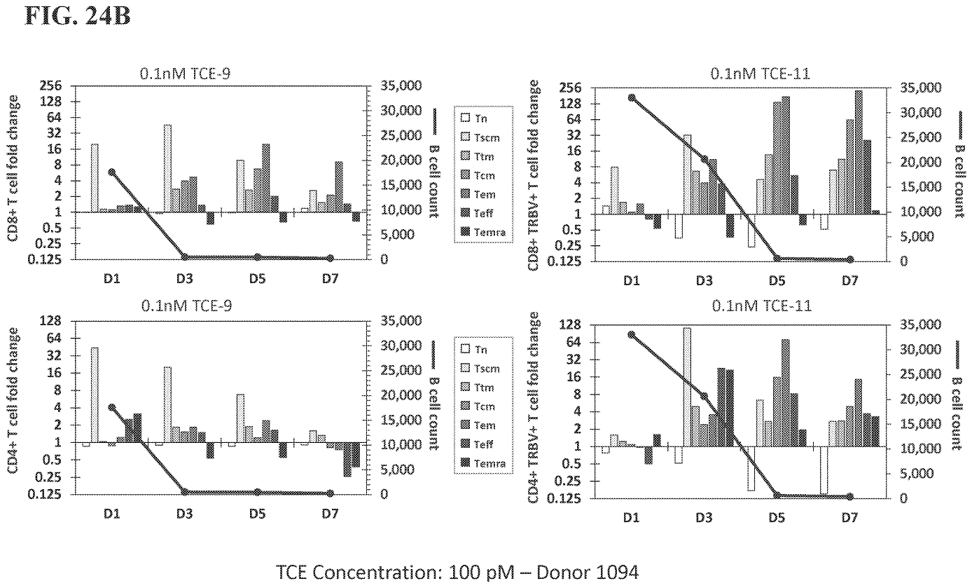

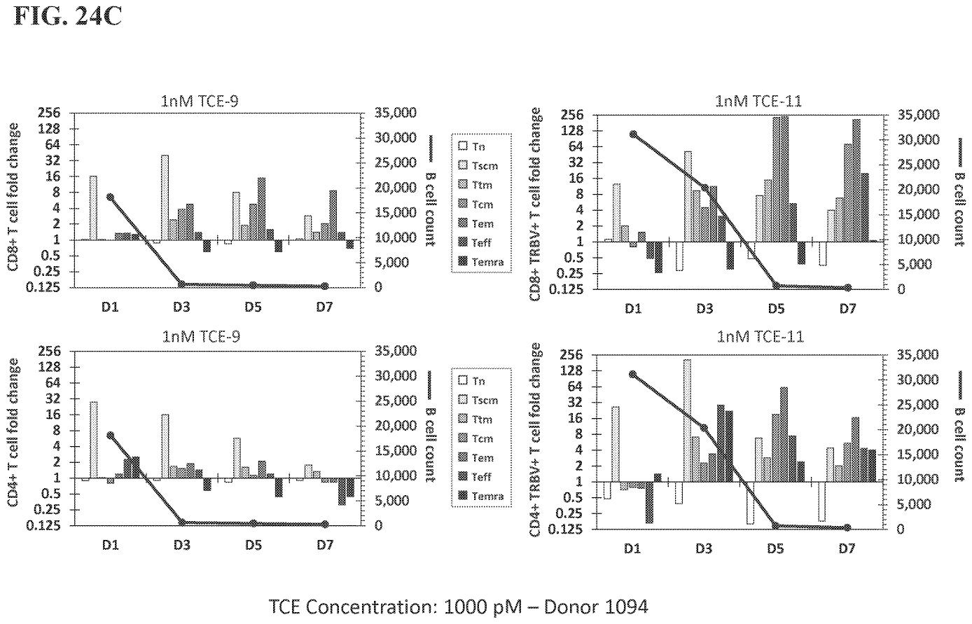

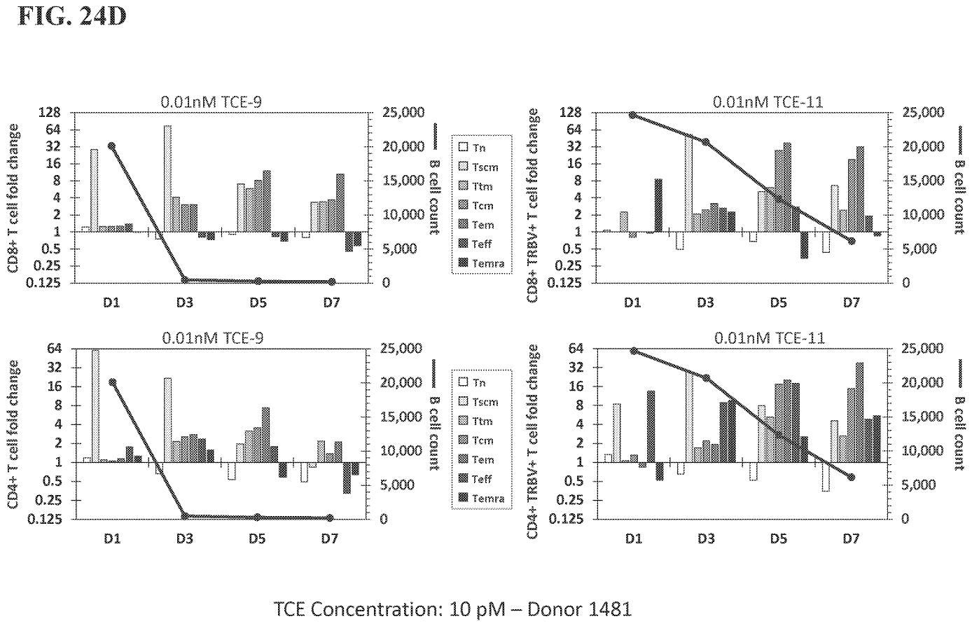

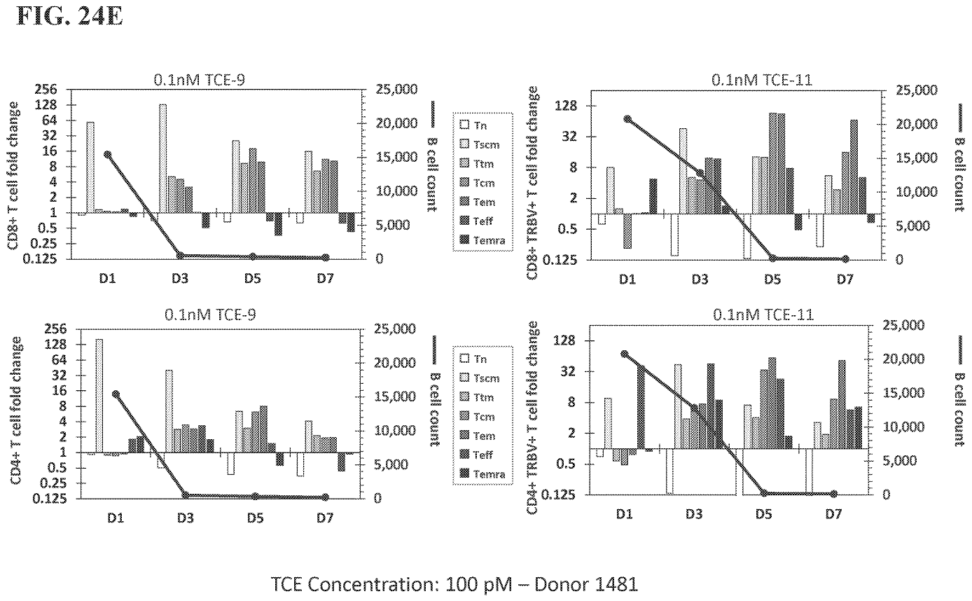

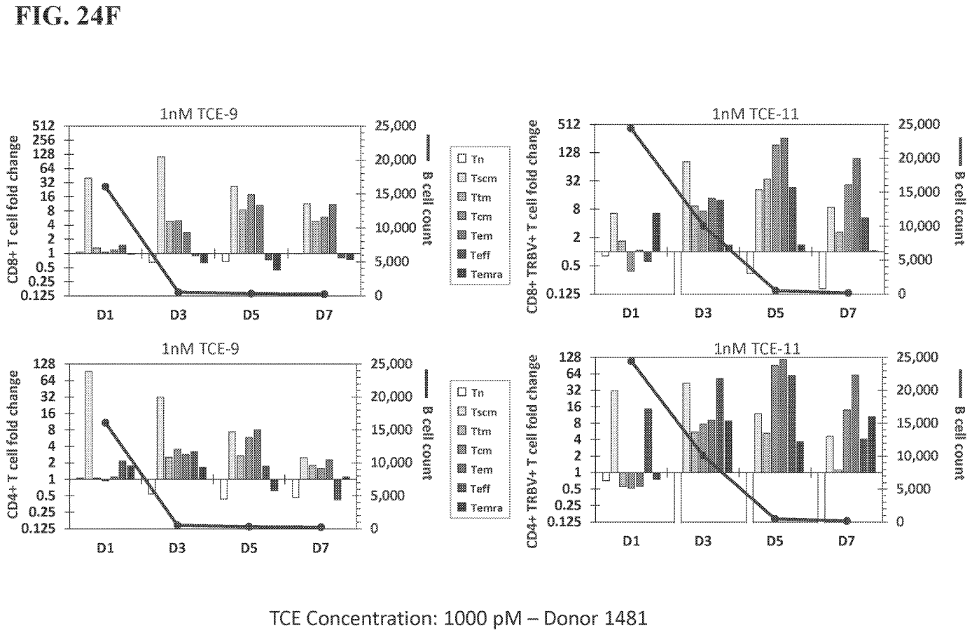

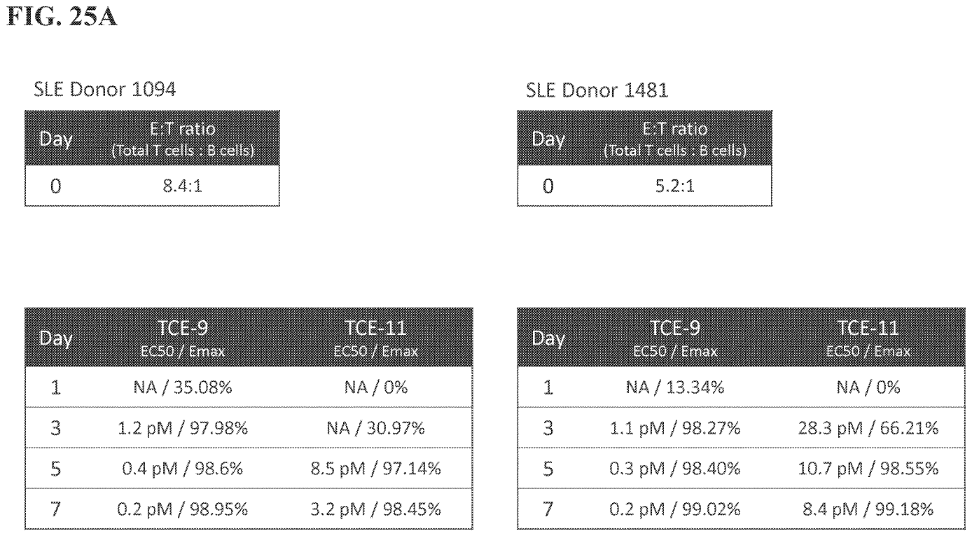

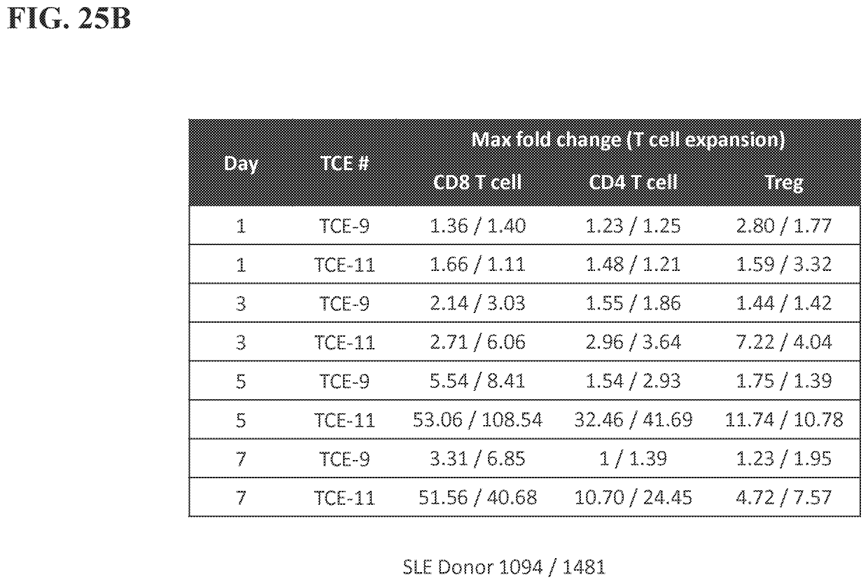

Similar to the studies conducted with PBMCs from healthy donors, T cell engagers targeting TCR beta variable domain are more potent at expanding memory and effector cell population in PBMCs collected from systemic Lupus erythematosus (SLE) patients, compared to T cell engagers targeting CD3. As shown in FIG. 23A-23H, the CD19×TRBV T cell engager (TCE-11) drove the expansion of target CD8+ T cells, CD4+ T cells, and Tregs in SLE patients PBMCs, whereas the CD19×CD3 T cell engager (TCE-9) failed to do so. As shown in FIG. 24A-24F, the CD19×TRBV T cell engager (TCE-11) increased numbers of Teff and Temra cells over time in SLE PBMC, whereas the CD19×CD3 T cell engager (TCE-9) reduced those populations. The CD19×TRBV T cell engager (TCE-11) induced larger increases in early memory populations that maintained or replenished effector populations. In contrast, the CD19×CD3 T cell engager (TCE-9) induced nominal increases in memory populations that failed to maintain effector populations over time. These results are summarized in FIG. 25A-25B.

### 2.4. Example 4: T Cell Engagers Binding to TRBV or TRAV Induce More Robust Expansion of Target T Cells Compared to T Cell Engagers Binding to CD3 in Disease State PBMCs Obtained from Subjects Having Cancer

Methods: T cell engagers were evaluated in flow cytometry based functional ex vivo assays using human disease state PBMCs collected from patients with Bruton's tyrosine kinase inhibitor (BTKi) treatment naïve chronic lymphoblastic leukemia (CLL). The assay was run using disease state PBMCs that comprise unmanipulated ratios of T cells and B cells. Titrated T cell engagers were incubated with freshly thawed 250,000 disease state PBMCs per well for up to 11 days. Readouts were taken on days 1, 3, 5, 7, 9 and/or 11 days as well as pre-treatment (day 0). At the indicated timepoints supernatants were harvested for cytokine measurements and cells were harvested for flow cytometry measurements. Supernatants from PBMCs were analyzed for cytokines using a Meso Scale Device kit following the manufacturer's instructions. Flow cytometry measurements included live/dead, CD3, CD8, CD4, Foxp3, TRBV, CD25, CCR7, CD45RA, CD95, CD20, and/or CD19 in the panel. Counting beads were included to accurately quantify absolute number of cells present in the samples. Flow cytometric readouts were plotted against the logarithmic concentration of T cell engagers. Where indicated the concentration of T cell engagers required to reach half maximum readout was calculated in GraphPad Prism and reported as an EC50. Readouts included T cell expansion (change in absolute number of T cells or T cell subsets), T cell activation, and killing of B cells (change in viable B cells counts). Fold change in T cell counts was calculated as T cell engager target positive T cells (CD3+ or TRBV+) post T cell engager treatment divided by the analogous T cell counts without treatment. The maximum B cell depletion at any T cell engager concentration, Emax, was calculated and reported as a percent depletion relative to the untreated samples. Flow cytometry gating was used to define cell populations as in the Examples above.

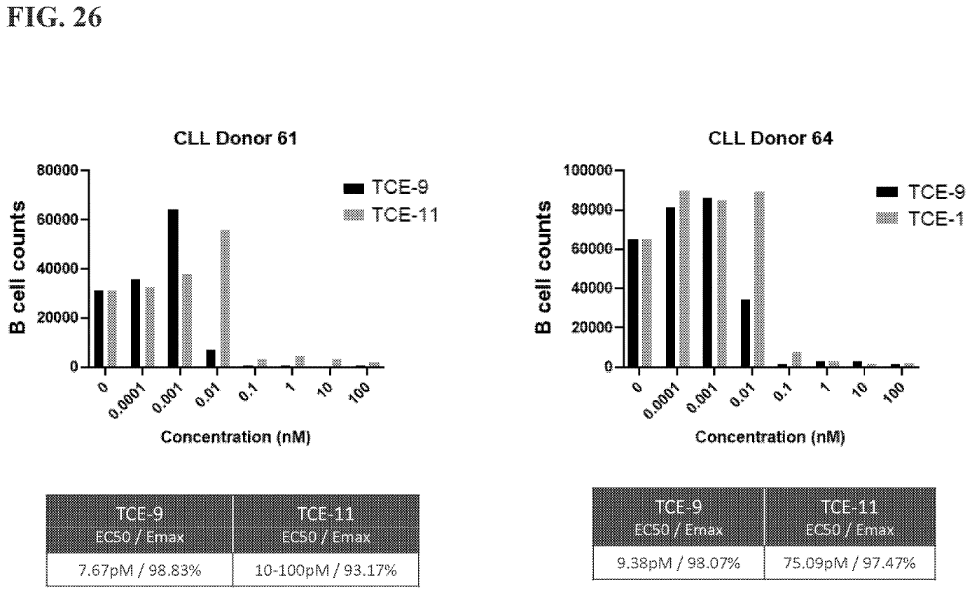

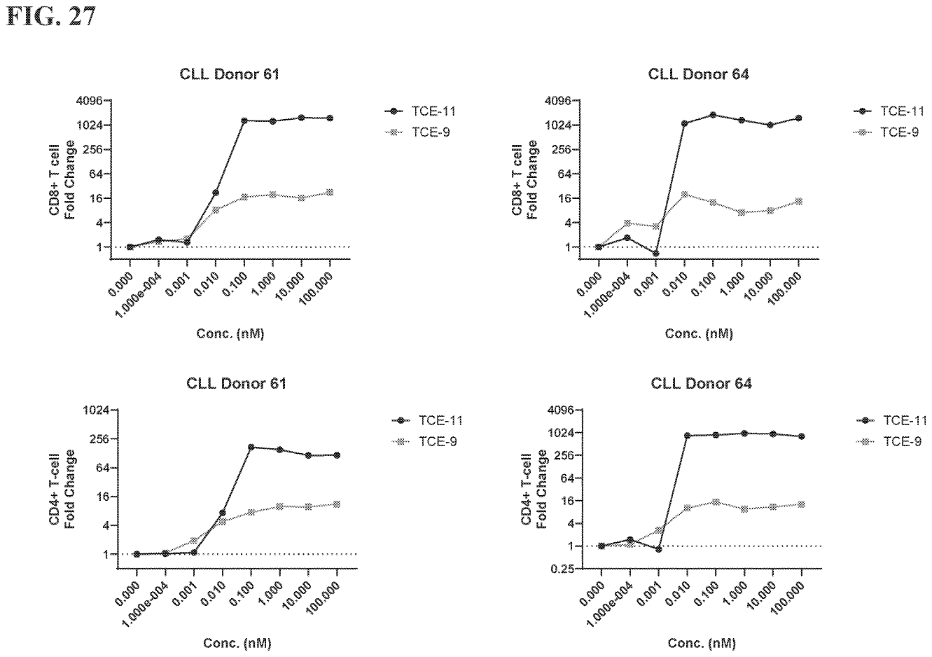

Similar to the studies conducted with PBMCs from healthy donors or patients with autoimmune diseases, T cell engagers targeting TCR beta variable domain are more potent at expanding target T cell population in PBMCs collected from chronic lymphoblastic leukemia (CLL) patients, compared to T cell engagers targeting CD3. As shown in FIG. 26, both types of T cell engagers effectively depleted B-cells in CLL patient PBMCs on Day 9. However, as shown in FIG. 27, the CD19×TRBV T cell engager (TCE-11) was more much more potent at stimulating the proliferation of target CD8+ and CD4+ T cells, compared to the CD19×CD3 T cell engager (TCE-9).

### 8.5. Example 5: Generation of Additional T Cell Engagers Binding to TRBV and CD19

T cell engagers binding to TRBV and CD19 were designed and generated according to the sequences in Table 20 below. The bispecific T cell engagers utilized the Fab fused to scFv configuration with a knob-in-hole Fc for half-life extension as shown in FIG. 2A.

TABLE 20
Exemplary CD19xTRBV T Cell Engagers

| | Bispecific | | SEQ ID NO | | |
|---|---|---|---|---|---|
| | Antigen | Antigen | Chain 2 | Chain 3 | Chain 4 |
| ID | 1 | 2 | (HC1) | (LC2) | (HC2) |
| CLY-018 | CD19 | TRBV6-5 | 1159 | 1166 | 1173 |
| CLY-019 | CD19 | TRBV6-5 | 1160 | 1167 | 1174 |
| CLY-020 | CD19 | TRBV6-5 | 1161 | 1168 | 1175 |
| CLY-021 | CD19 | TRBV6-5 | 1162 | 1169 | 1176 |
| CLY-022 | CD19 | TRBV6-5 | 1163 | 1170 | 1177 |
| CLY-023 | CD19 | TRBV6-5 | 1164 | 1171 | 1178 |
| CLY-024 | CD19 | TRBV6-5 | 1165 | 1172 | 1179 |

### ELISA Binding Assays

ELISA assays were conducted as described in Example 1.

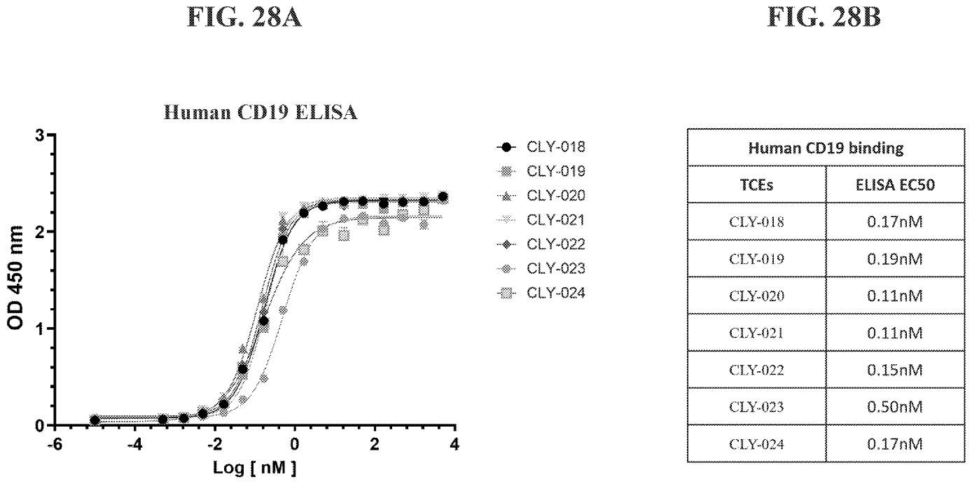

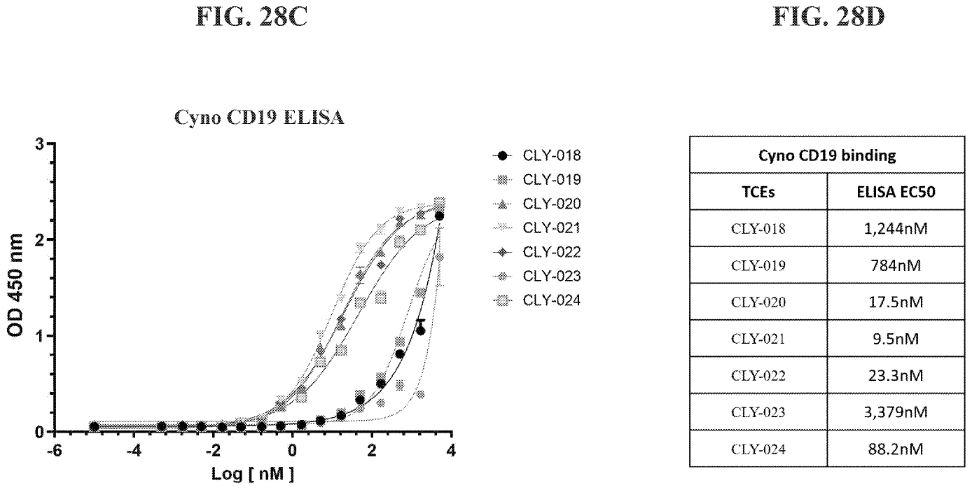

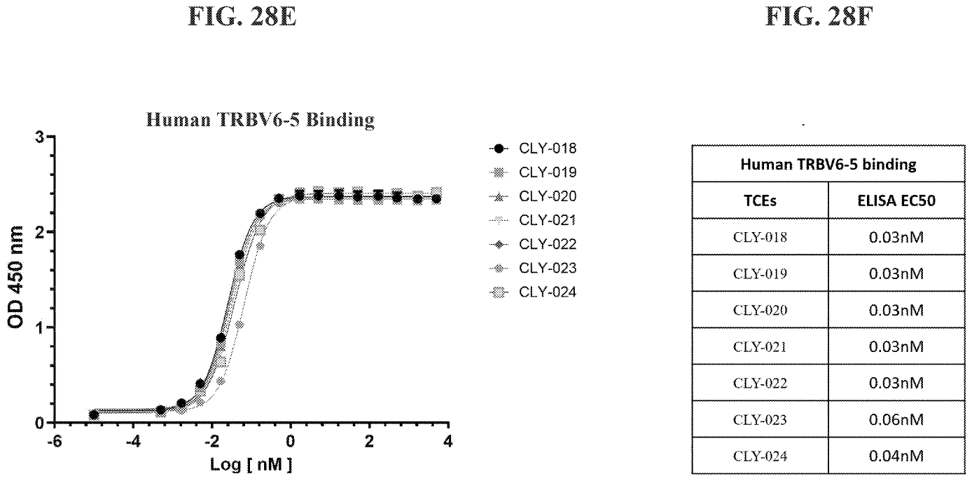

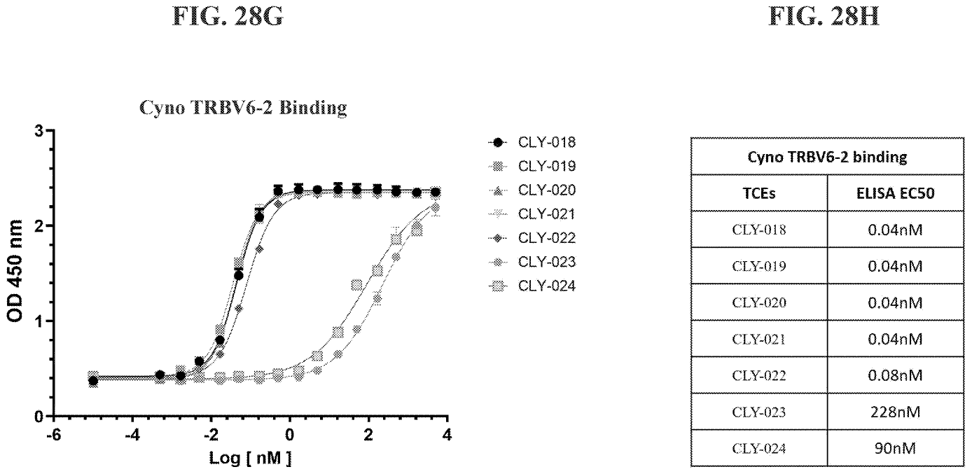

The T cell engagers CLY-018 to CLY-024 (CD19XTRBV) were tested for binding against human CD19 (FIGS. 28A-28B) and cyno CD19 (FIGS. 28C-28D). The same T cell engagers were also tested for binding against human TRBV6-5 (FIGS. 28E-28F) and cyno TRBV6-2 (FIGS. 28G-28H).

### Cell Binding by Flow Cytometry

Polypeptide complexes (TCRBDs or TCEs) were evaluated in flow cytometry based cell binding assays using human or cynomolgus monkey PBMCs that comprise unmanipulated physiologic ratios of T cells and B cells. Titrated polypeptide complexes were incubated with freshly thawed 250,000 human or cynomolgus monkey PBMCs for 1 hr in serum containing culture medium. Cells were washed and stained with live/dead dye, and CD3, CD8, CD4, TRBV, CD20, and/or CD19 fluorescent antibodies as well as a secondary fluorescent antibody that recognizes polypeptide complexes. Cells were washed again and fixed. Stained and fixed cells were stored at 4C until analysis. Mean fluorescent intensity (MFI) of the cell bound polypeptide complex on T cells or B cells was normalized to the maximum observed and plotted against the logarithmic polypeptide complex concentration. Where indicated the concentration of polypeptide complex required to reach half maximum readout was calculated in Graphpad Prism and reported as an EC50. Readouts included EC50 binding on T cells and EC50 binding on B cells from human and cynomolgus monkey.

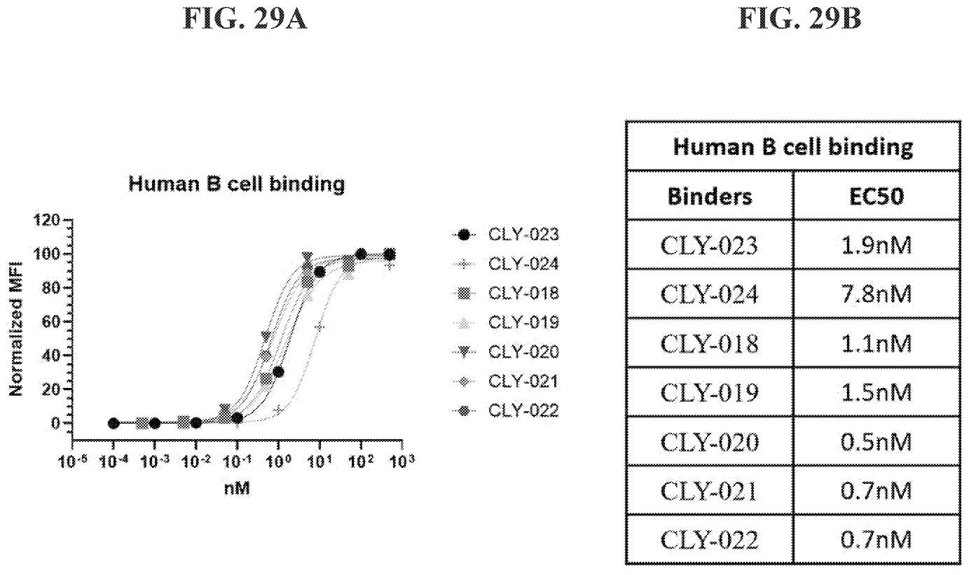

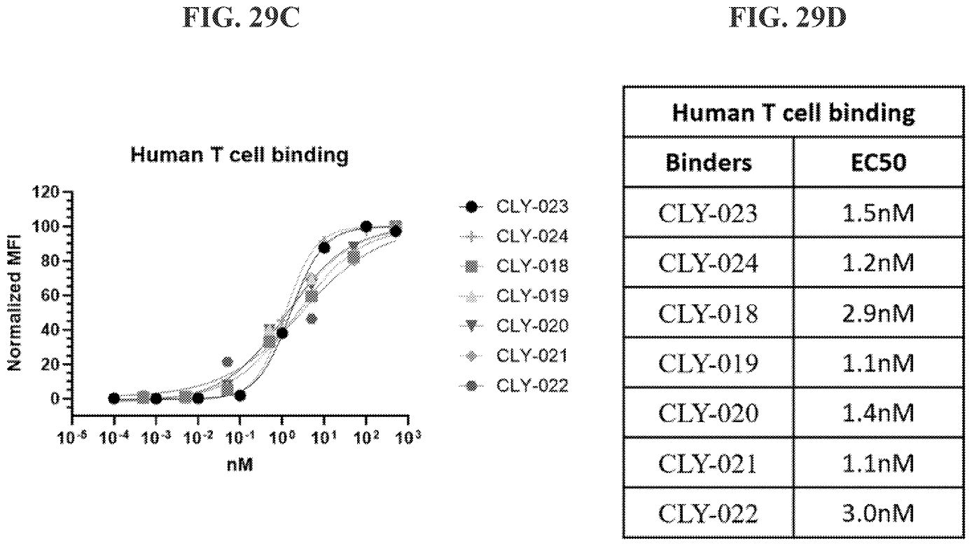

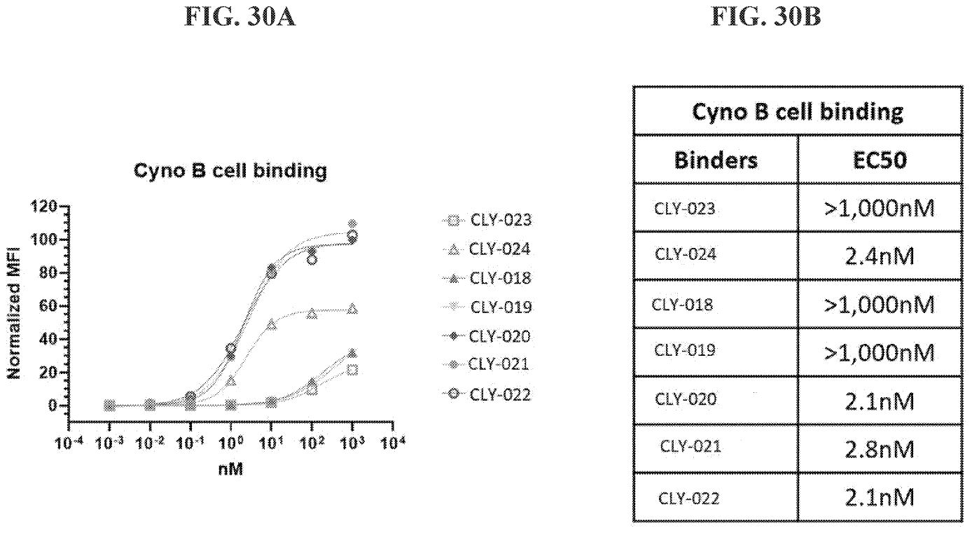

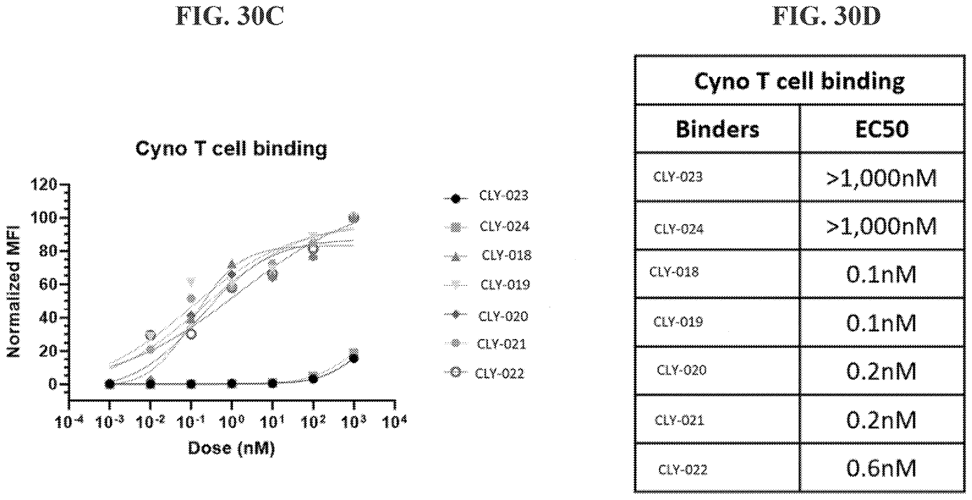

The results of CLY-018-CLY-024 binding to human B cells is shown in FIGS. 29A-29B and the results of CLY-018-CLY-024 binding to human T cells is shown in FIGS. 29C-29D. The same T cell engagers were also evaluated for binding to cyno B cells (FIGS. 30A-30B) and cyno T cells (FIGS. 30C-30D).

### 8.6. Example 6: In Vitro Activity of CD19×TRBV T Cell Engagers in Healthy Human and Cynomolgus Monkey Cells

B-cell depletion, T-cell expansion and phenotype, and cytokine induction of the aforementioned T cell engagers were evaluated in human and monkey cells.

Polypeptide complexes were evaluated in flow cytometry based functional ex vivo assays using human or cynomolgus monkey (Macaca fascicularis) PBMCs that comprise unmanipulated physiologic ratios of T cells and B cells. Titrated polypeptide complexes were incubated with freshly thawed 250,000 human or cynomolgus monkey PBMCs per well for up to 7 days. Readouts were taken on days 1, 2 3, 5, and/or 7 as well as pre-treatment (day 0). At the indicated timepoints, supernatants were harvested for cytokine measurements and cells were harvested for flow cytometry measurements. Supernatants from human PBMCs were analyzed for cytokines using a Meso Scale Device kit following the manufacturer's instructions. Supernatants from cynomolgus monkey PBMCs were analyzed using a cytometric bead array following the manufacturer's instructions. Flow cytometry measurements included live/dead, CD3, CD8, CD4, Foxp3, TRBV, CD25, GranzymeB, Ki-67, CCR7, CD45RA, CD95, CD20, and/or CD19 in the panel. Counting beads were included to accurately quantify absolute number of cells present in the samples. Flow cytometric readouts were plotted against the logarithmic concentration of polypeptide complex. Where indicated the concentration of polypeptide complex required to reach half maximum readout was calculated in Graphpad Prism and reported as an EC50. Readouts included T cell expansion (change in absolute number of viable T cells or T cell subsets), T cell activation, as well as killing of B cells (change in viable B cell counts). Fold change in T cell counts was calculated as polypeptide complex target positive T cells (CD3+ or TRBV+) post polypeptide complex treatment divided by the analogous T cell counts without treatment.

Flow cytometry parameters were established: All viable cells were gated with live/dead stain; and as follows: B cells (CD3− CD20+); CD8+ T cells (CD3+ CD8+); CD4+ T cells (CD3+ CD4+ FoxP3−); Tregs (CD3+ CD4+ FoxP3+); Tn (CD3+ CD8+ (or CD4+) CCR7+ CD45RA+ CD95−); Tscm (CD3+ CD8+ (or CD4+) CCR7+ CD45RA+ CD95+); Ttm (CD3+ CD8+ (or CD4+) CCR7+ CD45RA+/−); Tcm (CD3+ CD8+ (or CD4+) CCR7+ CD45RA−); Tem (CD3+ CD8+ (or CD4+) CCR7− CD45RA−); Teff (CD3+ CD8+ (or CD4+) CCR7− CD45RA+/−); and Temra (CD3+ CD8+ (or CD4+) CCR7− CD45RA+).

B Cell Killing and TRBV+ Expansion in Human PBMCs with T Cell Engagers

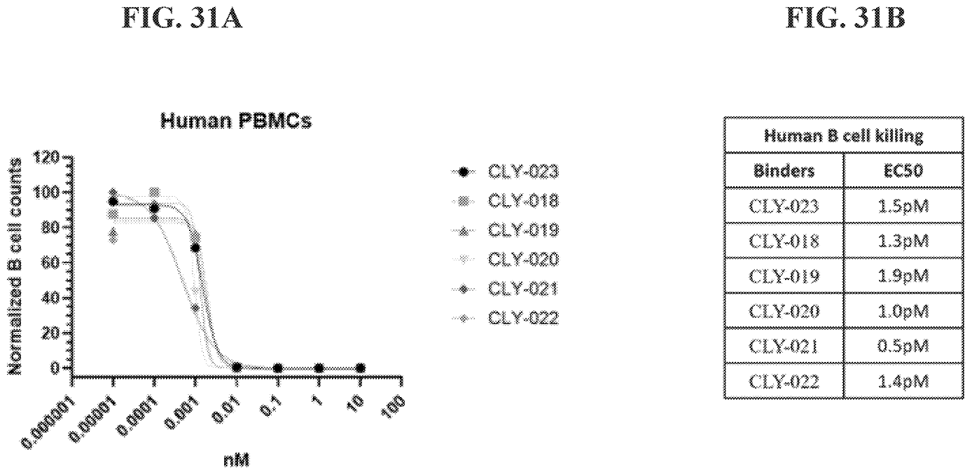

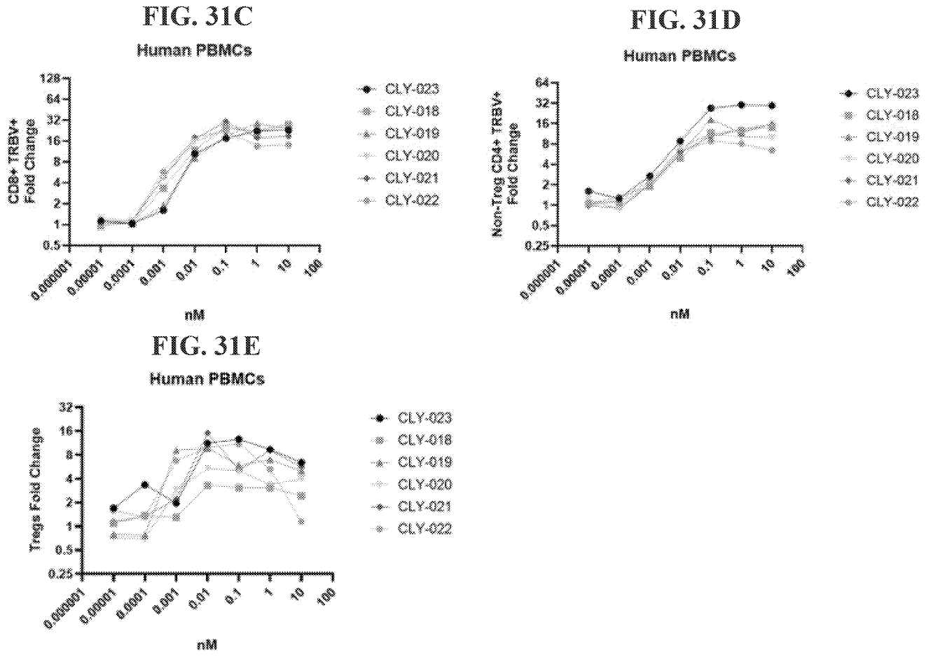

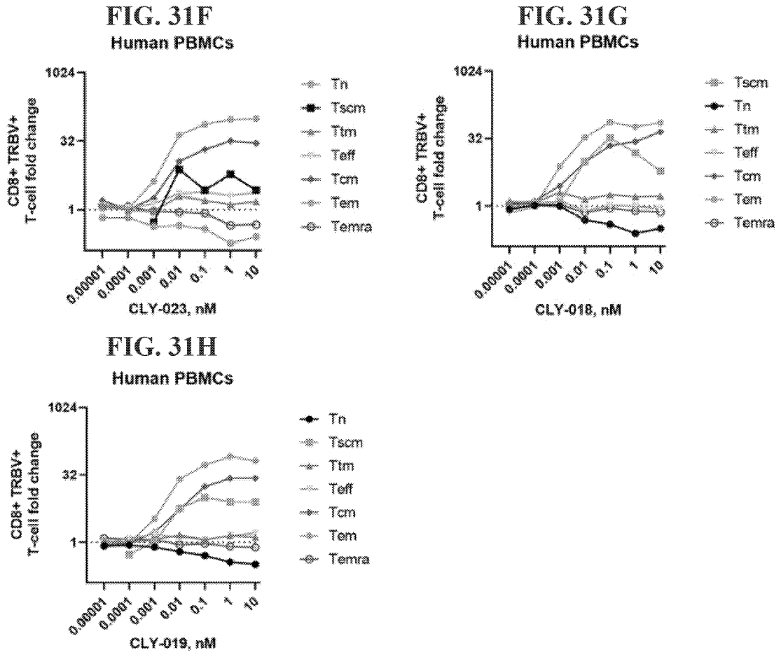

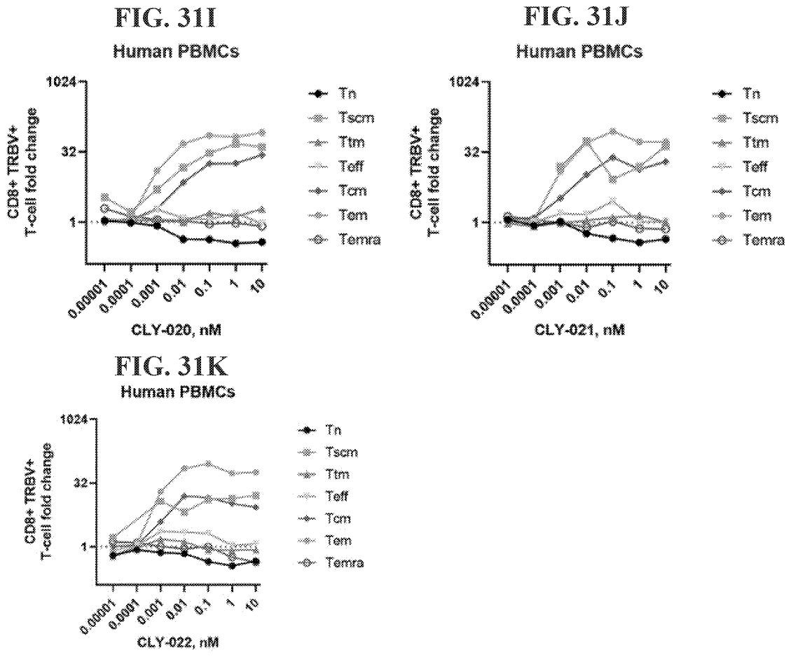

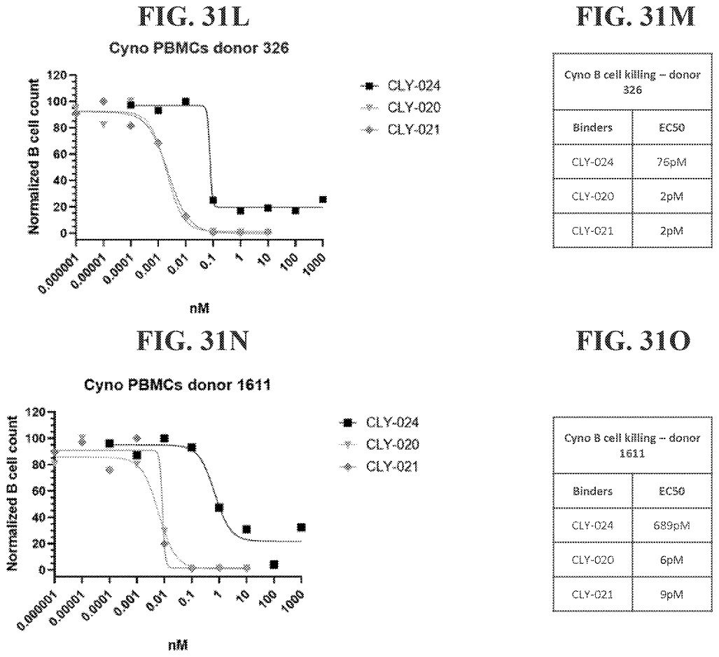

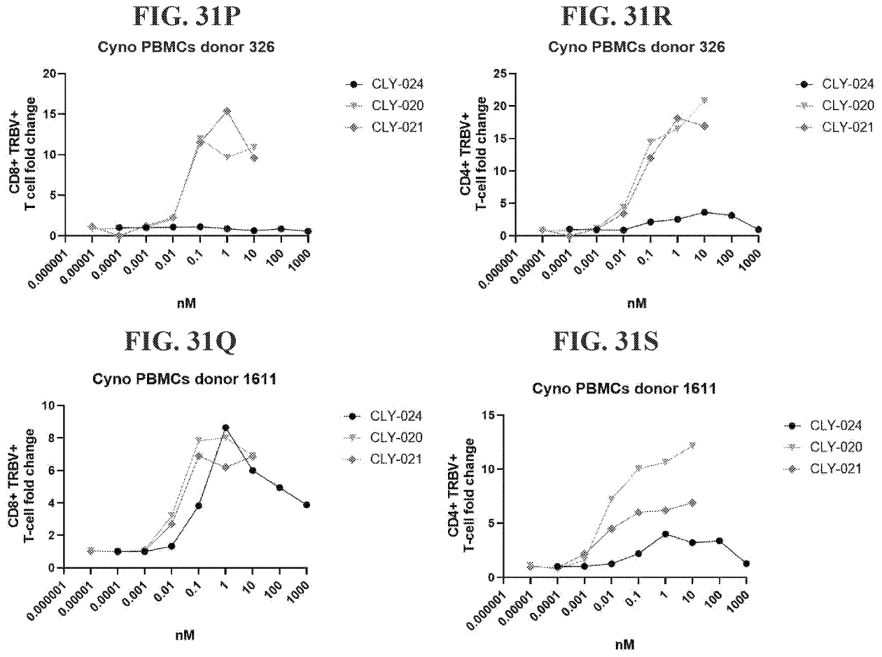

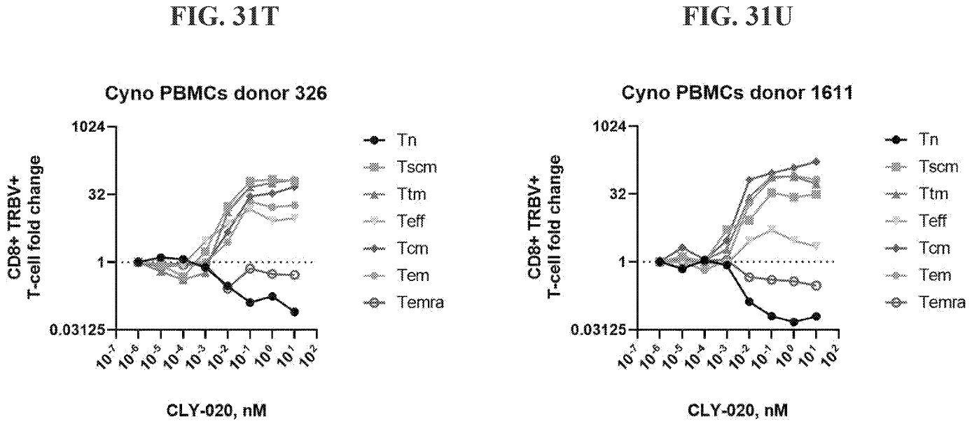

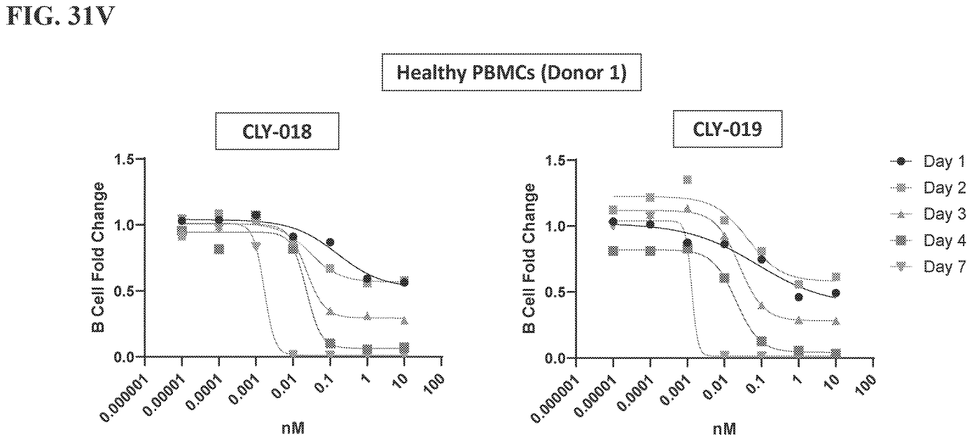

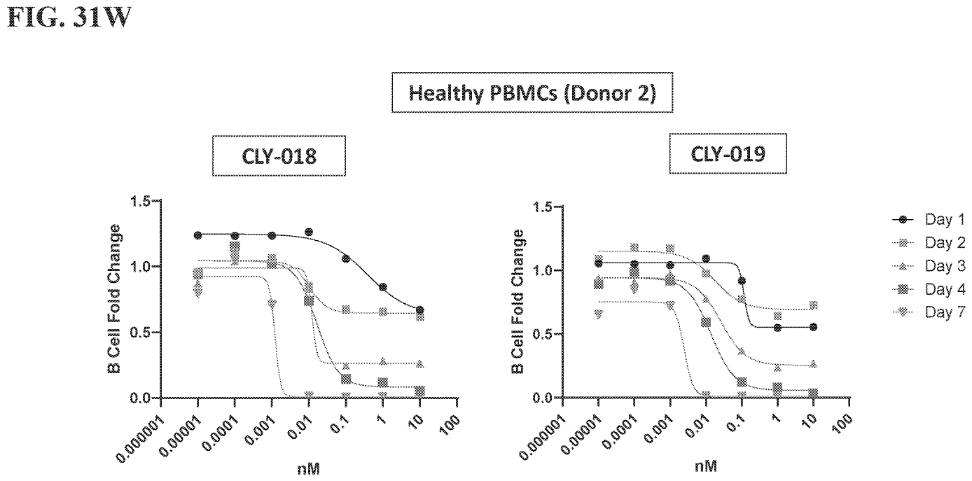

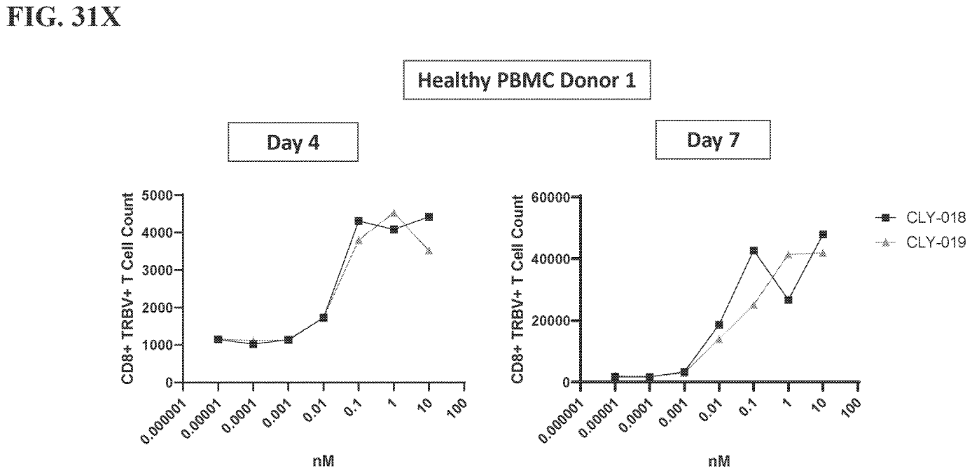

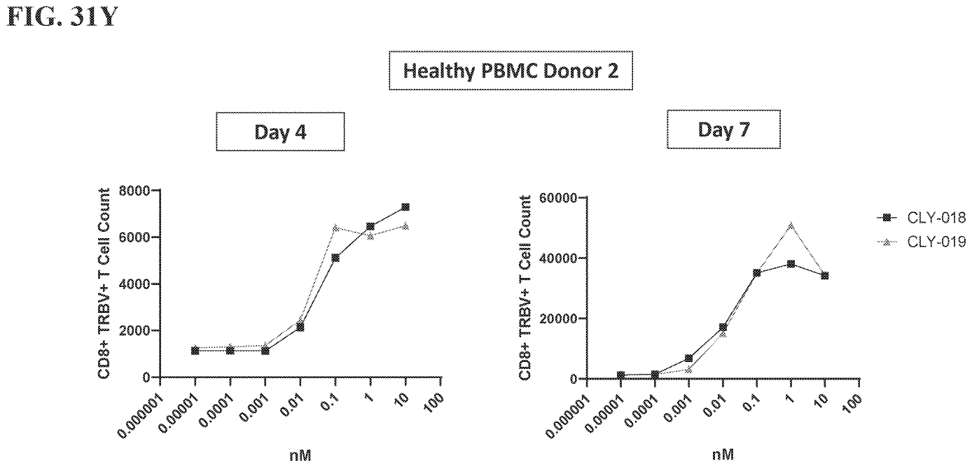

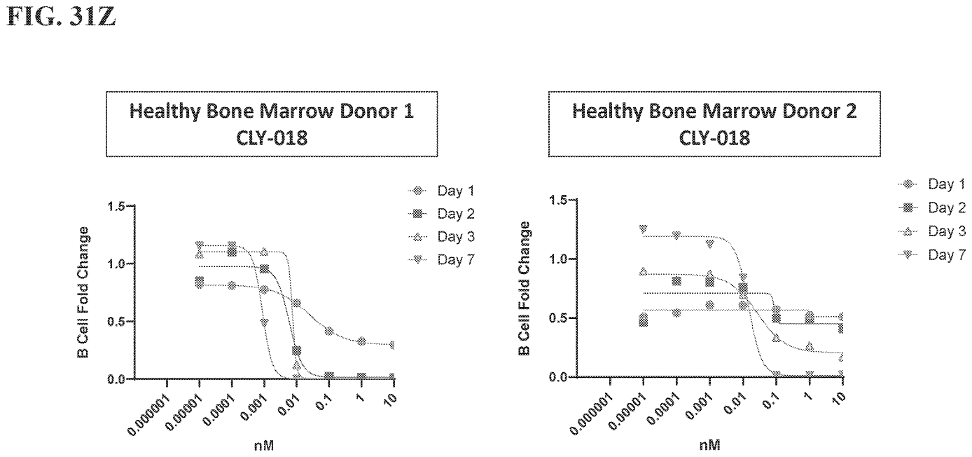

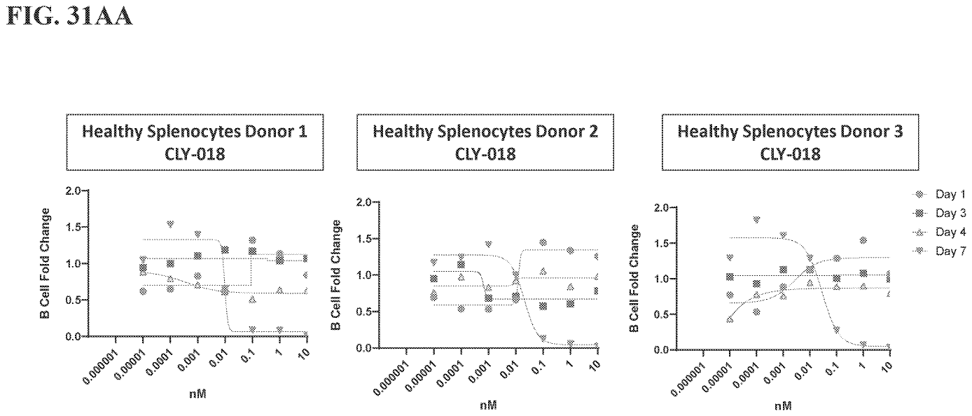

The T cell engagers CLY-018-CLY-023 were evaluated for B cell killing and expansion using human PBMCs. B cell killing results are displayed at FIG. 31A. EC50 values for the human B cell killing assay are displayed at FIG. 31B. The same T cell engagers were also evaluated for expansion. Results are displayed for CD8+TRBV+ (FIG. 31C), non-Treg CD4+TRBV+ (FIG. 31D), and Tregs (FIG. 31E). Displayed results are averages of N=2.

Additional experiments were performed to assess B cell killing and TRBV+ T cell expansion in human PBMCs with the T cell engagers CLY-018 and CLY-019. B cell killing results using PBMCs from healthy donor “1” and donor “2” are displayed at FIG. 31V and FIG. 31W, respectively. Expansion data for CD8+TRBV+ T cells in human PBMCs from healthy donor “1” and donor “2” are displayed at FIG. 31X and FIG. 31Y, respectively.

Expansion of CD8+TRBV+ T cell Subsets in Human PBMCs with T Cell Engagers

Expansion data for CD8+TRBV+ cells treated with CLY-023 are shown at FIG. 31F. Expansion data for CD8+TRBV+ cells treated with CLY-018 are shown at FIG. 31G. Expansion data for CD8+TRBV+ cells treated with CLY-019 are shown at FIG. 31H. Expansion data for CD8+TRBV+ cells treated with CLY-020 are shown at FIG. 31I. Expansion data for CD8+TRBV+ cells treated with CLY-021 are shown at FIG. 31J. Expansion data for CD8+TRBV+ cells treated with CLY-022 are shown at FIG. 31K. Displayed results are averages of N=2.

B cell Killing in Human Bone Marrow Mononuclear Cells (BMMCs) with T Cell Engagers

The T cell engager CLY-018 was evaluated for B cell killing using human bone marrow mononuclear cells from healthy donors and the experimental protocol described above. B cell killing results are displayed at FIG. 31Z.

B cell Killing in Human Splenocytes with T Cell Engagers

The T cell engager CLY-018 was evaluated for B cell killing using human splenocytes from healthy donors and the experimental protocol described above. B cell killing results are displayed at FIG. 31AA.

B Cell Killing and TRBV+ T Cell Expansion in Cynomolgus Monkey PBMCs with T Cell Engagers

The T cell engagers CLY-020, CLY-021, and CLY-024 were evaluated for B cell killing and TRBV+ T cell expansion using cynomolgus monkey PBMCs from donors “326” and “1611.”

B cell killing results are displayed at FIGS. 31L-31M for donor “326” and at FIGS. 31N-31O for donor “1611.” CD8+TRBV+ T cell expansion data for CLY-020, CLY-021, and CLY-024 is displayed at FIG. 31P for donor “326” and at FIG. 31Q for donor “1611.” CD4+TRBV+ T cell expansion data for CLY-020, CLY-021, and CLY-024 is displayed at FIG. 31R for donor “326” and at FIG. 31S for donor “1611.”

Cellular Phenotypic Analysis of Cynomolgus Monkey Cells Treated with T Cell Engagers

The CD8+TRBV+ memory phenotype of cynomolgus monkey PBMCs cells treated with the T cell engager CLY-020 is displayed at FIG. 31T for donor “326” and at FIG. 31U for donor “1611.” The populations: Tn (CD3+ CD8+ (or CD4+) CCR7+ CD45RA+ CD95−); Tscm− CD3+ CD8+ (or CD4+) CCR7+ CD45RA+ CD95+; Ttm− CD3+ CD8+ (or CD4+) CCR7+ CD45RA+/−; Tcm− CD3+ CD8+ (or CD4+) CCR7+ CD45RA−; Tem− CD3+ CD8+ (or CD4+) CCR7− CD45RA−; Teff− CD3+ CD8+ (or CD4+) CCR7− CD45RA+/−; and Temra− CD3+ CD8+ (or CD4+) CCR7− CD45RA+ were identified for the two cyno donors.

TRBV Specificity: RNA Sequencing Following T Cell Expansion with T Cell Engagers

Polypeptide complexes were evaluated in TRBV specificity assays based on RNA sequencing of expanded human CD4+ or CD8+ T cells from three different donors. Plate bound polypeptide complexes were incubated with freshly thawed CD4 or CD8 T cells for 7 days. A no stimulation control was used where T cells were not treated with polypeptide complexes. After 7 days all cells were harvested, counted by flow cytometry, and processed to RNA using a kit following the manufacturer's instructions. RNA was then sequenced using Nanostring with kits focused on the TCR complex including all TRBV and TRAV sequence families. The number of transcripts measured for each individual TRBV or TRAV gene was counted and plotted relative to the no stimulation control. An increase in specific TRBV transcripts relative to the no stimulation control was an indicator of which T cells expanded in the wells during the 7 day polypeptide treatment. The TRBV transcript signal was furthermore an indicator of the specificity of the polypeptide complex to activate and expand specific TRBV expressing T cells.

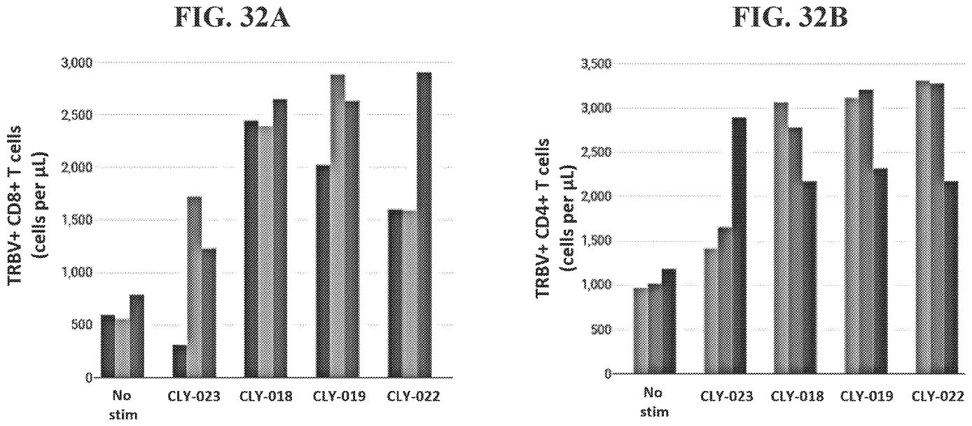

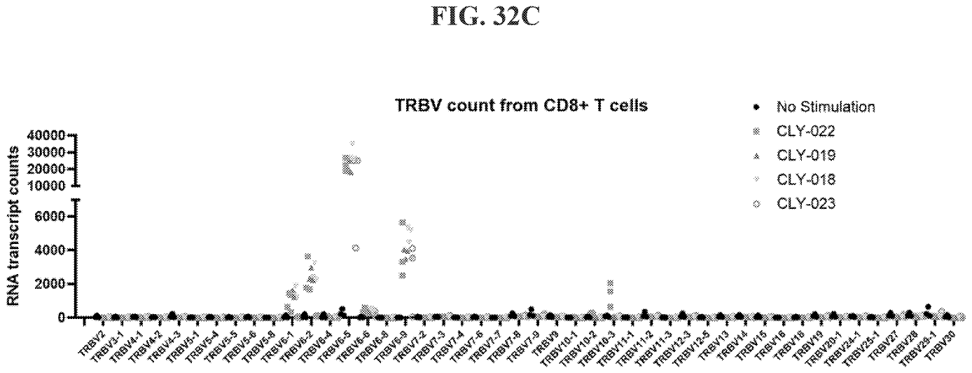

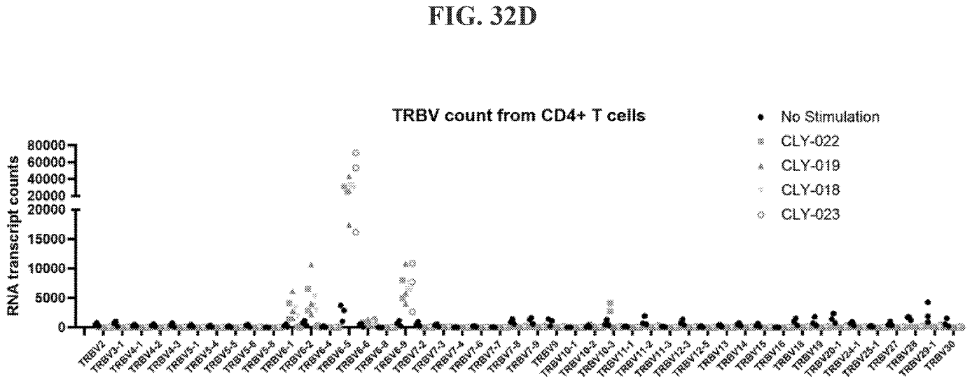

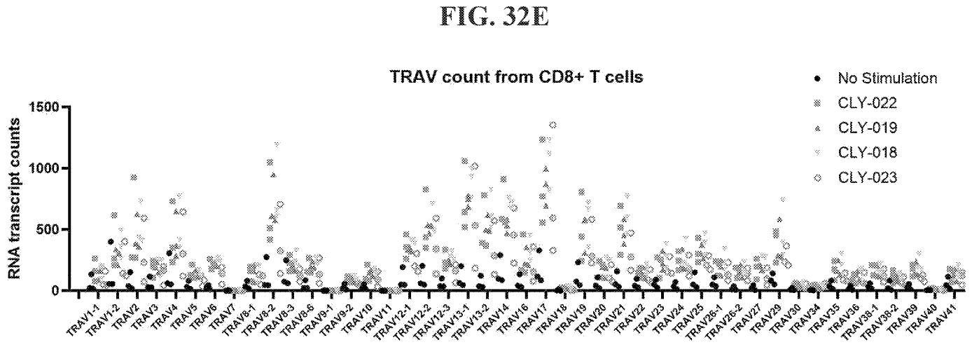

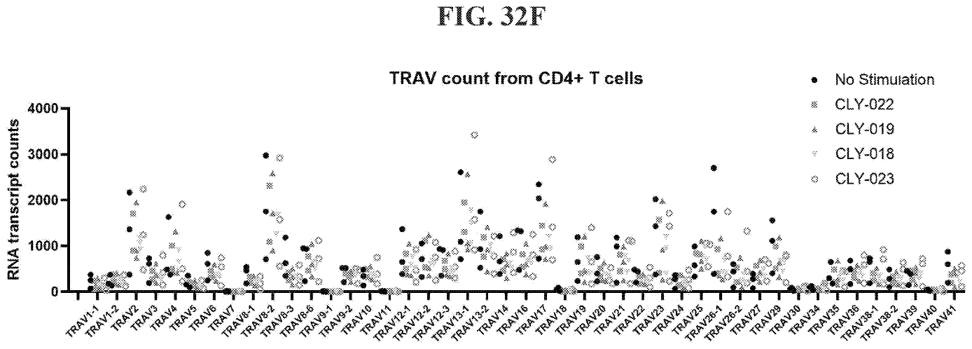

Results of CD4+ or CD8+ T cells using plate-bound CLY-023, CLY-018, CLY-019, or CLY-022 are shown in FIG. 32A (TRBV+CD8+ T cells) or FIG. 32B (TRBV+CD4+ T cells). Results show that plate-bound binding CLY-023, CLY-018, CLY-019, and CLY-022 induced CD8+ and CD4+ T cell expansion in a 7-day culture.

The expansion for TRBV+CD8+ T cells after 7-day culture with plate-bound binding CLY-023, CLY-018, CLY-019, and CLY-022 is shown at FIG. 32C. The expansion was specific to expansion of TRBV6-1, 6-2, 6-5, and 6-9 (n=3 donor CD8+ T cells). Results demonstrate that CD8+ T cells that expanded during the 7 day culture with plate bound polypeptide complexes were enriched for TRBV6-1, 6-2, 6-5, and 6-9.

The expansion for the TRBV+CD4 T cells after 7 day culture with plate bound CLY-023, CLY-018, CLY-019, or CLY-022 are shown in FIG. 32D. Results demonstrate that CD4+ T cells that expanded during the 7 day culture with plate bound polypeptide complexes were enriched for TRBV6-1, 6-2, 6-5, and 6-9.

Expansion of TRAV+CD8+ T cells after 7 day culture with plate bound CLY-023, CLY-018, CLY-019, or CLY-022, where no change in relative abundance of TRAVs with TRBV stimulation (n=3 donor CD8+ T cells) is shown in FIG. 32E. Results show that the CD8+ T cells that expanded during the 7 day culture with plate-bound T cell engagers exhibited no bias or enrichment for TRAV. Data is consistent with a lack of bias in TRAV pairing with TRBV6 or TRBV10.

A similar experiment was completed to demonstrate expansion of TRAV+CD4+ T cells after 7 day culture with plate-bound CLY-023, CLY-018, CLY-019, or CLY-022, where no change in relative abundance of TRAVs with TRBV stimulation (n=2-3 donor CD4+ T cells) is shown in FIG. 32F. Results show that the CD4+ T cells that expanded during the 7 day culture with plate-bound T cell engagers exhibited no bias or enrichment for TRAV. Data consistent with a lack of bias in TRAV pairing with TRBV6 or TRBV10.

### 8.7. Example 7: In Vivo B Cell Depletion and Cytokine Induction with CD19×TRBV T Cell Engagers

Pharmacokinetics and exploratory safety of polypeptide molecules were evaluated in cynomolgus monkeys. Briefly, cynomolgus monkeys of approximately 3 kg bodyweight were administered polypeptides intravenously or subcutaneously and observed daily for signs of adverse events. No in-life adverse events were observed at any timepoint for any polypeptide complex administered at any dose by any route. After dosing, blood was collected in K2 EDTA tubes at specific timepoints and processed to plasma for pharmacokinetic analysis. Blood samples were also stained and processed within a couple hours after collection and used for counting B cells by flow cytometry using standard techniques. Plasma was stored frozen until pharmacokinetic (PK) and cytokine analysis. Concentration of polypeptide molecules in plasma was measured via standard ELISA techniques relative to a reference standard diluted in control cyno plasma. Plasma concentration curves were fit to a standard two compartment model. Fitting of pharmacokinetics enabled the calculation of Cmax, half-life, volume of distribution, clearance, and area under the curve (AUC). Plasma was also analyzed for cytokines using a non-human primate cytometric Th1/Th2 bead array kit from BD biosciences following the manufacturer's instructions. IFNγ, TNFα, IL-6, IL-5, IL-4, and IL-2 levels in plasma were calculated relative to reference standards provided with the bead array kit.

### CLY-023 Cynomolgus Monkey Data

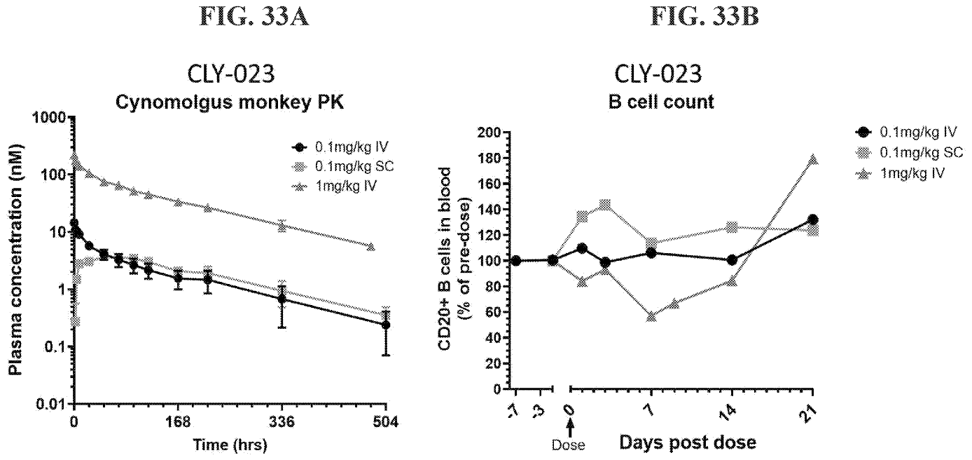

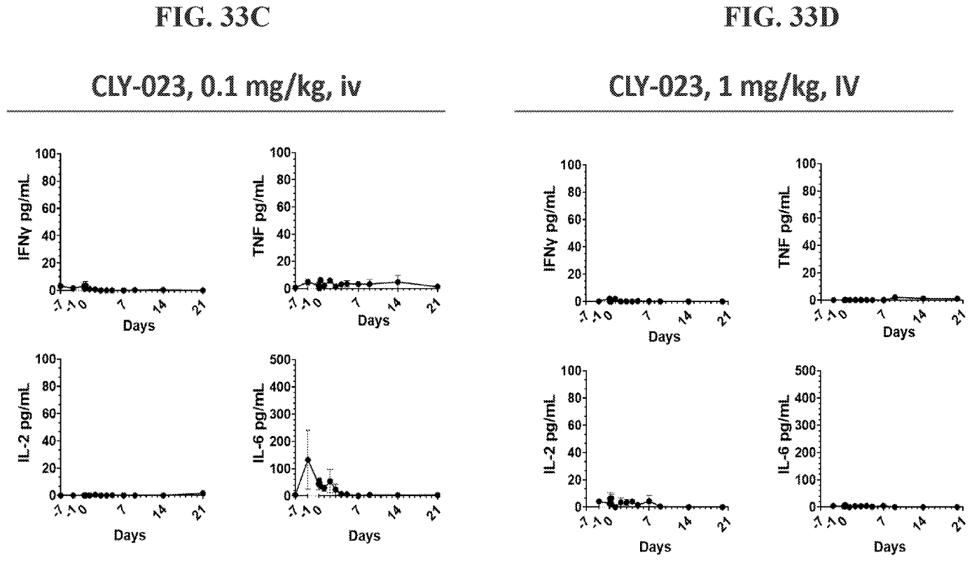

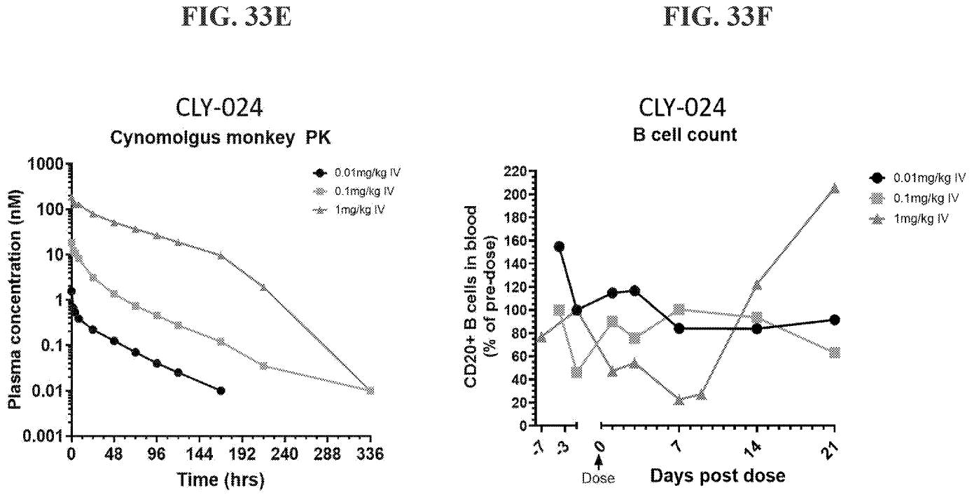

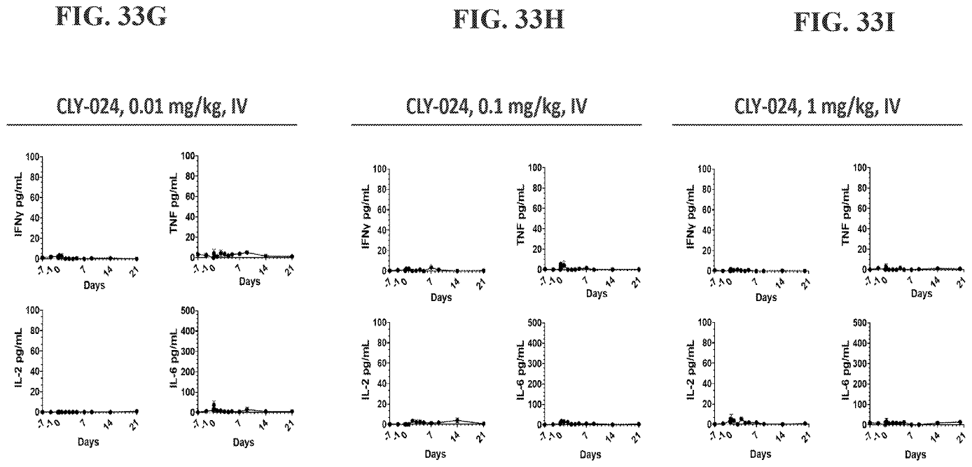

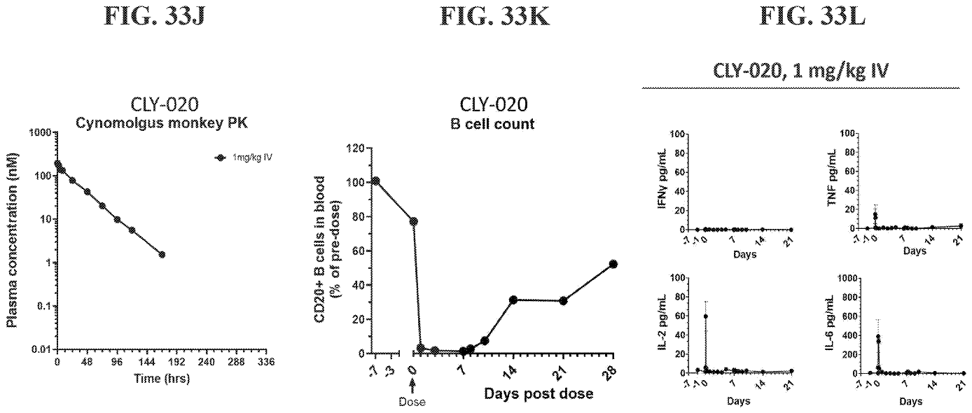

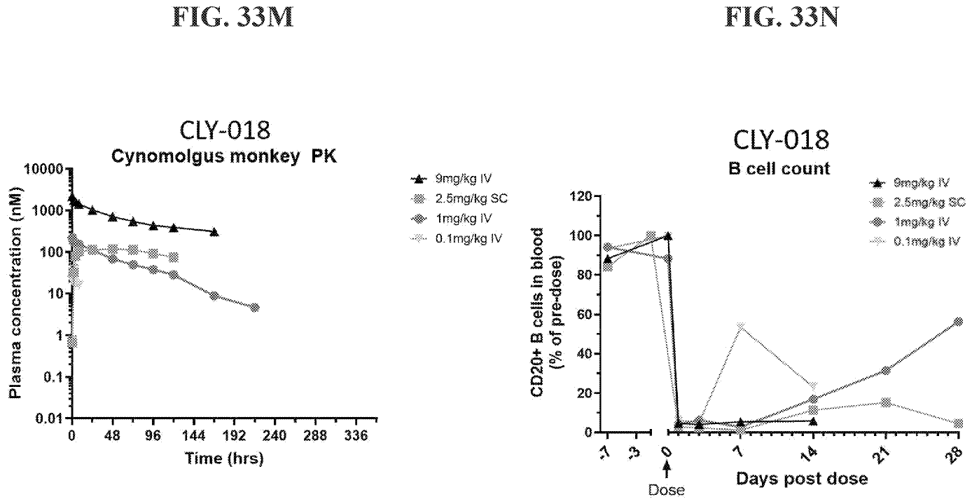

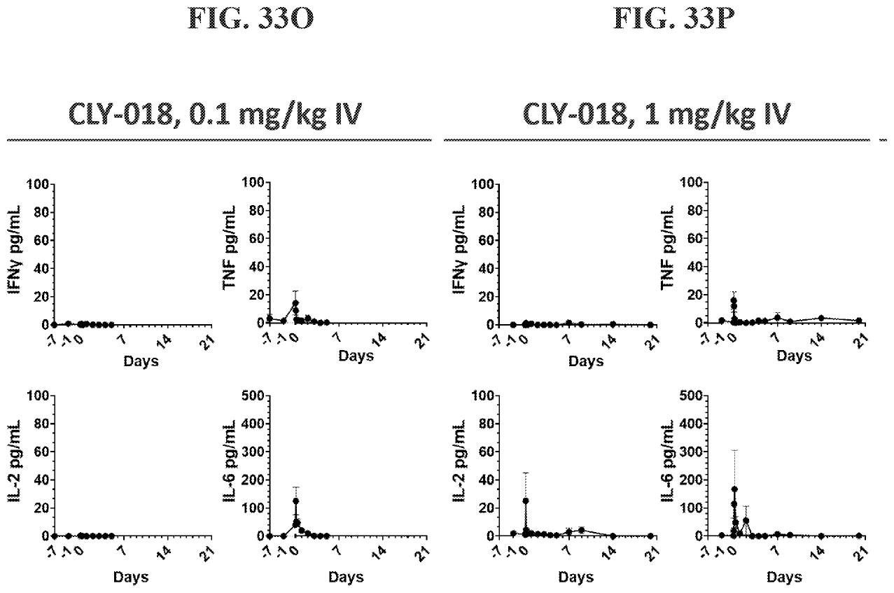

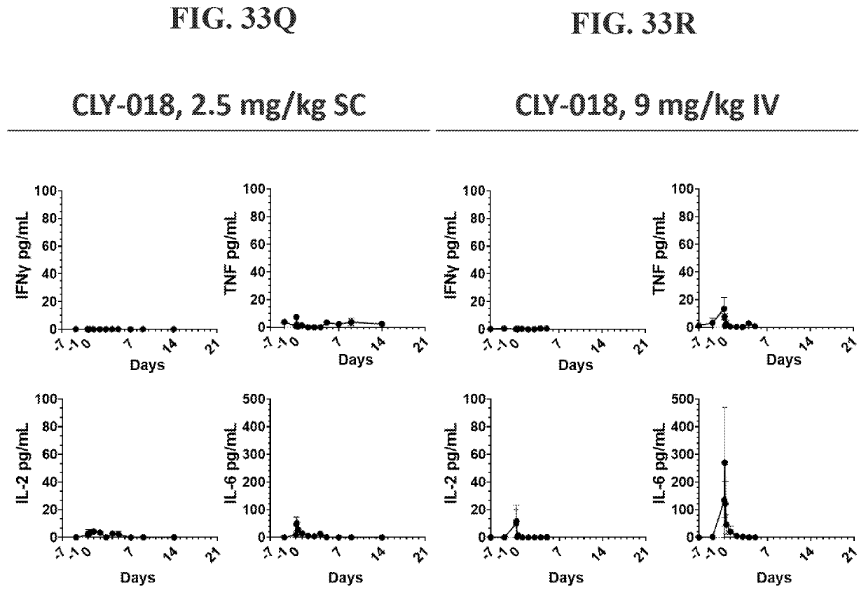

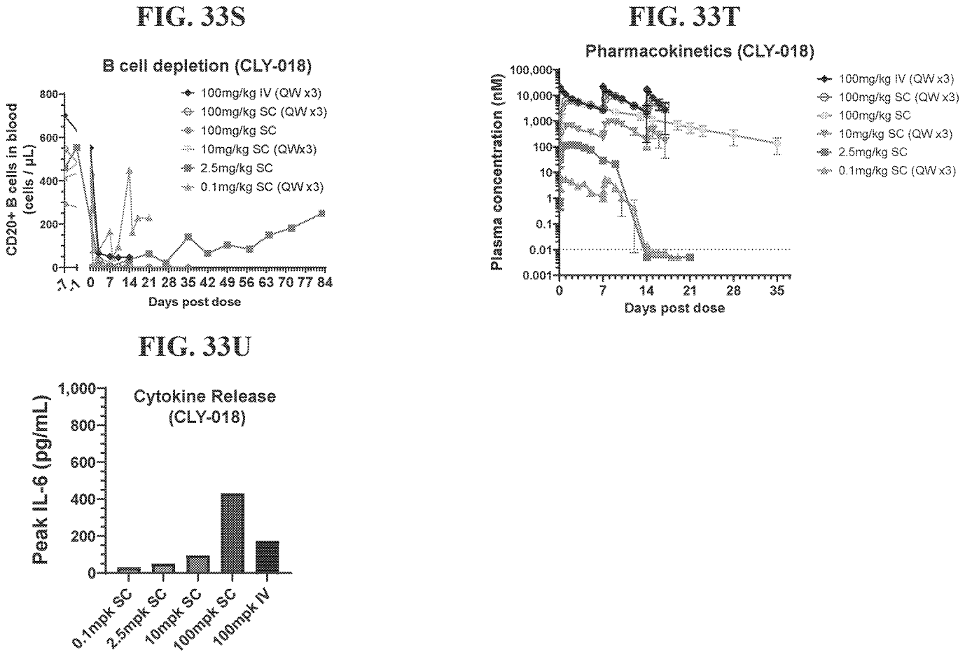

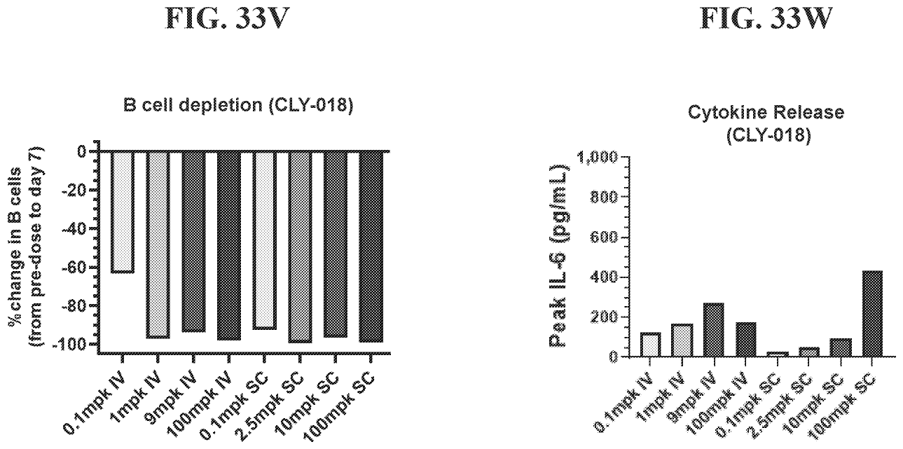

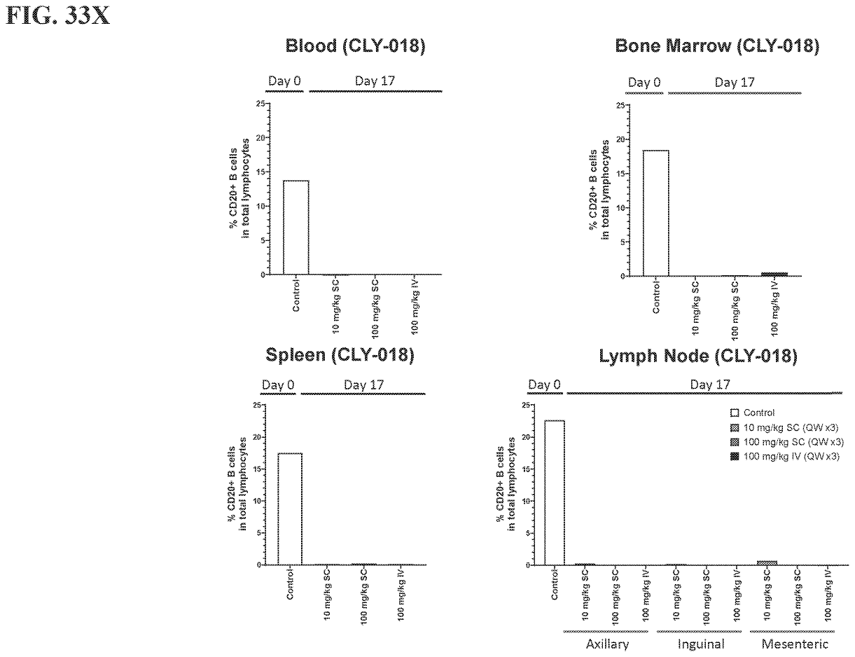

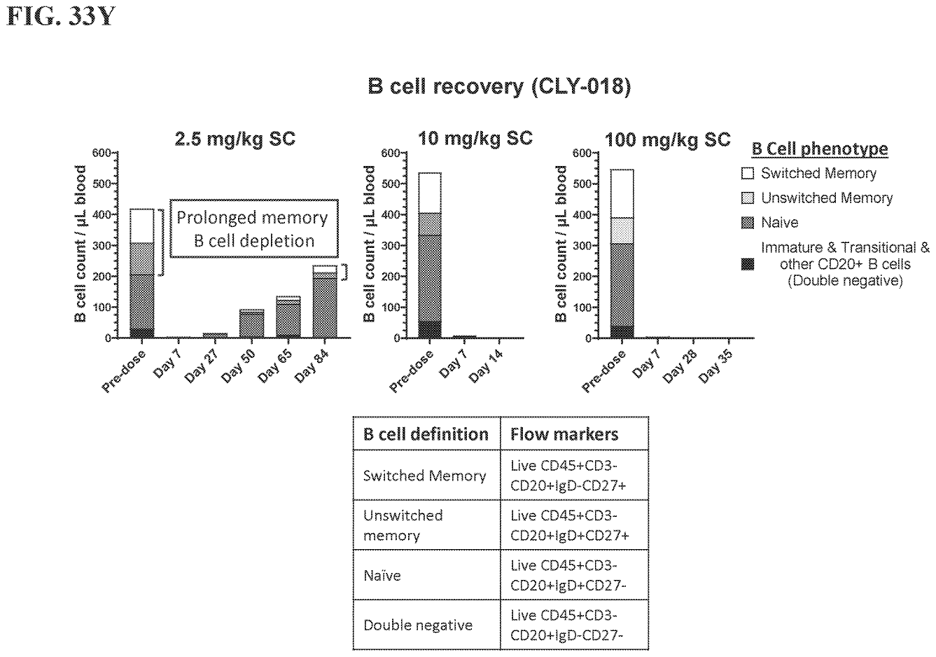

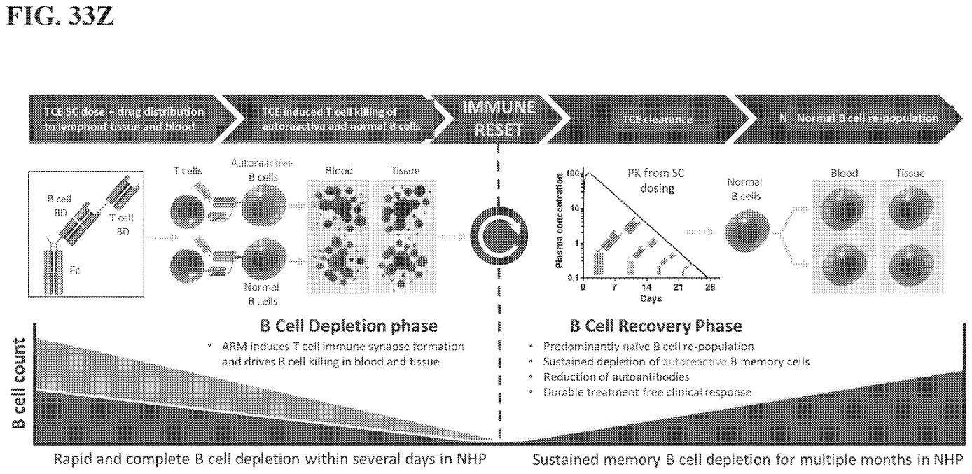

PK and B cell count data for monkeys dosed with CLY-023 is displayed at FIGS. 33A-33B. Minimal B cell depletion was observed in cynomolgus monkey dosed up to 1 mg/kg IV (FIG. 33B). Plasma concentration of CLY-023 as a function of time is displayed at FIG. 33A.

Cytokine induction data evaluating presence of IFNγ, TNF, IL-2, and IL-6 for the same monkeys is displayed at FIGS. 33C-33D. No in life observations or signs of cytokine release syndrome were observed in cynomolgus monkeys dosed up to 1 mg/kg IV.

### CLY-024 Cynomolgus Monkey Data

PK and B cell count data for monkeys dosed with CLY-024 is displayed at FIGS. 33E-33F. As shown in FIG. 33F, moderate B cell depletion observed in cynomolgus monkey dosed at 1 mg/kg IV; minimal B cell depletion observed at lower doses (<1 mg/kg IV). Plasma concentration of CLY-024 as a function of time is displayed at FIG. 33E.

Cytokine induction data for the same monkeys is displayed at FIGS. 33G-33I. No in life observations or signs of cytokine release syndrome were observed in cynomolgus monkeys dosed up to 1 mg/kg IV.

### CLY-020 Cynomolgus Monkey Data

PK, B cell count, and cytokine induction data for monkeys dosed with CLY-020 is displayed at FIGS. 33J-33L. As can be seen, the clearance of CLY-020 is faster than the aforementioned binders (FIG. 33J). Deep B cell depletion was observed in cynomolgus monkeys dosed at 1 mg/kg IV (FIG. 33K). No in life observations or signs of cytokine release syndrome in cynomolgus monkeys dosed at 1 mg/kg IV were detected (FIG. 33L).

### CLY-018 Cynomolgus Monkey Data (Round 1)

PK and B cell count data for monkeys dosed with CLY-018 is displayed at FIGS. 33M-33N. Deep B cell depletion was observed in cynomolgus monkeys dosed at 0.1 mg/kg IV or above (FIG. 33N).

Cytokine induction data for the same monkeys is displayed at FIGS. 33O-33R. No in life observations or signs of cytokine release syndrome were observed in cynomolgus monkeys dosed up to 9 mg/kg IV.

### CLY-018 Cynomolgus Monkey Data (Round 2)

Additional efficacy, PK, and safety studies were performed with the CLY-018 T cell engager in non-human primates using various dosing regimens and administration routes.

CLY-018 was administered to adult cynomolgus monkeys by the intravenous or subcutaneous route at the indicated doses and dosing regimen (single dose or once weekly on Day 0, Day 7 and Day 14). B cells in the blood were taken at various timepoints and analyzed for total B cells and B cell phenotypes. Blood samples were also processed to serum or plasma and analyzed for drug levels (pharmacokinetics (PK)) or cytokines, respectively. Animals were monitored twice daily for in life observations or clinical signs of toxicity. No clinical signs were observed at any dose level at any time during the studies. Some animals were euthanized on Day 17 and lymphoid tissues harvested in order to count B cells residing in tissues. Tissue samples were processed to single cell suspensions using standard techniques, stained with live/dead and surface markers for B cells, and analyzed by flow cytometry.

B cell depletion, PK, and IL-6 cytokine release for monkeys subcutaneously or intravenously administered a single dose or repeat doses of CLY-018 is shown at FIGS. 33S-W. Administration of CLY-18 resulted in deep and prolonged B cell depletion with no treatment-related clinical signs or cytokine release syndrome observed. All animals exhibited normal body temperature throughout study (data not shown). These data demonstrate >100× safety multiple where no cytokine release syndrome or adverse events were observed at doses (100 mg/kg IV) whose exposures are >100× higher than lower doses (2.5 mg/kg SC) where complete B cell depletion was observed with a prolonged immune reset (FIG. 33Y).

The percentage of CD20+ B cells in the blood, bone marrow, spleen, and lymph nodes before (Day 0) and after (Day 17) treatment with CLY-018 was determined. CLY-018 fully depleted B cells in blood and lymphoid tissues at Day 17, which was 3 days after the last dose (FIG. 33X). There were minimal to no detectable B cells in the bone marrow, spleen, and lymph nodes for monkeys dosed ≥10 mg/kg with no clinical signs or cytokine release syndrome observed (FIG. 33X).

B cell recovery following subcutaneous administration of CLY-018 at 2.5 mg/kg, 10 mg/kg, and 100 mg/kg was also determined. There was prolonged B cell depletion from a single subcutaneous dose with no treatment-related clinical signs or cytokine release syndrome observed (FIG. 33Y). B cell repopulation was primarily of the naïve B cell phenotype with sustained depletion of memory B cell populations for multiple months following a single dose of CLY-018 (FIG. 33Y).

Taken together, the CD19×TRBV T cell engagers fully and rapidly depleted B cells in blood and tissues without cytokine release syndrome. Prolonged memory B cell depletion was followed by an immune reset of primarily naïve B cells, which lasted at least 3 months after a single CD19×TRBV dose (FIG. 33Z). Pharmacokinetic and pharmacodynamic data supported administration once every two weeks (Q2W) using intravenous or subcutaneous routes. Collectively, these data demonstrate that CD19×TRBV T cell engagers have a best-in-class 100× safety multiple in non-human primates relative to CD19×CD3 T cell engagers or in vivo CAR T cells.

### 8.8. Example 8: B Cell Depletion, T Cell Expansion, and T Cell Phenotyping with CD19×TRBV T Cell Engagers in Healthy and Autoimmune Diseased Human PBMCs

Activity of the exemplary CLY-018 T cell engager was evaluated in healthy human donor PBMCS, as well as PBMCs from rheumatoid arthritis (RA), Systemic lupus erythematosus (SLE), and Myasthenia gravis (MG) autoimmune patients.

Polypeptide binding complexes were evaluated in flow cytometry based functional ex vivo assays using human PBMCs from autoimmune patients (RA, SLE, MG) that comprise unmanipulated physiologic ratios of T cells and B cells. Titrated polypeptide complexes were incubated with freshly thawed 250,000 human or cynomolgus monkey PBMCs per well for up to 7 days. Readouts were taken on days 1, 3, 4, 5, and/or 7 as well as pre-treatment (day 0). At the indicated timepoints supernatants were harvested for cytokine measurements and cells were harvested for flow cytometry measurements. Supernatants from human PBMCs were analyzed for cytokines using a Meso Scale Device kit following the manufacturer's instructions. Supernatants from cynomolgus monkey PBMCs were analyzed using a cytometric bead array following the manufacturer's instructions. Flow cytometry measurements included live/dead, CD3, CD8, CD4, Foxp3, TRBV, CD25, Granzyme B, Ki-67, CCR7, CD45RA, CD95, CD20, and/or CD19 in the panel. Counting beads were included to accurately quantify absolute number of cells present in the samples. Flow cytometric readouts were plotted against the logarithmic concentration of polypeptide complex. Where indicated the concentration of polypeptide complex required to reach half maximum readout was calculated in Graphpad Prism and reported as an EC50. Readouts included T cell expansion (change in absolute number of viable T cells or T cell subsets), T cell activation, as well as killing of B cells (change in viable B cell counts). Fold change in T cell counts was calculated as polypeptide complex target positive T cells (CD3+ or TRBV+) post polypeptide complex treatment divided by the analogous T cell counts without treatment.

Assays implemented the below gating parameters (All viable cells were gated with live/dead stain): B cells (CD3−CD20+); CD8+ T cells (CD3+CD8+); CD4+ T cells-(CD3+CD4+FoxP3−); Tregs (CD3+CD4+FoxP3+); Tn (CD3+CD8+ (or CD4+)CCR7+CD45RA+CD95−); Tscm−(CD3+CD8+ (or CD4+) CCR7+CD45RA+CD95+); Ttm (CD3+CD8+ (or CD4+) CCR7+CD45RA+/−); Tcm (CD3+CD8+ (or CD4+) CCR7+CD45RA−); Tem (CD3+CD8+ (or CD4+) CCR7−CD45RA−); Teff (CD3+CD8+ (or CD4+) CCR7−CD45RA+/−); and Temra (CD3+CD8+(or CD4+) CCR7−CD45RA+).

### Healthy Donor

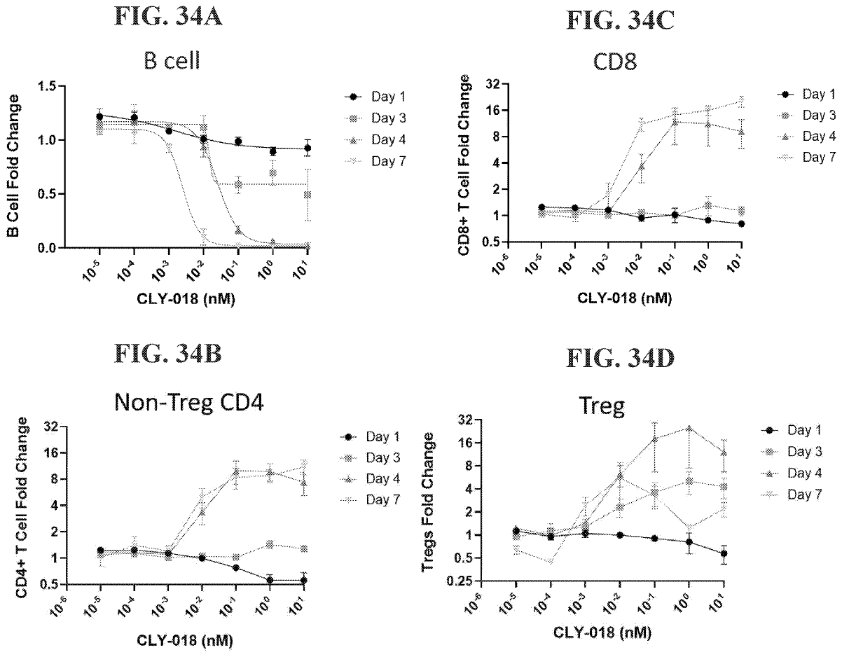

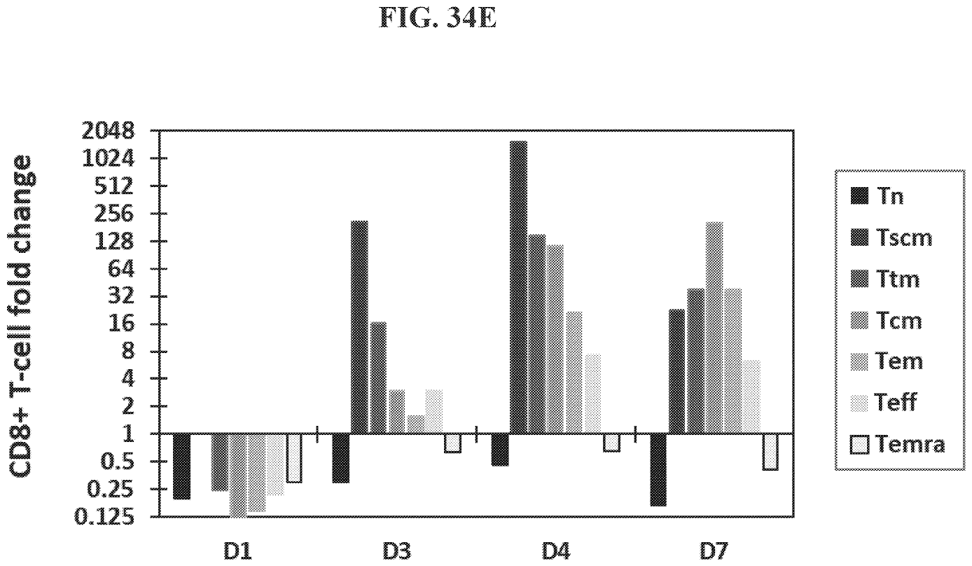

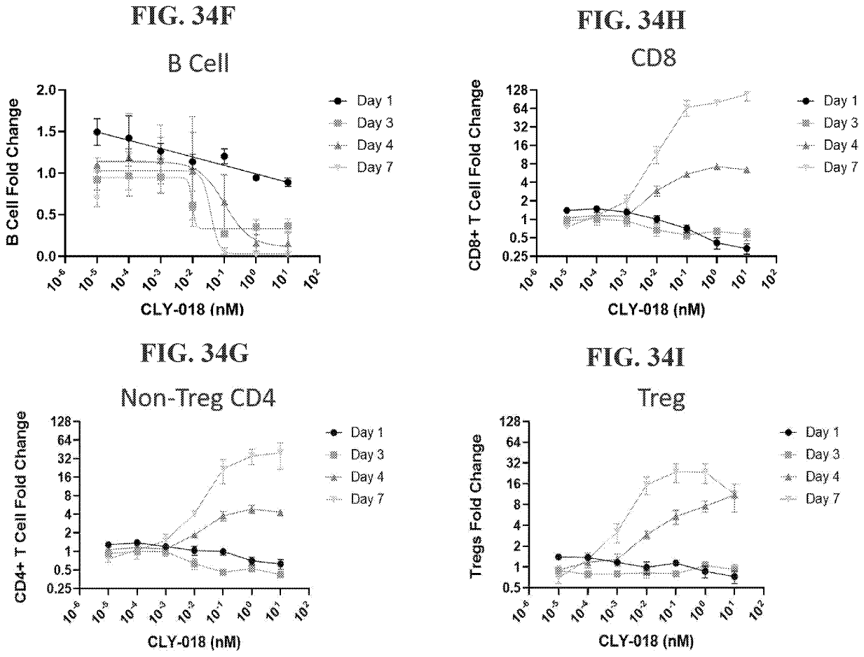

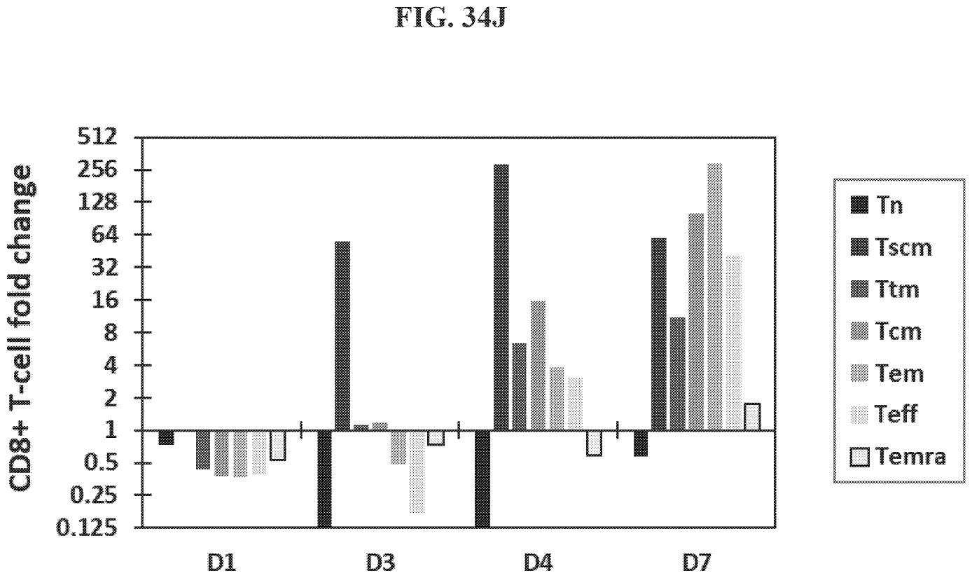

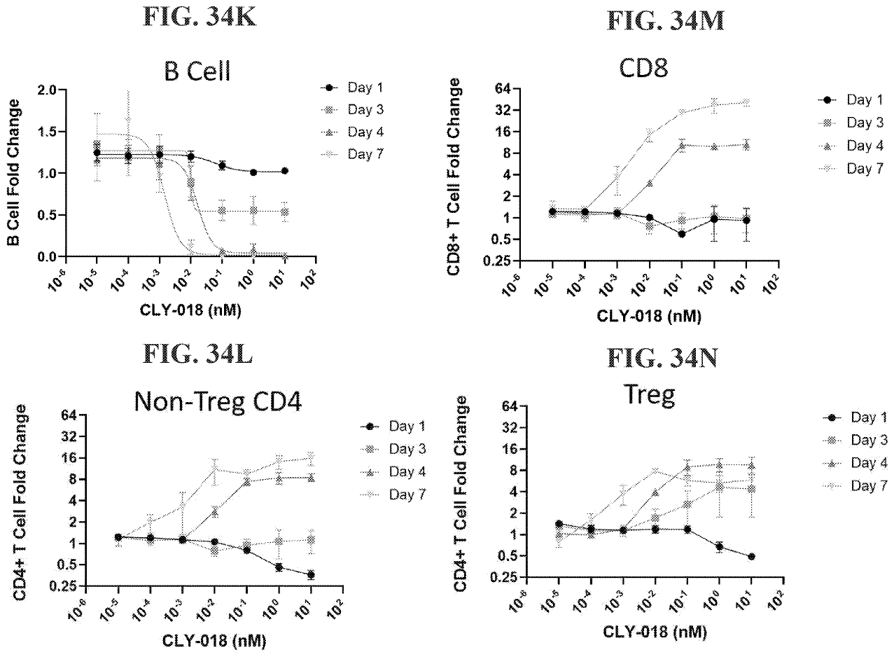

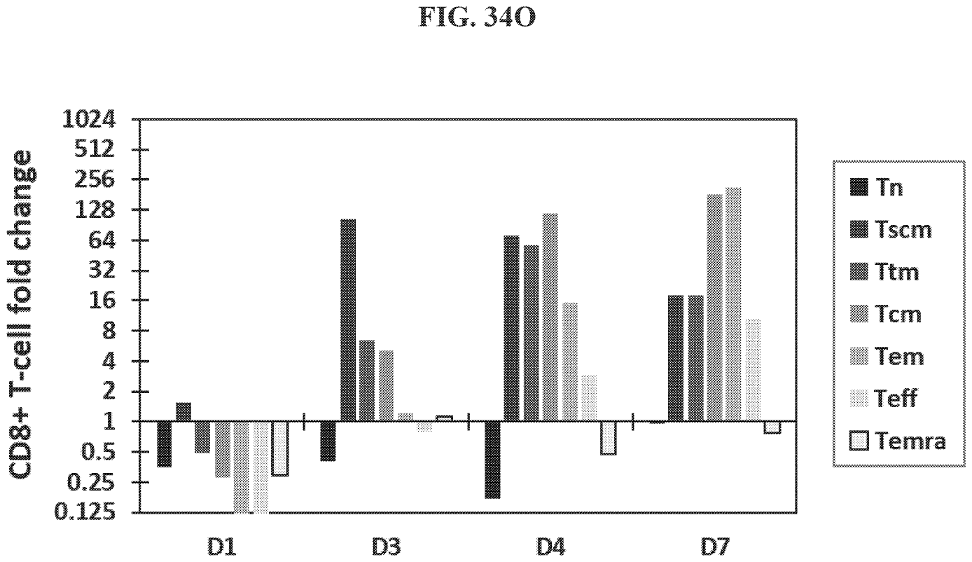

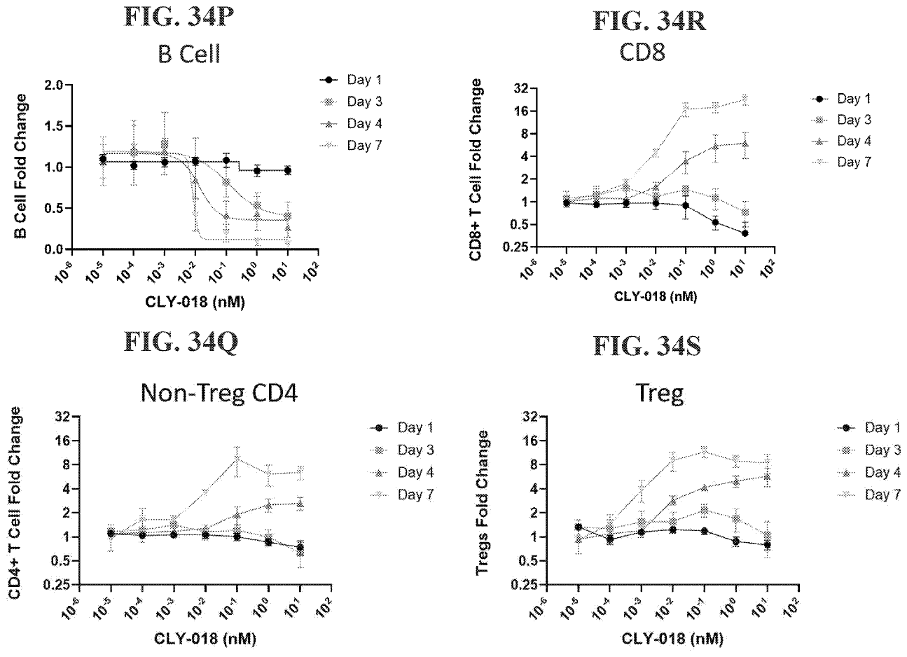

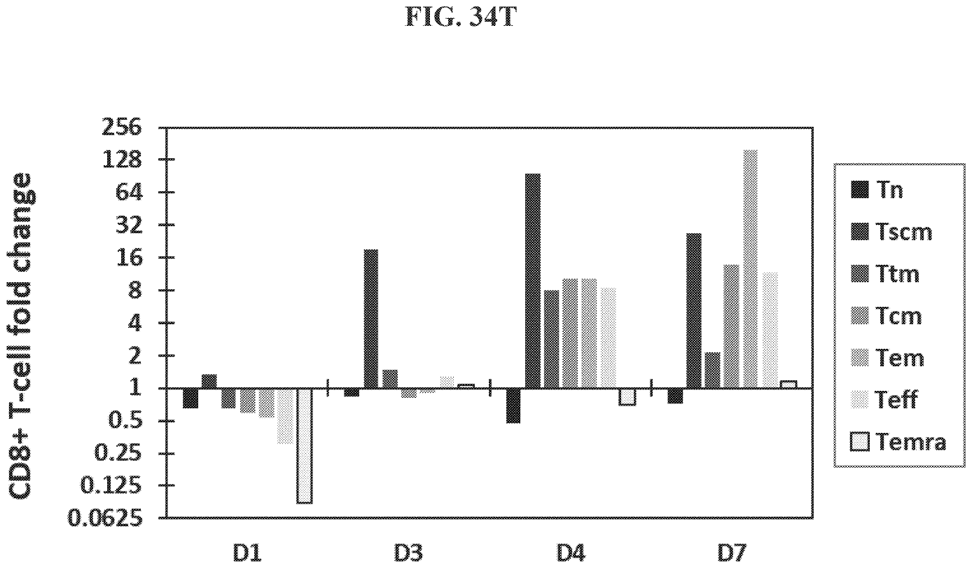

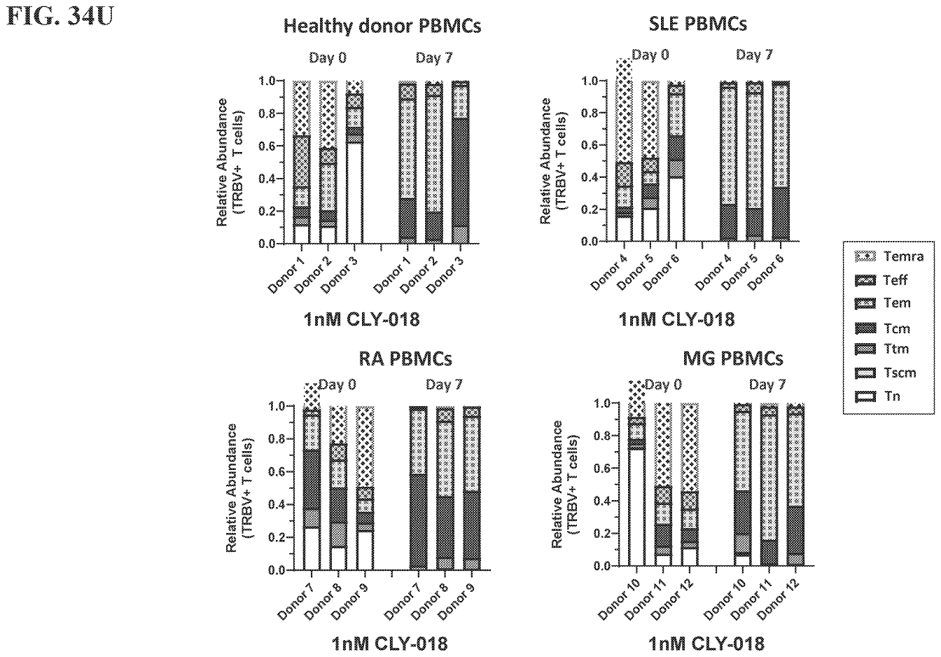

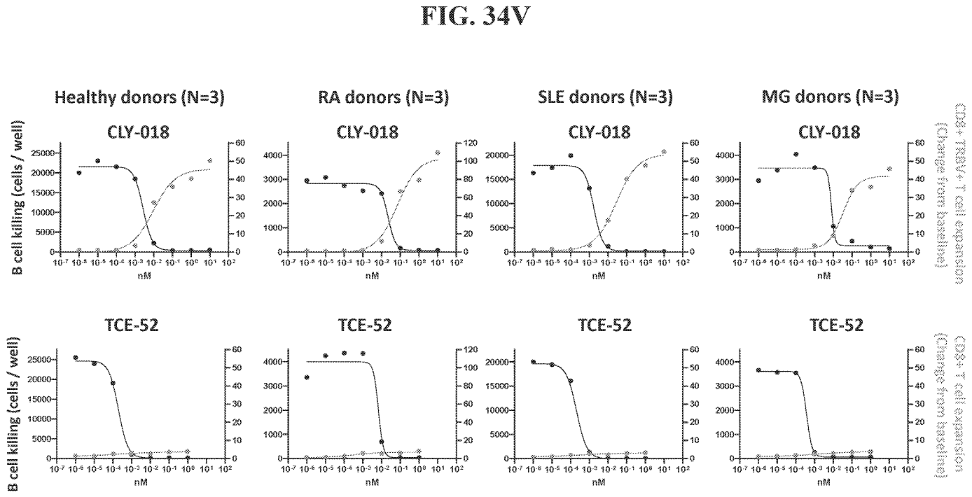

B cell killing and T cell expansion results for human healthy donor PBMCs incubated with CLY-018 are displayed at FIG. 34A (B cell fold change), FIG. 34B (non-Treg CD4+ T cell fold change), FIG. 34C (CD8+ T cell fold change), and FIG. 34D (Treg fold change). Results displayed are for an average of 3 healthy donors (N=3). Expansion according to Tn, Tscm, Ttm, Tcm, Tem, Teff, and Temra CD8+ T cell subsets is shown at FIG. 34E.

### Rheumatoid Arthritis (RA)

PBMCs from 3 rheumatoid arthritis subjects were collected and incubated with CLY-018. Results of B cell killing is shown at FIG. 34F. Results for T cell expansion for CD8+, Treg, and non-Treg CD4+ populations is provided in FIG. 34H, FIG. 34I, and FIG. 34G, respectively. Expansion according to Tn, Tscm, Ttm, Tcm, Tem, Teff, and Temra CD8 T cell subsets is shown at FIG. 34J.

### Systemic Lupus Erythematosus (SLE)

PBMCs from 3 SLE subjects were collected and incubated with CLY-018. Results of B cell killing is provided at FIG. 34K. Results of T cell expansion for CD8+, Treg, and non-Treg CD4+ populations is provided in FIG. 34M, FIG. 34N, and FIG. 34L, respectively. Expansion according to Tn, Tscm, Ttm, Tcm, Tem, Teff, and Temra CD8 T cell subsets is shown at FIG. 34O.

### Myasthenia Gravis (MG)

PBMCs from 3 MG subjects were collected and incubated with CLY-018. Results of B cell killing is provided at FIG. 34P. Results of T cell expansion for CD8+, Treg, and non-Treg CD4+ populations is provided in FIG. 34R, FIG. 34S, and FIG. 34Q, respectively. Expansion according to Tn, Tscm, Ttm, Tcm, Tem, Teff, and Temra CD8 T cell subsets is shown at FIG. 34T.

T Cell Subsets in Healthy and Autoimmune PBMCs Treated with CD19×TRBV T Cell Engagers

The relative abundance of TRBV+ T cell subsets in healthy and autoimmune disease PBMCs treated with the CD19×TRBV T cell engager CLY-018 was determined as described above. Briefly, relative T cell abundance was measured in PBMCs before (Day 0) or following a 7 day incubation with 1 nM of CLY-018. Relative abundance was calculated as the fraction of TRBV+ T cells of the indicated phenotype within the total TRBV+ T cells in the well. Expansion of Tn, Tscm, Ttm, Tcm, Tem, Teff, and Temra TRBV+ T cell subsets is shown at FIG. 34U. These data show memory T cell population expansion with maintenance of effector T cell populations.

### Comparison to CD19×CD3 T Cell Engagers

Additional experiments were performed to assess the activity of the CD19×TRBV T cell engager (CLY-018) compared to the CD19×CD3 T cell engager (TCE-52, Blinatumomab) in healthy human donor PBMCS, as well as PBMCs from RA, SLE, and MG autoimmune patients. B cell killing and T cell expansion were determined using the experimental protocol described above.

B cell killing and CD8+ T cell expansion data following treatment with CLY-018 or TCE-52 is shown at FIG. 34V. Overall, the CD19×TRBV T cell engager drives complete B cell killing with significant T cell expansion compared to the CD19×CD3 T cell engager in both healthy and autoimmune disease PBMCs.

### 8.9. Example 9: Continual T Cell Stimulation with the CD19×TRBV T Cell Engager Maintains T Cell Cytolytic Function

The aim of this study was to assess durability of cytolytic T cell function following continual T cell stimulation with CD19×TRBV and CD19×CD3 T cell engagers.

Pan T cells were co-cultured with Raji cells at a ratio of (2:1) in the presence or absence of 1 nM CD19×TRBV or 1 nM CD19×CD3 T cell engager in a 96 well plate format. T cells (Live CD3+) and Raji (Live CD3-CD20+) cells were counted daily by flow cytometry. If Raji cells decreased in number, additional Raji cells were added to the wells daily to maintain continual T stimulation and a T cell to Raji cell ratio of 2:1 throughout the study. If T cells expanded more, Raji cells were added to maintain a T cell to Raji cell ratio of 2:1 throughout the study. Medium was changed every other day. Specific lysis of Raji cells on Day 6 through Day 16 was calculated based on the number of live Raji cells counted on the day of interest relative to the number of live Raji cells counted from the previous day plus any additional Raji cells added to the well. 100% specific cell lysis means no live Raji cells were detected on that day. 50% specific lysis of Raji cells means that live Raji cell counts were half in number compared to that counted and/or added the previous day.

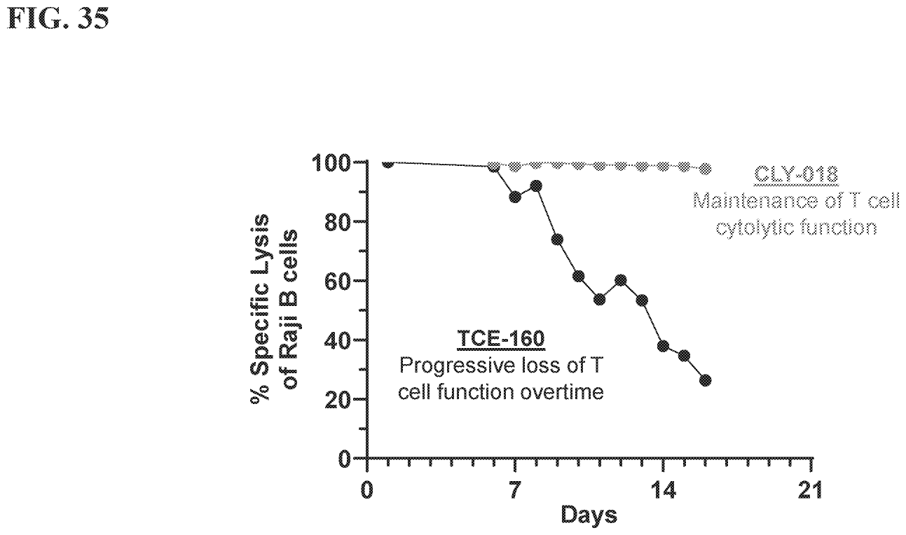

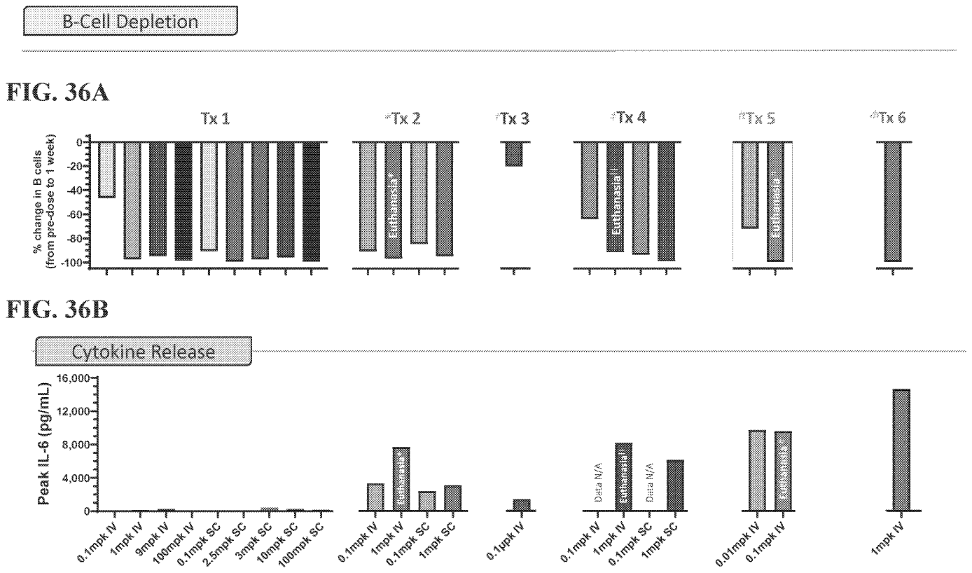

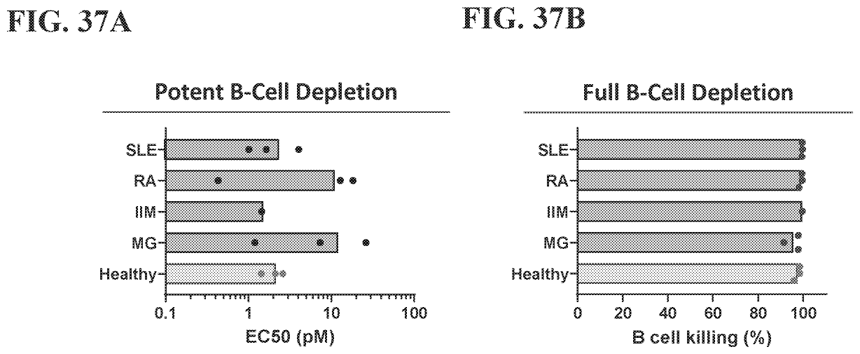

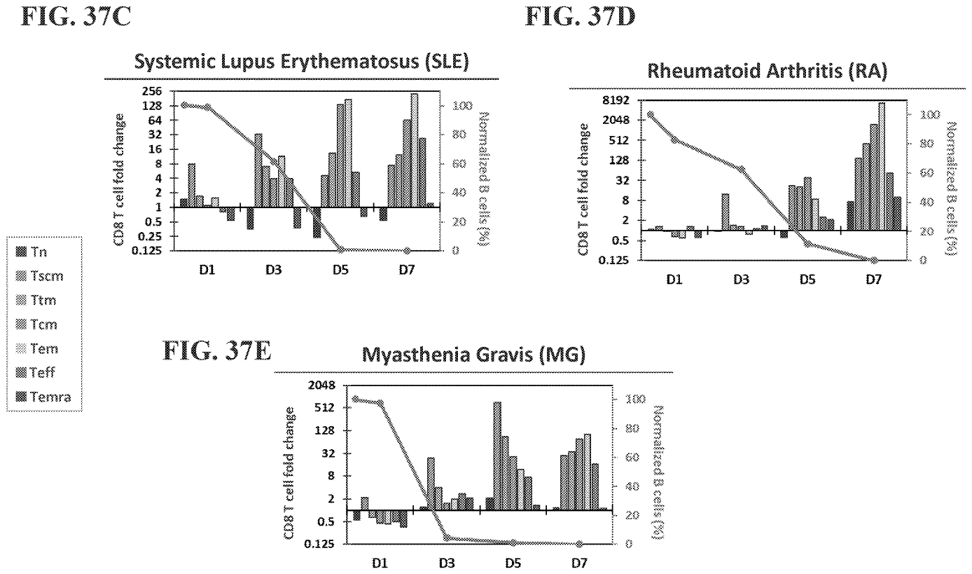

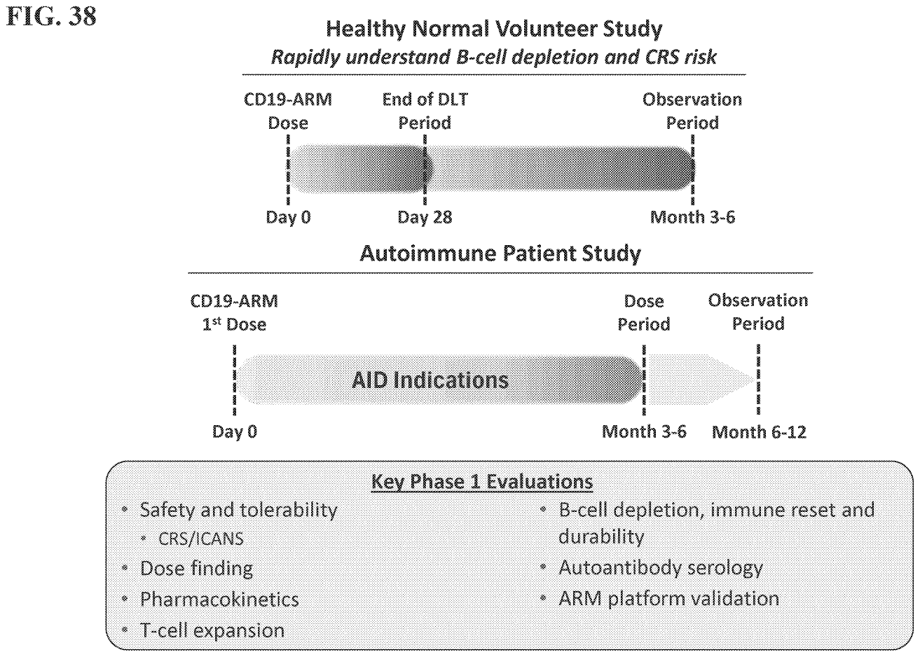

The CD19×CD3 T cell engager induced loss of T cell function whereas the CD19×TRBV T cell engager maintained full T cell cytolytic activity over the same time period (FIG. 35). This data demonstrates that CD19×TRBV T cell engagers maintain more durable T cell responses and are less prone to T cell exhaustion compared to CD19×CD3 T cell engagers.

### 8.10. Example 10: CD19×TRBV T Cell Engager Clinical Trial

CD19×TRBV T cell engagers of the present disclosure will be tested in a Phase I clinical trial. Healthy normal volunteers will be administered a CD19×TRBV T cell engager of the present disclosure at day zero. Various doses will be tested, including doses ranging from about 0.0001 mg/kg to about 0.1 mg and about 0.07 mg/kg to about 0.017 mg/kg. Various modes of administration will be evaluated, including IV, IP, and subcutaneous. The design includes a dose limiting trial period of 28 days, followed by an additional 2-5 months of observation. Separately, patients with autoimmune disorders will receive a CD19×TRBV T cell engager of the present disclosure at day zero (e.g., at about 0.07 mg/kg to about 0.017 mg/kg). Additional doses may be regularly administered over a 3-6 dosing period followed by a 6-12 month observation period. Disorders that will be tested include, but are not limited to: rheumatoid arthritis, Systemic lupus erythematosus, Multiple Sclerosis (MS), Type 1 Diabetes, Inflammatory Bowel Disease (IBD), Psoriasis, Hashimoto's Thyroiditis, Graves' Disease, Celiac Disease, Ankylosing Spondylitis, Sjögren's Syndrome, Myasthenia Gravis, Scleroderma, Autoimmune Hepatitis, Addison's Disease, Vasculitis, Pernicious anemia, immune thrombocytopenia purpura (ITP), ulcerative colitis (UC), Felty's syndrome, autoimmune hemolytic anemia, bullous pemphigoid, autoimmune neutropenia, autoimmune polyglandular syndrome 1, Goodpasture's disease, pemphigus vulgaris, polychondritis, epidermolysis bullosa acquisita, and pemphigus foliaceus

The trial will measure safety and tolerability of the therapeutic. The trial will validate ideal doses. Pharmacokinetics will be measured, as well as T cell expansion and B cell depletion. For autoimmune patients, autoantibody serology will be measured.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

What is claimed:

1. A multi-specific polypeptide comprising:
(a) a first binding domain that specifically binds T cell receptor beta variable region (TCRβV), wherein the first binding domain comprises:
(i) an IMGT heavy chain complementary determining region 1 (HCDR1) that comprises an amino acid sequence selected from the group consisting of: GYSFTTYY (SEQ ID NO: 17); GYSFRLTY (SEQ ID NO: 23); GHDFRLTY (SEQ ID NO: 24); and GYSFTLYY (SEQ ID NO: 1143);
(ii) an IMGT heavy chain complementary determining region 2 (HCDR2) that comprises an amino acid sequence selected from the group consisting of: FFPGSGNI (SEQ ID NO: 18); VSAGSGNV (SEQ ID NO: 25); FFPRSGNI (SEQ ID NO: 1147); IFPGSGNI (SEQ ID NO: 1184); and IYPGSGNI (SEQ ID NO: 1185);
(iii) an IMGT heavy chain complementary determining region 3 (HCDR3) that comprises an amino acid sequence selected from the group consisting of:

| (SEQ ID NO: 19) | |
|---|---|
| | AGSYYSYDVLDY; |
| (SEQ ID NO: 26) | |
| | AVSYYSYDVLDY; |
| (SEQ ID NO: 1144) | |
| | AASYYSFDVLDY; |
| | and |
| (SEQ ID NO: 1186) | |
| | ARSYYSYDVLDY; |

(iv) an IMGT light chain complementary determining region 1 (LCDR1) that comprises an amino acid sequence selected from the group consisting of: QNVGIN (SEQ ID NO: 20); QNVADR (SEQ ID NO: 27); QNVGIR (SEQ ID NO: 1145); and QNVGNN (SEQ ID NO: 1187);
(v) an IMGT light chain complementary determining region 2 (LCDR2) that comprises an amino acid sequence of SSS; and
(vi) an IMGT light chain complementary determining region 3 (LCDR3) that comprises an amino acid sequence selected from the group consisting of: QQFKSYPLT (SEQ ID NO: 22) and QQFKSYPIT (SEQ ID NO: 1146); and
(b) a second binding domain that specifically binds CD19.

2. The multi-specific polypeptide of claim 1, wherein the first binding domain comprises:
(i) the HCDR1, the HCDR2, and the HCDR3 comprising the amino acid sequences of SEQ ID NOs: 17, 18, and 19, respectively, and the LCDR1, the LCDR2, and the LCDR3 comprising the amino acid sequences of SEQ ID NO: 20, SSS, and SEQ ID NO: 22, respectively;
(ii) the HCDR1, the HCDR2, and the HCDR3 comprising the amino acid sequences of SEQ ID NOs: 23, 18, and 19, respectively, and the LCDR1, the LCDR2, and the LCDR3 comprising the amino acid sequences of SEQ ID NO: 20, SSS, and SEQ ID NO: 22, respectively;
(iii) the HCDR1, the HCDR2, and the HCDR3 comprising the amino acid sequences of SEQ ID NOs: 24, 25, and 26, respectively, and the LCDR1, the LCDR2, and the LCDR3 comprising the amino acid sequences of SEQ ID NO: 27, SSS, and SEQ ID NO: 22, respectively;
(iv) the HCDR1, the HCDR2, and the HCDR3 comprising the amino acid sequences of SEQ ID NOs: 1143, 18, and 1144, respectively, and the LCDR1, the LCDR2, and the LCDR3 comprising the amino acid sequences of SEQ ID NO: 1145, SSS, and SEQ ID NO: 1146, respectively;
(v) the HCDR1, the HCDR2, and the HCDR3 comprising the amino acid sequences of SEQ ID NOs: 17, 1147, and 1144, respectively, and the LCDR1, the LCDR2, and the LCDR3 comprising the amino acid sequences of SEQ ID NO: 1145, SSS, and SEQ ID NO: 1146, respectively;
(vi) the HCDR1, the HCDR2, and the HCDR3 comprising the amino acid sequences of SEQ ID NOs: 17, 1184, and 19, respectively, and the LCDR1, the LCDR2, and the LCDR3 comprising the amino acid sequences of SEQ ID NO: 1187, SSS, and SEQ ID NO: 22, respectively;
(vii) the HCDR1, the HCDR2, and the HCDR3 comprising the amino acid sequences of SEQ ID NOs: 17, 1185, and 19, respectively, and the LCDR1, the LCDR2, and the LCDR3 comprising the amino acid sequences of SEQ ID NO: 20, SSS, and SEQ ID NO: 22, respectively; or
(viii) the HCDR1, the HCDR2, and the HCDR3 comprising the amino acid sequences of SEQ ID NOs: 17, 1185, and 1186, respectively, and the LCDR1, the LCDR2, and the LCDR3 comprising the amino acid sequences of SEQ ID NO: 1187, SSS, and SEQ ID NO: 22, respectively.

3. The multi-specific polypeptide of claim 1, wherein the first binding domain comprises an immunoglobulin heavy chain variable (VH) region and/or an immunoglobulin light chain variable (VL) region.

4. The multi-specific polypeptide of claim 3, wherein the VH region of the first binding domain comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 1009-1016, 1148, 1150, 1190, and 1191; and/or the VL region of the first binding domain comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 1001-1008, 1149, and 1192.

5. The multi-specific polypeptide of claim 1, wherein the first binding domain is a single chain variable fragment (scFv), Fab, CrossFab, Fab′, or F(ab′)₂.

6. The multi-specific polypeptide of claim 1, wherein the first binding domain is an scFv comprising a VH region and a VL region.

7. The multi-specific polypeptide of claim 6, wherein the C-terminus of the VH region of the first binding domain is linked to the N-terminus of the VL region of the first binding domain; or the C-terminus of the VL region of the first binding domain is linked to the N-terminus of the VH region of the first binding domain.

8. The multi-specific polypeptide of claim 6, wherein the VH region and VL region of the first binding domain are linked by a peptide linker.

9. The multi-specific polypeptide of claim 8, wherein the peptide linker comprises the formula of (Gly₄Ser)ₙ, wherein n=1-5.

10. The multi-specific polypeptide of claim 6, wherein the VH region and the VL region of the first binding domain are linked by one or more engineered disulfide bonds.

11. The multi-specific polypeptide of claim 8, wherein the positions of the one or more engineered disulfide bonds are selected from the group consisting of: VH44-VL100, VH100B-VL49, VH100-VL50, VH101-VL46, and VH105-VL43, according to EU numbering.

12. The multi-specific polypeptide of claim 5, wherein the first binding domain is an scFv, and the scFv comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 1017-1054 and 1151-1154.

13. The multi-specific polypeptide of claim 1, wherein the second binding domain comprises:
(i) an IMGT HCDR1 that comprises an amino acid sequence selected from the group consisting of: GYAFSSYW (SEQ ID NO: 337); GYTFTSNW (SEQ ID NO: 338); GYTFTSYV (SEQ ID NO: 339); GGSISTSGMG (SEQ ID NO: 340); GFTFSSSW (SEQ ID NO: 341); GGSISTSTMG (SEQ ID NO: 342); and GGSIASGMG (SEQ ID NO: 343);
(ii) an IMGT HCDR2 that comprises an amino acid sequence selected from the group consisting of: IWPGDGDT (SEQ ID NO: 344); IDPSDSYT (SEQ ID NO: 345); INPYNDGT (SEQ ID NO: 346); IWWDDDK (SEQ ID NO: 347); IYPGDGDT (SEQ ID NO: 348);
(iii) an IMGT HCDR3 that comprises an amino acid sequence selected from the group consisting of: ARRETTTVGRYYYAMDY (SEQ ID NO: 349); ARGSNPYYYAMDY (SEQ ID NO: 350); ARGTYYYGTRVFDY (SEQ ID NO: 351); ARMELWSYYFDY (SEQ ID NO: 352); ARSGFITTVRDFDY (SEQ ID NO: 353); ARMELWSYYFDP (SEQ ID NO: 354);
(iv) an IMGT LCDR1 that comprises an amino acid sequence selected from the group consisting of: QSVDYDGDSY (SEQ ID NO: 355); SGVNY (SEQ ID NO: 356); KSLQNVNGNTY (SEQ ID NO: 357); SSVSY (SEQ ID NO: 358); ESVDTFGISF (SEQ ID NO: 359); SSVGY (SEQ ID NO: 360);
(v) an IMGT LCDR2 that comprises an amino acid sequence selected from the group consisting of: DAS; DTS; RMS; and EAS; and
(vi) an IMGT LCDR3 that comprises an amino acid sequence selected from the group consisting of: QQSTEDPWT (SEQ ID NO: 332); HQRGSYT (SEQ ID NO: 333); MQHLEYPIT (SEQ ID NO: 334); FQGSVYPFT (SEQ ID NO: 335); and QQSKEVPFT (SEQ ID NO: 336).

14. The multi-specific polypeptide of claim 1, wherein the second binding domain comprises:
(i) the HCDR1, the HCDR2, and the HCDR3 comprising the amino acid sequences of SEQ ID NOs: 337, 344, and 349, respectively, and the LCDR1, the LCDR2, and the LCDR3 comprising the amino acid sequences of SEQ ID NO: 355, DAS, and SEQ ID NO: 332, respectively;
(ii) the HCDR1, the HCDR2, and the HCDR3 comprising the amino acid sequences of SEQ ID NOs: 338, 345, and 350, respectively, and the LCDR1, the LCDR2, and the LCDR3 comprising the amino acid sequences of SEQ ID NO: 356, DTS, and SEQ ID NO: 333, respectively;
(iii) the HCDR1, the HCDR2, and the HCDR3 comprising the amino acid sequences of SEQ ID NOs: 339, 346, and 351, respectively, and the LCDR1, the LCDR2, and the LCDR3 comprising the amino acid sequences of SEQ ID NO: 357, RMS, and SEQ ID NO: 334, respectively;
(iv) the HCDR1, the HCDR2, and the HCDR3 comprising the amino acid sequences of SEQ ID NOs: 340, 347, and 352, respectively, and the LCDR1, the LCDR2, and the LCDR3 comprising the amino acid sequences of SEQ ID NO: 358, DTS, and SEQ ID NO: 335, respectively;
(v) the HCDR1, the HCDR2, and the HCDR3 comprising the amino acid sequences of SEQ ID NOs: 341, 348, and 353, respectively, and the LCDR1, the LCDR2, and the LCDR3 comprising the amino acid sequences of SEQ ID NO: 359, EAS, and SEQ ID NO: 336, respectively;
(vi) the HCDR1, the HCDR2, and the HCDR3 comprising the amino acid sequences of SEQ ID NOs: 342, 347, and 352, respectively, and the LCDR1, the LCDR2, and the LCDR3 comprising the amino acid sequences of SEQ ID NO: 360, DTS, and SEQ ID NO: 335, respectively;
(vii) the HCDR1, the HCDR2, and the HCDR3 comprising the amino acid sequences of SEQ ID NOs: 343, 347, and 354, respectively, and the LCDR1, the LCDR2, and the LCDR3 comprising the amino acid sequences of SEQ ID NO: 360, DTS, and SEQ ID NO: 335, respectively; or
(viii) the HCDR1, the HCDR2, and the HCDR3 comprising the amino acid sequences of SEQ ID NOs: 342, 347, and 354, respectively, and the LCDR1, the LCDR2, and the LCDR3 comprising the amino acid sequences of SEQ ID NO: 360, DTS, and SEQ ID NO: 335, respectively.

15. The multi-specific polypeptide of claim 1, wherein the second binding domain comprises a VH region and/or a VL region.

16. The multi-specific polypeptide of claim 15, wherein the VH region of the second binding domain comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 365, 367, 369, 371, 373, 375, 377, 379, 381, and 383; and/or the VL region of the second binding domain comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 366, 368, 370, 372, 374, 376, 378, 380, 382, and 384.

17. The multi-specific polypeptide of claim 1, wherein the second binding domain is an scFv, Fab, CrossFab, Fab′, or F(ab′)₂.

18. The multi-specific polypeptide of claim 17, wherein the second binding domain is a Fab, and the Fab comprises the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region, wherein the CH1 region and the CL region are connected by a disulfide bond.

19. The multi-specific polypeptide of claim 18, wherein the second binding domain is a charge steering Fab, and the CH1 region of the charge steering Fab comprises mutations K147E/K213E according to EU numbering, and the CL region of the charge steering Fab comprises mutations E123R/Q124K according to EU numbering.

20. The multi-specific polypeptide of claim 17, wherein the second binding domain is a CrossFab, and the CrossFab comprises the VH region, the VL region, a CH1 region, and a CL region, wherein the C-terminus of the VH region is connected to the N-terminus of the CL region, and the C-terminus of the VL region is connected to the N-terminus of the CH1 region, wherein the CH1 region and the CL region are connected by a disulfide bond.

21. The multi-specific polypeptide of claim 20, wherein the second binding domain is a charge steering CrossFab, and the CH1 region of the charge steering CrossFab comprises mutations K147E/K213E according to EU numbering, and the CL region of the charge steering CrossFab comprises mutations E123R/Q124K according to EU numbering.

22. The multi-specific polypeptide of claim 17, wherein the second binding domain comprises a Fab or a CrossFab comprising (i) a heavy chain comprising an amino acid sequence that is at least 80%, at least 85%, at least 90%, at least 95%, at least 96%, at least 97%, at least 98%, at least 99%, or 100% identical to an amino acid sequence selected from the group consisting of: SEQ ID NOs: 387, 389, 391, 393, 395, 397, 399, 401, 403, and 405; and (ii) a light chain comprising an amino acid sequence that is at least 80%, at least 85%, at least 90%, at least 95%, at least 96%, at least 97%, at least 98%, at least 99%, or 100% identical to an amino acid sequence selected from the group consisting of: SEQ ID NOs: 388, 390, 392, 394, 396, 398, 400, 402, 404, and 406.

23. The multi-specific polypeptide of claim 1, wherein the first binding domain and the second binding domain are connected via a peptide linker (PLB).

24. The multi-specific polypeptide of claim 23, wherein the peptide linker (PLB) comprises the formula of (Gly₄Ser)ₙ, wherein n=1-5.

25. The multi-specific polypeptide of claim 1, further comprising a fragment crystallizable (Fc) region, wherein the Fc region comprises two polypeptides.

26. The multi-specific polypeptide of claim 25, wherein the Fc region has a knobs-into-holes format, wherein the first polypeptide of the Fc region comprises a hole mutation, and wherein second polypeptide of the Fc region comprises a knob mutation.

27. The multi-specific polypeptide of claim 26, wherein the first polypeptide of the Fc region comprises mutations T366S, L368A and Y407V according to EU numbering, and wherein the second polypeptide of the Fc region comprises a mutation T366W according to EU numbering.

28. The multi-specific polypeptide of claim 27, wherein the first polypeptide of the Fc region comprising the hole mutation further comprises a mutation Y349C according to EU numbering, wherein the second polypeptide of the Fc region comprising the knob mutation further comprises a mutation S354C according to EU numbering, and wherein the Fc region comprises an engineered disulfide bond formed between these two cysteines.

29. The multi-specific polypeptide of claim 25, wherein the first polypeptide of the Fc region comprises an amino acid sequence that is at least 80%, at least 85%, at least 90%, at least 95%, at least 97%, at least 98%, at least 99%, or 100% identical to SEQ ID NO: 154 or SEQ ID NO: 156, and wherein the second polypeptide of the Fc region comprises an amino acid sequence that is at least 80%, at least 85%, at least 90%, at least 95%, at least 97%, at least 98%, at least 99%, or 100% identical to SEQ ID NO: 155 or SEQ ID NO: 157.

30. The multi-specific polypeptide of claim 25, wherein the first binding domain or the second binding domain and the Fc region are connected via a peptide linker (PLE).

31. The multi-specific polypeptide of claim 30, wherein the peptide linker (PLE) comprises an amino acid sequence selected from the group consisting of: (Gly₄Ser)ₙ, wherein n=1-5; KTHTCP (SEQ ID NO: 158); GGGGSGGGGSKTHTCP (SEQ ID NO: 159); and DKTHTCP (SEQ ID NO: 160.

32. The multi-specific polypeptide of claim 25, wherein:
(a) the first binding domain is an scFv, wherein the second binding domain is a Fab, wherein the C-terminus of the first binding domain is connected to the N-terminus of the VH region of the second binding domain via a peptide linker (PLB), and wherein the C-terminus of the CH1 region of the second binding domain is connected to the N-terminus of the second polypeptide of the Fc region comprising a knob mutation via a peptide linker (PLE);
(b) the first binding domain is an scFv, wherein the second binding domain is a Fab, wherein the C-terminus of the first binding domain is connected to the N-terminus of the VL region of the second binding domain via a peptide linker (PLB), and wherein the C-terminus of the CH1 region of the second binding domain is connected to the N-terminus of the second polypeptide of the Fc region comprising a knob mutation via a peptide linker (PLE); or
(c) the first binding domain is an scFv, wherein the second binding domain is a Fab, wherein the N-terminus of the first binding domain is connected to the C-terminus of the CH1 region of the second binding domain via a peptide linker (PLB), and wherein the C-terminus of the first binding domain is connected to the N-terminus of the second polypeptide of the Fc region comprising a knob mutation via a peptide linker (PLE).

33. The multi-specific polypeptide of claim 1, wherein the multi-specific polypeptide comprises three non-identical polypeptides.

34. The multi-specific polypeptide of claim 33, wherein the first polypeptide comprises an amino acid sequence that is at least 80%, at least 85%, at least 90%, at least 95%, at least 97%, at least 98%, at least 99%, or 100% identical to an amino acid sequence selected from the group consisting of: SEQ ID NOs: 1159-1165; the second polypeptide comprises an amino acid sequence that is at least 80%, at least 85%, at least 90%, at least 95%, at least 97%, at least 98%, at least 99%, or 100% identical to an amino acid sequence selected from the group consisting of: SEQ ID NOs: 1166-1172; and the third polypeptide comprises an amino acid sequence that is at least 80%, at least 85%, at least 90%, at least 95%, at least 97%, at least 98%, at least 99%, or 100% identical to an amino acid sequence selected from the group consisting of: SEQ ID NOs: 1173-1179.

35. A recombinant nucleic acid molecule encoding the multi-specific polypeptide of claim 1.

36. A vector comprising the recombinant nucleic acid molecule of claim 35.

37. A composition comprising the multi-specific polypeptide of claim 1.

38. A pharmaceutical composition comprising (a) the multi-specific polypeptide of claim 1, and (b) a pharmaceutically acceptable carrier, diluent, or excipient.

39. A method of producing a multi-specific polypeptide, comprising introducing the recombinant nucleic acid molecule of claim 35, into a host cell under conditions that express the multi-specific polypeptide in the host cell.

40. A method of depleting B cells in a subject having a disease or disorder related to the B cells, comprising administering to the subject an effective amount of the multi-specific polypeptide of claim 1.

41. A method of expanding a T cell population, wherein the method comprises contacting the T cell population with the multi-specific polypeptide of claim 1, wherein the T cell population expresses a TCRβV antigen on the cell surface, and wherein the multi-specific polypeptide specifically binds to the TCRβV antigen.

42. A method of treating a disease, disorder, or condition in a subject in need thereof, comprising administering to the subject an effective amount of the multi-specific polypeptide of claim 1.

43. A multi-specific polypeptide comprising:
(a) a first binding domain that specifically binds T cell receptor beta variable region (TCRβV), wherein the first binding domain comprises:
(i) a Kabat HCDR1 that comprises an amino acid sequence selected from the group consisting of: TYYIH (SEQ ID NO: 1); LTYIH (SEQ ID NO: 7); and LYYIH (SEQ ID NO: 1138);
(ii) a Kabat HCDR2 that comprises an amino acid sequence selected from the group consisting of: WFFPGSGNIKYNEKFKG (SEQ ID NO: 2); RVSAGSGNVKYNEKFKG (SEQ ID NO: 8); WFFPRSGNIKYNEKFKG (SEQ ID NO: 1142); WIFPGSGNIKYNEKFKG (SEQ ID NO: 1180); and WIYPGSGNIKYNEKFKG (SEQ ID NO: 1181);
(iii) a Kabat HCDR3 that comprises an amino acid sequence selected from the group consisting of: SYYSYDVLDY (SEQ ID NO: 3); and SYYSFDVLDY (SEQ ID NO: 1139);
(iv) a Kabat LCDR1 that comprises an amino acid sequence selected from the group consisting of: KASQNVGINVV (SEQ ID NO: 4); KASQNVADRVV (SEQ ID NO: 9); KASQNVGIRVV (SEQ ID NO: 1140); KASQNVGNNVA (SEQ ID NO: 1182); and RASQNVGNNVA (SEQ ID NO: 1183);
(v) a Kabat LCDR2 that comprises an amino acid sequence selected from the group consisting of: SSSHRYS (SEQ ID NO: 5) and SSSHRYK (SEQ ID NO: 10); and
(vi) a Kabat LCDR3 that comprises an amino acid sequence selected from the group consisting of: QQFKSYPLT (SEQ ID NO: 6) and QQFKSYPIT (SEQ ID NO: 1141); and
(b) a second binding domain that specifically binds CD19.

44. A multi-specific polypeptide comprising:
(a) a first binding domain that specifically binds TCRβV, wherein the first binding domain comprises:
(i) an IMGT HCDR1 that comprises an amino acid sequence selected from the group consisting of: GFTFSNFG (SEQ ID NO: 28) and GFTFSNYG (SEQ ID NO: 226; an IMGT HCDR2 that comprises an amino acid sequence of ISSGSSTI (SEQ ID NO: 29); an IMGT HCDR3 that comprises an amino acid sequence of ARRGEGAMDY (SEQ ID NO: 30); an IMGT LCDR1 that comprises an amino acid sequence selected from the group consisting of: SSVNY (SEQ ID NO: 31) and QSVNY (SEQ ID NO: 227); an IMGT LCDR2 that comprises an amino acid sequence of YTS; and an IMGT LCDR3 that comprises an amino acid sequence selected from the group consisting of: QQFTSSPFT (SEQ ID NO: 33) and QQYTSSPFT (SEQ ID NO: 224); or
(ii) a Kabat HCDR1 that comprises an amino acid sequence selected from the group consisting of: NFGMH (SEQ ID NO: 11) and NYGMH (SEQ ID NO: 221); a Kabat HCDR2 that comprises an amino acid sequence selected from the group consisting of: YISSGSSTIYYADTLKG (SEQ ID NO: 12) and YISSGSSTIYYADALKG (SEQ ID NO: 222); a Kabat HCDR3 that comprises an amino acid sequence of RGEGAMDY (SEQ ID NO: 13); a Kabat LCDR1 that comprises an amino acid sequence selected from the group consisting of: RASSSVNYIY (SEQ ID NO: 14); RASQSVNYIY (SEQ ID NO: 223); and RASQSVNYLY (SEQ ID NO: 225); a Kabat LCDR2 that comprises an amino acid sequence of YTSNLAP (SEQ ID NO: 15; and a Kabat LCDR3 that comprises an amino acid sequence selected from the group consisting of: QQFTSSPFT (SEQ ID NO: 16) and QQYTSSPFT (SEQ ID NO: 224); and
(b) a second binding domain that specifically binds CD19.

45. A multi-specific polypeptide comprising:
(a) a first binding domain that specifically binds TCRβV, wherein the first binding domain comprises:
(i) an IMGT HCDR1 that comprises an amino acid sequence of: GFNIKSAY (SEQ ID NO: 234) or a Kabat HCDR1 that comprises an amino acid sequence of: SAYMH (SEQ ID NO: 228);
(ii) an IMGT HCDR2 that comprises an amino acid sequence of: IDPATGKT (SEQ ID NO: 235) or a Kabat HCDR2 that comprises an amino acid sequence of: RIDPATGKTKYAPKFQA (SEQ ID NO: 229);
(iii) an IMGT HCDR3 that comprises an amino acid sequence of: TRSLNWDYGLDY (SEQ ID NO: 236) or a Kabat HCDR3 that comprises an amino acid sequence of: SLNWDYGLDY (SEQ ID NO: 230);
(iv) an IMGT LCDR1 that comprises an amino acid sequence of: KSVSILGTHL (SEQ ID NO: 237) or a Kabat LCDR1 that comprises an amino acid sequence of: RASKSVSILGTHLIH (SEQ ID NO: 231);
(v) an IMGT LCDR2 that comprises an amino acid sequence of: AAS or a Kabat LCDR2 that comprises an amino acid sequence of: AASNLES (SEQ ID NO: 232); and
(vi) an IMGT LCDR3 that comprises an amino acid sequence of: QQSIEDPWT (SEQ ID NO: 239) or a Kabat LCDR3 that comprises an amino acid sequence of: QQSIEDPWT (SEQ ID NO: 233); and
(b) a second binding domain that specifically binds CD19.

46. A multi-specific polypeptide comprising:
(a) a first binding domain that specifically binds T cell receptor alpha variable region (TCRαV), wherein the first binding domain comprises:
(i) an IMGT HCDR1 that comprises an amino acid sequence of: GFNIKDTH (SEQ ID NO: 140) or a Kabat HCDR1 that comprises an amino acid sequence of: DTHMH (SEQ ID NO: 134);
(ii) an IMGT HCDR2 that comprises an amino acid sequence of: TDPASGDT (SEQ ID NO: 141) or a Kabat HCDR2 that comprises an amino acid sequence of: RTDPASGDTKYDPKFQG (SEQ ID NO: 135);
(iii) an IMGT HCDR3 that comprises an amino acid sequence of: AHYYRDDVNYAMDY (SEQ ID NO: 142) or a Kabat HCDR3 that comprises an amino acid sequence of: YYRDDVNYAMDY (SEQ ID NO: 136);
(iv) an IMGT LCDR1 that comprises an amino acid sequence of: QNVGSN (SEQ ID NO: 143) or a Kabat LCDR1 that comprises an amino acid sequence of: KARQNVGSNVA (SEQ ID NO: 137);
(v) an IMGT LCDR2 that comprises an amino acid sequence of SSS or a Kabat LCDR2 that comprises an amino acid sequence of: SSSFRYS (SEQ ID NO: 138); and
(vi) an IMGT LCDR3 that comprises an amino acid sequence of: QQYNTYPYT (SEQ ID NO: 145) or a Kabat LCDR3 that comprises an amino acid sequence of: QQYNTYPYT (SEQ ID NO: 139); and
(b) a second binding domain that specifically binds CD19.
